(19) United States
(12) Patent Application Publication
(10) Pub. No.: US 2021/0015933 A1
(43) Pub. Date: Jan. 21, 2021

(54) BIOACTIVE CARBOXYLIC ACID TYPE COMPOUND-POLYMER CONJUGATE, AND METHOD FOR MANUFACTURING THE SAME

(71) Applicant: Seikagaku Corporation, Tokyo (JP)

(72) Inventors: Nobuo Kobayashi, Tokyo (JP); Kenta Adachi, Tokyo (JP); Kenichi Namatsu, Tokyo (JP)

(73) Assignee: Seikagaku Corporation, Tokyo (JP)

(21) Appl. No.: 17/044,230

(22) PCT Filed: Mar. 29, 2019
(86) PCT No.: PCT/JP2019/014328

§ 371 Date: Sep. 30, 2020

(30) Foreign Application Priority Data
Mar. 30, 2018 (JP) 2018-068341

(51) Int. Cl. A61K 47/61

(52) U.S. Cl. CPC A61K 47/61

(57) ABSTRACT

Provided are a novel conjugate of a bioactive carboxylic acid type compound with a polymer having a carboxy group, represented by Formula (I) as defined in the specification, capable of finely controlling a sustained release rate, and capable of sustainably releasing even a drug in which the vicinity of a carbonyl group of the bioactive carboxylic acid type compound is sterically crowded, and a method for manufacturing the same.

### TECHNICAL FIELD

The present invention relates to a novel conjugate of a bioactive carboxylic acid type compound with a polymer, and a method for manufacturing the same. Specifically, the present invention relates to a novel conjugate of a bioactive carboxylic acid type compound with a polymer using, as a linker, an aminoacyloxymethyl group whose release rate can be controlled.

### BACKGROUND ART

JP H07-505177 A describes a low water-soluble or water-insoluble biodegradable non-crosslinked polymer in which a lipophilic group is introduced into a non-polypeptide polymer.

Furthermore, a conjugate of a drug with a polymer has been widely studied in the field of a prodrug or a drug delivery system (DDS), and is one of important means for imparting a function such as release control, absorption improvement, stabilization in a living body, or targeting to a target tissue.

For example, a conjugate using polyglutamic acid that is one of polyamino acids has been reported in JP 2003-511423 A. A conjugate with gossypol using carboxymethyl cellulose (CMC) used as a pharmaceutical additive has been reported in JP 5690944 B2. Alginic acid that is one of dietary fibers in polysaccharides has also been studied, and conjugates thereof with various drugs have been reported in JP H08-24325 A. Furthermore, a conjugate of hyaluronic acid or chondroitin sulfate with a peptide also has been reported in U.S. Pat. No. 5,955,578. Furthermore, a conjugate using heparin has been reported in WO 1993/18793 A. Furthermore, a conjugate with a drug using hyaluronic acid has been also actively studied (WO 2005/085294 A).

Meanwhile, when a structure of a drug to be bonded to and conjugated with a polymer is considered, a drug having an amino group, a carboxy group, or a hydroxy group as a functional group in a molecule thereof is utilized. In particular, a large number of drugs each having a carboxy group exist as therapeutic agents for a wide variety of diseases, and conjugates using the drugs have been also widely studied. As a method for bonding a drug having a carboxy group to a linker, 1) a method for forming an ester bond with a carboxylic acid type drug (WO 2007/004675 A), and 2) a method for forming an amide bond with a carboxy group (WO 2005/085294 A) are known.

### PRIOR ART DOCUMENT

### Patent Document

### Patent Literature 1: JP H07-505177 A

### Patent Literature 2: JP 2003-511423 A

### Patent Literature 3: JP 5690944 B2

### Patent Literature 4: JP H08-24325 A

Patent Literature 5: U.S. Pat. No. 5,955,578

### Patent Literature 6: WO 1993/18793 A

### Patent Literature 7: WO 2005/085294 A

### Patent Literature 8: WO 2007/004675 A

### SUMMARY OF THE INVENTION

### Problems to be Solved by the Invention

A polymer having a carboxy group (hereinafter, also referred to as “carboxylic acid type polymer”) is a very attractive carrier. It is very useful to conjugate a drug having a carboxy group with a carboxylic acid type polymer in order to impart a sustained release property to the drug for development of medical drugs, medical devices, agricultural chemicals, and the like. However, in the conventional method described in WO 2005/085294 A or WO 2007/004675 A, a functional group to be hydrolyzed in a conjugate, such as an ester bond or an amide bond, is bonded to a drug only at a single bonding point. Therefore, there is a limit in finely controlling release of a drug having a carboxy group. Furthermore, when a bulky carboxylic acid type drug is used, it is expected that steric hindrance near the functional group to be hydrolyzed will be extremely high. Since it is known that a hydrolysis rate is largely affected by steric hindrance, it is expected that in such a case, it will be difficult to impart sufficient drug releasing ability by these conventional methods. As described above, in the conventional technology, there is a limitation in controlling the sustained release rate of a drug having a carboxy group. There is a demand for finding a novel method capable of finely controlling the sustained release rate, and a method capable of sustainably releasing even a bulky drug having a carboxy group, which is considered to be resistant to hydrolysis, into the body.

An object of the present invention is to provide a novel conjugate of a bioactive carboxylic acid type compound with a polymer having a carboxy group, capable of finely controlling a sustained release rate and capable of sustainably releasing even a drug in which the vicinity of a carbonyl group of the bioactive carboxylic acid type compound is sterically crowded, and a method for manufacturing the same.

The present inventors made intensive studies on a novel linker capable of forming a conjugate of a carboxylic acid type compound with a polymer having a carboxy group, and as a result, have found an aminoacyloxymethyl group type linker having characteristics not observed in a conventional linker. The present invention is based on finding of a linker capable of forming a bond in such a manner that a sustained release rate can be controlled more widely and more finely, and further capable of sustainably releasing even a carboxylic acid type compound having a bulky structure in the vicinity of a carboxy group, which has been difficult to sustainably release, and provides a novel conjugate of a bioactive carboxylic acid type compound with a polymer, and a method for manufacturing the same.

### Means to Solve the Problems

The present invention relates to inventions specified by the following items.

1. A compound represented by Formula (I) or a pharmaceutically acceptable salt thereof;

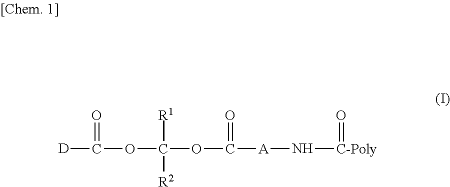

[In Formula (I), D is a residue of a carboxylic acid type compound D-CO₂H excluding a carboxy group wherein the carboxylic acid type compound itself or a pharmaceutically acceptable salt thereof is bioactive; R¹ and R² are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; A is a divalent hydrocarbon group optionally having a hetero atom selected from the group consisting of an oxygen atom, a nitrogen atom, and a sulfur atom; any two or three groups of R¹, R², and A may combine together to form a ring; and Poly is a residue derived from a polymer having a carboxy group].

2. The compound or a pharmaceutically acceptable salt thereof according to 1., wherein the polymer having a carboxy group and one or more amine forms represented by the following Formula (III) or salts thereof with an inorganic acid or an organic acid form an amide-bond;

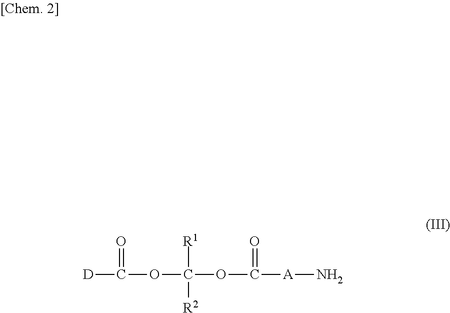

[In Formula (III), R¹, R², A, and D are as defined in 1.].

3. A compound represented by Formula (II) or a pharmaceutically acceptable salt thereof;

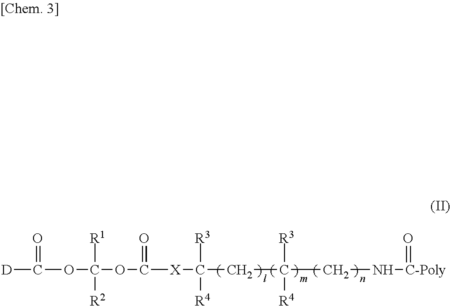

[In Formula (II), D, R¹, R², and Poly are as defined in 1.; X is a single bond, O, or NR⁷; R³, R⁴, R⁵, R⁶, and R⁷ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; any two or three groups of R¹, R², R³, R⁴, R⁵, R⁶, and R⁷ may combine together to form a ring; l and n are each independently 0, 1, or 2, and m is 0 or 1].

4. The compound or a pharmaceutically acceptable salt thereof according to 3., wherein the polymer having a carboxy group and one or more amine forms represented by the following Formula (XI) or salts thereof with an inorganic acid or an organic acid form an amide-bond;

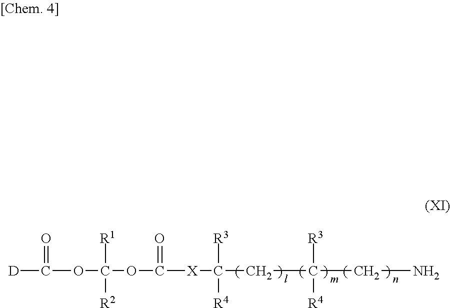

[In Formula (XI), R¹, R², R³, R⁴, R⁵, R⁶, l, m, n, X, and D are as defined in 3.].

5. The compound or a pharmaceutically acceptable salt thereof according to 3. or 4., wherein in Formula (II); R¹, R², R³, R⁴, R⁵, R⁶, and R⁷ are each independently a hydrogen atom, a substituted or unsubstituted linear or branched alkyl group having carbon number of 1 to 6, a substituted or unsubstituted cycloalkyl group having carbon number of 3 to 8, a substituted or unsubstituted linear or branched alkenyl group having carbon number of 2 to 6, a substituted or unsubstituted cycloalkenyl group having carbon number of 3 to 8, a substituted or unsubstituted linear or branched alkynyl group having carbon number of 2 to 6, a substituted or unsubstituted monocyclic or polycyclic aromatic group having carbon number of 6 to 14, or a substituted or unsubstituted 3- to 8-membered heterocyclic group containing at least one of a nitrogen atom, an oxygen atom, or a sulfur atom as a ring-constituting atom.

6. The compound or a pharmaceutically acceptable salt thereof according to any one of 3. to 5., wherein in Formula (II), a substituent of an alkyl, a substituent of a cycloalkyl group, a substituent of an alkenyl group, a substituent of a cycloalkenyl group, a substituent of an alkynyl group, a substituent of an aromatic group, and a substituent of a heterocyclic group in the groups represented by R¹, R², R³, R⁴, R⁵, R⁶, and R⁷ are groups selected from a hydroxy group, an alkyl group, a cycloalkyl group, an alkenyl group, a cycloalkenyl group, an alkynyl group, a halogen atom, an aromatic group, a heterocyclic group, an alkoxy group, a guanidino group, an alkylthio group, an alkoxycarbonyl group, an aryloxy group, an arylthio group, an acyl group, a substituted sulfonyl group, a heterocyclyloxy group, a heterocyclylthio group, an amide group, a ureido group, a carboxy group, a carbamoyl group, an oxo group, a thioxo group, a sulfamoyl group, a sulfo group, a cyano group, a nitro group, an acyloxy group, an azido group, a sulfonamide group, a mercapto group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, an alkoxysulfonyl group, an Rx(Ry)N group, and an Rx(Ry)(Rz)N⁺ group; Rx, Ry, and Rz are each independently selected from the group consisting of a hydrogen atom, an alkyl group, a cycloalkyl group, an alkenyl group, a cycloalkenyl group, an alkynyl group, an aromatic hydrocarbon group, and a heterocyclic group; at least two of Rx, Ry, and Rz may be bonded to each other to form a saturated or unsaturated heterocyclic ring, and the heterocyclic ring can also form a condensed ring or a spiro ring with an aliphatic ring or a heterocyclic ring and may also form a condensed ring with an aromatic ring.

7. The compound or a pharmaceutically acceptable salt thereof according to any one of 1. to 6., wherein the polymer having a carboxy group is a water-soluble polymer or a pharmaceutically acceptable salt thereof.

8. The compound or a pharmaceutically acceptable salt thereof according to any one of 1. to 6., wherein the polymer having a carboxy group is a polysaccharide or a pharmaceutically acceptable salt thereof.

9. The compound or a pharmaceutically acceptable salt thereof according to any one of 1. to 6., wherein the polymer having a carboxy group is glycosaminoglycan or a pharmaceutically acceptable salt thereof.

10. The compound or a pharmaceutically acceptable salt thereof according to any one of 1. to 6., wherein the polymer having a carboxy group is chondroitin, chondroitin sulfate, hyaluronic acid, or a pharmaceutically acceptable salt thereof.

11. A method for manufacturing a compound represented by the following Formula (I), the method including a step of condensing a compound represented by the following Formula (III) with a polymer having a carboxy group represented by the following Formula (IV).

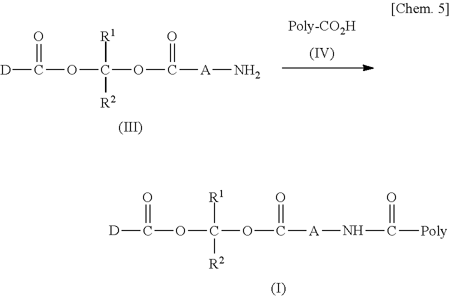

[In Formulae (I), (III), and (IV), D, A, R¹, R², and Poly are as defined in 1., and a compound represented by Formula (III) may form a salt with an inorganic acid or an organic acid.

12. A linker represented by the following Formula (V) for bonding a bioactive carboxylic acid type compound to a polymer having a carboxy group;

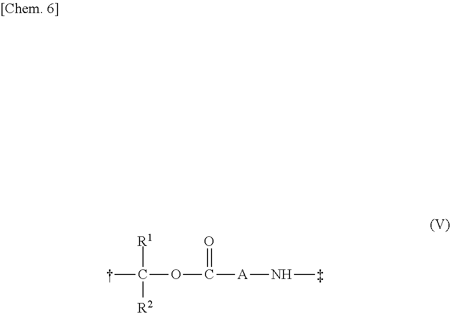

[In Formula (V), R¹, R², and A are as defined in 1., the symbol † is a bonding point with a structural moiety of a carboxy group of the bioactive carboxylic acid type compound excluding a hydrogen atom, and the symbol ‡ is a bonding point with a structural moiety of a carboxy group of the polymer having the carboxy group excluding a hydroxy group].

13. A method for manufacturing a compound represented by Formula (I) or a pharmaceutically acceptable salt thereof, the method including a step of bonding a carboxylic acid type compound in which the carboxylic acid type compound itself or a pharmaceutically acceptable salt thereof is bioactive to a polymer having a carboxy group via the linker according to 12.

A linker having the structure found in the present invention makes it possible to manufacture a novel conjugate of a bioactive carboxylic acid type compound and a polymer having a carboxy group. The conjugate can control more widely and more finely the sustained release rate of a conjugate of a carboxylic acid type compound with a polymer having a carboxy group, which has been conventionally limited, and can further sustainably release even a carboxylic acid type compound having a bulky structure in the vicinity of a carboxy group, which has been conventionally difficult to sustainably release. Contribution of the present invention to medical treatment and the like is enormous.

### BRIEF DESCRIPTION OF DRAWINGS

FIG. 1 is a graph illustrating a relationship between time and a drug release ratio in a buffer solution having a pH of 7.0 in Examples 1, 2, 3, and 4.

FIG. 2 is a graph illustrating a relationship between time and a drug release ratio in a buffer solution having a pH of 7.0 in Reference Example 3 and Examples 1, 5, 6, 8, 23, and 26.

FIG. 3 is a graph illustrating a relationship between time and a drug release ratio in a buffer solution having a pH of 7.0 in Examples 22, 24, 17, 28, 29, and 30.

FIG. 4 is a graph illustrating a relationship between time and a drug release ratio in a buffer solution having a pH of 7.0 in Examples 31, 35, 36, 39, 38, and 32.

FIG. 5 is a graph illustrating a relationship between time and a drug release ratio in a buffer solution having a pH of 7.0 in Examples 34, 37, 42, 40, 41, and 43.

FIG. 6 is a graph illustrating a relationship between time and a drug release ratio in a buffer solution having a pH of 7.0 in Examples 48, 47, 50, 45, 51, 49, and 44.

### EMBODIMENTS TO CARRY OUT THE INVENTION

A conjugate according to an aspect of the present invention is a compound having a structure represented by the following general Formula (I) or a pharmaceutically acceptable salt thereof.

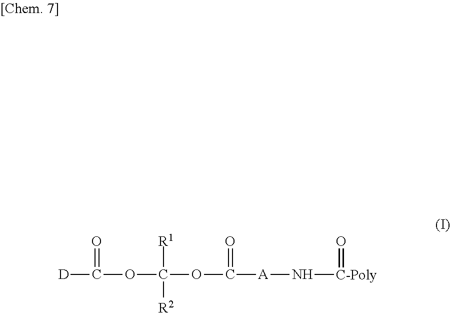

[In Formula, D, R¹, R², A, and Poly are as defined above].

In Formula (I), Poly-CO which represents a structure of a carboxy group excluding a OH moiety in the polymer having a carboxy group, Poly-CO₂H, used for condensation with an amine form, and D-COO which represents a structure excluding a hydrogen atom from a carboxylic acid type compound which itself or pharmaceutically acceptable salt thereof is bioactive, D-CO₂H, form a conjugate by bonding through a linker containing a hydrocarbon group A.

When the polymer having a carboxy group is a salt, the —CO₂H moiety of Poly-CO₂H may of course be a salt group. The conjugate is preferably a conjugate with a drug containing a carboxy group. Hereinafter, in the description of D, a carboxylic acid type compound having bioactivity or a pharmaceutically acceptable salt thereof having bioactivity may be collectively referred to as a “bioactive carboxylic acid type compound”.

The conjugate is bonded to a hydrocarbon chain of the linker by formation of amide bonds by at least some of carboxy groups of a polymer residue. The divalent hydrocarbon group represented by A in the general Formula (I) only needs to be a carbon chain having carbon number of 1 or more, and may have a branched structure or a cyclic structure. When the divalent hydrocarbon group A has a branched structure, an atom other than a hydrogen atom can be bonded to at least one point of a molecular chain coupling a carbonyl group and an NH group to which A is bonded in the general formula (I) with the minimum number of atoms. Examples of such a branched structure include 2-methyl-1,3-propylenyl and 1-phenyl-1,3-propylenyl. When a part of the divalent hydrocarbon group A has a cyclic structure, the ring can be bonded to the remaining part of A at any two points of the ring which may be the same atom. Examples of such a cyclic structure include 1,2-phenylene, 1,4-cyclohexylene, and 1,1-cyclopropenylene. In a case where the carbon number is 3 or more, a carbon atom may be substituted by a hetero atom selected from the group consisting of a nitrogen atom, an oxygen atom, and a sulfur atom. Furthermore, the carbon atoms can combine together with another partial structure, particularly a structure represented by —C(R¹)(R²)— in the above Formula (I) to form a ring. Furthermore, the divalent hydrocarbon group A may have a substituent at any position, and examples of the substituent may include those exemplified as the groups of R³, R⁴, R⁵, R⁶, and R⁷ described above. A is preferably a divalent hydrocarbon group represented by —X—C(R³)(R⁴)—(CH₂)ₗ-(C(R⁵)(R⁶))ₘ—(CH₂)ₙ— as included in the above general Formula (II) (herein, X, R³, R⁴, R⁵, R⁶, R⁷, l, m, and n are as defined above). A is preferably a linear or branched alkylene group having carbon number of 1 to 10 from a viewpoint of ease of design and availability of a raw material, and the carbon number of A is more preferably 1 to 6.

A terminal of the hydrocarbon group A at the opposite side of a terminal to form an amide bond to Poly is bonded to a substituted or unsubstituted methylene group represented by —C(R¹)(R²)— in the above Formula (I) via an ester bond, a carbonate bond, or a urethane bond. These three bonding modes correspond to cases where X is a single bond, 0, and NR⁷ in the above Formula (II), respectively. The methylene group forms a bond in the order of an oxygen atom of an ester bond, a carbonate bond, or a urethane bond—a carbon atom of the methylene group—an oxygen atom derived from the bioactive carboxylic acid type compound D-CO₂H. The methylene group may be unsubstituted or substituted. When the methylene group is substituted, two substituents may together form a ring, and may be bonded to at least one point of a part of the divalent hydrocarbon group A (that is, R³, R⁴, R⁵, R⁶, and R⁷ in general formula (II)) to form a ring. The ring may be a condensed ring or a spiro ring. The bioactive carboxylic acid type compound D-CO₂H is present as an ester in the structure of the conjugate via a linker.

As illustrated in Formulae (I) and (II), —(C═O)— adjacent to Poly is derived from a carboxy group of the polymer.

D-CO—O of the conjugate can release the bioactive carboxylic acid type compound D-CO₂H in the presence of water or the like. This mechanism will be described using a compound represented by the above Formula (I) as follows. The carboxylic acid type compound-polymer conjugate represented by the above Formula (I) has two hydrolyzable functional groups with a methylene group interposed therebetween, and hydrolysis of any one of these functional groups proceeds in the presence of water. When hydrolysis of an ester bond bonded to D proceeds, the conjugate is decomposed into a bioactive carboxylic acid type compound D-CO₂H, represented by Formula (VI), and a hydroxymethyl form represented by Formula (VII) (route A). Furthermore, the hydroxymethyl form represented by Formula (VII) is immediately decomposed into an aldehyde form or a ketone form represented by Formula (X) and a carboxylic acid form represented by Formula (IX). Meanwhile, when hydrolysis of an ester bond, a carbonate bond, or a urethane bond bonded to the divalent hydrocarbon group A which may have a hetero atom selected from the group consisting of an oxygen atom, a nitrogen atom and a sulfur atom, and/or have a cyclic structure proceeds, the conjugate is decomposed into a hydroxymethyl form represented by Formula (VIII) and a carboxylic acid form represented by Formula (IX) (route B). Furthermore, the hydroxymethyl form represented by Formula (VIII) is immediately decomposed into a bioactive carboxylic acid type compound D-CO₂H, represented by Formula (VI), and an aldehyde form or a ketone form represented by Formula (X). That is, even if hydrolysis of either functional group proceeds, the same products are obtained, and the function of the bioactive carboxylic acid type compound generated here is exhibited. Therefore, the bioactive carboxylic acid type compound-polymer conjugate represented by the above Formula (I) controls the hydrolysis rates of two hydrolyzable functional groups, thereby can control release of the bioactive carboxylic acid type compound, and can control sustainability of the function of the bioactive carboxylic acid type compound. The D-CO— moiety in Formula (I) is a functional group introduced for the purpose of releasing the bioactive carboxylic acid type compound D-CO₂H by hydrolysis. The conjugate according to the present invention is a kind of so-called prodrug.

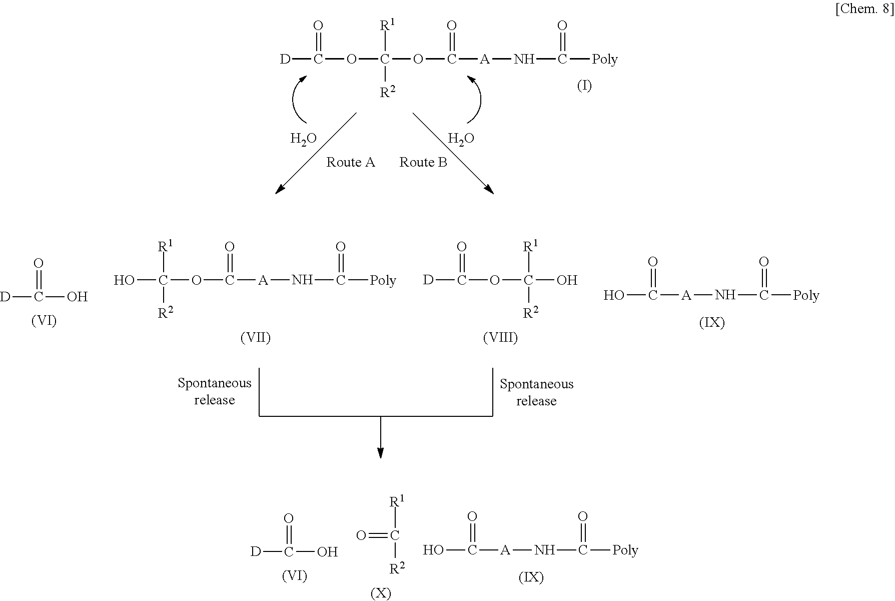

In general, ester hydrolysis is known to be largely affected by steric hindrance. Therefore, when steric hindrance of the residue D of the bioactive carboxylic acid type compound near a carboxy group is large, in a conjugate using a conventional linker having only one ester bond, it is assumed that it may be difficult to sufficiently release the bioactive carboxylic acid type compound D-CO₂H. Meanwhile, the bioactive carboxylic acid type compound-polymer conjugate represented by the above Formula (I) can control release of the bioactive carboxylic acid type compound D-CO₂H without being affected by steric hindrance of D because even if hydrolysis of route A is suppressed due to steric hindrance of D, there is route B based on another hydrolyzable functional group such as an ester group, a carbonate group, or a urethane group. It is considered that release of an aldehyde or a ketone represented by Formula (X) in the above route B occurs spontaneously in a molecule of Formula (VIII) and is not affected by steric hindrance of D. Furthermore, the bioactive carboxylic acid type compound-polymer conjugate represented by the above Formula (I) has two hydrolyzable ester groups (including a carbonate group or a urethane group in a case of route B). From this, the conjugate can control release of the bioactive carboxylic acid type compound D-CO₂H in more detail than a conjugate using a conventional linker having only one ester bond.

A release ratio of the carboxylic acid type compound from the bioactive carboxylic acid type compound-polymer conjugate represented by the above Formula (I) or (II) is not particularly limited and can be appropriately selected depending on a purpose and the like. The release ratio of the carboxylic acid type compound can be, for example, 0.2% or more, 0.4% or more, 0.5% or more, 1% or more, 5% or more, 8% or more, 10% or more, 12% or more, or 15% or more, for example, in a phosphate buffer solution having a pH of 7.0 at 36° C. at the time point of 48 hours (two days) after start of storage. The release ratio can be, for example, 80% or less, 75% or less, 50% or less, or 20% or less. In one embodiment, the release ratio is between 0.2% and 80%. In one embodiment, the release ratio is between 0.4% and 80%. In one embodiment, the release ratio is between 0.5% and 80%. In one embodiment, the release ratio is between 1% and 80%. In one embodiment, the release ratio is between 5% and 80%. In one embodiment, the release ratio is between 5% and 80%. In one embodiment, the release ratio is between 8% and 80%. In one embodiment, the release ratio is between 10% and 80%. In one embodiment, the release ratio is between 12% and 80%. In one embodiment, the release ratio is between 15% and 80%. In one embodiment, the release ratio is between 0.2% and 10%. In one embodiment, the release ratio is between 0.2% and 8%. In one embodiment, the release ratio is between 0.2% and 5%. In one embodiment, the release ratio is more than 0.4% and 10% or less. In one embodiment, the release ratio is more than 0.4% and 8% or less. In one embodiment, the release ratio is more than 0.4% and 5% or less. In one embodiment, the release ratio is between 0.5% and 10%. In one embodiment, the release ratio is between 0.5% and 8%. In one embodiment, the release ratio is between 0.5% and 5%. In one embodiment, the release ratio is between 1% and 10%. In one embodiment, the release ratio is between 1% and 8%. In one embodiment, the release ratio is between 1% and 5%.

Here, the release ratio (%) of the carboxylic acid type compound at each time point can be determined by an abundance ratio (molar ratio) between “carboxylic acid type compound present as a conjugate” and “released carboxylic acid type compound” using a high performance liquid chromatography (HPLC) with a UV wavelength specific to the carboxylic acid type compound, and is represented by the following formula.

Release ratio (%)=[Amount of carboxylic acid type compound released at each time point/(Amount of carboxylic acid type compound present as conjugate at each time point+Amount of carboxylic acid type compound released at each time point)]×100

One embodiment of the bioactive carboxylic acid type compound-polymer conjugate of the present invention is a compound represented by the above Formula (I) or (II), and an amine form that is an important intermediate of the compound represented by (I) or (II) is a compound represented by the above Formula (III) or (XI). Here, in the above Formula (III) or (XI), D, X, R¹, R², R³, R⁴, R⁵, R⁶, R⁷, A, l, m, and n are as defined above. A compound represented by the above Formula (III) or (XI) may further form a salt with an inorganic acid or an organic acid.

Specific examples of the alkyl group, the cycloalkyl group, the alkenyl group, the cycloalkenyl group, the alkynyl group, the aromatic group, and the heterocyclic group included in groups represented by the substituents R¹, R², R³, R⁴, R⁵, R⁶, and R⁷ in Formulae (I), (II), (III), and (XI) include the following groups.

As the alkyl group, either a linear alkyl group or a branched alkyl group may be used, and the carbon number is preferably 1, 2, 3, 4, 5, or 6. Examples of the alkyl group may include a methyl group, an ethyl group, a n-propyl group, a 2-propyl, a n-butyl group, a 1-methylpropyl group, a 1,1-dimethylethyl group, a 2-methylpropyl group, a n-pentyl group, a 1-methylbutyl group, a 2-methylbutyl group, a 3-methylbutyl group, a 1-ethylpropyl group, a 1,1-dimethylpropyl group, a 1,2-dimethylpropyl group, a 2,2-dimethylpropyl group, a n-hexyl group, a 1-methylpentyl group, a 2-methylpentyl group, a 3-methylpentyl group, a 4-methylpentyl group, a 1-ethylbutyl group, a 2-ethylbutyl group, a 1,1-dimethylbutyl group, a 1,2-dimethylbutyl group, a 1,3-dimethylbutyl group, a 2,2-dimethylbutyl group, a 2,3-dimethylbutyl group, a 3,3-dimethylbutyl group, a 1,1,2-trimethylpropyl group, a 1-ethyl-1-methylpropyl group, and a 1-ethyl-2-methylpropyl group.

The cycloalkyl group may be any group as long as a carbon atom at a bonding point is included as a ring-constituting atom, and may be condensed with a cycloalkane, a cycloalkene, an aromatic ring, or a heterocyclic ring, or may form a spiro ring. The carbon number of the cycloalkyl group is preferably 3, 4, 5, 6, 7, or 8. Examples of the cycloalkyl group may include a cyclopropyl group, a cyclobutyl group, a cyclopentyl group, a cyclohexyl group, a cycloheptyl group, and a cyclooctyl group.

As the alkenyl group, either a linear alkenyl group or a branched alkenyl group may be used, and the carbon number is preferably 2, 3, 4, 5, or 6. Examples of the alkenyl group may include a vinyl group, a 1-propenyl group, a 2-propenyl group, a 1-methylvinyl group, a 1-butenyl group, a 2-butenyl group, a 3-butenyl group, a 1-ethylvinyl group, a 1-methyl-1-propenyl group, a 1-methyl-2-propenyl group, a 2-methyl-1-propenyl group, a 2-methyl-2-propenyl group, a 1-pentenyl group, a 2-pentenyl group, a 3-pentenyl group, a 4-pentenyl group, a 1-propylvinyl group, a 1-methyl-1-butenyl group, a 1-methyl-2-butenyl group, a 1-methyl-3-butenyl group, a 2-methyl-1-butenyl group, a 2-methyl-2-butenyl group, a 2-methyl-3-butenyl group, a 3-methyl-1-butenyl group, a 3-methyl-2-butenyl group, a 3-methyl-3-butenyl group, a 1-ethyl-1-propenyl group, a 1-ethyl-2-propenyl group, a 1-(2-methylethyl) vinyl group, a 1,2-dimethyl-1-propenyl group, a 1,2-dimethyl-2-propenyl group, a 1,1-dimethyl-2-propenyl group, a 1-hexenyl group, a 2-hexenyl group, a 3-hexenyl group, a 4-hexenyl group, a 5-hexenyl group, a 1-butyl vinyl group, a 1-methyl-1-pentenyl group, a 1-methyl-2-pentenyl group, a 1-methyl-3-pentenyl group, a 1-methyl-4-pentenyl group, a 2-methyl-1-pentenyl group, a 2-methyl-2-pentenyl group, a 2-methyl-3-pentenyl group, a 2-methyl-4-pentenyl group, a 3-methyl-1-pentenyl group, a 3-methyl-2-pentenyl group, a 3-methyl-3-pentenyl group, a 3-methyl-4-pentenyl group, a 4-methyl-1-pentenyl group, a 4-methyl-2-pentenyl group, a 4-methyl-3-pentenyl group, a 4-methyl-4-pentenyl group, a 1-propyl-1-propenyl group, a 1-propyl-2-propenyl group, a 1-ethyl-1-butenyl group, a 1-ethyl-2-butenyl group, a 1-ethyl-3-butenyl group, a 2-ethyl-1-butenyl group, a 2-ethyl-2-butenyl group, a 2-ethyl-3-butenyl group, a 1-(2-methylpropyl) vinyl group, a 1,2-dimethyl-1-butenyl group, a 1,2-dimethyl-2-butenyl group, a 1,2-dimethyl-3-butenyl group, a 1-(3-methylpropyl) vinyl group, a 1,3-dimethyl-1-butenyl group, a 1,3-dimethyl-2-butenyl group, a 1,3-dimethyl-3-butenyl group, a 2,3-dimethyl-1-butenyl group, a 2,3-dimethyl-2-butenyl group, a 2,3-dimethyl-3-butenyl group, a 3,3-dimethyl-1-butenyl group, a 2,2-dimethyl-3-butenyl group, a 1,1-dimethyl-2-butenyl group, a 1,1-dimethyl-3-butenyl group, a 1,1,2-trimethyl-2-propenyl group, a 1-ethyl-1-methyl-2-propenyl group, a 1-ethyl-2-methyl-1-propenyl group, a 1-ethyl-2-methyl-2-propenyl group, a 1-(1-methylethyl)-1-propenyl group, and a 1-(1-methylethyl)-2-propenyl group.

The cycloalkenyl group may be any group as long as a carbon atom and a C═C double bond at a bonding point are included as ring-constituting atoms, and may be condensed with a cycloalkane, a cycloalkene, an aromatic ring, or a heterocyclic ring, or may form a spiro ring. The carbon number of the cycloalkenyl group is preferably 3, 4, 5, 6, 7, or 8. Examples of the cycloalkenyl group may include a 1-cyclopropen-1-yl group, a 2-cyclopropen-1-yl group, a 1-cyclobuten-1-yl group, a 2-cyclobuten-1-yl group, a 1-cyclopenten-1-yl group, a 2-cyclopenten-1-yl group, a 3-cyclopenten-1-yl group, a 1-cyclohexen-1-yl group, a 2-cyclohexen-1-yl group, a 3-cyclohexen-1-yl group, a 1-cyclohepten-1-yl group, a 2-cyclohepten-1-yl group, a 3-cyclohepten-1-yl group, a 4-cyclohepten-1-yl group, a 1-cycloocten-1-yl group, a 2-cycloocten-1-yl group, a 3-cycloocten-1-yl group, a 4-cycloocten-1-yl group, a 1,3-cyclopentadien-1-yl group, a 2,4-cyclopentadien-1-yl group, a 1,3-cyclohexadien-1-yl group, a 1,4-cyclohexadien-1-yl group, a 1,5-cyclohexadien-1-yl group, a 2,4-cyclohexadien-1-yl group, a 2,5-cyclohexadien-1-yl group, a 1,3-cycloheptadien-1-yl group, a 1,4-cycloheptadien-1-yl group, a 1,5-cycloheptadien-1-yl group, a 1,6-cycloheptadien-1-yl group, a 2,4-cycloheptadien-1-yl group, a 2,5-cycloheptadien-1-yl group, a 2,6-cycloheptadien-1-yl group, a 1,4-cycloheptadien-1-yl group, a 1,5-cycloheptadien-1-yl group, a 3,5-cycloheptadien-1-yl group, a 1,3-cyclooctadien-1-yl group, a 1,4-cyclooctadien-1-yl group, a 1,5-cyclooctadien-1-yl group, a 1,6-cyclooctadien-1-yl group, a 1,7-cyclooctadien-1-yl group, a 2,4-cyclooctadien-1-yl group, a 2,5-cyclooctadien-1-yl group, a 2,6-cyclooctadien-1-yl group, a 2,7-cyclooctadien-1-yl group, a 3,5-cyclooctadien-1-yl group, a 3,6-cyclooctadien-1-yl group, a 1,3,5-cycloheptatrien-1-yl group, a 1,3,6-cycloheptatrien-1-yl group, a 1,4,6-cycloheptatrien-1-yl group, a 2,4,6-cycloheptatrien-1-yl group, a 1,3,5-cyclooctatrien-1-yl group, a 1,3,6-cyclooctatrien-1-yl group, a 1,3,7-cyclooctatrien-1-yl group, a 1,4,6-cyclooctatrien-1-yl group, a 1,4,7-cyclooctatrien-1-yl group, a 1,5,7-cyclooctatrien-1-yl group, a 2,4,6-cyclooctatrien-1-yl group, a 2,4,7-cyclooctatrien-1-yl group, and a cyclooctatetraen-1-yl group.

As the alkynyl group, any one of a linear alkynyl group, a branched alkynyl group, and a cyclic alkynyl group may be used, and the carbon number is preferably 2, 3, 4, 5, or 6. Examples of the alkynyl group may include an ethynyl group, a 1-propynyl group, a 2-propynyl group, a 1-butynyl group, a 2-butynyl group, a 3-butynyl group, a 1-methyl-2-propynyl group, a 1-pentynyl group, a 2-pentynyl group, a 3-pentynyl group, a 4-pentynyl group, a 1-methyl-2-butynyl group, a 1-methyl-3-butynyl group, a 2-methyl-3-butynyl group, a 3-methyl-1-butynyl group, a 1-ethyl-2-propynyl group, a 1,1-dimethyl-2-propynyl group, a 1-hexynyl group, a 2-hexynyl group, a 3-hexynyl group, a 4-hexynyl group, a 1-methyl-2-pentynyl group, a 1-methyl-3-pentynyl group, a 1-methyl-4-pentynyl group, a 2-methyl-3-pentynyl group, a 2-methyl-4-pentynyl group, a 3-methyl-1-pentynyl group, a 3-methyl-4-pentynyl group, a 4-methyl-1-pentynyl group, a 4-methyl-2-pentynyl group, a 1-butyl-2-propynyl group, a 1-ethyl-2-butynyl group, a 1-ethyl-3-butynyl group, a 2-ethyl-3-butynyl group, a 1,1-dimethyl-2-butynyl group, a 1,1-dimethyl-3-butynyl group, a 1,2-dimethyl-3-butynyl group, a 2,2-dimethyl-3-butynyl group, a 3,3-dimethyl-1-butynyl group, a 1-ethyl-1-methyl-2-propynyl group, a 1-(2-methylethyl)-2-propynyl group, a 2-cyclohexin-1-yl group, and a 3-cyclohexin-1-yl group.

As the aromatic group, either a monocyclic aromatic group or a polycyclic aromatic group may be used. The aromatic group may be condensed with a cycloalkane, a cycloalkene, an aromatic ring, or a heterocyclic ring, and the carbon number is preferably 6, 7, 8, 9, 10, 11, 12, 13, or 14. Examples of the aromatic group may include a phenyl group, a naphthyl group, and an anthracenyl group.

The heterocyclic group includes at least one heteroatom such as a nitrogen atom, an oxygen atom, or a sulfur atom as a ring-constituting atom, and these may be condensed with a cycloalkane, a cycloalkene, an aromatic ring, or a heterocyclic ring, or may form a spiro ring. The ring size is preferably 3-, 4-, 5-, 6-, 7-, or 8-membered ring. Examples of the heterocyclic group may include an aziridinyl group, an azetidinyl group, a diazetidinyl group, a pyrrolidinyl group, a piperidino group, a homopiperidino group, a pyrazolidinyl group, an imidazolidinyl group, a triazolidinyl group, a tetrazolidinyl group, an oxazolidinyl group, an isoxazolidinyl group, a thiazolidinyl group, an isothiazolidinyl group, an oxadiazolidinyl group, a thiadiazolidinyl group, a piperazinyl group, a homopiperazinyl group, a triazepanyl group, a morpholino group, a thiomorpholino group, a quinuclidinyl group, a tropanyl group, a pyrrolinyl group, a pyrazolinyl group, an imidazolinyl group, an oxazolinyl group, a thiazolinyl group, an isoxazolinyl group, an isothiazolinyl group, a pyrrolyl group, an imidazolyl group, a pyrazolyl group, an oxazolyl group, a dihydrooxazolyl group, a tetrahydrooxazolyl group, an isoxazolyl group, a dihydroisoxazolyl group, a tetrahydroisoxazolyl group, a thiazolyl group, a dihydrothiazolyl group, a tetrahydrothiazolyl group, an isothiazolyl group, a dihydroisothiazolyl group, a tetrahydroisothiazolyl group, a triazolinyl group, a triazolyl group, an oxodiazolyl group, a dihydrooxodiazolyl group, a tetrahydrooxodiazolyl group, a thiadiazolyl group, a dihydrothiadiazolyl group, a tetrahydrothiadiazolyl group, a tetrazolinyl group, a tetrazolyl group, a furazanyl group, a dihydrofurazanyl group, a tetrahydrofurazanyl group, a piperidinyl group, a triazinanyl group, a pyridyl group, a dihydropyridyl group, a tetrahydropyridyl group, a pyrazinyl group, a dihydropyrazinyl group, a tetrahydropyrazinyl group, a pyrimidinyl group, a dihydropyrimidinyl group, a tetrahydropyrimidinyl group, a perhydropyrimidinyl group, a pyridazinyl group, a dihydropyridazinyl group, a tetrahydropyridazinyl group, a perhydropyridazinyl group, a triazinyl group, a dihydrotriazinyl group, a tetrahydrotriazinyl group, an oxazinyl group, a dihydrooxazinyl group, a tetrahydrooxazinyl group, an oxadiazinyl group, a dihydrooxadiazinyl group, a tetrahydrooxadiazinyl group, a thiazinyl group, a dihydrothiazinyl group, a tetrahydrothiazinyl group, a thiadiazinyl group, a dihydrothiadiazinyl group, a tetrahydrothiadiazinyl group, an azepinyl group, a dihydroazepinyl group, a tetrahydroazepinyl group, a perhydroazepinyl group, a diazepinyl group, a dihydrodiazepinyl group, a tetrahydrodiazepinyl group, a perhydrodiazepinyl group, an oxazepinyl group, a dihydrooxazepinyl group, a tetrahydrooxazepinyl group, a perhydrooxazepinyl group, an oxadiazepinyl group, a dihydrooxadiazepinyl group, a tetrahydrooxadiazepinyl group, a perhydrooxadiazepinyl group, a thiazepinyl group, dihydrothiazepinyl group, a tetrahydrothiazepinyl group, a perhydrothiazepinyl group, thiadiazepinyl group, a dihydrothiadiazepinyl group, a tetrahydrothiadiazepinyl group, a perhydrothiadiazepinyl group, a triazepinyl group, a dihydrotriazepinyl group, a tetrahydrotriazepinyl group, a perhydrotriazepinyl group, an azocinyl group, a dihydroazocinyl group, a tetrahydroazocinyl group, an oxohydroazocinyl group, a perhydroazocinyl group, a morphanyl group, a benzazocinyl group, an azepinedolyl group, an indolinyl group, an indoleninyl group, an isoindolinyl group, an isoindoleninyl group, an indolyl group, a perhydroindolyl group, an isoindolyl group, a perhydroisoindolyl group, an indolizinyl group, an indolizidinyl group, an imidazopyridino group, an indazolyl group, a dihydroindazolyl group, a perhydroindazolyl group, a benzimidazolyl group, a dihydrobenzimidazolyl group, a perhydrobenzimidazolyl group, a benzoxazolyl group, a dihydrobenzoxazolyl group, a perhydrobenzoxazolyl group, a benzothiazolyl group, a dihydrobenzothiazolyl group, a perhydrobenzothiazolyl group, a benzooxadiazolyl group, a benzothiadiazolyl group, a benzotriazolyl group, a purinyl group, a quinolyl group, a dihydroquinolyl group, a tetrahydroquinolyl group, a perhydroquinolyl group, a quinolizinyl group, a dihydroquinolidinyl group, a tetrahydroquinolidinyl group, an isoquinolinyl group, a dihydroisoquinolinyl group, a tetrahydroisoquinolinyl group, a perhydroisoquinolinyl group, a cinnolinyl group, a dihydrocinnolinyl group, a tetrahydrocinnolinyl group, a perhydrocinnolinyl group, a quinazolinyl group, a dihydroquinazolinyl group, a tetrahydroquinazolinyl group, a perhydroquinazolinyl group, a phthalazinyl group, a dihydrophthalazinyl group, a tetrahydrophthalazinyl group, a perhydrophthalazinyl group, a quinoxalinyl group, a dihydroquinoxalinyl group, a tetrahydroquinoxalinyl group, a perhydroquinoxalinyl group, a naphthyridinyl group, a dihydronaphthyridinyl group, a tetrahydronaphthyridinyl group, a perhydronaphthyridinyl group, a pteridinyl group, a quinoliridinyl group, a dihydrobenzoxazinyl group, a dihydrobenzothiazinyl group, a benzazepinyl group, a dihydrobenzoazepinyl group, a tetrahydrobenzoazepinyl group, a benzodiazepinyl group, a dihydrobenzodiazepinyl group, a tetrahydrobenzodiazepinyl group, a benzoxazepinyl group, a dihydrobenzoxazepinyl group, a tetrahydrobenzoxazepinyl group, a benzothiazepinyl group, a dihydrobenzothiazepinyl group, a tetrahydrobenzothiazepinyl group, a benzooxadiazepinyl group, a benzothiazeazepinyl group, a benzazepinyl group, a pyridoazepinyl group, a carbazolyl group, a dihydrocarbazolyl group, a tetrahydrocarbazolyl group, a perhydrocarbazolyl group, a β-carbolinyl group, a dihydro β-carbolinyl group, a tetrahydro β-carbolinyl group, a perhydro β-carbolinyl group, an acridinyl group, a dihydroacridinyl group, a tetrahydroacridinyl group, a perhydroacridinyl group, a phenazinyl group, a dihydrophenazinyl group, a tetrahydrophenazinyl group, a perhydrophenazinyl group, a phenothiazinyl group, a dihydrohydrophenothiazinyl group, a tetrahydrophenothiazinyl group, a perhydrophenothiazinyl group, a phenoxazinyl group, a dihydrophenoxazinyl group, a tetrahydrophenoxazinyl group, a perhydrophenoxazinyl group, a phenalsazinyl group, a phenanthridinyl group, a dihydrophenanthridinyl group, a tetrahydrophenanthridinyl group, a perhydrophenanthridinyl group, a phenanthrolinyl group, a dihydrophenanthrolinyl group, a tetrahydrophenanthrolinyl group, a perhydrophenanthrolinyl group, a perimidinyl group, a dihydroperimidinyl group, a tetrahydroperimidinyl group, a perhydroperimidinyl group, a pterinyl group, a pyrrolidinyl group, a morphinanyl group, a hasvananyl group, a furyl group, a dihydrofuryl group, a tetrahydrofuryl group, a pyranyl group, a dihydropyranyl group, a tetrahydropyranyl group, an oxepinyl group, a dihydrooxepinyl group, a tetrahydrooxepinyl group, a perhydrooxepinyl group, a thienyl group, a dihydrothienyl group, a tetrahydrothienyl group, a thiopyranyl group, a dihydrothiopyranyl group, a tetrahydrothiopyranyl group, a thiepinyl group, a dihydrothiepinyl group, a tetrahydrothiepinyl group, a perhydrothiepinyl group, a benzofuryl group, a dihydrobenzofuryl group, a tetrahydrobenzofuryl group, a perhydrobenzofuryl group, an isobenzofuryl group, a dihydroisobenzofuryl group, a tetrahydroisobenzofuryl group, a perhydroisobenzofuryl group, a benzothienyl group, a dihydrobenzothienyl group, a tetrahydrobenzothienyl group, a perhydrobenzothienyl group, an isobenzothienyl group, a dihydroisobenzothienyl group, a tetrahydroisobenzothienyl group, a perhydroisobenzothienyl group, a benzopyranyl group, a dihydrobenzopyranyl group, a perhydrobenzopyranyl group, a benzothiopyranyl group, a dihydrobenzothiopyranyl group, a perhydrobenzothiopyranyl group, a benzooxepinyl group, a dihydrobenzooxepinyl group, a tetrahydrobenzooxepinyl group, a perhydrobenzooxepinyl group, a benzothiepynyl group, a dihydrobenzothiepynyl group, a tetrahydrobenzothiepynyl group, a perhydrobenzothiepynyl group, a benzofuryl group, a dihydrodibenzofuryl group, a tetrahydrodibenzofuryl group, a perhydrodibenzofuryl group, a xanthenyl group, a dihydroxanthenyl group, a tetrahydroxanthenyl group, a perhydroxanthenyl group, a benzothienyl group, a dihydrodibenzothienyl group, a tetrahydrodibenzothienyl group, a perhydrodibenzothienyl group, a thioxanthenyl group, a dihydrothioxanthenyl group, a tetrahydrothioxanthenyl group, a perhydrothioxanthenyl group, a phenoxathiinyl group, a dihydrophenoxathiinyl group, a tetrahydrophenoxathiinyl group, a perhydrophenoxathiinyl group, a dibenzodioxinyl group, a dihydrodibenzodioxinyl group, a tetrahydrodibenzodioxinyl group, a perhydrodibenzodioxinyl group, a thianthrenyl group, a dihydrothianthrenyl group, a tetrahydrothianthrenyl group, a perhydrothianthrenyl group, an oxiranyl group, an oxetanyl group, a thiylanyl group, a thietanyl group, an oxathiynyl group, a dihydrooxathiynyl group, a tetrahydrooxathiynyl group, a benzooxathiynyl group, a dihydrobenzooxathiynyl group, a tetrahydrobenzooxathiynyl group, a perhydrobenzooxathiynyl group, a benzodioxepanyl group, a dioxolanyl group, a dioxanyl group, a dithiolanyl group, a dithianyl group, a dioxoindanyl group, a benzodioxanyl group, a chromanyl group, a benzodithiolanyl group, and a benzodithianyl group. In a case of an unsaturated heterocyclic group, a heterocyclic group in which at least a part thereof is hydrogenated is also included.

Furthermore, any two or three of the substituents R¹, R², R³, R⁴, R⁵, R⁶, and R⁷ may combine together to form a ring, and examples of the ring include cyclopropane, cyclopropene, cyclobutane, cyclobutene, cyclopentane, cyclopentene, cyclopentadiene, cyclohexane, cyclohexene, cyclohexadiene, cycloheptane, cycloheptene, cycloheptadiene, cycloheptatriene, cyclooctane, cyclooctene, cyclooctadiene, cyclooctatriene, aziridine, azetidine, diazetidine, pyrrolidine, piperidine, homopiperidine, pyrazolidine, imidazolidine, triazolidine, tetrazolidine, oxazolidine, isoxazolidine, thiazolidine, isothiazolidine, oxazodiazolidine, thiadiazolidine, piperazine, homopiperazine, triazepan, morpholine, thiomorpholine, quinuclidine, tropane, pyrroline, pyrazoline, imidazoline, oxazoline, thiazoline, isoxazoline, isothiazoline, dihydrooxazole, tetrahydrooxazole, dihydroisoxazole, tetrahydroisoxazole, dihydrothiazole, tetrahydrothiazole, dihydroisothiazole, tetrahydroisothiazole, triazoline, dihydrooxadiazole, tetrahydrooxadiazole, dihydrothiadiazole, tetrahydrothiadiazole, dihydrofurazan, tetrahydrofurazan, piperidin, triazinan, dihydropyridine, tetrahydropyridine, dihydropyrazine, tetrahydropyrazine, dihydropyrimidine, tetrahydropyrimidine, perhydropyrimidine, dihydropyridazine, tetrahydropyridazine, perhydropyridazine, oxazine, dihydrooxazine, tetrahydrooxazine, oxadiazine, dihydrooxadiazine, tetrahydrooxadiazine, thiazine, dihydrothiazine, tetrahydrothiazine, thiadiazine, dihydrothiadiazine, tetrahydrothiadiazine, dihydroazepine, tetrahydroazepine, perhydroazepine, dihydrodiazepine, tetrahydrodiazepine, perhydrodiazepine, oxazepine, dihydrooxazepine, tetrahydrooxazepine, perhydrooxazepine, oxadiazepine, dihydrooxadiazepine, tetrahydrooxadiazepine, perhydrooxadiazepine, thiazepine, dihydrothiazepine, tetrahydrothiazepine, perhydrothiazepine, thiadiazepine, dihydrothiadiazepine, tetrahydrothiadiazepine, perhydrothiadiazepine, triazepine, dihydrotriazepine, tetrahydrotriazepine, perhydrotriazepine, azocine, dihydroazocine, tetrahydroazocine, oxohydroazocine, perhydroazocine, morphane, azepindole, indoline, indolenine, isoindoline, isoindolenin, perhydroindole, perhydroisoindole, perhydroisoindole, indolizidine, dihydroindazole, perhydroindazole, dihydrobenzimidazole, perhydrobenzimidazole, dihydrobenzooxazole, perhydrobenzoxazole, dihydrobenzothiazole, perhydrobenzothiazole, dihydroquinoline, tetrahydroquinoline, perhydroquinoline, quinolidine, dihydroquinolidine, tetrahydroquinolidine, dihydroisoquinoline, tetrahydroisoquinoline, perhydroisoquinoline, dihydrocinnoline, tetrahydrocinnoline, perhydrocinnoline, dihydroquinazoline, tetrahydroquinazoline, perhydroquinazoline, dihydrophthalazine, tetrahydrophthalazine, perhydrophthalazine, dihydroquinoxaline, tetrahydroquinoxaline, perhydroquinoxaline, dihydronaphthyridine, tetrahydronaphthyridine, perhydronaphthyridine, quinolylidine, dihydrobenzoxazine, dihydrobenzothiazine, dihydrobenzoazepine, tetrahydrobenzoazepine, perhydrobenzoazepine, dihydrobenzodiazepine, tetrahydrobenzodiazepine, perhydrobenzodiazepine, dihydrobenzoxazepine, tetrahydrobenzoxazepine, perhydrobenzoxazepine, dihydrobenzothiazepine, tetrahydrobenzothiazepine, perhydrobenzothiazepine, dihydrocarbazole, tetrahydrocarbazole, perhydrocarbazole, dihydro β-carboline, tetrahydro β-carboline, perhydro β-carboline, dihydroacridine, tetrahydroacridine, perhydroacridine, dihydrophenazine, tetrahydrophenazine, perhydrophenazine, dihydrophenothiazine, tetrahydrophenothiazine, perhydrophenothiazine, dihydrophenoxazine, tetrahydrophenoxazine, perhydrophenoxazine, dihydrophenanthridine, tetrahydrophenanthridine, perhydrophenanthridine, dihydrophenanthroline, tetrahydrophenanthroline, perhydrophenanthroline, dihydroperimidine, tetrahydroperimidine, perhydroperimidine, pyrrolilidine, morphinan, hasvanan, dihydrofuran, tetrahydrofuran, pyran, dihydropyran, tetrahydropyran, dihydrooxepin, tetrahydrooxepin, perhydrooxepin, dihydrothiophene, tetrahydrothiophene, thiopyran, dihydrothiopyran, tetrahydrothiopyran, dihydrothiepin, tetrahydrothiepine, perhydrothiepine, dihydrobenzofuran, tetrahydrobenzofuran, perhydrobenzofuran, dihydroisobenzofuran, tetrahydroisobenzofuran, perhydroisobenzofuran, dihydrobenzothiophene, tetrahydrobenzothiophene, perhydrobenzothiophene, dihydroisobenzothiophene, tetrahydrobenzothiophene, perhydrobenzothiophene, benzopyran, dihydrobenzopyran, perhydrobenzopyran, benzothiopyran, dihydrobenzothiopyran, perhydrobenzothiopyran, dihydrobenzoxepin, tetrahydrobenzooxepin, perhydrobenzooxepin, dihydrobenzothiepine, tetrahydrobenzothiepine, perhydrobenzothiepine, dihydrodibenzofuran, tetrahydrodibenzofuran, perhydrodibenzofuran, xanthene, dihydroxanthene, tetrahydroxanthene, perhydroxanthene, dihydrodibenzothiophene, tetrahydrodibenzothiophene, perhydrodibenzothiophene, thioxanthene, dihydrothioxanthene, tetrahydrothioxanthene, perhydrothioxanthene, dihydrophenoxathiin, tetrahydrophenoxathiin, perhydrophenoxathiin, dihydrodibenzodioxin, tetrahydrodibenzodioxin, perhydrodibenzodioxin, dihydrothianthrene, tetrahydrothianthrene, perhydrothianthrene, oxirane, oxetane, thiirane, thiethane, dihydrooxathiin, tetrahydrooxathiin, dihydrobenzooxathiin, tetrahydrobenzooxathiin, perhydrobenzooxathiin, benzodioxepane, dioxolane, dioxane, dithiolane, dithiane, dioxoindane, benzodioxane, chromane, benzodithiolane, benzodithiane, norbornane, 1-azanorbornane, 2-azanorbornane, 7-azanorbornene, norbornene, 1-azanorbornene, 2-azanorbomene, 7-azanorbomene, norbornadiene, bicyclo[2.2.2]octane, 1-azabicyclo[2.2.2]octane, 2-azabicyclo[2.2.2]octane, norpinane, norpinene, norcanane, and norcanene. In a case of an unsaturated ring, a ring in which at least a part thereof is hydrogenated is also included. Furthermore, when a ring is formed, it is preferable to form a ring with any two or three substituents of R³, R⁴, R⁵, R⁶, and R⁷.

Furthermore, examples of a substituent which the alkyl group, the cycloalkyl group, the alkenyl group, the cycloalkenyl group, the alkynyl group, the aromatic group, and the heterocyclic group may have include groups selected from a hydroxy group, an alkyl group, a cycloalkyl group, an alkenyl group, a cycloalkenyl group, an alkynyl group, a halogen atom, an aromatic group, a heterocyclic group, an alkoxy group, a guanidino group, an alkylthio group, an alkoxycarbonyl group, an aryloxy group, an arylthio group, an acyl group, a substituted sulfonyl group, a heterocyclyloxy group, a heterocyclyl thio group, an amide group, a ureido group, a carboxy group, a carbamoyl group, an oxo group, a thioxo group, a sulfamoyl group, a sulfo group, a cyano group, a nitro group, an acyloxy group, an azido group, a sulfonamide group, a mercapto group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, an alkoxysulfonyl group, an Rx(Ry)N group, and an Rx(Ry)(Rz)N⁺ group (Rx, Ry, and Rz are each independently a hydrogen atom, an alkyl group, a cycloalkyl group, an alkenyl group, a cycloalkenyl group, an alkynyl group, an aromatic hydrocarbon group, or a heterocyclic group. Furthermore, at least two of Rx, Ry, and Rz may be bonded to each other to form a saturated or unsaturated heterocyclic ring, and this ring can also form a condensed ring or a spiro ring with an aliphatic ring or a heterocyclic ring and can also form a condensed ring with an aromatic ring).

Note that Rx, Ry, and Rz excluding the case of a hydrogen atom and the alkyl group, the cycloalkyl group, the alkenyl group, the cycloalkenyl group, the alkynyl group, the aromatic group, and the heterocyclic group as substituents, which are described herein, include groups similar to the groups represented by R¹, R², R³, R⁴, R⁵, R⁶, and R⁷. Furthermore, the alkyl group of the alkoxy group and the alkylthio group as substituents has the same meaning as defined in the alkyl group in R¹, R², R³, R⁴, R⁵, R⁶, and R⁷ described above, and the aryl group of the aryloxy group and the arylthio group has the same meaning as defined in the aromatic group in R¹, R², R³, R⁴, R⁵, R⁶, and R⁷ described above.

Furthermore, examples of the guanidino group, the acyl group, the substituted sulfonyl group, the heterocyclyloxy group, the heterocyclyl thio group, the carbamoyl group, the ureido group, the amide group, the sulfamoyl group, the acyloxy group, the sulfonamide group, the alkoxycarbonyl amino group, the aminocarbonyloxy group, the substituted sulfinyl group, the sulfamide group, the aminosulfonyloxy group, the alkoxysulfonyl amino group, the substituted sulfonyloxy group, the alkoxycarbonyl group, the alkoxycarbonyloxy group, and the alkoxysulfonyl group as substituents are as follows.

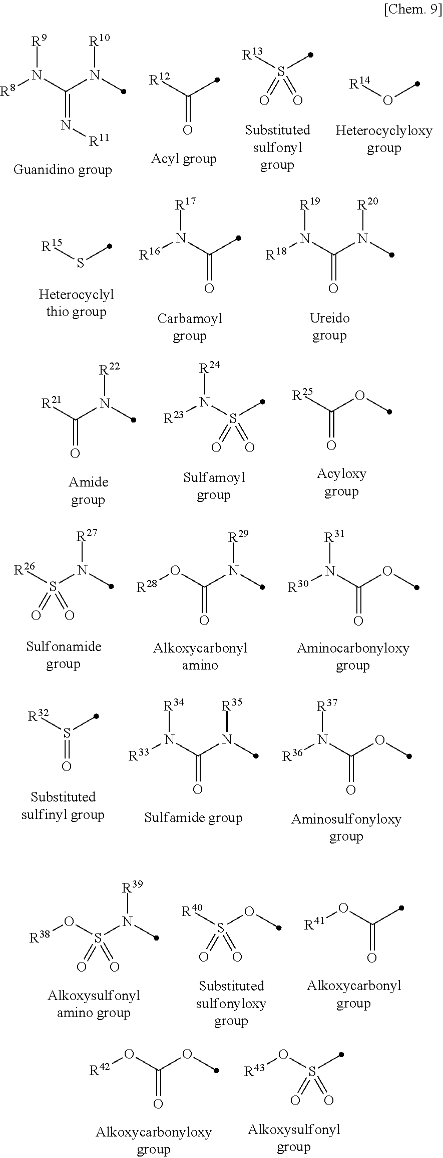

(In the exemplary groups, R⁸, R⁹, R¹⁰, R¹¹, R¹², R¹³, R¹⁶, R¹⁷, R¹⁸, R²⁵, R²⁷, R²⁹, R³⁰, R³¹, R³², R³³, R³⁴, R³⁵, R³⁶, R³⁷, R³⁹, and R⁴⁰ are each a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group. R²⁶, R²⁸, R³⁸, R⁴¹, R⁴², and R⁴³ are each a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group. R¹⁴ and R¹⁵ are each a substituted or unsubstituted heterocyclic group. Furthermore, examples of substituents of those substituted alkyl group, substituted cycloalkyl group, substituted alkenyl group, substituted cycloalkenyl group, substituted alkynyl group, substituted aromatic group, and substituted heterocyclic group include substituents similar to substituents of those groups in R¹, R², R³, R⁴, R⁵, R⁶, and R⁷ described above.)

Preferably, the groups represented by R¹, R², R³, R⁴, R⁵, R⁶, and R⁷ are each independently a hydrogen atom or an alkyl group having carbon number of 1 to 6, or two of R³, R⁴, R⁵, R⁶, and R⁷ are coupled to form a cycloalkyl group having carbon number of 3 to 8 in terms of ease of availability of a raw material. Particularly preferably, both of R¹ and R² are hydrogen atoms or one of R¹ and R² is a methyl group.

In Formula (II), when X is a single bond, from a viewpoint of obtaining a high release ratio, in a preferred embodiment, (i) R³ and R⁴ are hydrogen atoms; or (ii) at least one of R³ and R⁴ is a hydrogen atom, and l, m, and n are 0.

In Formula (II), when X is a single bond, from a viewpoint of stability of the conjugate in a solution, in a preferred embodiment, (iii) R³ and R⁴ are each independently a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; or (iv) at least one of R³ and R⁴ is a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group, and at least one of l, m, and n is an integer other than 0.

In Formula (II), when X is O, from a viewpoint of obtaining a high release ratio, in a preferred embodiment, (v) R¹ and R² are hydrogen atoms; (vi) at least one of R¹ and R² is a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group, at least one of R³ and R⁴ is a substituted or unsubstituted linear or branched alkyl group having carbon number of 3 to 6, a substituted or unsubstituted cycloalkyl group (for example, a cycloalkyl group having carbon number of 3 to 8), a substituted or unsubstituted alkenyl group (for example, an alkenyl group having carbon number of 3 to 6), a substituted or unsubstituted cycloalkenyl group (for example, a cycloalkenyl group having carbon number of 3 to 8), a substituted or unsubstituted alkynyl group (for example, an alkynyl group having carbon number of 3 to 6), a substituted or unsubstituted aromatic group (for example, a monocyclic or polycyclic aromatic group having carbon number of 6 to 14), or a substituted or unsubstituted heterocyclic group (for example, a 3- to 8-membered heterocyclic group containing at least one heteroatom selected from the group consisting of a nitrogen atom, an oxygen atom, and a sulfur atom as a ring-constituting atom), and when R⁵ and R⁶ are present, R⁵ and R are hydrogen atoms; or (vii) R³ and R⁴ are each independently a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group. In Formula (II), when X is O, from a viewpoint of obtaining a high release ratio, in a more preferred embodiment, (viii) R¹ and R² are hydrogen atoms; (ix) R¹ is a hydrogen atom, R² is a substituted or unsubstituted linear alkyl group having carbon number of 1 to 6, at least one of R³ and R⁴ is a substituted or unsubstituted linear or branched alkyl group having carbon number of 4 to 6, or a substituted or unsubstituted monocyclic or polycyclic aromatic group having carbon number of 6 to 14, and when R and R⁶ are present, R⁵ and R⁶ are hydrogen atoms; or (x) R³ and R⁴ are each independently a substituted or unsubstituted linear or branched alkyl group having carbon number of 1 to 6, a substituted or unsubstituted cycloalkyl group having carbon number of 3 to 8, a substituted or unsubstituted linear or branched alkenyl group having carbon number of 2 to 6, a substituted or unsubstituted cycloalkenyl group having carbon number of 3 to 8, a substituted or unsubstituted linear or branched alkynyl group having carbon number of 2 to 6, a substituted or unsubstituted monocyclic or polycyclic aromatic group having carbon number of 6 to 14, or a 3- to 8-membered substituted or unsubstituted heterocyclic group containing at least one heteroatom selected from the group consisting of a nitrogen atom, an oxygen atom, and a sulfur atom as a ring-constituting atom.

In Formula (II), when X is O, from a viewpoint of stability of the conjugate in a solution, in a preferred embodiment, (xi) at least one of R¹ and R² is a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group, R³ is a hydrogen atom, and R⁴ is a hydrogen atom, a methyl group, or an ethyl group; or (xii) m is 1, and R⁵ is a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group.

In Formula (II), when R⁷ is a group other than a hydrogen atom, from a viewpoint of obtaining a high release ratio, in a preferred embodiment, R¹ and R² are each independently a hydrogen atom, a methyl group, or an ethyl group.

In the bioactive carboxylic acid type compound-polymer conjugate represented by Formula (I) or (II) and the amine form which is an important intermediate represented by Formula (III) or (XI) according to the present invention, D has a structure representing a residue of a carboxylic acid type compound which itself or a pharmaceutically acceptable salt thereof is bioactive D-CO₂H excluding a carboxy group (—CO₂H in D-CO₂H).

Specifically, D is a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group, or has a structure obtained by combining these groups. Here, the alkyl group, the cycloalkyl group, the alkenyl group, the cycloalkenyl group, the alkynyl group, the aromatic group, or the heterocyclic group has the same meaning as defined in the above R¹, R², R³, R⁴, R⁵, R⁶, and R⁷. Note that here, the —CO₂H moiety in D-CO₂H means a carboxy group to form an ester bond with a linker represented by Formula (V).

Here, “bioactivity” refers to a property of acting on a living body by a compound or a pharmaceutically acceptable salt thereof. The property of acting on a living body includes a case where the property is exhibited by derivatization of the compound or a salt thereof. Furthermore, “acting on a living body” refers to all actions generally understood to have an effect on a living body, such as a therapeutic effect as a drug.

Examples of an embodiment of the bioactive compound or a pharmaceutically acceptable salt thereof include a pharmacologically active compound.

Furthermore, D has a structure representing a residue of a carboxylic acid type compound which itself or a pharmaceutically acceptable salt thereof is bioactive D-CO₂H excluding a carboxy group.

Examples of the bioactive compound may include a medical drug, a quasi-drug, a medical device, an in-vitro diagnostic medical drug, a tissue-engineered medical product, a medical drug for animals, an agricultural chemical, and active ingredients of a supplement. There is no limitation in the structure of the compound as long as the released bioactive carboxylic acid type compound D-CO₂H is bioactive and can form an ester bond by a carboxy group of the compound. A known compound available as a bioactive compound can be used as the bioactive carboxylic acid type compound D-CO₂H.

In particular, when glycosaminoglycan, more preferably chondroitin sulfate or hyaluronic acid is adopted as the polymer having a carboxy group, the release ratio of the bioactive carboxylic acid type compound D-CO₂H (for example, a carboxylic acid type compound having a bulky structure in the vicinity of a carboxy group) can be increased.

Furthermore, here, “carboxylic acid type compound having a bulky structure in the vicinity of a carboxy group” is, for example, a compound in which a carbon atom adjacent to a carboxy group is a tertiary or quaternary carbon atom, and is more specifically a carboxylic acid type compound in which a carbon atom adjacent to a carboxy group is substituted by a linear or branched alkyl group having carbon number of 1 to 10, a cycloalkyl group having carbon number of 3 to 10, a linear or branched alkenyl group having carbon number of 2 to 10, a cycloalkenyl group having carbon number of 3 to 10, an alkynyl groups having carbon number of 2 to 10, an aromatic group, or a heterocyclic group.

The amine form represented by Formula (III) or (XI) may form a salt with an inorganic acid or an organic acid. Examples of the inorganic acid include hydrochloric acid, sulfuric acid, and nitric acid. Examples of the organic acid include trifluoroacetic acid, methanesulfonic acid, toluenesulfonic acid, benzenesulfonic acid, and trifluoromethanesulfonic acid.

A structure derived from the polymer having a carboxy group is derived from a structure represented by Formula (IV):

Poly-CO₂H (IV)

which is a polymer having one or more carboxy groups in a molecule thereof. Here, “Poly” means a “residue of the polymer having a carboxy group”. The polymer may be obtained by introducing a carboxy group into a polymer having no carboxy group by chemical modification. Furthermore, when the polymer has a plurality of carboxy groups, the polymer may be condensed with one or more amine forms represented by Formula (III) or (XI).

That is, the compound represented by Formula (I) or (II) is a compound in which the polymer having a carboxy group, represented by the above Formula (IV), and one or more amine forms represented by Formula (III) or (XI) form an amide bond.

Furthermore, in a polymer chain, a unit represented by —COOH and a unit represented by —(═O)NH-A-C(═O)—O—C(—R¹)(R²)—O—C(═O)-D (—(═O)NH—(CH₂)ₙ—(C)ₘ(—R⁵)(—R⁶)—(CH₂)—C(—R³)(—R⁴)—X—C(═O)—O—C(—R¹)(R²)—O—C(═O)-D in Formula (II)) may be continuously arranged or may be randomly arranged in each polymer chain.

The degree of the amine forms represented by the above Formula (III) or (XI) to be condensed with one molecular chain of the carboxylic acid type polymer can be appropriately changed and adjusted according to the structure represented by D, the type of the carboxylic acid type polymer, and the like. The introduction degree of the structures represented by D may be indicated as “introduction ratio” here. When the introduction ratio is calculated as mol (%), the introduction ratio can be determined by calculating an integral ratio of ¹H-NMR. For example, when the amine form represented by the above Formula (III) or (XI) is introduced into all of the carboxy groups of the carboxylic acid type polymer, the introduction ratio is 100%. Furthermore, when the introduction ratio is calculated as a weight percent (wt %), the weight of the introduced D is calculated from the concentration of the introduced D with absorbance using a spectrophotometer, and the introduction ratio can be determined as the weight of D per the total weight of the compound represented by Formula (I) or (II) or a pharmaceutically acceptable salt thereof. For example, when the weight of an introduced drug is 10 mg and the total weight of the compound represented by Formula (I) or (II) or a pharmaceutically acceptable salt thereof is 100 mg, the introduction ratio is 10 wt %. Here, the introduction ratio calculated as a molar ratio is simply expressed by (%), and the introduction ratio based on absorbance measurement using a spectrophotometer is expressed by (wt %).

In the polymer, a carboxy group remaining without being condensed with the amine form represented by Formula (III) or (XI) may exist as a free carboxy group, may form a salt with a metal such as lithium, sodium, potassium, magnesium, or calcium, or an organic base such as triethylamine, tributylamine, or pyridine, or may form a salt using tetrabutylammonium hydroxide.

Examples of the polymer having a carboxy group include: synthetic polymers such as polyacrylic acid, polymethacrylic acid, polymaleic acid, polylactic acid (PLA), polyglycolic acid (PGA), a lactic acid-glycolic acid copolymer (PLGA), polycaprolactone, polycarboxyisopropylacrylamide, polyethylene terephthalate, polybutylene terephthalate, and carboxy group-modified polyethylene glycol; naturally existing polysaccharides such as alginic acid, hyaluronic acid, heparin, chondroitin, chondroitin sulfate (A, B, C, D, and E), keratan sulfate, heparan sulfate, dermatan sulfate, pectin (homogalacturonan and rhamnogalacturonan), xanthane gum, xylan, and sacran; semisynthetic polymers such as carboxymethyl cellulose, carboxymethyl chitin, carboxymethyl chitosan, carboxymethyl dextran, carboxymethyl amylose, succinyl chitosan, and polyethylene glycol into which a carboxy group is inserted; polyamino acids such as polyasparaginic acid, polyglutamic acid, and protein; and nucleic acids such as deoxyribonucleic acid into which a carboxy group is introduced. Examples of a water-soluble polymer having a carboxy group include: synthetic polymers such as polyacrylic acid, polymethacrylic acid, polymaleic acid, polycarboxyisopropylacrylamide, and carboxy group-modified polyethylene glycol; naturally existing polysaccharides such as alginic acid, hyaluronic acid, heparin, chondroitin, chondroitin sulfate (A, B, C, D, and E), keratan sulfate, heparan sulfate, dermatan sulfate, pectin(homogalacturonan and rhamnogalacturonan), xanthane gum, xylan, and sacran; semisynthetic polymers such as carboxymethyl cellulose, carboxymethyl chitin, carboxymethyl chitosan, carboxymethyl dextran, carboxymethyl amylose, succinyl chitosan, and polyethylene glycol into which a carboxy group is inserted; polyamino acids such as polyasparaginic acid, polyglutamic acid, and protein; and nucleic acids such as deoxyribonucleic acid into which a carboxy group is introduced. Examples of the polysaccharides include naturally existing polysaccharides such as alginic acid, hyaluronic acid, heparin, chondroitin, chondroitin sulfate (A, B, C, D, and E), keratan sulfate, heparan sulfate, dermatan sulfate, pectin (homogalacturonan and ramgalacturonan), xanthan gum, xylan, and sacran, carboxymethyl cellulose, carboxymethyl chitin, carboxymethyl chitosan, carboxymethyl dextran, carboxymethyl amylose, and succinyl chitosan. Examples of glycosaminoglycan include hyaluronic acid, heparin, chondroitin, chondroitin sulfate (A, B, C, D, and E), keratan sulfate, heparan sulfate, and dermatan sulfate. The polymer having a carboxy group may be cross-linked or chemically modified by various methods in advance, and may be further cross-linked or chemically modified after being formed into a bioactive carboxylic acid type compound-polymer conjugate. Furthermore, the polymer having a carboxy group may be a pharmaceutically acceptable salt, for example, a salt with a metal such as lithium, sodium, potassium, magnesium, or calcium, a salt with an organic base such as triethylamine, tributylamine, or pyridine, or a salt with tetrabutylammonium hydroxide.

Poly which is a residue of the polymer having a carboxy group means a partial structure of the polymer having a carboxy group represented by the above Formula (IV) excluding a carboxy group moiety used in condensation with the amine form represented by Formula (III) or (XI). As Poly which is a residue of the polymer, a water-soluble polymer residue, a polysaccharide residue, a glycosaminoglycan residue, a chondroitin residue, a chondroitin sulfate residue, and a hyaluronic acid residue can be exemplified as a preferable embodiment. Among these residues, from a viewpoint of the release ratio of the carboxylic acid type compound (for example, a carboxylic acid type compound having a bulky structure in the vicinity of a carboxy group) and water solubility of the conjugate, the glycosaminoglycan residue is preferable, and the chondroitin residue, the chondroitin sulfate residue, or the hyaluronic acid residue is more preferable. These residues each mean a partial structure of water-soluble polymer, polysaccharide, glycosaminoglycan, chondroitin, chondroitin sulfate, or hyaluronic acid excluding a carboxy group condensed with compound (III) or (XI).

An example of a method for manufacturing a bioactive carboxylic acid type compound-polymer conjugate represented by Formula (I) is described below.

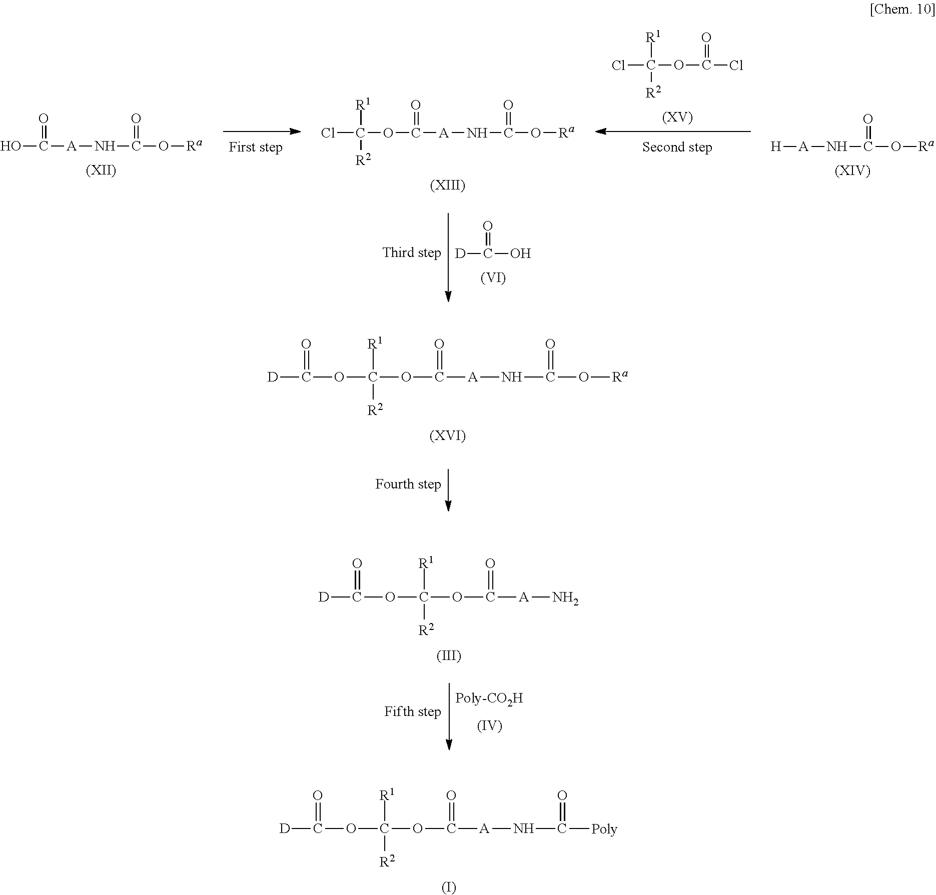

(In Formula, R^{a} is a benzyl group or a t-butyl group, and R¹, R², R³, R⁴, R⁵, R⁶, R⁷, D, X, A, l, m, n, and Poly are as defined above.)

First Step

This step is to manufacture a chloroalkyl ester form represented by the above Formula (XIII) from a protection amino acid represented by the above Formula (XII). This step can be performed by causing a reaction of a chloroalkylchloro sulfonate in the presence of a base. As the base, for example, sodium hydrogen carbonate, sodium carbonate, potassium hydrogen carbonate, potassium carbonate, sodium hydroxide, potassium hydroxide, or lithium hydroxide can be used. As the chloroalkylsulfonyl chloride, for example, chloromethyl chlorosulfonate or 1-chloroethyl chlorosulfonate can be used.

Upon performing this step, this step is preferably performed in a solvent. For example, an organic solvent such as methylene chloride, chloroform, dichloroethane, ethyl acetate, acetone, benzene, toluene, xylene, dimethylformamide, acetonitrile, tetrahydrofuran, dioxane, diethyl ether, diisopropyl ether, or dimethoxyethane can be used. A mixed solvent of an organic solvent and water can be used, if necessary. Furthermore, a phase transfer catalyst can be used, if necessary. As the phase transfer catalyst, for example, tetrabutylammonium hydrogen sulfate, tetrabutylammonium chloride, tetrabutylammonium bromide, or tetrabutylammonium iodide can be used. As a reaction temperature, the step can be proceeded generally in a range of −30° C. to 200° C. and preferably in a range of −15° C. to 80° C.

Second Step

This step is to manufacture a chloroalkyl ester form represented by the above Formula (XIII) from a protection amine form represented by the above Formula (XIV). At this time, when a terminal of A on a side bonded to a hydrogen atom in the above Formula (XIV) is an oxygen atom, a carbonate bond is formed as a product represented by the above Formula (XIII). When the terminal of A on a side bonded to a hydrogen atom in the above Formula (XIV) is a nitrogen atom, a urethane bond is formed. This step can be performed by causing a protection amine form represented by the above Formula (XIV) to react with a chloroalkyl chloroformate represented by the above Formula (XV), and a base can be added, if necessary.

Upon performing this step, this step is preferably performed in a solvent. For example, an organic solvent such as methylene chloride, chloroform, dichloroethane, ethyl acetate, acetone, benzene, toluene, xylene, dimethylformamide, acetonitrile, tetrahydrofuran, dioxane, diethyl ether, diisopropyl ether, or dimethoxyethane can be used. As the base, pyridine, N,N-diisopropylethylamine, triethylamine, 2,6-lutidine, 4-dimethylaminopyridine, diazabicycloundecene, 1,8-bis(dimethylamino) naphthalene, metal bis(trimethylsilyl) amide, or lithium diisopropylamide can be used. As a reaction temperature, the step can be proceeded generally in a range of −78° C. to 200° C. and preferably in a range of −20° C. to 80° C.

Third Step

This step is to manufacture an ester form represented by Formula (XVI) by causing a chloroalkyl ester form represented by the above Formula (XIII) to react with a carboxylic acid type compound represented by the above Formula (VI). This step can be performed by causing a bioactive carboxylic acid type compound represented by the above Formula (VI) to react with a base to form a carboxylate, and then causing the carboxylate to react with a chloroalkyl ester form represented by the above Formula (XIII), or by causing a bioactive carboxylic acid type compound represented by the above Formula (VI) to react with a chloroalkyl ester form represented by the above Formula (XIII) in the presence of a base.

Upon forming the salt, this step is preferably performed in a solvent. For example, an organic solvent such as methanol, ethanol, methylene chloride, chloroform, dichloroethane, ethyl acetate, acetone, benzene, toluene, xylene, acetonitrile, tetrahydrofuran, dioxane, diethyl ether, diisopropyl ether, or dimethoxyethane can be used. Furthermore, as the base, for example, cesium carbonate, sodium hydrogen carbonate, sodium carbonate, potassium hydrogen carbonate, potassium carbonate, sodium hydroxide, potassium hydroxide, or lithium hydroxide can be used. As a reaction temperature, the step can be proceeded generally in a range of −30° C. to 200° C. and preferably in a range of −15° C. to 80° C.

Upon performing the esterification, the esterification is preferably performed in a solvent. For example, an organic solvent such as dimethylformamide, methylene chloride, chloroform, dichloroethane, ethyl acetate, acetone, benzene, toluene, xylene, acetonitrile, tetrahydrofuran, dioxane, dimethyl sulfoxide, diethyl ether, diisopropyl ether, or dimethoxyethane can be used. As the base to coexist, for example, cesium carbonate, sodium hydrogen carbonate, sodium carbonate, potassium hydrogen carbonate, potassium carbonate, sodium hydroxide, potassium hydroxide, lithium hydroxide, pyridine, N,N-diisopropylethylamine, triethylamine, 2,6-lutidine, 4-dimethylaminopyridine, diazabicycloundecene, 1,8-bis(dimethylamino) naphthalene, metal bis(trimethylsilyl) amide, or lithium diisopropylamide can be used. As a reaction temperature, the step can be proceeded generally in a range of −30° C. to 200° C. and preferably in a range of 0° C. to 80° C.

Fourth Step

This step is to manufacture an amine form represented by the above Formula (III) by deprotecting an ester represented by the above Formula (XVI).

In this step, in a case where R^{a} is a benzyl group, the amine form represented by the above Formula (III) can be manufactured by deprotection by catalytic hydrogen addition. As a metal catalyst to be used, for example, a platinum catalyst such as platinum oxide or platinum carbon, a palladium catalyst such as palladium carbon, palladium black, or palladium oxide, or a nickel catalyst such as Raney nickel can be used. Upon performing this step, this step is preferably performed in a solvent. For example, methanol, ethanol, isopropyl alcohol, tetrahydrofuran, dimethylformamide, dioxane, or water can be used. As a reaction temperature, the step can be proceeded generally in a range of −50° C. to 200° C. and preferably in a range of 10° C. to 100° C.

In this step, in a case where R^{a} is a t-butyl group, the amine form represented by the above Formula (III) can be manufactured by deprotection using an acid. As the acid, for example, hydrogen chloride, hydrochloric acid, sulfuric acid, nitric acid, methanesulfonic acid, benzenesulfonic acid, p-toluenesulfonic acid, trifluoromethanesulfonic acid, or trifluoroacetic acid can be used. The amine form represented by the above Formula (III) which is obtained in this step is manufactured by forming salts with these acids. In this step, the reaction can be proceeded in the absence of a solvent or in a solvent, and as the solvent, for example, ethyl acetate, dioxane, methanol, ethanol, 1-propanol, 2-propanol, diethyl ether, or water can be used. As a reaction temperature, the step can be proceeded generally in a range of −50° C. to 200° C. and preferably in a range of 0° C. to 80° C.

Fifth Step

This step is to manufacture a bioactive carboxylic acid type compound-polymer conjugate represented by the above Formula (I) by condensing an amine form represented by the above Formula (III) with a polymer having a carboxy group represented by the above Formula (IV). As the polymer having a carboxy group represented by the above Formula (IV) used in this step, a polymer which has previously formed a salt with a metal or an organic base may be used. As a condensing agent to be used for the condensation reaction, for example, 1-ethyl-3-(3-dimethylaminopropyl) carbodiimide hydrochloride (EDC or WSC), 4-(4,6-dimethoxy-1,3,5-triazin-2-yl)-4-methylmorpholinium chloride (DMT-MM), tetramethylfluoroformamidinium hexafluorophosphate (TFFH), or bis(tetramethylene) fluoroformamidinium hexafluorophosphate (BTFFH) can be used. Furthermore, in a case where a carboxy group of the polymer having the carboxy group is derivatized into an active ester such as N-hydroxysuccinimide ester or p-nitrophenyl ester, it is not necessary to add a condensing agent, and condensation can also be performed by only mixing with an amine form represented by Formula (III), or if necessary, adding a base.

This step is preferably performed in a solvent, and for example, water or an organic solvent such as methylene chloride, chloroform, dichloroethane, toluene, ethyl acetate, acetone, dimethylformamide, formamide, N-methylpyrrolidone, acetonitrile, tetrahydrofuran, dioxane, diethyl ether, dimethoxyethane, dimethyl sulfoxide, methanol, ethanol, 1-propanol, 2-propanol, or ethylene glycol can be used. Furthermore, a mixed solvent obtained by mixing any one of these organic solvents with water at an arbitrary ratio can also be used.

Another embodiment of the present invention is a linker represented by the following Formula (V) for bonding a bioactive carboxylic acid type compound to a polymer having a carboxy group.

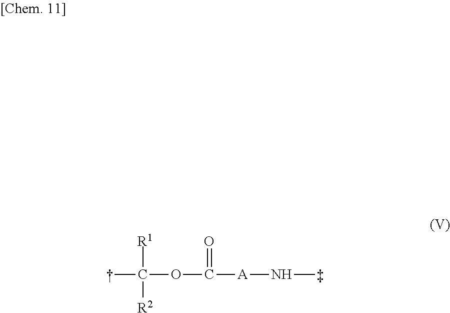

(herein, R¹, R², and A are as defined above. Furthermore, the symbol t is a bonding point with a moiety of a carboxy group of a bioactive carboxylic acid type compound excluding a hydrogen atom, and the symbol is a bonding point with a moiety of a carboxy group of a polymer having the carboxy group excluding a hydroxy group).

The conjugate of the present invention can be obtained by the methods exemplified in the above-described steps 1 to 5 using a linker represented by Formula (V). Therefore, another embodiment of the present invention is a method for manufacturing a compound represented by Formula (I) or a pharmaceutically acceptable salt thereof, the method including a step of bonding a bioactive carboxylic acid type compound to a polymer having a carboxy group via a linker represented by Formula (V).

The properties of a composition containing a bioactive carboxylic acid type compound-polymer conjugate represented by Formula (I) or (II) of the present invention are not particularly limited, but are preferably in a form of an aqueous solution. More specifically, the composition is preferably used in a form of an aqueous solution for medical drugs, medical devices, agricultural chemicals, and the like.

That is, one embodiment of the present invention is an aqueous solution containing a compound represented by Formula (I) or (II).

Here, the aqueous solution refers to a liquid in which water is a solvent and the entire liquid is visually uniform and transparent. The aqueous solution may contain an aqueous solvent other than water as long as having the property. Furthermore, in consideration of preservation of medical drugs and medical devices, for example, the aqueous solution is preferably in a form of an aqueous solution at 25° C.

“Water-soluble” here means a property of a compound represented by Formula (I) or (II) or a pharmaceutically acceptable salt thereof that the entire liquid is visually uniform and transparent at a concentration of 1.5 mg/mL when water at 25° C. is used as a solvent.

The carboxylic acid type compound-polymer conjugate of the present invention is a conjugate whose release rate can be controlled and is expected to be used in medical drugs and the like, as clearly illustrated in Test Example described later.

### EXAMPLES

Hereinafter, the present invention will be described in more detail by means of Reference Examples and Examples. However, the present invention is not limited to those examples without departing from the scope thereof.

Furthermore, synthesis examples of a chloroalkyl ester form represented by the above Formula (XIII), an ester form represented by the above Formula (XVI), and an amine form represented by the above Formula (III), which are intermediates for manufacturing the bioactive carboxylic acid type compound-polymer conjugate of the present invention, are described as Reference Examples.

### Reference Example 1

### 2-[4-[2-[(4-Chlorobenzoyl) amino] ethyl] phenoxy]-2-methylpropanoic Acid

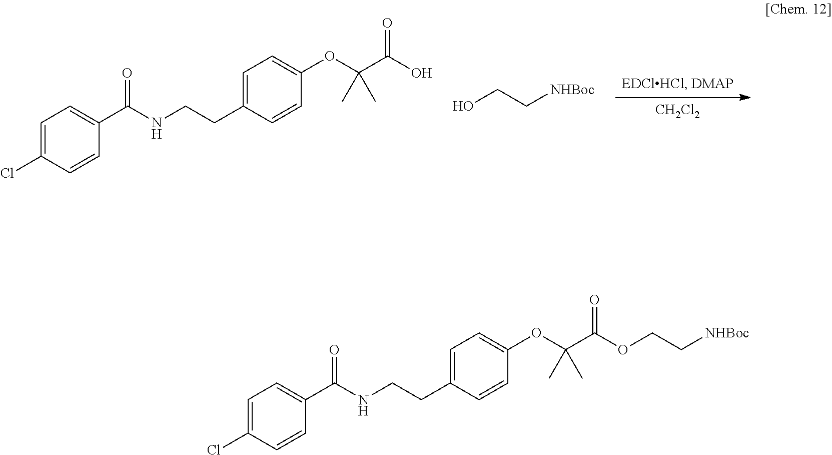 [SMILES: CC(C)(Oc1ccc(CCNC(=O)c2ccc(Cl)cc2)cc1)C(=O)O ; CC(C)(C)OC(=O)NCCO ; "CH2Cl2" ; "EDCl·HCl, DMAP" ; CC(C)(C)OC(=O)NCCOC(=O)C(C)(C)Oc1ccc(CCNC(=O)c2ccc(Cl)cc2)cc1]

To a methylene chloride (4 mL) solution of 500 mg (1.38 mmol) of bezafibrate and 267 mg (1.66 mmol) of 2-[[(1,1-dimethylethoxy) carbonyl] amino]ethanol, 253 mg (2.07 mmol) of 4-dimethylaminopyridine and 344 mg (1.80 mmol) of 1-(3-dimethylaminopropyl)-3-ethylcarbodiimide hydrochloride were added under cooling on ice, and the resulting mixture was stirred overnight. A saturated ammonium chloride aqueous solution was added to the reaction solution, and the resulting mixture was extracted with ethyl acetate. The obtained organic layer was washed with saturated sodium bicarbonate water and saturated saline, and then dried with anhydrous magnesium sulfate, and the solvent was distilled off under reduced pressure. The residue was purified by silica gel column chromatography to obtain 650 mg (91%) of the title compound.

¹H-NMR (DMSO-d₆, δ): 1.36 (9H, s), 1.49 (6H, s), 2.77 (2H, t, J=8 Hz), 3.19 (2H, q, J=6 Hz), 3.41-3.45 (2H, m), 4.10 (2H, t, J=6 Hz), 6.74 (2H, d, J=9 Hz), 6.91 (1H, t, J=6 Hz), 7.12 (2H, d, J=9 Hz), 7.53 (2H, d, J=9 Hz), 7.83 (2H, d, J=9 Hz), 8.61 (1H, t, J=6 Hz)

### Reference Example 2

### 2-[4-[2-[(4-Chlorobenzoyl) amino] ethyl] phenoxy]-2-methylpropanoic Acid 2-aminoethyl Ester Hydrochloride

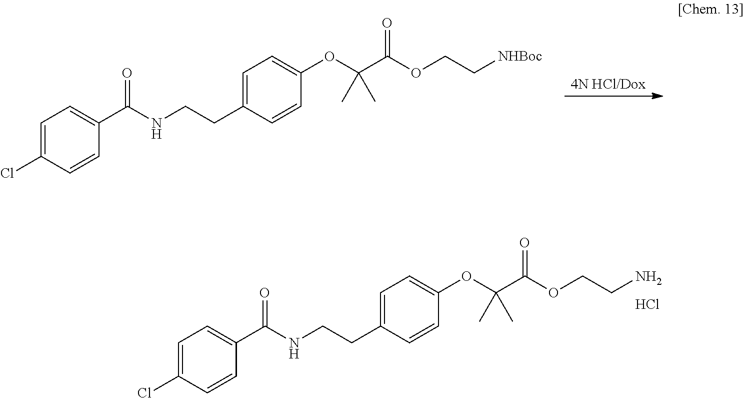 [SMILES: CC(C)(C)OC(=O)NCCOC(=O)C(C)(C)Oc1ccc(CCNC(=O)c2ccc(Cl)cc2)cc1 ; "4N HCl/Dox" ; CC(C)(Oc1ccc(CCNC(=O)c2ccc(Cl)cc2)cc1)C(=O)OCCN ; Cl]

To 200 mg (0.355 mmol) of 2-[4-[2-[(4-chlorobenzoyl) amino] ethyl]phenoxy]-2-methylpropanoic acid 2-[[(1,1-dimethylethoxy) carbonyl] amino] ethyl ester, 2 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and the precipitated solid was collected by filtration to obtain 169 mg (quant.) of the title compound.

¹H-NMR (DMSO-d₆, δ): 1.54 (6H, s), 2.78 (2H, t, J=8 Hz), 3.10 (2H, br-s), 3.42-3.46 (2H, m), 4.30 (2H, t, J=6 Hz), 6.77 (2H, d, J=9 Hz), 7.13 (2H, d, J=9 Hz), 7.53 (2H, d, J=9 Hz), 7.85 (2H, d, J=9 Hz), 8.26 (3H, br-s), 8.69 (1H, t, J=6 Hz)

### Reference Example 3

### (2-Bezafibrate-ethyl) amino-chondroitin Sulfate Conjugate

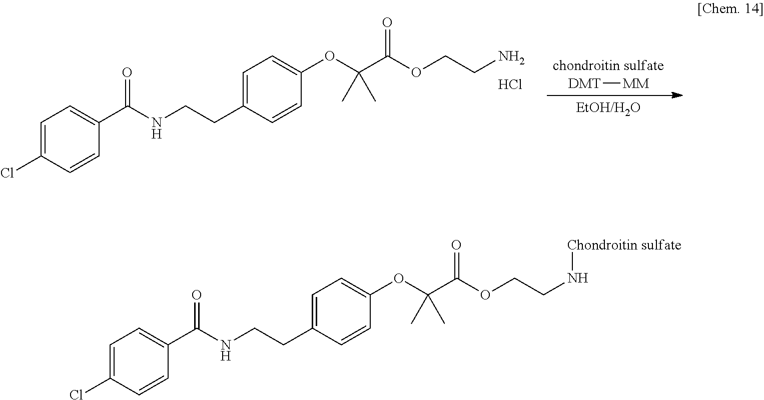

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 35 mg (0.080 mmol) of 2-[4-[2-[(4-chlorobenzoyl) amino]ethyl] phenoxy]-2-methylpropanoic acid 2-aminoethyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 152 mg of the title compound. Based on values of integral in ¹H-NMR, the introduction ratio of bezafibrate per unit of whole disaccharide (glucuronic acid) of chondroitin sulfate was 16%.

### Reference Example 4

### N-[(1,1-Dimethylethoxy) carbonyl]-β-alanine Chloromethyl Ester

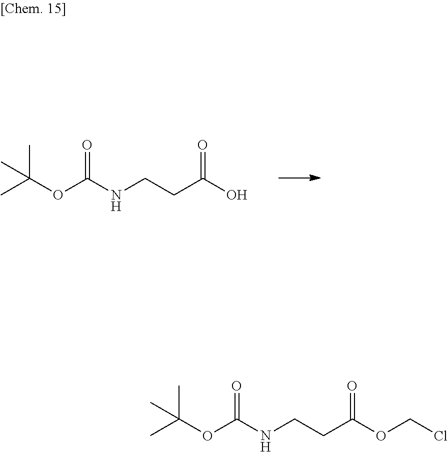

Under cooling on ice, a methylene chloride solution of 1.15 g (7.0 mmol) of chloromethyl chlorosulfonate was added dropwise to a water 10 mL-methylene chloride 10 mL mixed solution of 1.10 g (5.8 mmol) of N-[(1,1-dimethylethoxy) carbonyl]-β-alanine, 197 mg (0.58 mmol) of tetrabutylammonium hydrogen sulfate, and 1.95 g (23.2 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate, and then the solvent was distilled off under reduced pressure. The residue was dissolved again in diethyl ether and washed with water. The water layer was extracted with a small amount of diethyl ether, and the extracted product was put together with the organic layer. The organic layer was washed with saturated saline and dried with anhydrous sodium sulfate, and then the solvent was distilled off under reduced pressure to obtain 1.20 g (87%) of the title compound.

¹H-NMR (CDCl₃, δ): 1.44 (9H, s), 2.62 (2H, t, J=6 Hz), 3.37-3.46 (2H, m), 4.95 (1H, br-s), 5.71 (2H, s)

### Reference Example 5

### 2-[4-[2-[(4-Chlorobenzoyl) amino] ethyl] phenoxy]-2-methylpropanoic Acid [3-[[(1,1-dimethylethoxy) carbonyl] amino]-1-oxopropoxy] methyl Ester

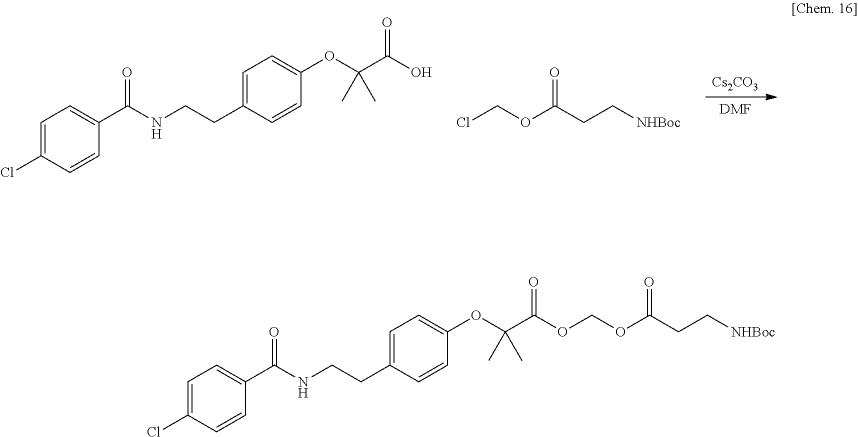

To a methanol suspension of 500 mg (1.38 mmol) of bezafibrate, 225 mg (0.691 mmol) of cesium carbonate was added, and the resulting mixture was stirred at room temperature for 30 minutes. The solvent of the mixed liquid was distilled off under reduced pressure, and the residue was dried using a vacuum pump overnight. The residue was dissolved in N,N-dimethylformamide, and an N,N-dimethylformamide solution of 225 mg (0.691 mmol) of N-[(1,1-dimethylethoxy) carbonyl]-β-alanine chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 550 mg (71%) of the title compound.

¹H-NMR (DMSO-d₆, δ): 1.36 (9H, s), 1.49 (6H, s), 2.47 (2H, t, J=7 Hz), 2.77 (2H, t, J=8 Hz), 3.14 (2H, q, J=7 Hz), 3.44 (2H, dt, J=6, 8 Hz), 5.78 (2H, s), 6.74 (2H, d, J=9 Hz), 6.86 (1H, br-s), 7.13 (2H, d, J=9 Hz), 7.53 (2H, d, J=9 Hz), 7.82 (2H, d, J=9 Hz), 8.61 (1H, t, J=6 Hz)

### Reference Example 6

### 2-[4-[2-[(4-Chlorobenzoyl) amino] ethyl] phenoxy]-2-methylpropanoic Acid (3-amino-1-oxopropoxy) Methyl Ester Hydrochloride

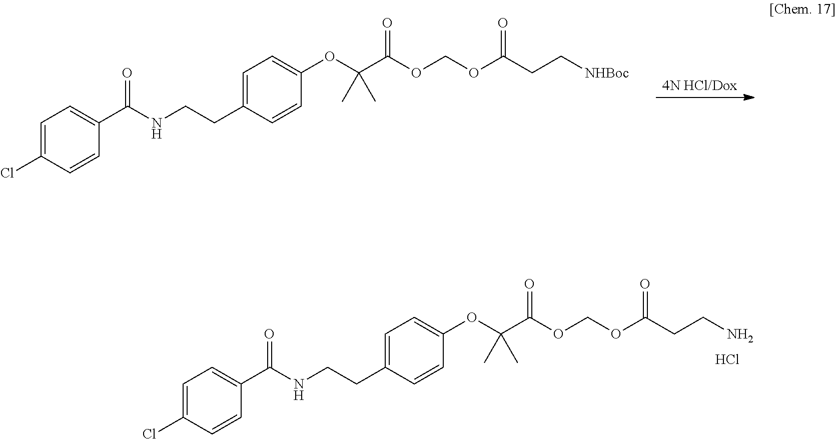 [SMILES: CC(C)(C)OC(=O)NCCC(=O)OCOC(=O)C(C)(C)Oc1ccc(CCNC(=O)c2ccc(Cl)cc2)cc1 ; "4N HCl/Dox" ; CC(C)(Oc1ccc(CCNC(=O)c2ccc(Cl)cc2)cc1)C(=O)OCOC(=O)CCN ; Cl]

To 380 mg (0.675 mmol) of 2-[4-[2-[(4-chlorobenzoyl) amino] ethyl]phenoxy]-2-methylpropanoic acid [3-[[(1,1-dimethylethoxy) carbonyl] amino]-1-oxopropoxy] methyl ester, 4 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and the precipitated solid was collected by filtration to obtain 265 mg (79%) of the title compound.

¹H-NMR (DMSO-d₆, δ): 1.50 (6H, s), 2.75-2.81 (4H, m), 3.01 (2H, t, J=7 Hz), 3.42-3.47 (2H, m), 5.82 (2H, s), 6.75 (2H, d, J=9 Hz), 7.15 (2H, d, J=9 Hz), 7.53 (2H, d, J=9 Hz), 7.85 (2H, d, J=9 Hz), 8.06 (3H, br-s), 8.96 (1H, t, J=6 Hz)

### Example 1

### [3-(Bezafibrate-methoxy)-3-oxopropyl] Amino-Chondroitin Sulfate Conjugate

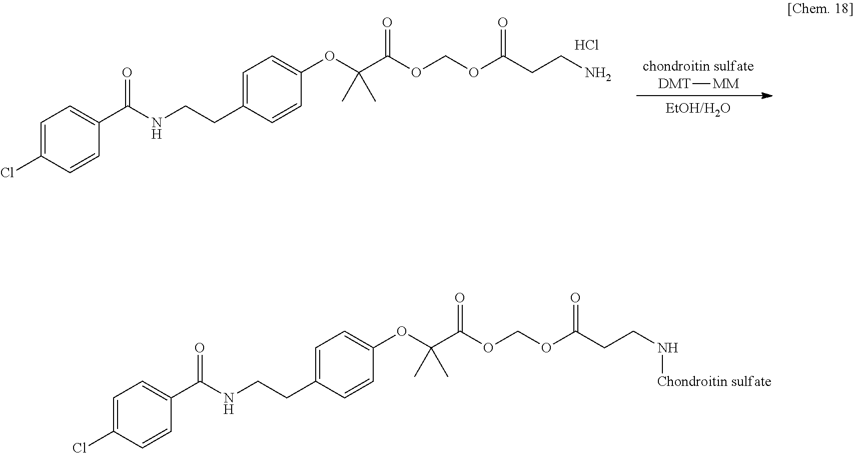

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 36 mg (0.080 mmol) of 2-[4-[2-[(4-chlorobenzoyl) amino]ethyl] phenoxy]-2-methylpropanoic acid (3-amino-1-oxopropoxy) methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 8 mL of 90% ethanol. To the mixed liquid, 9 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 218 mg of the title compound. Based on values of integral in ¹H-NMR, the introduction ratio of bezafibrate per unit of whole disaccharide (glucuronic acid) of chondroitin sulfate was 16%.

Furthermore, an attempt was made to prepare a conjugate in which the introduction ratio of bezafibrate was changed by adjusting a quantitative ratio between chondroitin sulfate and a bezafibrate derivative.

To 2.0 g (0.20 mmol) of a 5% sodium chondroitin sulfate aqueous solution, 0.8 mL of ethanol was added dropwise under stirring. To the mixed liquid, an ethanol 1 mL-water 1 mL solution of 79 mg (0.16 mmol) of 2-[4-[2-[(4-chlorobenzoyl) amino]ethyl] phenoxy]-2-methylpropanoic acid (3-amino-1-oxopropoxy) methyl ester hydrochloride was added. Subsequently, an ethanol 1.2 mL solution of 66 mg (0.24 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 50 μL of a 20% sodium chloride aqueous solution was added, and ethanol was further added dropwise (about 4 mL) until immediately before the reaction solution became cloudy. The reaction solution was added dropwise under stirring to 5 mL of 90% ethanol. To the mixed liquid, 2.5 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 145 mg of the title compound. Based on values of integral in ¹H-NMR, the introduction ratio of bezafibrate per unit of whole disaccharide (glucuronic acid) of chondroitin sulfate was 50%.

In Test Example 1, a conjugate having an introduction ratio of 16% was used.

### Reference Example 7

### (2E)-3-[4-(1H-Imidazol-1-ylmethyl) phenyl]-2-propenoic Acid [3-[[(1,1-dimethylethoxy) carbonyl] amino]-1-oxopropoxy] Methyl Ester

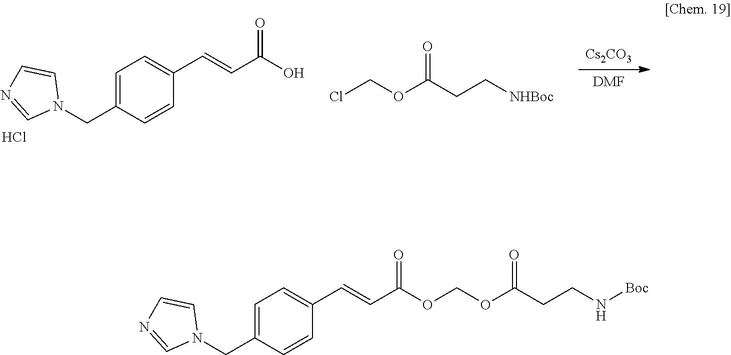

To a methanol solution of 400 mg (1.51 mmol) of ozagrel hydrochloride, 492 mg (1.51 mmol) of cesium carbonate was added, and the resulting mixture was stirred at room temperature for 30 minutes. The solvent of the mixed suspension was distilled off under reduced pressure, and the residue was dried using a vacuum pump overnight. The residue was suspended in N,N-dimethylformamide, and an N,N-dimethylformamide solution of 413 mg (1.51 mmol) of N-[(1,1-dimethylethoxy) carbonyl]-β-alanine chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 351 mg (54%) of the title compound.

¹H-NMR (CD₃OD, δ): 1.40 (9H, s), 2.56 (2H, t, J=7 Hz), 3.12 (2H, t, J=7 Hz), 5.26 (2H, s), 5.87 (2H, s), 6.55 (1H, d, J=16 Hz), 7.00 (1H, t, J=1 Hz), 7.11 (1H, t, J=1 Hz), 7.28 (2H, d, J=9 Hz), 7.63 (2H, d, J=9 Hz), 7.74-7.77 (2H, m)

### Reference Example 8

### (2E)-3-[4-(1H-Imidazol-1-ylmethyl) phenyl]-2-propenoic Acid (3-amino-1-oxopropoxy) Methyl Ester Dihydrochloride

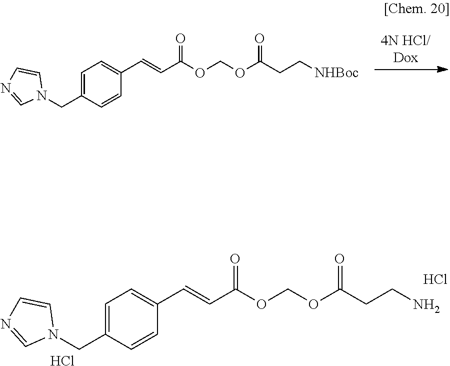 [SMILES: CC(C)(C)OC(=O)NCCC(=O)OCOC(=O)/C=C/c1ccc(Cn2ccnc2)cc1 ; "4N HCl/Dox" ; NCCC(=O)OCOC(=O)/C=C/c1ccc(Cn2ccnc2)cc1 ; Cl ; Cl]

To 140 mg (0.326 mmol) of (2E)-3-[4-(1H-imidazol-1-ylmethyl) phenyl]-2-propenoic acid [3-[[(1,1-dimethylethoxy) carbonyl] amino]-1-oxopropoxy] methyl ester, 4 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and then the precipitated solid was collected by filtration to obtain 103 mg (79%) of the title compound.

¹H-NMR (DMSO-d₆, δ): 2.80 (2H, t, J=7 Hz), 3.00-3.06 (2H, m), 5.49 (2H, s), 5.86 (2H, s), 6.72 (1H, d, J=16 Hz), 7.47 (2H, d, J=9 Hz), 7.07 (1H, t, J=2 Hz), 7.76 (1H, d, J=16 Hz), 7.81-7.82 (3H, m), 8.11 (3H, br-s), 9.33 (1H, s), 14.77 (1H, br-s)

### Example 2

### [3-(Ozagrel) methoxy-3-oxopropyl] Amino-Chondroitin Sulfate Conjugate

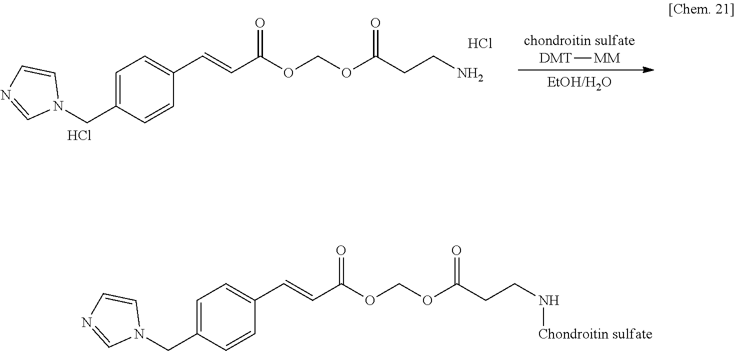

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 32 mg (0.080 mmol) of (2E)-3-[4-(1H-imidazol-1-ylmethyl) phenyl]-2-propenoic acid (3-amino-1-oxopropoxy) methyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 13 mL of 94% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 180 mg of the title compound. Based on values of integral in ¹H-NMR, the introduction ratio of ozagrel per unit of whole disaccharide (glucuronic acid) of chondroitin sulfate was 8%.

### Reference Example 9

### 5-(Aminosulfonyl)-4-chloro-2-[(2-furanylmethyl) amino] benzoic Acid [3-[[(1,1-dimethylethoxy) carbonyl] amino]-1-oxopropoxy] methyl Ester

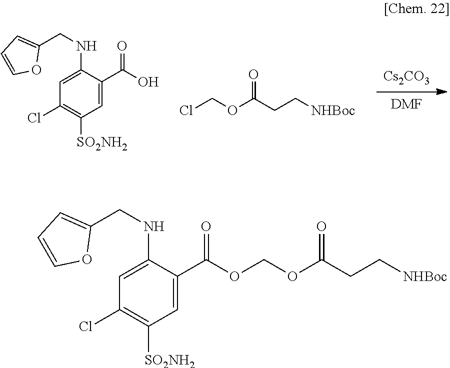

To a methanol solution of 500 mg (1.51 mmol) of furosemide, 246 mg (0.756 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 413 mg (1.51 mmol) of N-[(1,1-dimethylethoxy) carbonyl]-β-alanine chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 630 mg (78%) of the title compound.

¹H-NMR (CD₃OD, δ): 1.42 (9H, s), 2.58 (2H, t, J=7 Hz), 3.32 (2H, t, J=7 Hz), 4.50 (2H, s), 5.95 (2H, s), 6.34 (1H, dd, J=1, 4 Hz), 6.37 (1H, dd, J=2, 4 Hz), 7.01 (1H, s), 7.45 (1H, dd, J=1, 2 Hz), 8.51 (1H, s)

### Reference Example 10

### 5-(Aminosulfonyl)-4-chloro-2-[(2-furanylmethyl (amino] benzoic Acid (3-amino-1-oxopropoxy) Methyl Ester Dihydrochloride

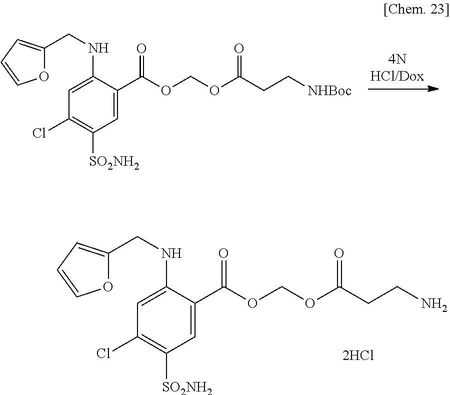 [SMILES: CC(C)(C)OC(=O)NCCC(=O)OCOC(=O)c1cc(S(N)(=O)=O)c(Cl)cc1NCc1ccco1 ; NCCC(=O)OCOC(=O)c1cc(S(N)(=O)=O)c(Cl)cc1NCc1ccco1]

To 350 mg (0.658 mmol) of 5-(aminosulfonyl)-4-chloro-2-[(2-furanylmethyl) amino] benzoic acid [3-[[(1,1-dimethylethoxy) carbonyl] amino]-1-oxopropoxy]methyl ester, 4 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and the precipitated solid was collected by filtration to obtain 212 mg (64%) of the title compound.

¹H-NMR (DMSO-d₆, δ): 2.79 (2H, t, J=7 Hz), 3.04 (2H, t, J=7 Hz), 4.63 (2H, d, J=6 Hz), 5.96 (2H, s), 6.39 (1H, dd, J=1 Hz, 4 Hz), 6.43 (1H, dd, J=2, 4 Hz), 7.15 (1H, s), 7.38 (2H, s), 7.63 (1H, dd, J=1, 2 Hz), 8.03 (3H, br-s), 8.38-8.40 (2H, m)

### Example 3

### [3-[(Furosemide) methoxy]-3-oxopropyl] Amino-Chondroitin Sulfate Conjugate

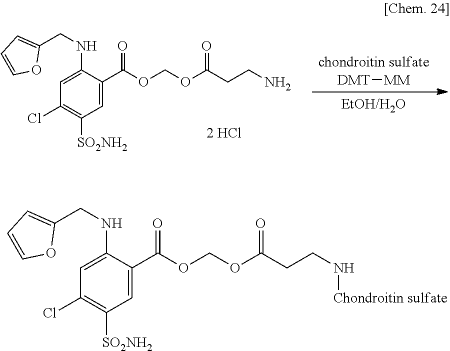

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 40 mg (0.080 mmol) of 5-(aminosulfonyl)-4-chloro-2-[(2-furanylmethyl) amino] benzoic acid (3-amino-1-oxopropoxy) methyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 227 mg of the title compound. Based on values of integral in ¹H-NMR, the introduction ratio of furosemide per unit of whole disaccharide (glucuronic acid) of chondroitin sulfate was 14%.

### Reference Example 11

### 3-Pyridine Carboxylic Acid [3-[[(1,1-dimethylethoxy) carbonyl] amino]-1-oxopropoxy] methyl Ester

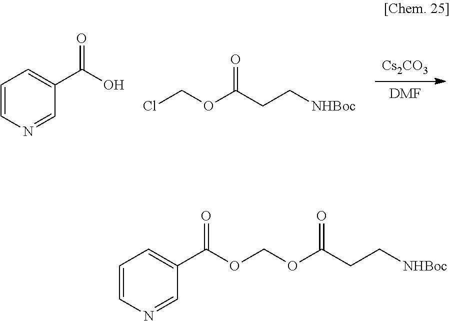

To a methanol solution of 180 mg (1.46 mmol) of nicotinic acid, 285 mg (0.877 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 400 mg (1.46 mmol) of N-[(1,1-dimethylethoxy) carbonyl]-β-alanine chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 385 mg (81%) of the title compound.

¹H-NMR (CDCl₃, δ): 1.42 (9H, s), 2.63 (2H, t, J=6 Hz), 3.43 (2H, q, J=6 Hz), 4.97 (1H, br-s), 6.04 (2H, s), 7.42 (1H, ddd, J=1, 5, 8 Hz), 8.33 (1H, td, J=2, 8 Hz), 8.82 (1H, dd, J=2, 5 Hz), 9.26 (1H, dd, J=1, 2 Hz)

### Reference Example 12

### 3-Pyridine Carboxylic Acid (3-amino-1-oxopropoxy) methyl Ester Dihydrochloride

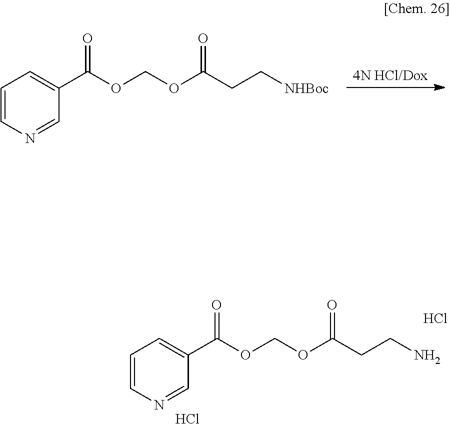 [SMILES: CC(C)(C)OC(=O)NCCC(=O)OCOC(=O)c1cccnc1 ; "4N HCl/Dox" ; NCCC(=O)OCOC(=O)c1cccnc1 ; Cl ; Cl]

To 312 mg (0.962 mmol) of 3-pyridine carboxylic acid [3-[[(1,1-dimethylethoxy) carbonyl] amino]-1-oxopropoxy] methyl ester, 3 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and then the precipitated solid was collected by filtration to obtain 189 mg (66%) of the title compound.

¹H-NMR (DMSO-d₆, δ): 2.85 (2H, t, J=7 Hz), 3.02-3.06 (2H, m), 6.10 (2H, s), 7.64 (1H, br-s), 7.74 (1H, dd, J=5, 8 Hz), 8.25 (3H, br-s), 8.47 (1H, td, J=2, 8 Hz), 8.94 (1H, dd, J=2, 5 Hz), 9.10 (1H, dd, J=1, 2 Hz)

### Example 4

### [3-[(Nicotinic acid)-methoxy]-3-oxopropyl] Amino-Chondroitin Sulfate Conjugate

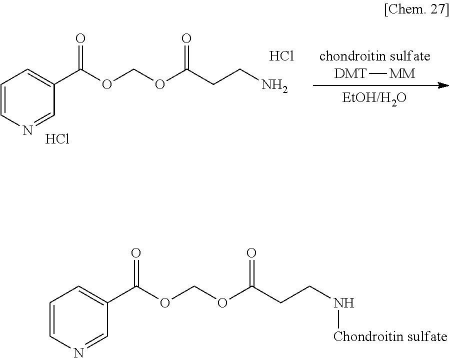

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 24 mg (0.080 mmol) of 3-pyridine carboxylic acid (3-amino-1-oxopropoxy) methyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 8 mL of 90% ethanol. To the mixed liquid, 9 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 200 mg of the title compound. Based on values of integral in ¹H-NMR, the introduction ratio of nicotinic acid per unit of whole disaccharide (glucuronic acid) of chondroitin sulfate was 2%.

### Reference Example 13

### N-[(1,1-Dimethylethoxy) carbonyl]-L-alanine Chloromethyl Ester

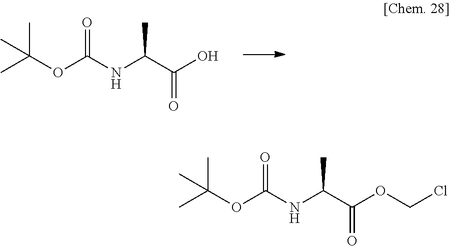

Under cooling on ice, a methylene chloride solution of 3.96 g (24 mmol) of chloromethyl chlorosulfonate was added dropwise to a water 40 mL-methylene chloride 40 mL mixed solution of 3.78 g (20 mmol) of N-[(1,1-dimethylethoxy) carbonyl]-L-alanine, 679 mg (2.0 mmol) of tetrabutylammonium hydrogen sulfate, and 6.72 g (80 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate, and the solvent was distilled off under reduced pressure. The residue was dissolved again in diethyl ether and washed with water. The water layer was extracted with a small amount of diethyl ether, and the extracted product was put together with the organic layer. The organic layer was washed with saturated saline and dried with anhydrous sodium sulfate, and then the solvent was distilled off under reduced pressure to obtain 4.33 g (91%) of the title compound.

¹H-NMR (CDCl₃, δ): 1.41 (3H, d, J=7 Hz), 1.44 (9H, s), 4.31-4.42 (1H, m), 4.96 (1H, br-s), 5.65 (1H, d, J=5 Hz), 5.84 (1H, d, J=5 Hz)

### Reference Example 14

### 2-[4-[2-[(4-Chlorobenzoyl) amino] ethyl] phenoxy]-2-methylpropanoic Acid [(S)-2-[[(1,1-dimethylethoxy) carbonyl] amino]-1-oxopropoxy] methyl Ester

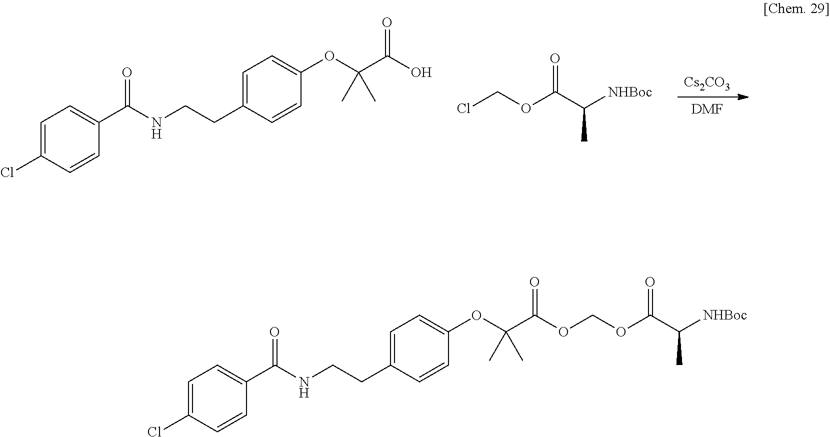

To a methanol suspension of 500 mg (1.38 mmol) of bezafibrate, 225 mg (0.691 mmol) of cesium carbonate was added, and the resulting mixture was stirred at room temperature for 30 minutes. The solvent of the mixed liquid was distilled off under reduced pressure, and the residue was dried using a vacuum pump overnight. The residue was dissolved in N,N-dimethylformamide, and an N,N-dimethylformamide solution of 378 mg (1.38 mmol) of N-[(1,1-dimethylethoxy) carbonyl]-L-alanine chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 682 mg (88%) of the title compound.

¹H-NMR (CDCl₃, δ): 1.33 (3H, d, J=7 Hz), 1.43 (9H, s), 1.59 (6H, s), 2.87 (2H, t, J=7 Hz), 2.67 (2H, q, J=7 Hz), 4.27-4.30 (1H, m), 4.95 (1H, br-s), 5.83 (1H, d, J=6 Hz), 5.91 (1H, d, J=6 Hz), 6.15 (1H, br-s), 6.81 (2H, d, J=9 Hz), 7.10 (2H, d, J=9 Hz), 7.38 (2H, d, J=9 Hz), 7.62 (2H, d, J=9 Hz)

### Reference Example 15

### 2-[4-[2-[(4-Chlorobenzoyl) amino] ethyl] phenoxy]-2-methylpropanoic Acid [(S)-2-amino-1-oxopropoxy] methyl Ester Hydrochloride

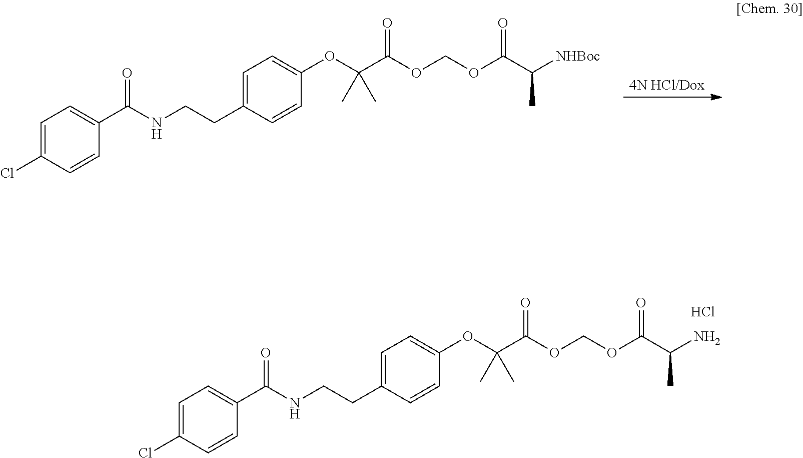 [SMILES: C[C@H](NC(=O)OC(C)(C)C)C(=O)OCOC(=O)C(C)(C)Oc1ccc(CCNC(=O)c2ccc(Cl)cc2)cc1 ; "4N HCl/Dox" ; C[C@H](N)C(=O)OCOC(=O)C(C)(C)Oc1ccc(CCNC(=O)c2ccc(Cl)cc2)cc1 ; Cl]

To 682 mg (1.21 mmol) of 2-[4-[2-[(4-chlorobenzoyl) amino] ethyl]phenoxy]-2-methylpropanoic acid [(S)-2-[[(1,1-dimethylethoxy) carbonyl] amino]-1-oxopropoxy] methyl ester, 4 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and then the precipitated solid was collected by filtration to obtain 580 mg (96%) of the title compound.

¹H-NMR (DMSO-d₆, δ): 1.39 (3H, d, J=7 Hz), 1.51 (6H, s), 2.78 (2H, d, J=8 Hz), 3.43-3.46 (2H, m), 4.17 (1H, q, J=7 Hz), 5.89 (1H, d, J=6 Hz), 5.96 (1H, d, J=6 Hz), 6.76 (2H, d, J=9 Hz), 7.15 (2H, d, J=9 Hz), 7.53 (2H, J=9 Hz), 7.85 (2H, d, J=9 Hz), 8.62 (3H, br-s), 8.70 (1H, t, J=6 Hz)

### Example 5

### [(S)-[2-(Bezafibrate-methoxy)-1-methyl-2-oxo] ethyl] amino-chondroitin sulfate Conjugate

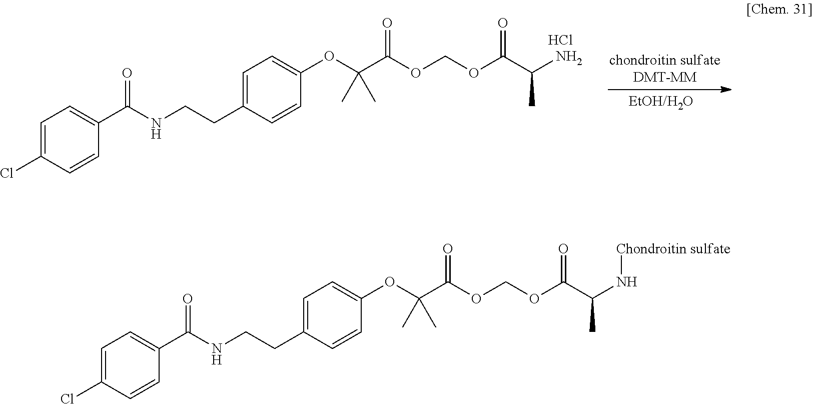

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 40 mg (0.080 mmol) of 2-[4-[2-[(4-chlorobenzoyl) amino]ethyl] phenoxy]-2-methylpropanoic acid [(S)-2-amino-1-oxopropoxy) methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 225 mg of the title compound. Based on values of integral in ¹H-NMR, the introduction ratio of bezafibrate per unit of whole disaccharide (glucuronic acid) of chondroitin sulfate was 10%.

### Reference Example 16

### 2-[[(1,1-Dimethylethoxy) carbonyl] amino]-2-methylpropanoic Acid Chloromethyl Ester

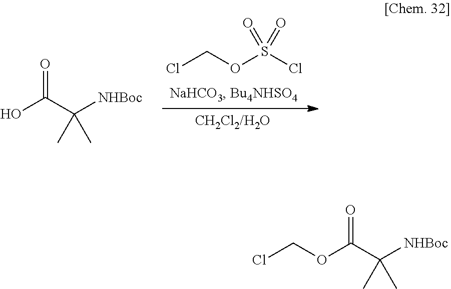 [SMILES: CC(C)(C)OC(=O)NC(C)(C)C(=O)O ; O=S(=O)(Cl)OCCl ; CC(C)(C)OC(=O)NC(C)(C)C(=O)OCCl]

Under cooling on ice, a methylene chloride solution of 1.95 g (11.8 mmol) of chloromethyl chlorosulfonate was added dropwise to a methylene chloride 15 mL-water 15 mL mixed solvent of 2.00 g (9.84 mmol) of 2-[[(1,1-dimethylethoxy) carbonyl]amino]-2-methylpropanoic acid, 3.31 g (39.4 mmol) of sodium hydrogen carbonate, and 334 mg (0.984 mmol) of tetrabutylammonium hydrogen sulfate. The temperature of the reaction solution was returned to room temperature, and the reaction solution was stirred overnight, and then extracted with diethyl ether. The obtained organic layer was washed with saturated saline, and then dried with anhydrous magnesium sulfate, and the solvent was distilled off under reduced pressure. The obtained residue was recrystallized from a diethyl ether-hexane mixed solvent to obtain 2.06 g (73%) of the title compound.

¹H-NMR (CDCl₃, δ): 1.43 (9H, s), 1.51 (6H, s), 4.90 (1H, br-s), 5.75 (2H, s)

### Reference Example 17

### 2-[4-[2-[(4-Chlorobenzoyl) amino] ethyl] phenoxy]-2-methylpropanoic Acid [2-[[(1,1-dimethylethoxy) carbonyl] amino]-2-methyl-1-oxopropoxy] methyl Ester

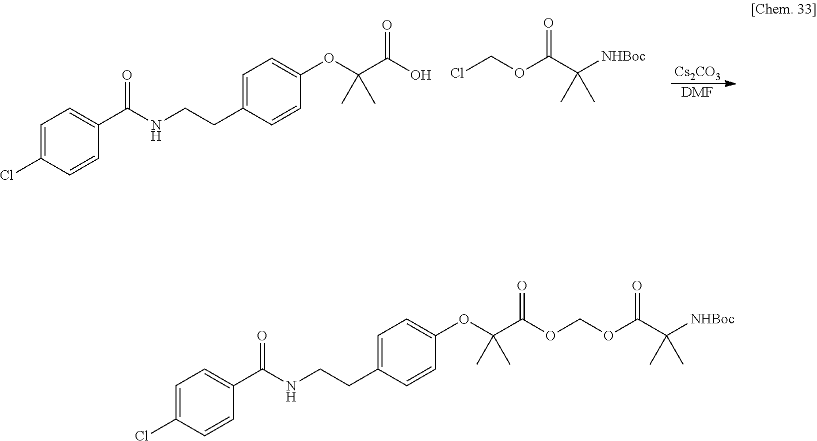

To a methanol suspension of 500 mg (1.38 mmol) of bezafibrate, 225 mg (0.691 mmol) of cesium carbonate was added, and the resulting mixture was stirred at room temperature for 30 minutes. The solvent of the mixed liquid was distilled off under reduced pressure, and the residue was dried using a vacuum pump overnight. The residue was dissolved in N,N-dimethylformamide, and an N,N-dimethylformamide solution of 397 mg (1.38 mmol) of 2-[[(1,1-dimethylethoxy) carbonyl] amino]-2-methylpropanoic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 655 mg (82%) of the title compound.

¹H-NMR (CDCl₃, δ): 1.42 (9H, s), 1.45 (6H, s), 1.59 (6H, s), 2.86 (2H, t, J=7 Hz), 3.67 (2H, q, J=7 Hz), 4.91 (1H, br-s), 5.86 (2H, s), 6.09 (1H, br-s), 6.83 (2H, d, J=9 Hz), 7.09 (2H, d, J=9 Hz), 7.38 (2H, d, J=9 Hz), 7.62 (2H, d, J=9 Hz)

### Reference Example 18

### 2-[4-[2-[(4-Chlorobenzoyl) amino] ethyl] phenoxy]-2-methylpropanoic Acid (2-amino-2-methyl-1-oxopropoxy) methyl Ester Hydrochloride

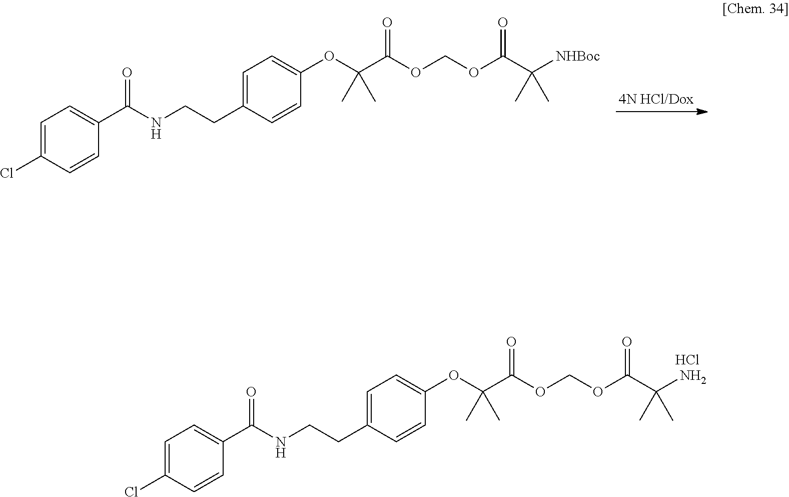 [SMILES: CC(C)(C)OC(=O)NC(C)(C)C(=O)OCOC(=O)C(C)(C)Oc1ccc(CCNC(=O)c2ccc(Cl)cc2)cc1 ; CC(C)(N)C(=O)OCOC(=O)C(C)(C)Oc1ccc(CCNC(=O)c2ccc(Cl)cc2)cc1 ; Cl]

To 655 mg (1.36 mmol) of 2-[4-[2-[(4-chlorobenzoyl) amino] ethyl]phenoxy]-2-methylpropanoic acid [2-[[(1,1-dimethylethoxy) carbonyl] amino]-2-methyl-1-oxopropoxy] methyl ester, 4 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and then the precipitated solid was collected by filtration to obtain 508 mg (87%) of the title compound.

¹H-NMR (DMSO-d₆, δ): 1.47 (6H, s), 1.51 (6H, s), 2.78 (2H, t, J=8 Hz), 3.41-3.46 (2H, m), 5.93 (2H, s), 6.76 (2H, d, J=9 Hz), 7.14 (2H, d, J=9 Hz), 7.53 (2H, J=9 Hz), 7.84 (2H, d, J=9 Hz), 8.68 (1H, br-s), 8.78 (3H, br)

### Example 6

### [2-(Bezafibrate-methoxy)-1,1-dimethyl-2-oxoethyl] amino-chondroitin sulfate Conjugate

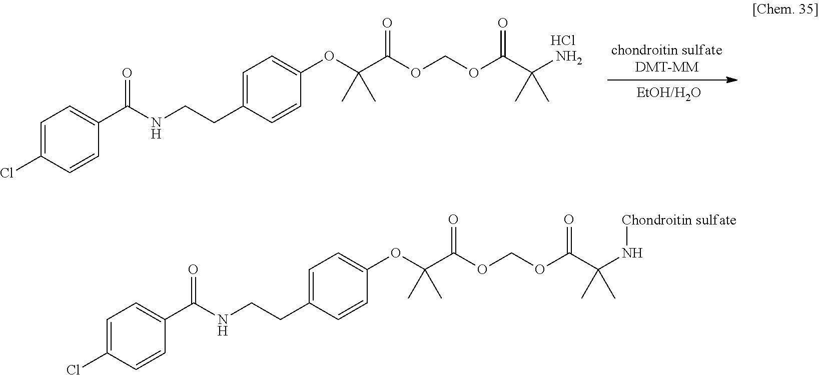

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 41 mg (0.080 mmol) of 2-[4-[2-[(4-chlorobenzoyl) amino]ethyl] phenoxy]-2-methylpropanoic acid (2-amino-2-methyl-1-oxopropoxy) methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 140 mg of the title compound. Based on values of integral in ¹H-NMR, the introduction ratio of bezafibrate per unit of whole disaccharide (glucuronic acid) of chondroitin sulfate was 3%.

### Reference Example 19

### (2E)-3-[4-(1H-Imidazol-1-ylmethyl) phenyl]-2-propenoic Acid [2-[[(1,1-dimethylethoxy) carbonyl] amino]-2-methyl-1-oxopropoxy] methyl Ester

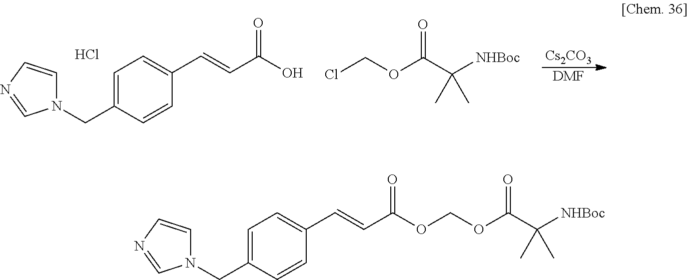

To a methanol solution of 300 mg (1.13 mmol) of ozagrel hydrochloride, 369 mg (1.13 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 285 mg (1.13 mmol) of 2-[[(1,1-dimethylethoxy) carbonyl] amino]-2-methylpropanoic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 471 mg (94%) of the title compound.

¹H-NMR (CDCl₃, δ): 1.40 (9H, s), 1.50 (6H, s), 4.95 (1H, br-s), 5.15 (2H, s), 5.92 (2H, s), 6.43 (1H, d, J=16 Hz), 6.90 (1H, t, J=2 Hz), 7.11 (1H, s), 7.16 (2H, d, J=9 Hz), 7.51 (2H, d, J=9 Hz), 7.56 (1H, s), 7.71 (1H, d, J=16 Hz)

### Reference Example 20

### (2E)-3-[4-(1H-Imidazol-1-ylmethyl) phenyl]-2-propenoic Acid (2-amino-2-methyl-1-oxopropoxy) methyl Ester Dihydrochloride

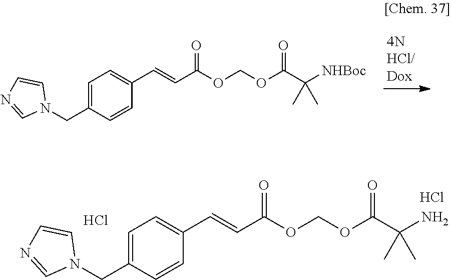 [SMILES: CC(C)(C)OC(=O)NC(C)(C)C(=O)OCOC(=O)/C=C/c1ccc(Cn2ccnc2)cc1 ; CC(C)(N)C(=O)OCOC(=O)/C=C/c1ccc(Cn2ccnc2)cc1 ; Cl ; Cl]

To 465 mg (1.05 mmol) of (2E)-3-[4-(1H-imidazol-1-ylmethyl) phenyl]-2-propenoic acid [2-[[(1,1-dimethylethoxy) carbonyl] amino]-2-methyl-1-oxopropoxy]methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 232 mg (53%) of the title compound.

¹H-NMR (DMSO-d₆, δ): 1.51 (6H, s), 5.51 (2H, s), 5.95 (2H, s), 6.74 (1H, d, J=16 Hz), 7.49 (2H, d, J=9 Hz), 7.70 (1H, t, J=2 Hz), 7.78 (1H, d, J=16 Hz), 7.82-7.84 (3H, m), 8.93 (3H, br-s), 9.40 (1H, s), 15.01 (1H, br)

### Example 7

### [1,1-Dimethyl-2-oxo-2-(ozagrel-methoxy) ethyl] amino-chondroitin Sulfate Conjugate

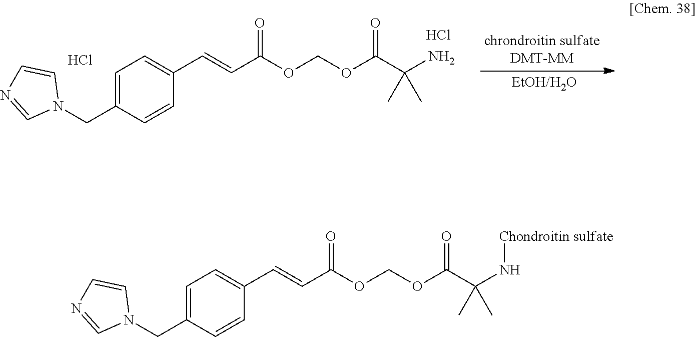

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 33 mg (0.080 mmol) of (2E)-3-[4-(1H-imidazol-1-ylmethyl) phenyl]-2-propenoic acid (2-amino-2-methyl-1-oxopropoxy) methyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 13 mL of 94% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 186 mg of the title compound. Based on values of integral in H-NMR, the introduction ratio of ozagrel per unit of whole disaccharide (glucuronic acid) of chondroitin sulfate was 1%.

### Reference Example 21

### N-[(1,1-Dimethylethoxy) carbonyl]-glycine Chloromethyl Ester

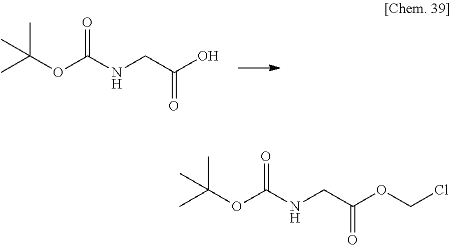

Under cooling on ice, a methylene chloride solution of 1.98 g (12 mmol) of chloromethyl chlorosulfonate was added dropwise to a water 20 mL-methylene chloride 20 mL mixed solution of 1.75 g (10 mmol) of N-[(1,1-dimethylethoxy) carbonyl]-glycine, 340 mg (1 mmol) of tetrabutylammonium hydrogen sulfate, and 3.36 g (40 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate. The solvent was condensed under reduced pressure, and the residue was purified by silica gel column chromatography (10% to 20% ethyl acetate/hexane) to obtain 1.85 g (83%) of the title compound.

¹H-NMR (CDCl₃, δ): 1.46 (9H, s), 4.00 (2H, d, J=6 Hz), 4.98 (1H, br-s), 5.75 (2H, s)

### Reference Example 22

### 2-[4-[2-[(4-Chlorobenzoyl) amino] ethyl] phenoxy]-2-methylpropanoic Acid [2-[[(1,1-dimethylethoxy) carbonyl] amino]-1-oxoethoxy] methyl Ester

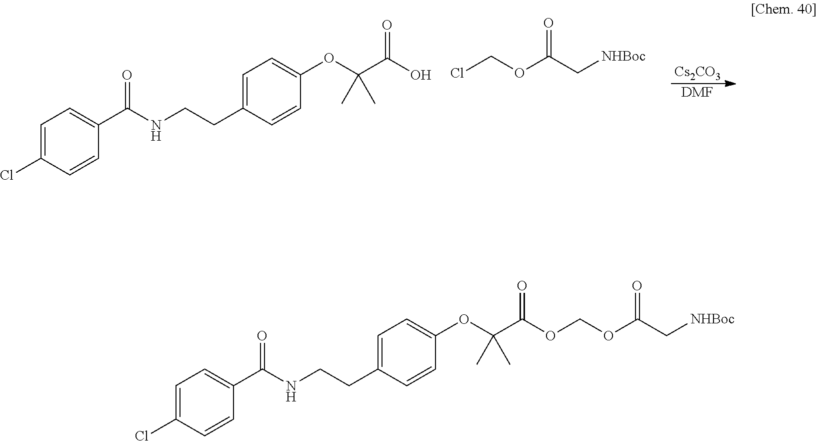

To a methanol suspension of 300 mg (0.829 mmol) of bezafibrate, 135 mg (0.415 mmol) of cesium carbonate was added, and the resulting mixture was stirred at room temperature for 30 minutes. The solvent of the mixed liquid was distilled off under reduced pressure, and the residue was dried using a vacuum pump overnight. The residue was dissolved in N,N-dimethylformamide, and an N,N-dimethylformamide solution of 185 mg (0.829 mmol) of N-[(1,1-dimethylethoxy) carbonyl]-glycine chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 160 mg (35%) of the title compound.

¹H-NMR (CDCl₃, δ): 1.43 (9H, s), 1.60 (6H, s), 2.87 (2H, t, J=7 Hz), 3.68 (2H, q, J=7 Hz), 3.86 (2H, d, J=6 Hz), 4.96 (1H, br-s), 5.87 (2H, s), 6.19 (1H, br-s), 6.80 (2H, d, J=9 Hz), 7.10 (2H, d, J=9 Hz), 7.38 (2H, d, J=9 Hz), 7.62 (2H, d, J=9 Hz)

### Reference Example 23

### 2-[4-[2-[(4-Chlorobenzoyl) amino] ethyl] phenoxy]-2-methylpropanoic Acid (2-amino-1-oxoethoxy) methyl Ester Hydrochloride

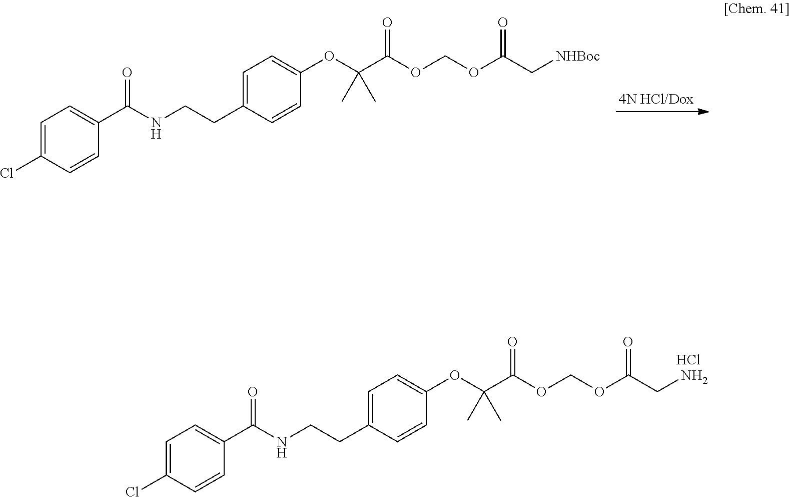 [SMILES: CC(C)(C)OC(=O)NCC(=O)OCOC(=O)C(C)(C)Oc1ccc(CCNC(=O)c2ccc(Cl)cc2)cc1 ; CC(C)(Oc1ccc(CCNC(=O)c2ccc(Cl)cc2)cc1)C(=O)OCOC(=O)CN ; Cl]

To 160 mg (0.291 mmol) of 2-[4-[2-[(4-chlorobenzoyl) amino] ethyl]phenoxy]-2-methylpropanoic acid [2-[[(1,1-dimethylethoxy) carbonyl] amino]-1-oxoethoxy] methyl ester, 4 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and then the precipitated solid was collected by filtration to obtain 95 mg (67%) of the title compound.

¹H-NMR (DMSO-d₆, δ): 1.50 (6H, s), 2.78 (2H, t, J=8 Hz), 3.43-3.47 (2H, m), 3.91 (2H, s), 5.92 (2H, s), 6.77 (2H, d, J=9 Hz), 7.15 (2H, d, J=9 Hz), 7.53 (2H, J=9 Hz), 7.84 (2H, d, J=91 Hz), 8.43 (3H, br-s), 8.67 (1H, t, J=6 Hz)

### Example 8

### [2-(Bezafibrate-methoxy)-2-oxoethyl] amino-chondroitin Sulfate Conjugate

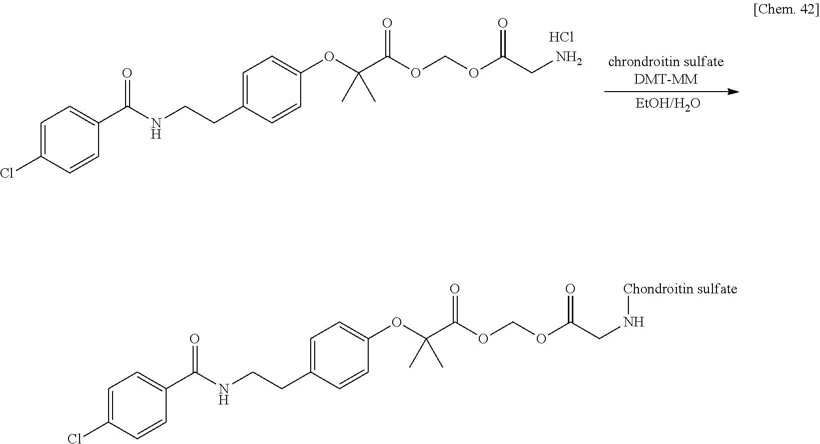

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 39 mg (0.080 mmol) of 2-[4-[2-[(4-chlorobenzoyl) amino]ethyl] phenoxy]-2-methylpropanoic acid (2-amino-1-oxoethoxy) methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 164 mg of the title compound. Based on values of integral in ¹H-NMR, the introduction ratio of bezafibrate per unit of whole disaccharide (glucuronic acid) of chondroitin sulfate was 16%.

### Reference Example 24

### 3-[[(1,1-Dimethylethoxy) carbonyl] amino] butanoic Acid Chloromethyl Ester

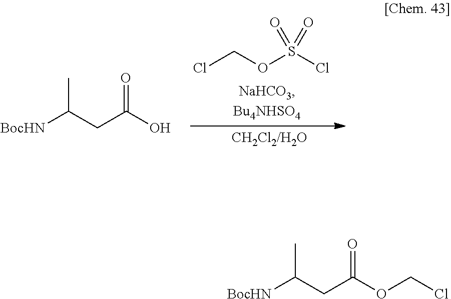 [SMILES: O=S(=O)(Cl)OCCl ; CC(CC(=O)O)NC(=O)OC(C)(C)C ; CC(CC(=O)OCCl)NC(=O)OC(C)(C)C]

Under cooling on ice, a methylene chloride solution of 1.95 g (11.8 mmol) of chloromethyl chlorosulfonate was added dropwise to a methylene chloride 20 mL-water 20 mL mixed solvent of 2.00 g (9.84 mmol) of 3-[[(1,1-dimethylethoxy) carbonyl]amino]-butanoic acid, 3.31 g (39.4 mmol) of sodium hydrogen carbonate, and 334 mg (0.984 mmol) of tetrabutylammonium hydrogen sulfate. The temperature of the reaction solution was returned to room temperature, and the reaction solution was stirred overnight, and then extracted with diethyl ether. The obtained organic layer was washed with saturated saline, and then dried with anhydrous magnesium sulfate, and the solvent was distilled off under reduced pressure. The residue was purified by silica gel column chromatography to obtain 1.95 g (79%) of the title compound.

¹H-NMR (CDCl₃, δ): 1.24 (3H, d, −J=7 Hz), 1.44 (9H, s), 2.56 (1H, dd, J=7, 16 Hz), 2.63 (1H, dd, J=6, 16 Hz), 4.03-4.10 (1H, m), 4.77 (1H, br-s), 5.69 (1H, d, J=6 Hz), 5.71 (1H, d, J=6 Hz)

### Reference Example 25

### (2E)-3-[4-(1H-Imidazol-1-ylmethyl) phenyl]-2-propenoic Acid [3-[[(1,1-dimethylethoxy) carbonyl] amino]-1-oxobutoxy] methyl Ester

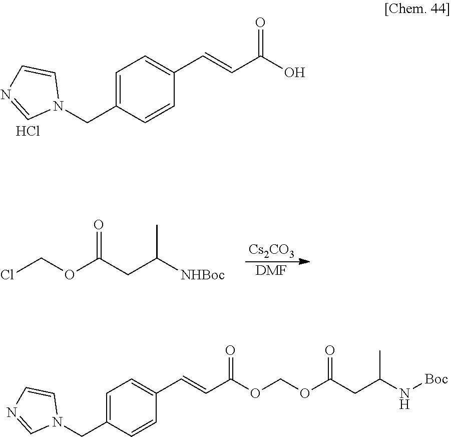

To a methanol solution of 350 mg (1.32 mmol) of ozagrel hydrochloride, 430 mg (1.32 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 332 mg (1.32 mmol) of 3-[[(1,1-dimethylethoxy) carbonyl] amino] butanoic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 480 mg (82%) of the title compound.

¹H-NMR (CDCl₃, δ): 1.23 (3H, d, J=7 Hz), 1.43 (9H, s), 2.55 (1H, dd, J=6, 16 Hz), 2.62 (1H, dd, J=5, 16 Hz), 4.06 (1H, br-s), 4.82 (1H, br-s), 5.15 (2H, s), 5.88 (2H, s), 6.43 (1H, d, J=16 Hz), 6.90 (1H, s), 7.11 (1H, s), 7.17 (2H, d, J=9 Hz), 7.52 (2H, d, J=9 Hz), 7.56 (1H, s), 7.74 (1H, d, J=16 Hz)

### Reference Example 26

### (2E)-3-[4-(1H-Imidazol-1-ylmethyl) phenyl]-2-propenoic Acid (3-amino-1-oxobutoxy) methyl Ester Dihydrochloride

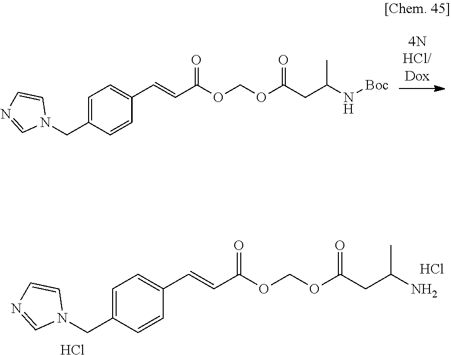 [SMILES: CC(CC(=O)OCOC(=O)/C=C/c1ccc(Cn2ccnc2)cc1)NC(=O)OC(C)(C)C ; CC(N)CC(=O)OCOC(=O)/C=C/c1ccc(Cn2ccnc2)cc1 ; Cl ; Cl]

To 450 mg (1.02 mmol) of (2E)-3-[4-(1H-imidazol-1-ylmethyl) phenyl]-2-propenoic acid [3-[[(1,1-dimethylethoxy) carbonyl] amino]-1-oxobutoxy] methyl ester, 3 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and then the precipitated solid was collected by filtration to obtain 370 mg (88%) of the title compound.

¹H-NMR (DMSO-d₆, δ): 1.26 (3H, d, J=7 Hz), 2.70 (1H, dd, J=8, 17 Hz), 2.92 (1H, dd, J=5, 17 Hz), 3.48-3.51 (1H, m), 5.50 (2H, s), 5.85 (1H, d, J=6 Hz), 5.87 (1H, d, J=6 Hz), 6.74 (1H, d, J=16 Hz), 7.48 (2H, d, J=8 Hz), 7.70 (1H, t, J=2 Hz), 7.76 (1H, d, J=16 Hz), 7.81-7.82 (3H, m), 8.35 (3H, br-s), 9.37 (1H, s), 14.94 (1H, br-s)

### Example 9

### [1-Methyl-3-oxo-3-(ozagrel-methoxy) propyl] amino-chondroitin Sulfate Conjugate

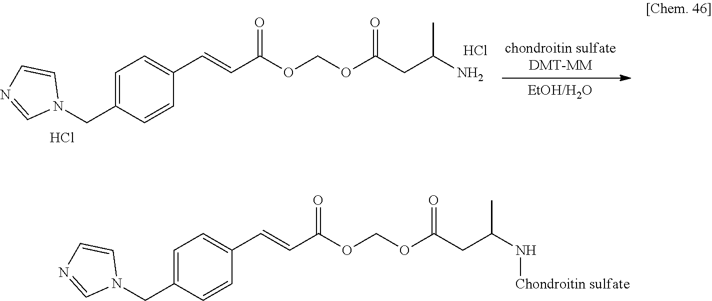

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 33 mg (0.080 mmol) of (2E)-3-[4-(1H-imidazol-1-ylmethyl) phenyl]-2-propenoic acid (3-amino-1-oxobutoxy) methyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 13 mL of 94% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 118 mg of the title compound. Based on values of integral in H-NMR, the introduction ratio of ozagrel per unit of whole disaccharide (glucuronic acid) of chondroitin sulfate was 5%.

### Reference Example 27

### 1-[[(1,1-Dimethylethoxy) carbonyl] amino] cyclopropane Carboxylic Acid Chloromethyl Ester

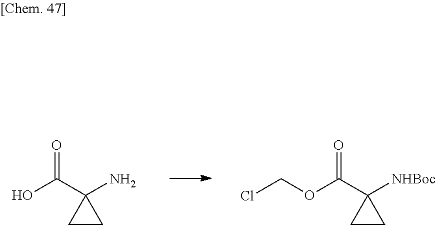 [SMILES: NC1(C(=O)O)CC1 ; CC(C)(C)OC(=O)NC1(C(=O)OCCl)CC1]

To a water-dioxane mixed solution of 5.0 g (49.5 mmol) of 1-aminocyclopropane carboxylic acid and 10.0 g (98.9 mmol) of triethylamine, 10.79 g (49.5 mmol) of tert-butyl dicarbonate was added, and the resulting mixture was stirred at room temperature overnight. The reaction solution was condensed under reduced pressure, and the residue was dissolved in ethyl acetate and washed with a 10% potassium hydrogen sulfate solution and saturated saline. The organic layer was dried with anhydrous sodium sulfate, and then the solvent was distilled off under reduced pressure. Hexane was added to the residue, and the resulting mixture was stirred. The precipitated crystals were collected by filtration to obtain 9.23 g (93%) of 1-[[(1,1-dimethylethoxy) carbonyl] amino] cyclopropane carboxylic acid. Under cooling on ice, a methylene chloride solution of 1.98 g (12 mmol) of chloromethyl chlorosulfonate was added dropwise to a water 20 mL-methylene chloride 20 mL mixed solution of 2.01 g (10 mmol) of the obtained 1-[[(1,1-dimethylethoxy) carbonyl] amino] cyclopropane carboxylic acid, 340 mg (1 mmol) of tetrabutylammonium hydrogen sulfate, and 3.36 g (40 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate, and the solvent was distilled off under reduced pressure. The residue was dissolved again in diethyl ether and washed with water. The water layer was extracted with a small amount of diethyl ether, and the extracted product was put together with the organic layer. The organic layer was washed with saturated saline and dried with anhydrous sodium sulfate, and then the solvent was distilled off under reduced pressure to obtain 2.41 g (96%) of the title compound.

¹H-NMR (CDCl₃, δ): 1.27 (2H, br-s), 1.45 (9H, s), 1.61 (2H, br-s), 5.13 (1H, br-s), 5.71 (2H, s)

### Reference Example 28

### (2E)-3-[4-(1H-Imidazol-1-ylmethyl) phenyl]-2-propenoic Acid [[[1-[[(1,1-dimethylethoxy) carbonyl] amino] cyclopropyl] carbonyl] oxyl Methyl Ester

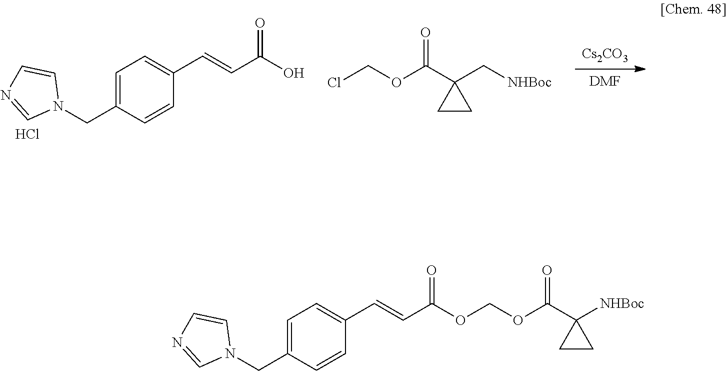

To a methanol solution of 350 mg (1.32 mmol) of ozagrel hydrochloride, 430 mg (1.32 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 348 mg (1.32 mmol) of 1-[[(1,1-dimethylethoxy) carbonyl] amino]cyclopropane carboxylic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 590 mg (98%) of the title compound.

¹H-NMR (CDCl₃, δ): 1.23-1.26 (2H, m), 1.44 (9H, s), 1.58-1.60 (2H, m), 5.14-5.15 (3H, m), 5.88 (2H, s), 6.42 (1H, d, J=16 Hz), 6.91 (1H, s), 7.11 (1H, s), 7.17 (2H, d, J=8 Hz), 7.51 (2H, d, J=8 Hz), 7.56 (1H, s), 7.72 (1H, d, J=16 Hz)

### Reference Example 29

### (2E)-3-[4-(1H-Imidazol-1-ylmethyl) phenyl]-2-propenoic Acid [[(1-aminocyclopropyl) carbonyl] oxy] methyl Ester Dihydrochloride

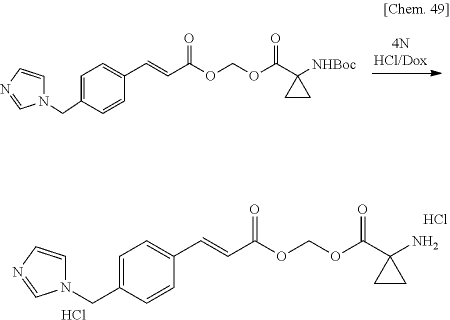 [SMILES: CC(C)(C)OC(=O)NC1(C(=O)OCOC(=O)/C=C/c2ccc(Cn3ccnc3)cc2)CC1 ; NC1(C(=O)OCOC(=O)/C=C/c2ccc(Cn3ccnc3)cc2)CC1 ; Cl ; Cl]

To 450 mg (1.02 mmol) of (2E)-3-[4-(1H-imidazol-1-ylmethyl) phenyl]-2-propenoic acid [[[1-[[(1,1-dimethylethoxy) carbonyl] amino] cyclopropyl] carbonyl]oxy] methyl ester, 3 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and then the precipitated solid was collected by filtration to obtain 370 mg (88%) of the title compound.

¹H-NMR (DMSO-d₆, δ): 1.43-1.46 (2H, m), 1.51-1.52 (2H, m), 5.48 (2H, s), 5.90 (2H, s), 6.73 (111, d, J=16 Hz), 7.47 (2H, d, J=8 Hz), 7.69 (1H, d, J=2 Hz), 7.75-7.83 (4H, m), 9.14 (3H, br-s), 9.28 (1H, s)

### Example 10

### [1-[(Ozagrel)-methoxy] carbonyl] cyclopropyl] amino-chondroitin Sulfate Conjugate

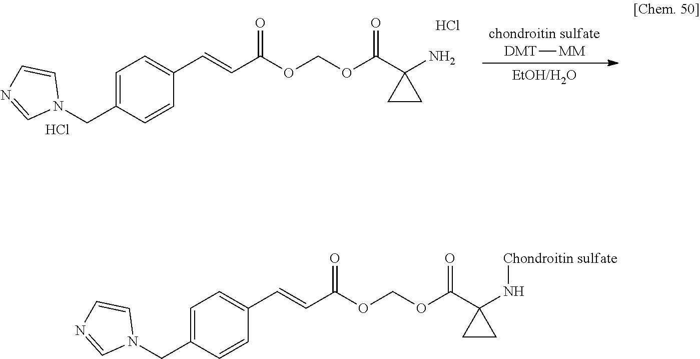

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 33 mg (0.080 mmol) of (2E)-3-[4-(1H-imidazol-1-ylmethyl) phenyl]-2-propenoic acid [[(1-aminocyclopropyl) carbonyl] oxy] methyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 13 mL of 94% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 135 mg of the title compound. Based on values of integral in ¹H-NMR, the introduction ratio of ozagrel per unit of whole disaccharide (glucuronic acid) of chondroitin sulfate was 5%.

### Reference Example 30

### N-[(1,1-Dimethylethoxy) carbonyl)]-L-tert-leucine Chloromethyl Ester

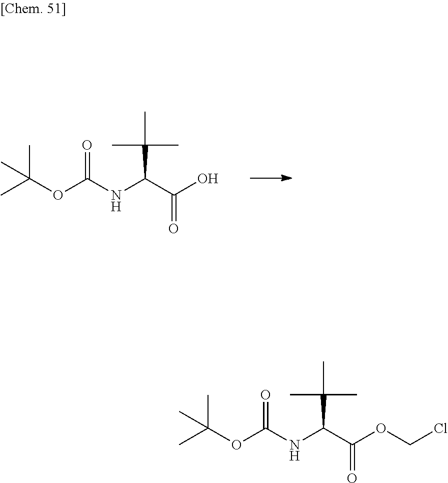 [SMILES: CC(C)(C)OC(=O)N[C@H](C(=O)O)C(C)(C)C ; CC(C)(C)OC(=O)N[C@H](C(=O)OCCl)C(C)(C)C]

Under cooling on ice, a methylene chloride solution of 825 mg (6.0 mmol) of chloromethyl chlorosulfonate was added dropwise to a water 10 mL-methylene chloride 10 mL mixed solution of 1.16 (5.0 mmol) of N-[(1,1-dimethylethoxy) carbonyl]-L-tert-leucine, 170 mg (0.5 mmol) of tetrabutylammonium hydrogen sulfate, and 1.68 g (20.0 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate, and the solvent was distilled off under reduced pressure. The residue was dissolved again in diethyl ether and washed with water. The water layer was extracted with a small amount of diethyl ether, and the extracted product was put together with the organic layer. The organic layer was washed with saturated saline and dried with anhydrous sodium sulfate, and then the solvent was distilled off under reduced pressure to obtain 1.31 gg (94%) of the title compound.

¹H-NMR (CDCl₃, δ): 1.02 (9H, s), 1.45 (9H, s), 4.13 (1H, d, J=8 Hz), 5.05 (1H, d, J=8 Hz), 5.61 (1H, d, J=6 Hz), 5.88 (1H, d, J=6 Hz)

### Reference Example 31

### (2E)-3-[4-(1H-Imidazol-1-ylmethyl) phenyl]-2-propenoic Acid [(2S)-3,3-dimethyl-2-[[(1,1-dimethylethoxy) carbonyl] amino]-1-oxobutoxy] methyl Ester

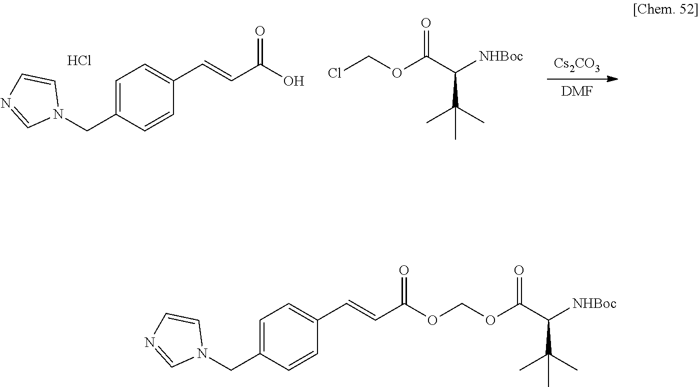

To a methanol solution of 300 mg (1.13 mmol) of ozagrel hydrochloride, 369 mg (1.13 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 317 mg (1.13 mmol) of N-[(1,1-dimethylethoxy) carbonyl]-L-tert-leucine chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 352 mg (66%) of the title compound.

¹H-NMR (CDCl₃, δ): 0.98 (9H, s), 1.43 (9H, s), 4.13 (1H, d, J=9 Hz), 5.08 (1H, d, J=9 Hz), 5.15 (2H, s), 5.87 (1H, d, J=6 Hz), 5.97 (1H, d, J=6 Hz), 6.42 (1H, d, J=16 Hz), 6.90 (1H, s), 7.11 (1H, s), 7.17 (2H, d, J=8 Hz), 7.52 (2H, d, J=8 Hz), 7.55 (1H, s), 7.73 (1H, d, J=16 Hz)

### Reference Example 32

### (2E)-3-[4-(1H-Imidazol-1-ylmethyl) phenyl]-2-propenoic Acid [(2S)-2-amino-3,3-dimethyl-1-oxobutoxy] methyl Ester Dihydrochloride

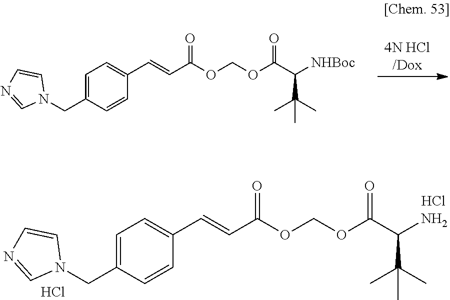 [SMILES: CC(C)(C)OC(=O)N[C@H](C(=O)OCOC(=O)/C=C/c1ccc(Cn2ccnc2)cc1)C(C)(C)C ; "4N HCl/Dox" ; CC(C)(C)[C@H](N)C(=O)OCOC(=O)/C=C/c1ccc(Cn2ccnc2)cc1 ; Cl ; Cl]

To 352 mg (0.746 mmol) of (2E)-3-[4-(1H-imidazol-1-ylmethyl) phenyl]-2-propenoic acid [(2S)-3,3-dimethyl-2-[[(1,1-dimethylethoxy) carbonyl] amino]-1-oxobutoxy] methyl ester, 3 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and then the precipitated solid was collected by filtration to obtain 282 mg (85%) of the title compound.

¹H-NMR (DMSO-d₆, δ): 1.00 (9H, s), 3.83 (1H, s), 5.49 (2H, s), 5.91 (1H, d, J=6 Hz), 5.99 (1H, d, J=6 Hz), 6.75 (1H, d, J=16 Hz), 7.48 (2H, d, J=8 Hz), 7.69 (1H, s), 7.77-7.83 (4H, m), 8.62 (3H, br-s), 9.31 (1H, s), 14.75 (1H, br-s)

### Example 11

### [(S)-2,2-Dimethyl-1-[(ozagrel) methoxy) carbonyl] propyl] amino-chondroitin Sulfate Conjugate

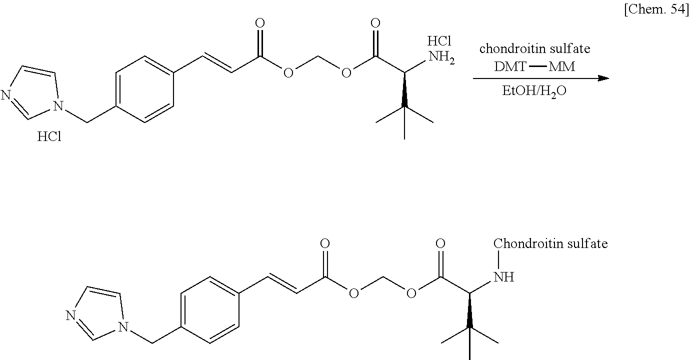

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 35 mg (0.080 mmol) of (2E)-3-[4-(H-imidazol-1-ylmethyl) phenyl]-2-propenoic acid [(2S)-2-amino-3,3-dimethyl-1-oxobutoxy] methyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 13 mL of 94% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 148 mg of the title compound. Based on values of integral in ¹H-NMR, the introduction ratio of ozagrel per unit of whole disaccharide (glucuronic acid) of chondroitin sulfate was 7%.

### Reference Example 33

### N-[(1,1-Dimethylethoxy) carbonyl)]-L-valine Chloromethyl Ester

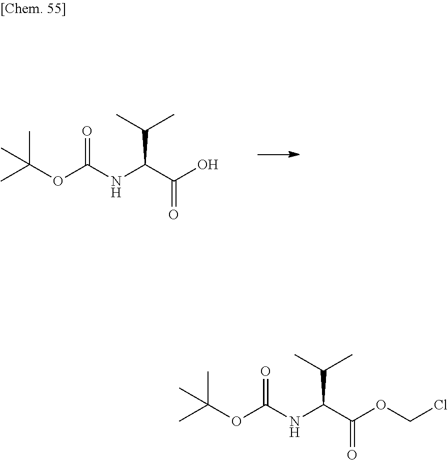 [SMILES: CC(C)[C@H](NC(=O)OC(C)(C)C)C(=O)O ; CC(C)[C@H](NC(=O)OC(C)(C)C)C(=O)OCCl]

Under cooling on ice, a methylene chloride solution of 825 mg (6.0 mmol) of chloromethyl chlorosulfonate was added dropwise to a water 10 mL-methylene chloride 10 mL mixed solution of 1.09 (5.0 mmol) of N-[(1,1-dimethylethoxy) carbonyl]-L-valine, 170 mg (0.5 mmol) of tetrabutylammonium hydrogen sulfate, and 1.68 g (20.0 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate, and the solvent was distilled off under reduced pressure. The residue was dissolved again in diethyl ether and washed with water. The water layer was extracted with a small amount of diethyl ether, and the extracted product was put together with the organic layer. The organic layer was washed with saturated saline and dried with anhydrous sodium sulfate, and then the solvent was distilled off under reduced pressure to obtain 1.26 gg (95%) of the title compound.

¹H-NMR (CDCl₃, δ): 0.93 (3H, d, J=7 Hz), 1.01 (3H, d, J=7 Hz), 1.45 (9H, s), 2.11-2.24 (1H, m), 4.21-4.31 (1H, m), 4.97 (1H, br-s), 5.62 (1H, d, J=6 Hz), 5.88 (1H, d, J=6 Hz)

### Reference Example 34

### (2E)-3-[4-(1H-Imidazol-1-ylmethyl) phenyl]-2-propenoic Acid [(2S)-2-[[(1,1-dimethylethoxy) carbonyl] amino]-3-methyl-1-oxobutoxy] methyl Ester

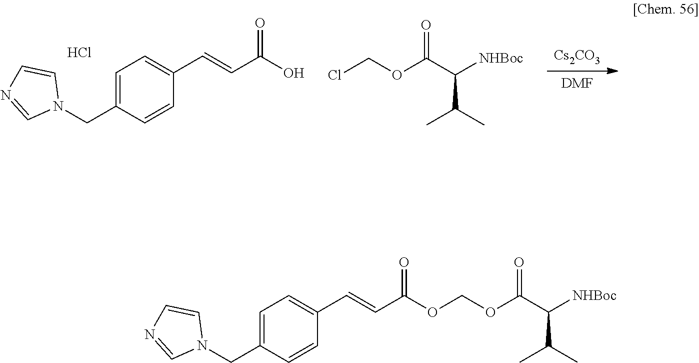

To a methanol solution of 300 mg (1.13 mmol) of ozagrel hydrochloride, 369 mg (1.13 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 301 mg (1.13 mmol) of N-[(1,1-dimethylethoxy) carbonyl]-L-valine chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 286 mg (55%) of the title compound.

¹H-NMR (CDCl₃, δ): 0.89 (3H, d, J=7 Hz), 0.97 (3H, d, J=7 Hz), 1.44 (9H, s), 2.15-2.18 (1H, m), 4.12 (1H, dd, J=5, 9 Hz), 4.99 (1H, d, J=9 Hz), 5.15 (2H, s), 5.88 (1H, d, J=6 Hz), 5.97 (1H, d, J=6 Hz), 6.42 (1H, d, J=16 Hz), 6.90 (1H, t, J=1 Hz), 7.11 (1H, s), 7.17 (2H, d, J=7 Hz), 7.52 (2H, J=9 Hz), 7.56 (1H, s), 7.73 (1H, d, J=16 Hz)

### Reference Example 35

### (2E)-3-[4-(1H-Imidazol-1-ylmethyl) phenyl]-2-propenoic Acid [(2S)-2-amino-3-methyl-1-oxobutoxy] methyl Ester Dihydrochloride

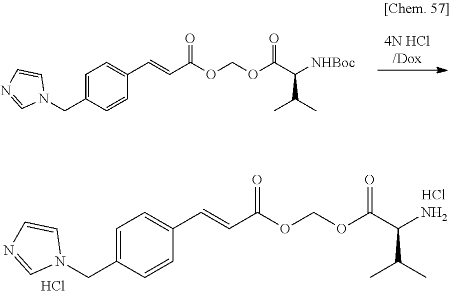 [SMILES: CC(C)[C@H](NC(=O)OC(C)(C)C)C(=O)OCOC(=O)/C=C/c1ccc(Cn2ccnc2)cc1 ; "4N HCl/Dox" ; CC(C)[C@H](N)C(=O)OCOC(=O)/C=C/c1ccc(Cn2ccnc2)cc1 ; Cl ; Cl]

To 286 mg (0.652 mmol) of (2E)-3-[4-(H-imidazol-1-ylmethyl) phenyl]-2-propenoic acid [(2S)-2-[[(1,1-dimethylethoxy) carbonyl] amino]-3-methyl-1-oxobutoxy] methyl ester, 3 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and then the precipitated solid was collected by filtration to obtain 217 mg (81%) of the title compound.

¹H-NMR (DMSO-d₆, δ): 0.94 (3H, d, J=7 Hz), 0.98 (3H, d, J=7 Hz), 2.19-2.21 (1H, m), 3.98 (1H, d, J=4 Hz), 5.49 (2H, s), 5.91 (1H, d, J=6 Hz), 6.00 (1H, d, J=6 Hz), 6.74 (1H, d, J=16 Hz), 7.48 (2H, d, J=9 Hz), 7.70 (1H, t, J=1 Hz), 7.77-7.83 (4H, m), 8.68 (3H, br-s), 9.34 (1H, t, J=1 Hz), 14.83 (1H, br-s)

### Example 12

### [(1S)-2-Methyl-1-[(ozagrel) methoxy) carbonyl] propyl] amino-chondroitin Sulfate Conjugate

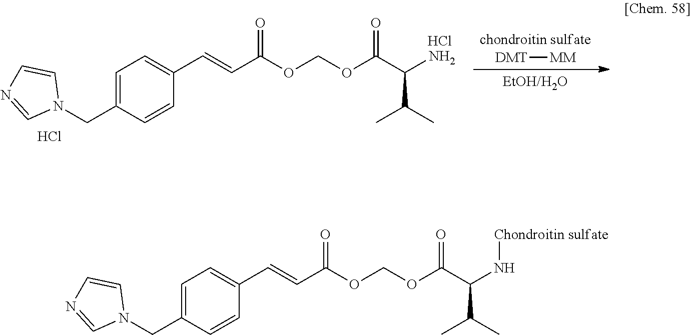

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 34 mg (0.080 mmol) of (2E)-3-[4-(1H-imidazol-1-ylmethyl) phenyl]-2-propenoic acid [(2S)-2-amino-3-methyl-1-oxobutoxy] methyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 13 mL of 94% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 150 mg of the title compound. Based on values of integral in ¹H-NMR, the introduction ratio of ozagrel per unit of whole disaccharide (glucuronic acid) of chondroitin sulfate was 6%.

### Reference Example 36

### 1-[[(1,1-Dimethylethoxy) carbonyl] amino] cyclopentane Carboxylic Acid Chloromethyl Ester

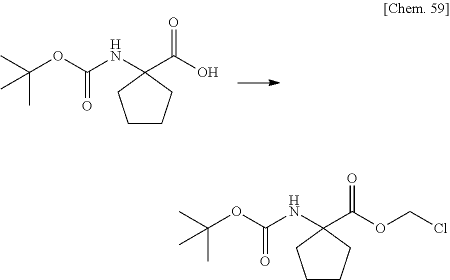 [SMILES: CC(C)(C)OC(=O)NC1(C(=O)O)CCCC1 ; CC(C)(C)OC(=O)NC1(C(=O)OCCl)CCCC1]

Under cooling on ice, a methylene chloride solution of 2.60 g (17.44 mmol) of chloromethyl chlorosulfonate was added dropwise to a water 20 mL-methylene chloride 20 mL mixed solution of 2.00 g (8.72 mmol) of 1-[[(1,1-dimethylethoxy) carbonyl] amino] cyclopentane carboxylic acid, 296 mg (0.87 mmol) of tetrabutylammonium hydrogen sulfate, and 2.93 g (34.88 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate. The solvent was condensed under reduced pressure, and the residue was purified by silica gel column chromatography (5% to 40% ethyl acetate/hexane) to obtain 2.25 g (93%) of the title compound.

¹H-NMR (CDCl₃, δ): 1.44 (9H, s), 1.77-1.80 (4H, m), 1.88-1.90 (2H, m), 2.22-2.28 (2H, m), 4.85 (1H, br-s), 5.75 (2H, s)

### Reference Example 37

### (2E)-3-[4-(1H-Imidazol-1-ylmethyl) phenyl]-2-propenoic Acid [[[1-[[(1,1-dimethylethoxy) carbonyl] amino] cyclopentyl] carbonyl] oxy] methyl Ester

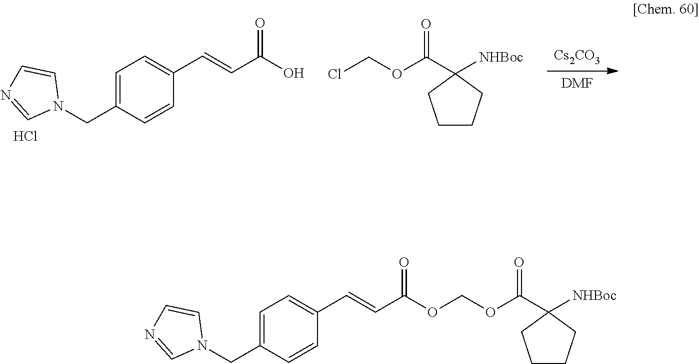

To a methanol solution of 300 mg (1.13 mmol) of ozagrel hydrochloride, 369 mg (1.13 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 314 mg (1.13 mmol) of 1-[[(1,1-dimethylethoxy) carbonyl] amino]cyclopentane carboxylic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 469 mg (88%) of the title compound.

¹H-NMR (CDCl₃, δ): 1.41 (9H, s), 1.76-1.79 (4H, m), 1.88-1.91 (2H, m), 2.21-2.27 (2H, m), 4.88 (1H, br-s), 5.15 (2H, s), 5.92 (2H, s), 6.42 (1H, d, J=16 Hz), 6.90-6.91 (1H, m), 7.11 (1H, s), 7.16 (2H, d, J=8 Hz), 7.51 (2H, d, J=8 Hz), 7.55 (1H, s), 7.72 (1H, d, J=16 Hz)

### Reference Example 38

### (2E)-3-[4-(1H-Imidazol-1-ylmethyl) phenyl]-2-propenoic Acid [[(1-aminocyclopentyl) carbonyl] oxy] methyl Ester Dihydrochloride

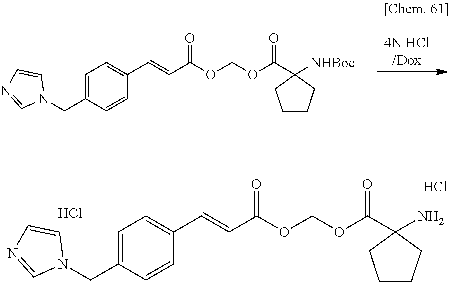 [SMILES: CC(C)(C)OC(=O)NC1(C(=O)OCOC(=O)/C=C/c2ccc(Cn3ccnc3)cc2)CCCC1 ; "4N HCl/Dox" ; NC1(C(=O)OCOC(=O)/C=C/c2ccc(Cn3ccnc3)cc2)CCCC1 ; Cl ; Cl]

To 469 mg (1.00 mmol) of (2E)-3-[4-(1H-imidazol-1-ylmethyl) phenyl]-2-propenoic acid [[[1-[[(1,1-dimethylethoxy) carbonyl] amino] cyclopentyl] carbonyl]oxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 360 mg (82%) of the title compound.

¹H-NMR (DMSO-d₆, δ): 1.69-1.76 (2H, m), 1.85-1.99 (4H, m), 2.10-2.15 (2H, m), 5.50 (2H, s), 5.94 (2H, s), 6.74 (1H, d, J=16 Hz), 7.48 (2H, d, J=8 Hz), 7.69 (1H, s), 7.78 (1H, d, J=16 Hz), 7.81-7.83 (3H, m), 8.88 (3H, br-s), 9.36 (1H, s), 14.92 (1H, br-s)

### Example 13

### [1-[(Ozagrel)-methoxy] carbonyl] cyclopentyl] amino-chondroitin Sulfate Conjugate

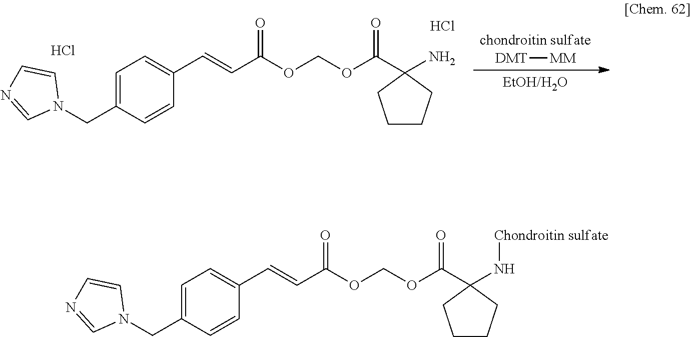

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 35 mg (0.080 mmol) of (2E)-3-[4-(1H-imidazol-1-ylmethyl) phenyl]-2-propenoic acid [[(1-aminocyclopentyl) carbonyl] oxy] methyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 13 mL of 94% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 138 mg of the title compound. Based on values of integral in ¹H-NMR, the introduction ratio of ozagrel per unit of whole disaccharide (glucuronic acid) of chondroitin sulfate was 2%.

### Reference Example 39

### 2-[[[(1,1-Dimethylethoxy) carbonyl] amino] methyl]-3-methylbutanoic Acid Ethyl Ester

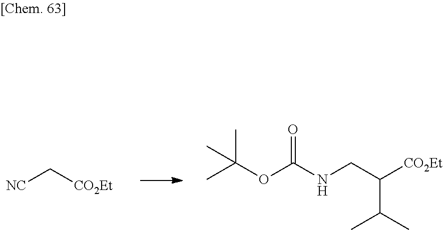 [SMILES: CCOC(=O)CC#N ; CCOC(=O)C(CNC(=O)OC(C)(C)C)C(C)C]

To 60 mL of dry ethanol, 1.38 g (60 mmol) of metallic sodium was added and dissolved therein. Thereafter, 5.66 g (50 mmol) of cyano ethyl acetate was added thereto at room temperature, and the resulting mixture was stirred for 15 minutes. To the reaction solution, a dry ethanol 15 mL solution of 10.71 g (63 mmol) of 2-iodopropane was slowly added at room temperature, and the resulting mixture was stirred for three hours. The reaction solution was heated under reflux for one hour. Thereafter, the temperature of the reaction solution was returned to room temperature, and 10% sodium hydrogen sulfate was added thereto to stop the reaction. Diethyl ether was added to the reaction solution, and the water layer was extracted with diethyl ether. The extracted product was put together with the organic layer, and the resulting mixture was washed with 10% sodium thiosulfate and saturated saline. The organic layer was dried with anhydrous sodium sulfate, and then the solvent was distilled off under reduced pressure to obtain a crude 2-cyano-3-methylbutanoic acid ethyl ester. The obtained crude 2-cyano-3-methylbutanoic acid ethyl ester was dissolved in 200 mL of methanol, and 23.8 g (0.1 mol) of cobalt chloride hexahydrate was added thereto. While the mixed liquid was cooled in a water bath, 18.9 g (0.5 mol) of sodium borohydride was added in small portions to the mixed liquid, and then the resulting mixture was stirred at room temperature for 30 minutes. Under cooling on ice, 200 mL of 6 N hydrochloric acid and 225 mL of 2 N hydrochloric acid were added to the reaction solution, and the resulting mixture was stirred at room temperature for two hours. To the reaction solution, 200 g (1.98 mol) of triethylamine was added, and the resulting mixture was stirred for one hour. Thereafter, 11.35 g (52 mmol) of tert-butyl dicarbonate was added thereto, and the resulting mixture was stirred at room temperature overnight. The insolubles were collected by filtration and washed with ethyl acetate three times. The obtained filtrates were put together, and the organic layer was isolated. Thereafter, the water layer portion was extracted with ethyl acetate. The extracted product was put together with the organic layer, and the resulting mixture was washed with saturated saline. The organic layer was dried with anhydrous sodium sulfate, and then the solvent was distilled off under reduced pressure. The residue was purified by silica gel column chromatography (3% to 10% ethyl acetate/hexane) to obtain 7.02 g (54%) of the title compound.

¹H-NMR (CDCl₃, δ): 0.95 (3H, d, J=7 Hz), 0.98 (3H, d, J=7 Hz), 1.27 (3H, d, J=7 Hz), 1.44 (9H, s), 1.90-2.00 (1H, m), 2.35-2.48 (1H, m), 3.13-3.47 (2H, m), 4.11-4.23 (2H, m), 4.82 (1H, br-s)

### Reference Example 40

### 2-[[[(1,1-Dimethylethoxy) carbonyl] amino] methyl]-3-methylbutanoic Acid Chloromethyl Ester

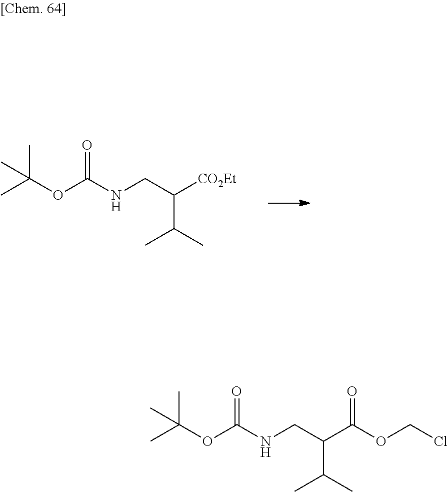 [SMILES: CCOC(=O)C(CNC(=O)OC(C)(C)C)C(C)C ; CC(C)C(CNC(=O)OC(C)(C)C)C(=O)OCCl]

To a tetrahydrofuran 50 mL solution of 7.02 g (27 mmol) of 2-[[[(1,1-dimethylethoxy) carbonyl] amino] methyl]-3-methylbutanoic acid ethyl ester, 56 mL of a 2 N sodium hydroxide aqueous solution was added, and the resulting mixture was heated under reflux overnight. The reaction solution was condensed under reduced pressure, and then diethyl ether was added to the residue. The resulting mixture was extracted with water two times. Potassium hydrogen sulfate was added to the water layer such that the resulting mixture became acidic, and then the mixture was extracted with ethyl acetate. The organic layer was washed with saturated saline, and then the solvent was distilled off under reduced pressure. Hexane was added to the residue, and the resulting mixture was stirred. The precipitated crystals were collected by filtration to obtain 4.19 g (68%) of 2-[[[(1,1-dimethylethoxy) carbonyl] amino] methyl]-3-methylbutanoic acid. Under cooling on ice, a methylene chloride solution of 3.58 g (22 mmol) of chloromethyl chlorosulfonate was added dropwise to a water 40 mL-methylene chloride 40 mL mixed solution of 4.19 g (18 mmol) of the obtained 2-[[[(1,1-dimethylethoxy) carbonyl] amino] methyl]-3-methylbutanoic acid, 615 mg (1.8 mmol) of tetrabutylammonium hydrogen sulfate, and 6.08 g (72 mmol) of sodium hydrogen carbonate. The temperature of the reaction solution was returned to room temperature, and the reaction solution was stirred overnight. The methylene chloride layer of the reaction solution was isolated and washed with saturated saline. Thereafter, the organic layer was dried with anhydrous sodium sulfate, and the solvent was distilled off under reduced pressure. The residue was dissolved again in diethyl ether and washed with water. The water layer was extracted with a small amount of diethyl ether, and the extracted product was put together with the organic layer. The organic layer was washed with saturated saline and dried with anhydrous sodium sulfate, and the solvent was distilled off under reduced pressure to obtain 4.78 g (94%) of the title compound.

¹H-NMR (CDCl₃, δ): 0.98 (3H, d, J=7 Hz), 0.99 (3H, d, J=7 Hz), 1.43 (9H, s), 1.94-2.06 (1H, m), 2.45-2.59 (1H, m), 3.18-3.52 (2H, m), 4.77 (1H, br-s), 5.68 (1H, d, J=6 Hz), 5.79 (1H, d, J=6 Hz)

### Reference Example 41

### (2E)-3-[4-(1H-Imidazol-1-ylmethyl) phenyl]-2-propenoic Acid [2-[[[(1,1-dimethylethoxy) carbonyl] amino] methyl]-3-methyl-1-oxobutoxy] methyl Ester

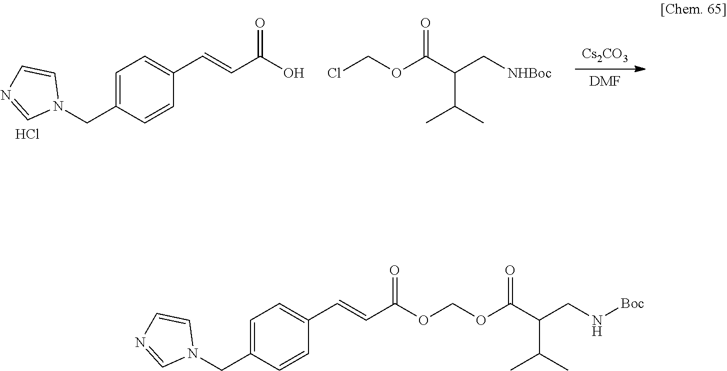

To a methanol solution of 300 mg (1.13 mmol) of ozagrel hydrochloride, 369 mg (1.13 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 317 mg (1.13 mmol) of 2-[[[(1,1-dimethylethoxy) carbonyl] amino] methyl]-3-methylbutanoic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 481 mg (90%) of the title compound.

¹H-NMR (CDCl₃, δ): 0.96 (3H, d, J=7 Hz), 0.97 (3H, d, J=7 Hz), 1.40 (9H, s), 1.97-2.04 (1H, m), 2.51-2.55 (1H, m), 3.24 (1H, ddd, J=6, 10, 14 Hz), 3.44-3.49 (1H, m), 4.84 (1H, br-s), 5.15 (2H, s), 5.88 (1H, d, J=6 Hz), 5.94 (1H, d, J=6 Hz), 6.43 (1H, d, J=16 Hz), 6.90 (1H, t, J=1 Hz), 7.11 (1H, s), 7.17 (2H, d, J=9 Hz), 7.52 (2H, d, J=9 Hz), 7.55 (1H, s), 7.75 (1H, d, J=16 Hz)

### Reference Example 42

### (2E)-3-[4-(1H-Imidazol-1-ylmethyl) phenyl]-2-propenoic Acid [2-(aminomethyl)-3-methyl-1-oxobutoxy] methyl Ester Dihydrochloride

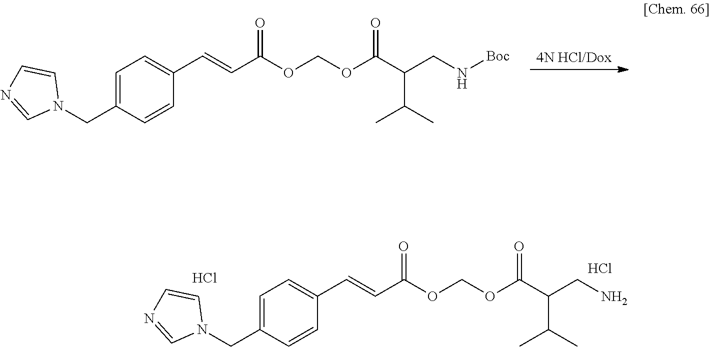 [SMILES: CC(C)C(CNC(=O)OC(C)(C)C)C(=O)OCOC(=O)/C=C/c1ccc(Cn2ccnc2)cc1 ; "4N HCl/Dox" ; CC(C)C(CN)C(=O)OCOC(=O)/C=C/c1ccc(Cn2ccnc2)cc1 ; Cl ; Cl]

To 441 mg (1.02 mmol) of (2E)-3-[4-(1H-imidazol-1-ylmethyl) phenyl]-2-propenoic acid [2-[[[(1,1-dimethylethoxy) carbonyl] amino] methyl]-3-methyl-1-oxobutoxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 292 mg (64%) of the title compound.

¹H-NMR (DMSO-d₆, δ): 0.86 (3H, d, J=7 Hz), 0.90 (3H, d, J=7 Hz), 1.96-2.04 (1H, m), 2.70-2.74 (1H, m), 2.94 (1H, br-s), 3.06 (1H, br-s), 5.49 (2H, s), 5.82 (1H, d, J=6 Hz), 5.93 (1H, d, J=6 Hz), 6.73 (1H, d, J=16 Hz), 7.48 (2H, d, J=8 Hz), 7.70 (1H, t, J=2 Hz), 7.76 (1H, d, J=16 Hz), 7.81-7.82 (3H, m), 8.25 (3H, br-s), 9.34 (1H, s), 14.89 (1H, br-s)

### Example 14

### [3-Methyl-2-[[(ozagrel-methoxy] carbonyl] butyl] amino-chondroitin Sulfate Conjugate

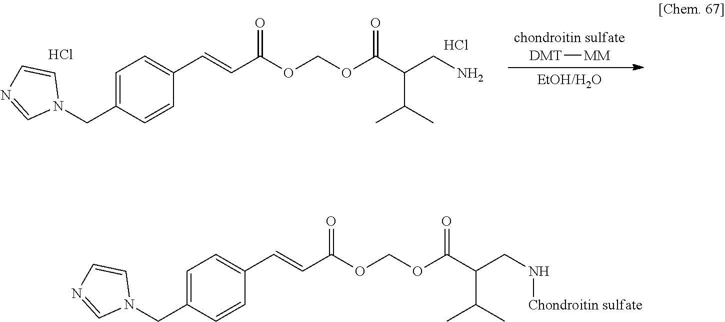

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 35 mg (0.080 mmol) of (2E)-3-[4-(1H-imidazol-1-ylmethyl) phenyl]-2-propenoic acid [2-(aminomethyl)-3-methyl-1-oxobutoxy] methyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 13 mL of 94% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 165 mg of the title compound. Based on values of integral in H-NMR, the introduction ratio of ozagrel per unit of whole disaccharide (glucuronic acid) of chondroitin sulfate was 8%.

### Reference Example 43

### 1-[[(1,1-Dimethylethoxy) carbonyl] amino] cyclohexane Carboxylic Acid Chloromethyl Ester

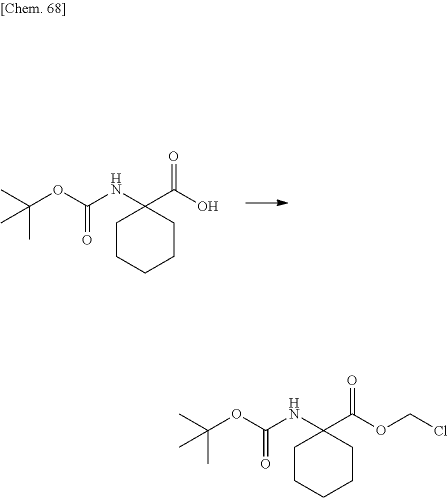 [SMILES: CC(C)(C)OC(=O)NC1(C(=O)O)CCCCC1 ; CC(C)(C)OC(=O)NC1(C(=O)OCCl)CCCCC1]

Under cooling on ice, a methylene chloride solution of 2.88 g (19.31 mmol) of chloromethyl chlorosulfonate was added dropwise to a water 20 mL-methylene chloride 20 mL mixed solution of 2.35 g (9.66 mmol) of 1-[[(1,1-dimethylethoxy) carbonyl] amino] cyclohexane carboxylic acid, 329 mg (0.97 mmol) of tetrabutylammonium hydrogen sulfate, and 23.24 g (38.64 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate. The solvent was condensed under reduced pressure, and the residue was purified by silica gel column chromatography (ethyl acetate/hexane) to obtain 2.59 g (92%) of the title compound.

¹H-NMR (CDCl₃, δ): 1.25-1.68 (15H, m), 1.86 (2H, dt, J=7, 13 Hz), 1.95-1.98 (2H, m), 4.73 (1H, br-s), 5.74 (2H, s)

### Reference Example 44

### (2E)-3-[4-(1H-Imidazol-1-ylmethyl) phenyl]-2-propenoic Acid [[[1-[[(1,1-dimethylethoxy) carbonyl] amino] cyclohexyl] carbonyl] oxy] methyl Ester

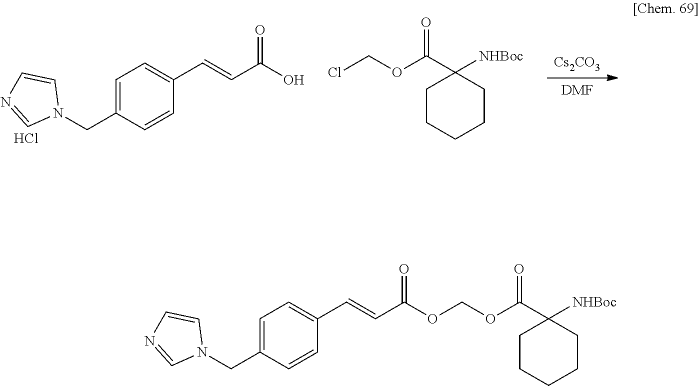

To a methanol solution of 300 mg (1.13 mmol) of ozagrel hydrochloride, 369 mg (1.13 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 330 mg (1.13 mmol) of 1-[[(1,1-dimethylethoxy) carbonyl] amino]cyclohexane carboxylic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 462 mg (84%) of the title compound.

¹H-NMR (CDCl₃, δ): 1.23-1.37 (1H, m), 1.40 (9H, s), 1.42-1.53 (2H, m), 1.58-1.65 (3H, m), 1.81-1.87 (2H, m), 1.96-1.98 (2H, m), 4.76 (1H, br-s), 5.15 (2H, s), 5.91 (2H, s), 6.42 (1H, d, J=16 Hz), 6.90 (1H, t, J=1 Hz), 7.11 (1H, s), 7.16 (2H, d, J=9 Hz), 7.50 (2H, d, J=9 Hz), 7.56 (1H, s), 7.72 (1H, d, J=16 Hz)

### Reference Example 45

### (2E)-3-[4-(1H-Imnidazol-1-ylmethyl) phenyl]-2-propenoic Acid [[(1-aminocyclohexyl) carbonyl] oxy] methyl Ester Dihydrochloride

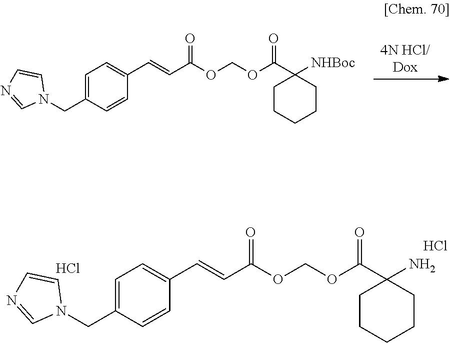 [SMILES: CC(C)(C)OC(=O)NC1(C(=O)OCOC(=O)/C=C/c2ccc(Cn3ccnc3)cc2)CCCCC1 ; "4N HCl/Dox" ; NC1(C(=O)OCOC(=O)/C=C/c2ccc(Cn3ccnc3)cc2)CCCCC1 ; Cl ; Cl]

To 453 mg (0.955 mmol) of (2E)-3-[4-(1H-imidazol-1-ylmethyl) phenyl]-2-propenoic acid [[[1-[[(1,1-dimethylethoxy) carbonyl] amino] cyclohexyl] carbonyl]oxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 361 mg (83%) of the title compound.

¹H-NMR (DMSO-d₆, δ): 1.36-1.44 (2H, m), 1.48-1.54 (2H, m), 1.65-1.71 (2H, m), 1.83 (2H, ddd, J=4, 8, 14 Hz), 1.95 (2H, ddd, J=4, 9, 14 Hz), 5.51 (2H, s), 5.95 (2H, s), 6.74 (1H, d, J=16 Hz), 7.49 (2H, d, J=8 Hz), 7.70 (1H, t, J=2 Hz), 7.79 (1H, d, J=16 Hz), 7.82-7.84 (3H, m), 8.88 (3H, br-s), 9.38 (1H, s), 14.92 (1H, br-s)

### Example 15

### [1-[(Ozagrel-methoxy)-carbonyl] cyclohexyl] amino-chondroitin Sulfate Conjugate

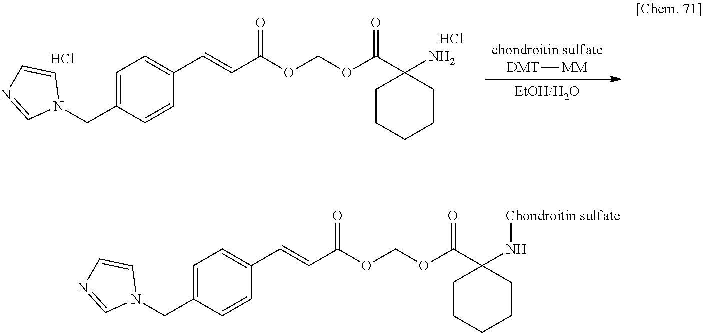

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 36 mg (0.080 mmol) of (2E)-3-[4-(1H-imidazol-1-ylmethyl) phenyl]-2-propenoic acid [[(1-aminocyclohexyl) carbonyl] oxy] methyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 13 mL of 94% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 145 mg of the title compound. Based on values of integral in ¹H-NMR, the introduction ratio of ozagrel per unit of whole disaccharide (glucuronic acid) of chondroitin sulfate was 1%.

### Reference Example 46

### 3-[[(1,1-Dimethylethoxy) carbonyl] amino]-2,2-dimethylpropanoic Acid Chloromethyl Ester

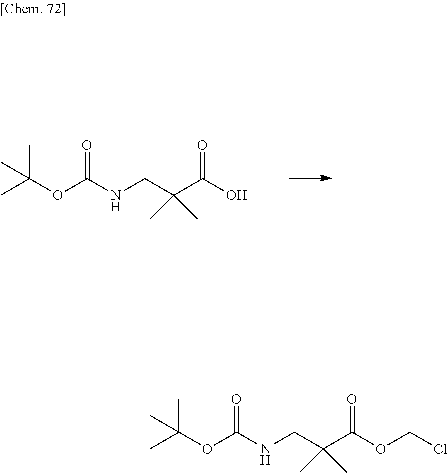 [SMILES: CC(C)(C)OC(=O)NCC(C)(C)C(=O)O ; CC(C)(C)OC(=O)NCC(C)(C)C(=O)OCCl]

Under cooling on ice, a methylene chloride solution of 447 mg (2.7 mmol) of chloromethyl chlorosulfonate was added dropwise to a water 5 mL-methylene chloride 5 mL mixed solution of 491 mg (2.3 mmol) of 3-[[(1,1-dimethylethoxy) carbonyl]amino]-2,2-dimethylpropanoic acid, 77 mg (0.2 mmol) of tetrabutylammonium hydrogen sulfate, and 759 mg (9.0 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate, and the solvent was distilled off under reduced pressure. The residue was dissolved again in diethyl ether and washed with water. The water layer was extracted with a small amount of diethyl ether, and the extracted product was put together with the organic layer. The organic layer was washed with saturated saline and dried with anhydrous sodium sulfate, and the solvent was distilled off under reduced pressure to obtain 563 mg (94%) of the title compound.

¹H-NMR (CDCl₃, δ): 1.25 (6H, s), 1.43 (9H, s), 3.28 (2H, d, J=7 Hz), 4.88 (1H, br-s), 5.72 (2H, s)

### Reference Example 47

### (2E)-3-[4-(1H-Imidazol-1-ylmethyl) phenyl]-2-propenoic Acid [3-[[(1,1-dimethylethoxy) carbonyl] amino]-2,2-dimethyl-1-oxopropoxy] methyl Ester

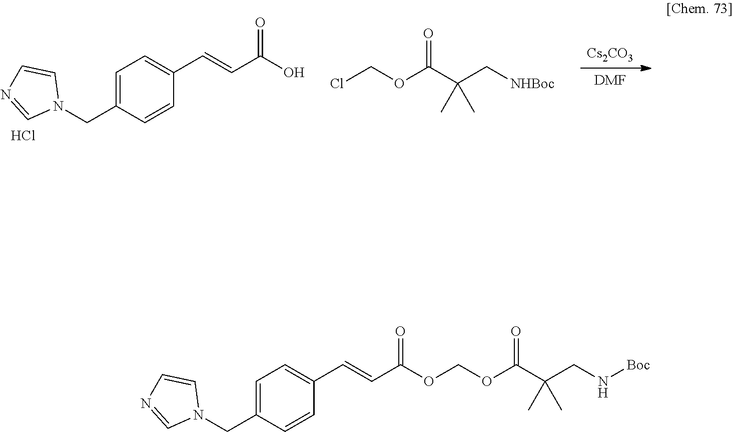

To a methanol solution of 300 mg (1.13 mmol) of ozagrel hydrochloride, 369 mg (1.13 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 301 mg (1.13 mmol) of 3-[[(1,1-dimethylethoxy) carbonyl] amino]-2,2-dimethylpropanoic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 389 mg (75%) of the title compound.

¹H-NMR (CDCl₃, δ): 1.21 (6H, s), 1.41 (9H, s), 2.26 (2H, d, J=7 Hz), 4.96 (1H, brt, J=7 Hz), 5.15 (2H, s), 5.88 (2H, s), 6.44 (1H, d, J=16 Hz), 6.90 (1H, t, J=1 Hz), 7.11 (1H, s), 7.17 (2H, d, J=9 Hz), 7.52 (2H, d, J=9 Hz), 7.56 (1H, s), 7.76 (1H, d, J=16 Hz)

### Reference Example 48

### (2E)-3-[4-(1H-Imidazol-1-ylmethyl) phenyl]-2-propenoic Acid (3-amino-2,2-dimethyl-1-okipropoxy) methyl Ester Dihydrochloride

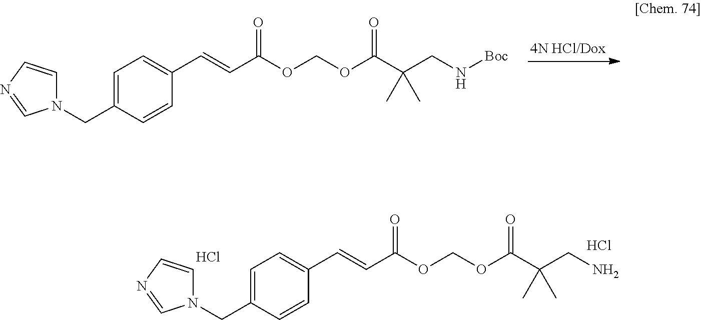 [SMILES: CC(C)(C)OC(=O)NCC(C)(C)C(=O)OCOC(=O)/C=C/c1ccc(Cn2ccnc2)cc1 ; CC(C)(CN)C(=O)OCOC(=O)/C=C/c1ccc(Cn2ccnc2)cc1 ; Cl ; Cl]

To 389 mg (0.850 mmol) of (2E)-3-[4-(1H-imidazol-1-ylmethyl) phenyl]-2-propenoic acid [3-[[(1,1-dimethylethoxy) carbonyl] amino]-2,2-dimethyl-1-oxopropoxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 148 mg (41%) of the title compound.

¹H-NMR (DMSO-d₆, δ): 1.22 (6H, s), 2.97 (2H, br-s), 5.49 (2H, s), 5.86 (2H, s), 6.73 (1H, d, J=16 Hz), 7.47 (211, d, J=9 Hz), 7.69 (1H, t, J=2 Hz), 7.76 (1H, d, J=16 Hz), 7.80-7.83 (3H, m), 8.25 (3H, br-s), 9.33 (1H, s), 14.85 (1H, br-s)

### Example 16

### [2,2-Dimethyl-3-oxo-3-(ozagrel-methoxy) propyl] amino-chondroitin Sulfate Conjugate

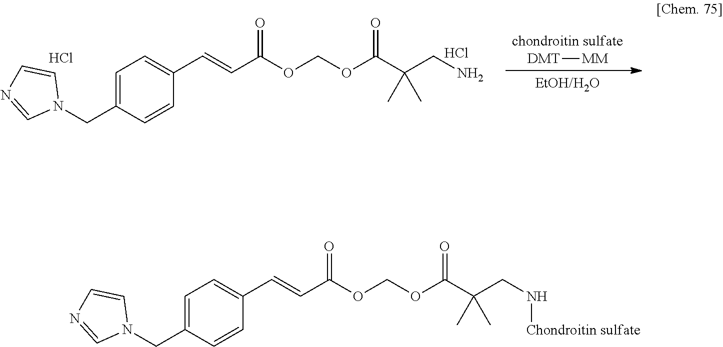

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 34 mg (0.080 mmol) of (2E)-3-[4-(1H-imidazol-1-ylmethyl) phenyl]-2-propenoic acid (3-amino-2,2-dimethyl-1-okipropoxy) methyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 13 mL of 94% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 158 mg of the title compound. Based on values of integral in H-NMR, the introduction ratio of ozagrel per unit of whole disaccharide (glucuronic acid) of chondroitin sulfate was 7%.

### Reference Example 49

### 1-[[[(1,1-Dimethylethoxy) carbonyl] amino] methyl] cyclopentane Carboxylic Acid

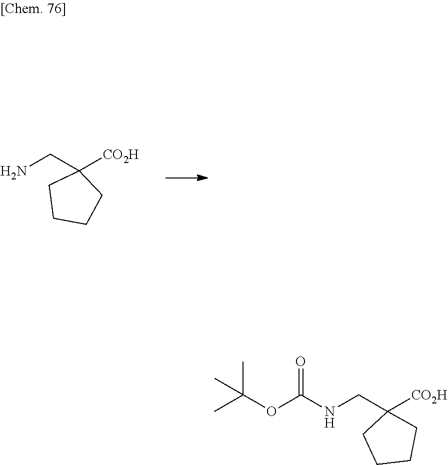 [SMILES: NCC1(C(=O)O)CCCC1 ; CC(C)(C)OC(=O)NCC1(C(=O)O)CCCC1]

To a water-dioxane mixed solution of 500 mg (2.8 mmol) of 1-(aminomethyl) cyclopentane carboxylic acid and 844 mg (8.3 mmol) of triethylamine, 601 mg (2.8 mmol) of tert-butyl dicarbonate was added, and the resulting mixture was stirred at room temperature overnight. The reaction solution was condensed under reduced pressure, and the residue was dissolved in ethyl acetate and washed with a 10% potassium hydrogen sulfate solution and saturated saline. The organic layer was dried with anhydrous sodium sulfate, and the solvent was distilled off under reduced pressure to obtain 691 mg (quantitative) of the title compound.

¹H-NMR (CDCl₃, δ): 1.44 (9H, s), 1.58-1.81 (6H, m), 1.96-2.07 (2H, m), 3.28 (2H, d, J=6 Hz), 5.10 (1H, br-s)

### Reference Example 50

### 1-[[[(1,1-Dimethylethoxy) carbonyl] amino] methyl] cyclopentane Carboxylic Acid Chloromethyl Ester

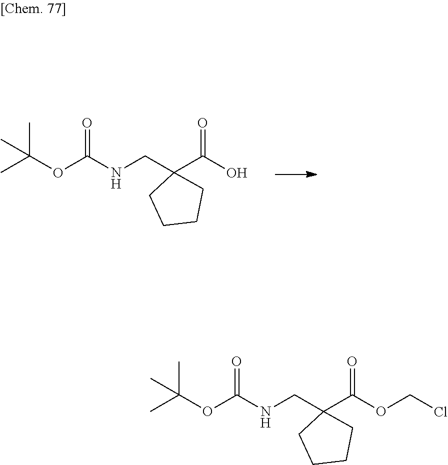 [SMILES: CC(C)(C)OC(=O)NCC1(C(=O)O)CCCC1 ; CC(C)(C)OC(=O)NCC1(C(=O)OCCl)CCCC1]

Under cooling on ice, a methylene chloride solution of 561 mg (3.4 mmol) of chloromethyl chlorosulfonate was added dropwise to a water 6 mL-methylene chloride 6 mL mixed solution of 610 mg (2.8 mmol) of 1-[[[(1,1-dimethylethoxy) carbonyl]amino] methyl] cyclopentane carboxylic acid, 95 mg (0.28 mmol) of tetrabutylammonium hydrogen sulfate, and 941 mg (11.2 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate, and then the solvent was distilled off under reduced pressure. The residue was dissolved again in diethyl ether and washed with water. The water layer was extracted with a small amount of diethyl ether, and the extracted product was put together with the organic layer. The organic layer was washed with saturated saline and dried with anhydrous sodium sulfate, and the solvent was distilled off under reduced pressure to obtain 756 mg (93%) of the title compound.

¹H-NMR (CDCl₃, δ): 1.43 (9H, s), 1.60-1.79 (6H, m), 1.92-2.03 (2H, m), 3.32 (2H, d, J=7 Hz), 4.96 (1H, s), 5.73 (2H, s)

### Reference Example 51

### 2-[4-[2-[(4-Chlorobenzoyl) amino] ethyl] phenoxy]-2-methylpropanoic Acid [[[1-[[[(1,1-dimethylethoxy) carbonyl] amino] methyl] cyclopentyl] carbonyl] oxy]methyl Ester

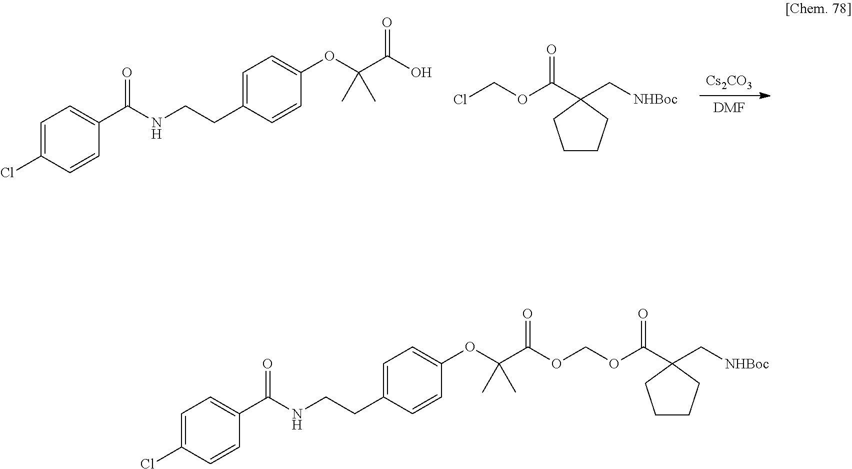

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 241 mg (0.829 mmol) of 1-[[[(1,1-dimethylethoxy) carbonyl] amino]methyl] cyclopentane carboxylic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 491 mg (96%) of the title compound.

¹H-NMR (CDCl₃, δ): 1.42 (9H, s), 1.58-1.60 (8H, m), 1.68-1.71 (4H, m), 1.89-1.94 (2H, m), 2.86 (2H, t, J=7 Hz), 3.24 (2H, d, J=6 Hz), 3.66 (2H, q, J=7 Hz), 4.94-4.97 (1H, m), 5.85 (2H, s), 6.14 (1H, br-s), 6.81 (2H, d, J=9 Hz), 7.09 (2H, d, J=9 Hz), 7.38 (2H, d, J=9 Hz), 7.63 (2H, d, J=9 Hz)

### Reference Example 52

### 2-[4-[2-[(4-Chlorobenzoyl) amino] ethyl] phenoxy]-2-methylpropanoic Acid [[[1-(aminomethyl) cyclopentyl] carbonyl] oxy] methyl Ester Hydrochloride

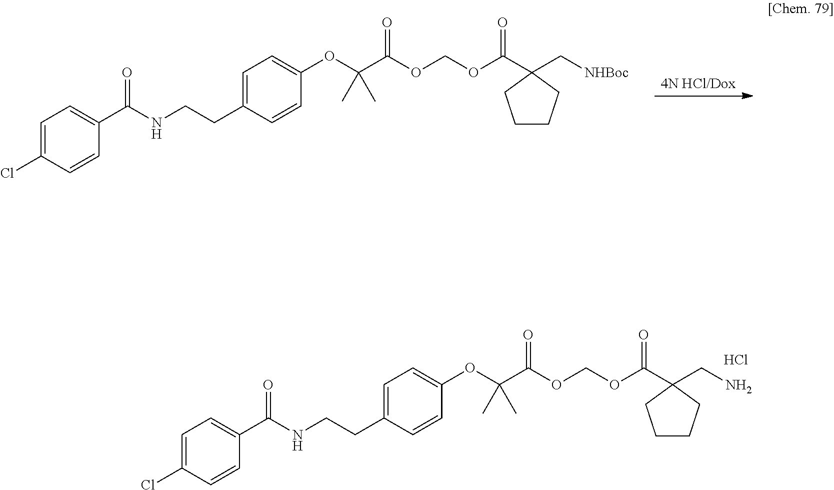 [SMILES: CC(C)(C)OC(=O)NCC1(C(=O)OCOC(=O)C(C)(C)Oc2ccc(CCNC(=O)c3ccc(Cl)cc3)cc2)CCCC1 ; "4N HCl/Dox" ; CC(C)(Oc1ccc(CCNC(=O)c2ccc(Cl)cc2)cc1)C(=O)OCOC(=O)C1(CN)CCCC1 ; Cl]

To 485 mg (0.786 mmol) of 2-[4-[2-[(4-chlorobenzoyl) amino] ethyl]phenoxy]-2-methylpropanoic acid [[[1-[[[(1,1-dimethylethoxy) carbonyl] amino]methyl] cyclopentyl] carbonyl] oxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 381 mg (88%) of the title compound.

¹H-NMR (DMSO-d₆, δ): 1.50 (6H, s), 1.59-1.68 (6H, m), 1.87-1.90 (2H, m), 2.78 (2H, t, J=8 Hz), 3.00 (2H, s), 3.43-3.46 (2H, m), 5.86 (2H, s), 6.75 (2H, d, J=9 Hz), 7.13 (2H, d, J=9 Hz), 7.53 (2H, d, J=9 Hz), 7.85 (2H, d, J=9 Hz), 8.13 (3H, br-s), 8.69 (1H, t, J=6 Hz)

### Example 17

[[1-[(Bezafibrate-methoxy) carbonyl] cyclopentyl] methyl] amino-chondroitin Sulfate Conjugate

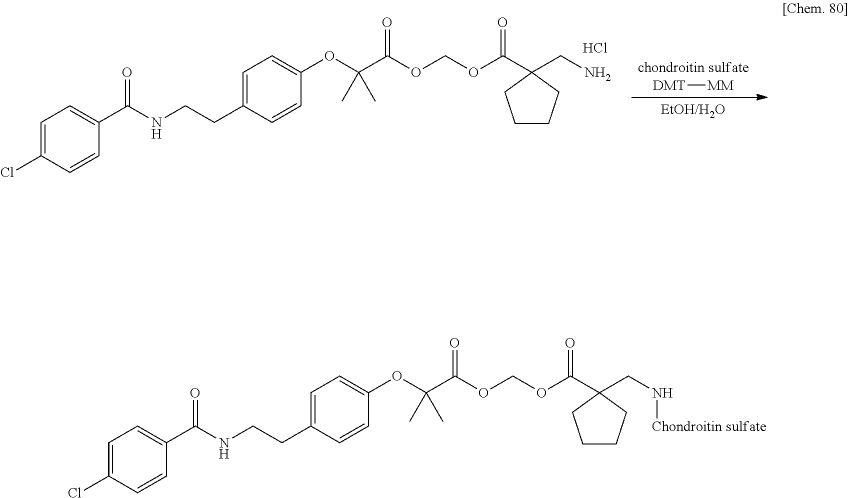

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 44 mg (0.080 mmol) of 2-[4-[2-[(4-chlorobenzoyl) amino]ethyl] phenoxy]-2-methylpropanoic acid [[[1-(aminomethyl) cyclopentyl] carbonyl]oxy] methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 4 mL) thereto until immediately before the reaction solution became cloudy. The reaction solution was added dropwise under stirring to 8 mL of 90% ethanol. To the mixed liquid, 7 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 218 mg of the title compound. Based on values of integral in ¹H-NMR, the introduction ratio of bezafibrate per unit of whole disaccharide (glucuronic acid) of chondroitin sulfate was 16%.

### Example 18

[[1-[(Bezafibrate-methoxy) carbonyl] cyclopentyl] methyl] amino-hyaluronic Acid

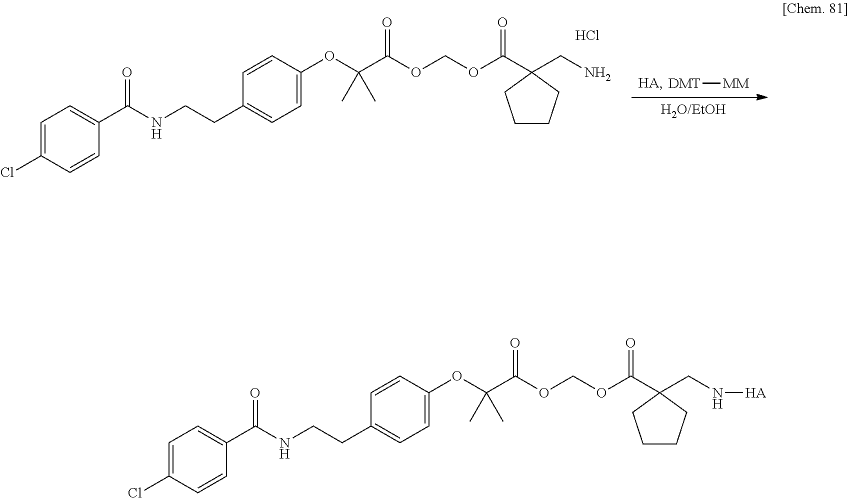

To 20.0 g (0.498 mmol) of a 1% sodium hyaluronate aqueous solution, 20 mL of ethanol was slowly added dropwise under stirring. To the mixed liquid, an ethanol 0.8 mL solution of 13 mg (0.025 mmol) of 2-[4-[2-[(4-chlorobenzoyl) amino] ethyl]phenoxy]-2-methylpropanoic acid [[[1-(aminomethyl) cyclopentyl] carbonyl] oxy]methyl ester hydrochloride was added. Subsequently, an ethanol 0.8 mL solution of 11.5 mg (0.042 mmol) of 4-(4,6-dimethoxy-1,3,5-triazin-2-yl)-4-methylmorpholinium chloride (DMT-MM) was added thereto, and 0.8 mL of EtOH and 2.5 mL of water were further added thereto. The resulting mixture was stirred overnight at room temperature. To the reaction solution, 1.5 mL of a 20% sodium chloride aqueous solution and 60 mL of EtOH were added to form precipitates, and the supernatant of the suspension was removed. 20 mL of ethanol was further added thereto, and the supernatant was removed. Thereafter, the residue was washed with 90% ethanol two times, washed with ethanol two times, and further washed with diethyl ether two times. The obtained precipitates were dried overnight using a vacuum pump to obtain 202 mg of the title compound. Based on values of integral in ¹H-NMR, the introduction ratio of bezafibrate per unit of whole disaccharide (glucuronic acid) of hyaluronic acid was 6%.

### Example 19

### [[1-[(Bezafibrate-methoxy) carbonyl] cyclopentyl] methyl] amino-carboxymethylcellulose Conjugate

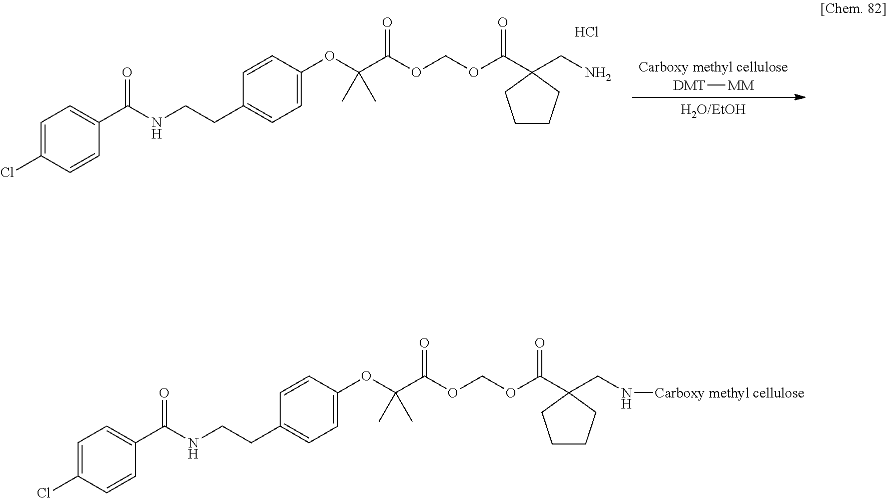

To 10 g (0.442 mmol) of a 1% sodium carboxymethylcellulose aqueous solution, 10 mL of ethanol was slowly added dropwise under stirring. To the mixed liquid, an ethanol 0.8 mL solution of 12.3 mg (0.022 mmol) of 2-[4-[2-[(4-chlorobenzoyl) amino] ethyl] phenoxy]-2-methylpropanoic acid [[[1-(aminomethyl) cyclopentyl] carbonyl] oxy] methyl ester hydrochloride was added. Subsequently, an ethanol 0.7 mL solution of 10.2 mg (0.037 mmol) of 4-(4,6-dimethoxy-1,3,5-triazin-2-yl)-4-methylmorpholinium chloride (DMT-MM) was added thereto, and 0.8 mL of EtOH and 2.5 mL of water were further added thereto. The resulting mixture was stirred overnight at room temperature. To the reaction solution, 1 mL of a 20% sodium chloride aqueous solution and 30 mL of EtOH were added to form precipitates, and the supernatant of the suspension was removed. 14 mL of ethanol was further added, and the supernatant was removed. Thereafter, the residue was washed with 90% ethanol two times, washed with ethanol two times, and further washed with diethyl ether two times. The obtained precipitates were dried overnight using a vacuum pump to obtain 103 mg of the title compound. Based on a measurement result (245 nm) of a spectrophotometer, the introduction ratio of bezafibrate per the total weight of the polymer conjugate was 6 wt %.

### Example 20

[[1-[(Bezafibrate-methoxy) carbonyl] cyclopentyl] methyl] amino-alginic Acid Conjugate

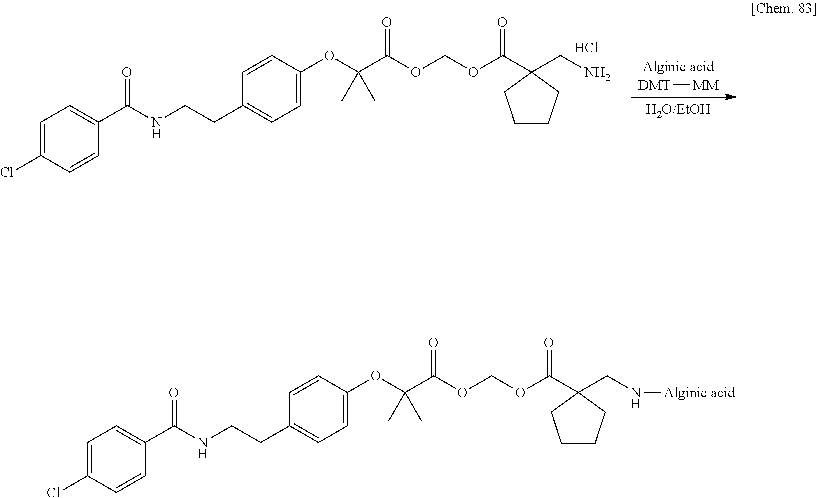

To 10 g (0.505 mmol) of a 1% sodium alginate aqueous solution, 3 mL of water and 10 mL of ethanol were slowly added dropwise under stirring. To the mixed liquid, an ethanol 0.8 mL solution of 14.0 mg (0.025 mmol) of 2-[4-[2-[(4-chlorobenzoyl) amino] ethyl] phenoxy]-2-methylpropanoic acid [[[1-(aminomethyl) cyclopentyl] carbonyl] oxy] methyl ester hydrochloride was added. Subsequently, an ethanol 0.8 mL solution of 11.7 mg (0.042 mmol) of 4-(4,6-dimethoxy-1,3,5-triazin-2-yl)-4-methylmorpholinium chloride (DMT-MM) was added thereto, and 0.8 mL of ethanol was further added thereto. The resulting mixture was stirred overnight at room temperature. To the reaction solution, 1.5 mL of a 20% sodium chloride aqueous solution was added, and the resulting mixture was stirred. To the reaction solution, 200 mL of acetone was added to form precipitates. Thereafter, the supernatant of the suspension was removed. 90 mL of a 90% acetone solution was further added thereto, and the supernatant was removed. Thereafter, the residue was washed with 90% acetone two times, washed with acetone two times, and further washed with diethyl ether two times. The obtained precipitates were dried overnight using a vacuum pump to obtain 97 mg of the title compound. Based on a measurement result (245 nm) of a spectrophotometer, the introduction ratio of bezafibrate per the total weight of the polymer conjugate was 6 wt %.

### Example 21

### [[1-[(Bezafibrate-methoxy) carbonyl] cyclopentyl] methyl] amino-polyglutamic Acid Conjugate

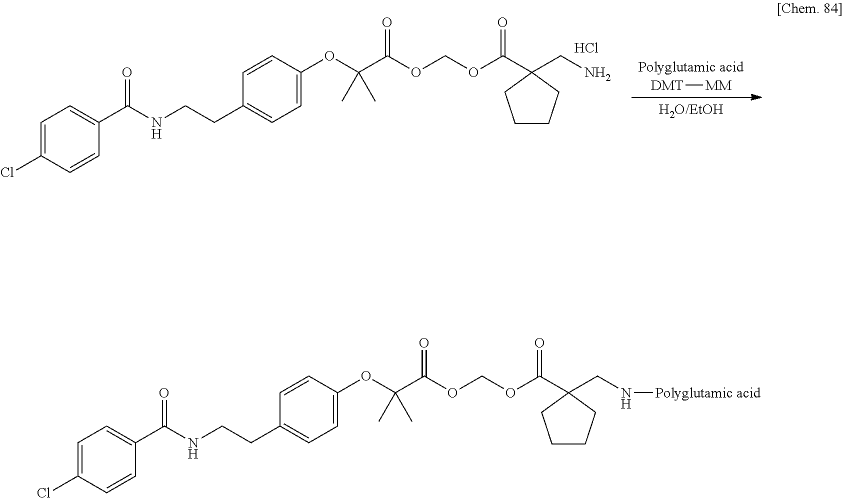

To 3.33 g (0.662 mmol) of a 3% sodium polyglutamate aqueous solution, 2 mL of ethanol was added dropwise under stirring. To the mixed liquid, an ethanol 0.8 mL solution of 18.3 mg (0.033 mmol) of 2-[4-[2-[(4-chlorobenzoyl) amino] ethyl]phenoxy]-2-methylpropanoic acid [[[1-(aminomethyl) cyclopentyl] carbonyl] oxy]methyl ester hydrochloride was added. Subsequently, an ethanol 1.1 mL solution of 15 mg (0.055 mmol) of 4-(4,6-dimethoxy-1,3,5-triazin-2-yl)-4-methylmorpholinium chloride (DMT-MM) was immediately added thereto, and 2.9 mL of ethanol and 3.7 mL of water were further added thereto. The resulting mixture was stirred overnight at room temperature. To the reaction solution, 100 μL of a 20% sodium chloride aqueous solution was added, and ethanol was further added dropwise (about 4 mL) thereto until immediately before the reaction solution became cloudy. The reaction solution was added dropwise under stirring to 10 mL of 90% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 106 mg of the title compound. Based on a measurement result (245 nm) of a spectrophotometer, the introduction ratio of bezafibrate per the total weight of the polymer conjugate was 6 wt %.

### Reference Example 53

### 2-[[[(1,1-Dimethylethoxy) carbonyl] amino] methyl]-2-ethylbutanoic Acid Chloromethyl Ester

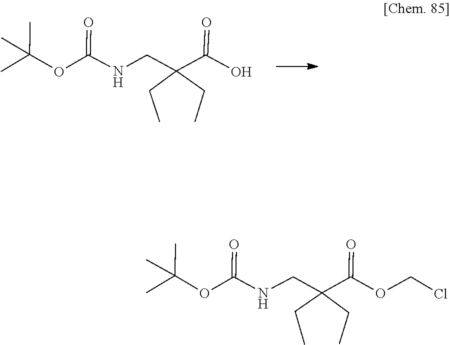 [SMILES: CCC(CC)(CNC(=O)OC(C)(C)C)C(=O)O ; CCC(CC)(CNC(=O)OC(C)(C)C)C(=O)OCCl]

Under cooling on ice, a methylene chloride solution of 1.49 g (10.02 mmol) of chloromethyl chlorosulfonate was added dropwise to a water 20 mL-methylene chloride 20 mL mixed solution of 1.23 g (5.01 mmol) of 2-[[[(1,1-dimethylethoxy) carbonyl] amino] methyl]-2-ethylbutanoic acid, 170 mg (0.50 mmol) of tetrabutylammonium hydrogen sulfate, and 3.4 g (40 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate. The solvent was condensed under reduced pressure, and the residue was purified by silica gel column chromatography (5% to 40% ethyl acetate/hexane) to obtain 1.31 g (89%) of the title compound.

¹H-NMR (CDCl₃, δ): 0.87 (6H, t, J=8 Hz), 1.43 (9H, s), 1.64 (4H, q, J=8 Hz), 3.36 (2H, d, J=7 Hz), 4.72 (1H, br-s), 5.74 (2H, s)

### Reference Example 54

### 2-[4-[2-[(4-Chlorobenzoyl) amino] ethyl] phenoxy]-2-methylpropanoic Acid [2-[[[(1,1-dimethylethoxy) carbonyl] amino] methyl]-2-ethyl-1-oxobutoxy] methyl Ester

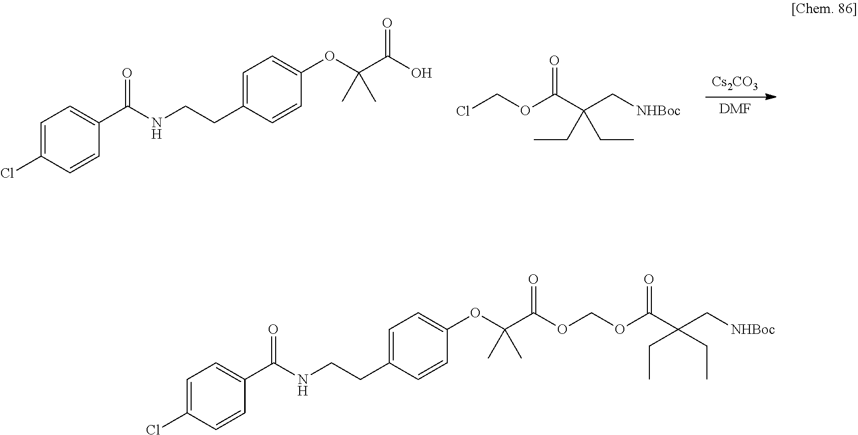

To a methanol solution of 200 mg (0.553 mmol) of bezafibrate, 90 mg (0.277 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 162 mg (0.553 mmol) of 2-[[[(1,1-dimethylethoxy) carbonyl] amino]methyl]-2-ethylbutanoic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 305 mg (89%) of the title compound.

¹H-NMR (CDCl₃, δ): 0.82 (6H, t, J=8 Hz), 1.42 (9H, s), 1.56-1.59 (10H, m), 2.87 (2H, t, J=7 Hz), 3.29 (2H, d, J=7 Hz), 3.67 (2H, q, J=7 Hz), 4.71 (1H, br-s), 5.86 (2H, s), 6.15 (1H, br-s), 6.82 (2H, d, J=9 Hz), 7.10 (2H, d, J=9 Hz), 7.38 (2H, d, J=9 Hz), 7.63 (2H, d, J=9 Hz)

### Reference Example 55

### 2-[4-[2-[(4-Chlorobenzoyl) amino] ethyl] phenoxy]-2-methylpropanoic Acid [2-(aminomethyl)-2-ethyl-1-oxobutoxy] methyl Ester Hydrochloride

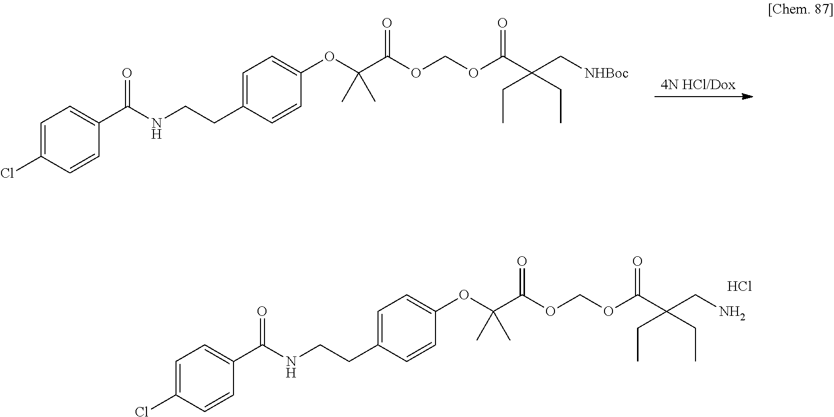 [SMILES: CCC(CC)(CNC(=O)OC(C)(C)C)C(=O)OCOC(=O)C(C)(C)Oc1ccc(CCNC(=O)c2ccc(Cl)cc2)cc1 ; "4N HCl/Dox" ; CCC(CC)(CN)C(=O)OCOC(=O)C(C)(C)Oc1ccc(CCNC(=O)c2ccc(Cl)cc2)cc1 ; Cl]

To 302 mg (0.488 mmol) of 2-[4-[2-[(4-chlorobenzoyl) amino] ethyl]phenoxy]-2-methylpropanoic acid [2-[[(1,1-dimethylethoxy) carbonyl] amino] methyl]-2-ethyl-1-oxobutoxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 231 mg (86%) of the title compound.

¹H-NMR (DMSO-d₆, δ): 0.73 (6H, t, J=8 Hz), 1.49 (6H, s), 1.60 (4H, q, J=8 Hz), 2.78 (2H, t, J=8 Hz), 2.98 (2H, s), 3.42-3.46 (2H, m), 5.87 (2H, s), 6.76 (2H, d, J=9 Hz), 7.13 (2H, d, J=9 Hz), 7.53 (2H, d, J=9 Hz), 7.84 (2H, d, J=9 Hz), 8.02 (3H, br-s), 8.65-8.67 (1H, m)

### Example 22

### [2-[(Bezafibrate-methoxy) carbonyl]-2-ethylbutyl] amino-chondroitin Sulfate Conjugate

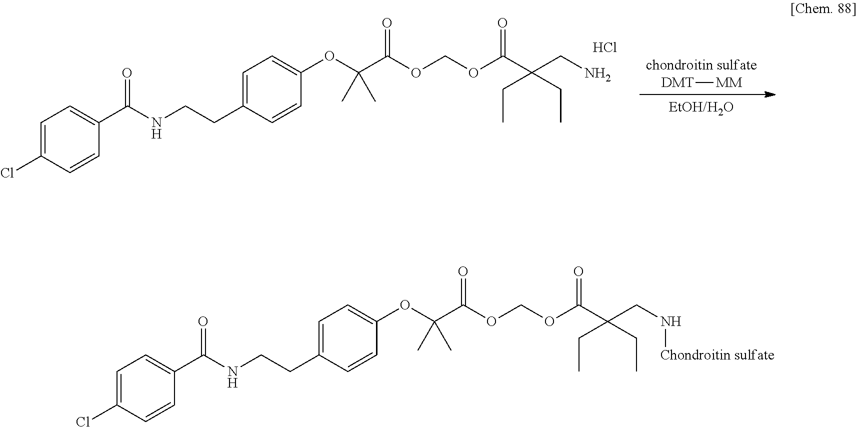

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 44 mg (0.080 mmol) of 2-[4-[2-[(4-chlorobenzoyl) amino]ethyl] phenoxy]-2-methylpropanoic acid [2-(aminomethyl)-2-ethyl-1-oxobutoxy]methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 4 mL) thereto until immediately before the reaction solution became cloudy. The reaction solution was added dropwise under stirring to 8 mL of 90% ethanol. To the mixed liquid, 7 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 219 mg of the title compound. Based on values of integral in ¹H-NMR, the introduction ratio of bezafibrate per unit of whole disaccharide (glucuronic acid) of chondroitin sulfate was 17%.

### Reference Example 56

### 2-[[(1,1-Dimethylethoxy) carbonyl] amino] butanoic Acid

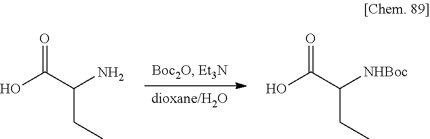

To a water-dioxane mixed solution of 2.00 g (19.4 mmol) of 2-aminobutanoic acid and 5.89 g (58.2 mmol) of triethylamine, 4.23 mg (19.4 mmol) of tert-butyl dicarbonate was added, and the resulting mixture was stirred at room temperature overnight. The reaction solution was condensed under reduced pressure, and the residue was dissolved in ethyl acetate and washed with a 10% potassium hydrogen sulfate solution and saturated saline. The organic layer was dried with anhydrous magnesium sulfate, and the solvent was distilled off under reduced pressure to obtain 4.20 g (quantitative) of the title compound.

¹H-NMR (CDCl₃, δ): 0.98 (3H, t, J=7 Hz), 1.45 (9H, s), 1.69-1.78 (1H, m), 1.89-1.93 (1H, m), 4.09 (1/3H, br-s), 4.28-4.29 (2/3H, m), 5.04 (2/3H, d, J=7 Hz), 6.09 (1/3H, br-s), 8.40 (1H, br-s)

### Reference Example 57

### 2-[[(1,1-Dimethylethoxy) carbonyl] amino] butanoic Acid Chloromethyl Ester

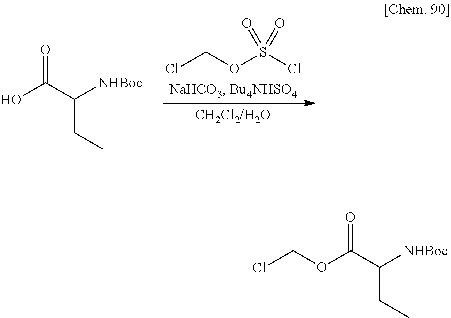 [SMILES: CCC(NC(=O)OC(C)(C)C)C(=O)O ; O=S(=O)(Cl)OCCl ; CCC(NC(=O)OC(C)(C)C)C(=O)OCCl]

Under cooling on ice, a methylene chloride solution of 3.85 g (25.9 mmol) of chloromethyl chlorosulfonate was added dropwise to a water 20 mL-methylene chloride 20 mL mixed solution of 2.58 g (12.9 mmol) of 2-[[(1,1-dimethylethoxy) carbonyl]amino] butanoic acid, 439 mg (1.29 mmol) of tetrabutylammonium hydrogen sulfate, and 4.34 g (51.72 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous magnesium sulfate. The solvent was condensed under reduced pressure, and the residue was purified by silica gel column chromatography to obtain 2.59 g (80%) of the title compound.

¹H-NMR (CDCl₃, δ): 0.97 (3H, t, J=7 Hz), 1.45 (9H, s), 1.68-1.77 (1H, m), 1.86-1.93 (1H, m), 4.30-4.31 (1H, m), 4.97-4.99 (1H, m), 5.63 (1H, d, J=6 Hz), 5.86 (1H, d, J=6 Hz)

### Reference Example 58

### 2-[4-[2-[(4-Chlorobenzoyl) amino] ethyl] phenoxy]-2-methylpropanoic Acid [2-[[(1,1-dimethylethoxy) carbonyl] amino]-1-oxobutoxy] methyl Ester

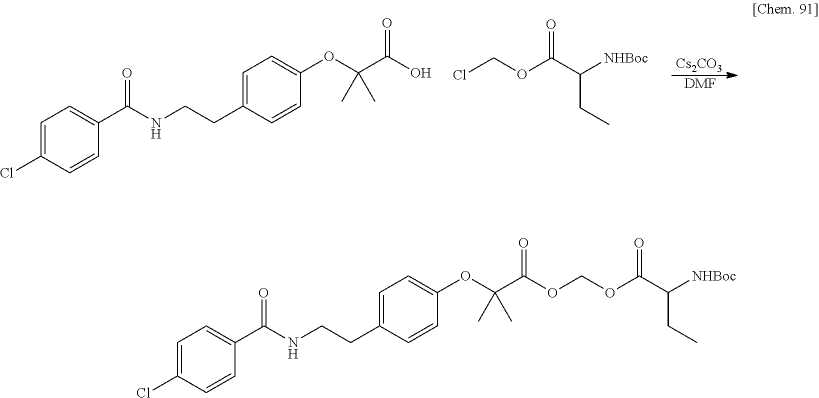

To a methanol solution of 200 mg (0.553 mmol) of bezafibrate, 90 mg (0.277 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 139 mg (0.553 mmol) of 2-[[(1,1-dimethylethoxy) carbonyl] amino]butanoic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 270 mg (84%) of the title compound.

¹H-NMR (CDCl₃, δ): 0.90 (3H, t, J=8 Hz), 1.43 (9H, s), 1.59-1.66 (7H, m), 1.78-1.83 (1H, m), 2.87 (2H, t, J=71 Hz), 3.67 (2H, q, J=7 Hz), 4.24-2.25 (1H, m), 4.95-4.96 (1H, m), 5.82 (1H, d, J=6 Hz), 5.93 (1H, d, J=6 Hz), 6.15 (1H, br-s), 6.81 (2H, d, J=9 Hz), 7.10 (2H, d, J=9 Hz), 7.38 (2H, d, J=9 Hz), 7.62 (2H, d, J=9 Hz)

### Reference Example 59

### 2-[4 [2-[(4-Chlorobenzoyl) amino] ethyl] phenoxy]-2-methylpropanoic Acid (2-amino-1-oxobutoxy) methyl Ester Hydrochloride

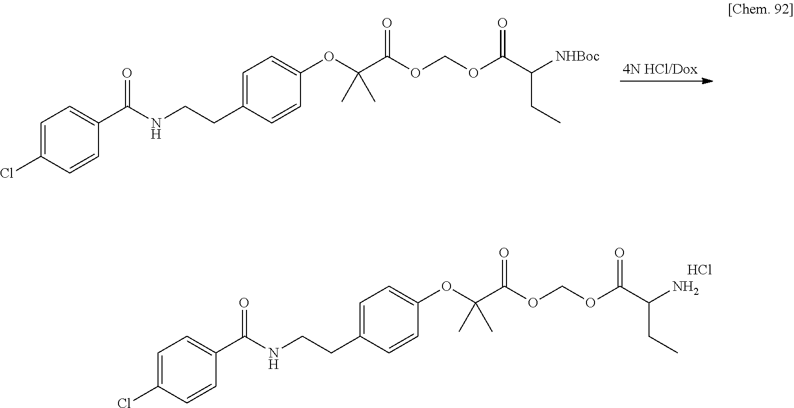 [SMILES: CCC(NC(=O)OC(C)(C)C)C(=O)OCOC(=O)C(C)(C)Oc1ccc(CCNC(=O)c2ccc(Cl)cc2)cc1 ; CCC(N)C(=O)OCOC(=O)C(C)(C)Oc1ccc(CCNC(=O)c2ccc(Cl)cc2)cc1 ; Cl]

To 267 mg (0.463 mmol) of 2-[4-[2-[(4-chlorobenzoyl) amino] ethyl]phenoxy]-2-methylpropanoic acid [2-[[(1,1-dimethylethoxy) carbonyl] amino]-1-oxobutoxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 152 mg (64%) of the title compound.

¹H-NMR (DMSO-d₆, δ): 0.90 (3H, t, J=8 Hz), 1.50 (6H, s), 1.79-1.85 (2H, m), 2.78 (2H, t, J=7 Hz), 3.44 (2H, q, J=7 Hz), 4.10 (1H, t, J=6 Hz), 5.88 (1H, d, J=6 Hz), 6.00 (1H, d, J=6 Hz), 6.77 (2H, d, J=9 Hz), 7.15 (2H, d, J=91 Hz), 7.53 (2H, d, J=9 Hz), 7.84 (2H, d, J=9 Hz), 8.61-8.67 (4H, m)

### Example 23

### [1-[(Bezafibrate-methoxy) carbonyl] propyl] amino-chondroitin Sulfate Conjugate

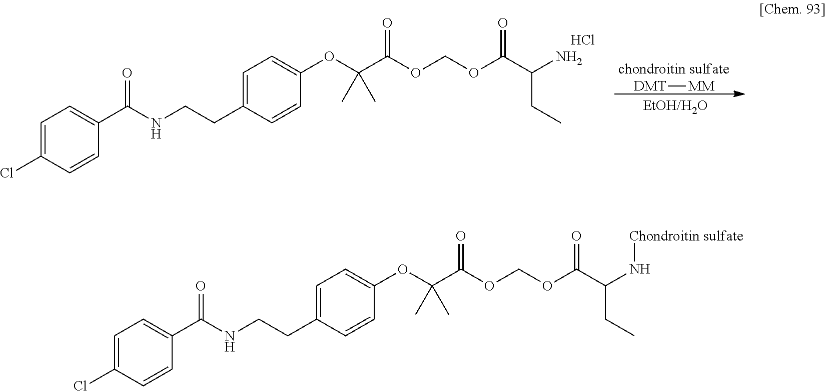

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 41 mg (0.080 mmol) of 2-[4-[2-[(4-chlorobenzoyl) amino]ethyl] phenoxy]-2-methylpropanoic acid (2-amino-1-oxobutoxy) methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 209 mg of the title compound. Based on values of integral in ¹H-NMR, the introduction ratio of bezafibrate per unit of whole disaccharide (glucuronic acid) of chondroitin sulfate was 13%.

### Reference Example 60

### 1-[[[(1,1-Dimethylethoxy) carbonyl] amino] methyl] cyclopropane Carboxylic Acid Ethyl Ester

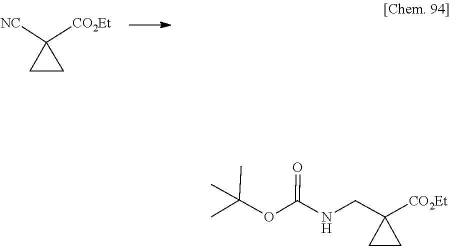 [SMILES: CCOC(=O)C1(C#N)CC1 ; CCOC(=O)C1(CNC(=O)OC(C)(C)C)CC1]

To 100 mL of methanol of 2.0 g (14.4 mmol) of 1-cyanocyclopropane carboxylic acid ethyl ester, 6.84 g (28.8 mmol) of cobalt chloride hexahydrate was added. While the mixed liquid was cooled in a water bath, 5.44 g (143.7 mmol) of sodium borohydride was added in small portions to the mixed liquid, and then the resulting mixture was stirred at room temperature for 30 minutes. To the reaction solution, 237 mL of 2 N hydrochloric acid was added, and the resulting mixture was stirred at room temperature for two hours. To the reaction solution, 57.7 g (569 mmol) of triethylamine was added, and the resulting mixture was stirred for one hour. Thereafter, 3.27 g (15 mmol) of tert-butyl dicarbonate was added thereto, and the resulting mixture was stirred at room temperature overnight. The insolubles were collected by filtration and washed with ethyl acetate three times. The obtained filtrates were put together, and the organic layer was isolated. Thereafter, the water layer portion was extracted with ethyl acetate. The extracted product was put together with the organic layer, and the resulting mixture was washed with saturated saline, and then dried with anhydrous sodium sulfate. The solvent was distilled off under reduced pressure. The residue was purified by silica gel column chromatography (5 to 10% ethyl acetate/hexane) to obtain 2.00 g (57%) of the title compound.

¹H-NMR (CDCl₃, δ): 0.88-0.97 (2H, m), 1.17-1.25 (2H, m), 1.23 (3H, t, J=7 Hz), 1.44 (9H, s), 3.28 (2H, d, J=6 Hz), 4.12 (2H, q, J=7 Hz), 5.16 (1H, br-s)

### Reference Example 61

### 1-[[[(1,1-Dimethylethoxy) carbonyl] amino] methyl] cyclopropane Carboxylic Acid

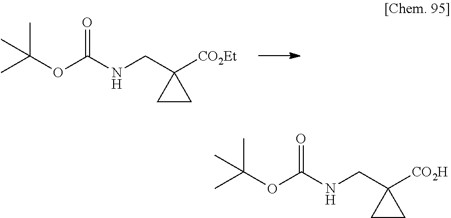 [SMILES: CCOC(=O)C1(CNC(=O)OC(C)(C)C)CC1 ; CC(C)(C)OC(=O)NCC1(C(=O)O)CC1]

To a tetrahydrofuran 30 mL solution of 2.00 g (8.2 mmol) of 1-[[[(1,1-dimethylethoxy) carbonyl] amino] methyl] cyclopropane carboxylic acid ethyl ester, 20 mL of a 2 N sodium hydroxide aqueous solution was added, and the resulting mixture was heated under reflux for four hours. The reaction solution was condensed under reduced pressure, and then diethyl ether was added to the residue. The resulting mixture was extracted with water two times. 6.13 g (45 mmol) of potassium hydrogen sulfate was added to the water layer such that the resulting mixture became acidic, and then the mixture was extracted with ethyl acetate. The organic layer was washed with saturated saline, and the solvent was distilled off under reduced pressure to obtain 1.73 g (98%) of the title compound.

¹H-NMR (CDCl₃, δ): 1.00-1.11 (2H, m), 1.27-1.34 (2H, m), 1.43 (9H, s), 3.28 (2H, d, J=6 Hz), 5.19 (1H, br-s)

### Reference Example 62

### 1-[[[(1,1-Dimethylethoxy) carbonyl] amino] methyl] cyclopropane Carboxylic Acid Chloromethyl Ester

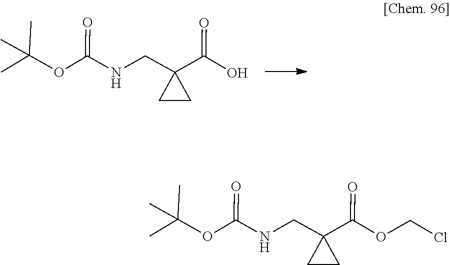 [SMILES: CC(C)(C)OC(=O)NCC1(C(=O)O)CC1 ; CC(C)(C)OC(=O)NCC1(C(=O)OCCl)CC1]

Under cooling on ice, a methylene chloride solution of 1.59 g (9.6 mmol) of chloromethyl chlorosulfonate was added dropwise to a water 8 mL-methylene chloride 8 mL mixed solution of 1.73 g (8 mmol) of 1-[[[(1,1-dimethylethoxy) carbonyl] amino]methyl] cyclopropane carboxylic acid, 272 mg (0.8 mmol) of tetrabutylammonium hydrogen sulfate, and 2.69 g (32 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate, and the solvent was distilled off under reduced pressure. The residue was dissolved again in diethyl ether and washed with water. The water layer was extracted with a small amount of diethyl ether, and the extracted product was put together with the organic layer. The organic layer was washed with saturated saline and dried with anhydrous sodium sulfate, and the solvent was distilled off under reduced pressure to obtain 1.95 g (92%) of the title compound.

¹H-NMR (CDCl₃, δ): 1.03-1.12 (2H, m), 1.29-1.36 (2H, m), 1.44 (9H, s), 3.32 (2H, d, J=6 Hz), 5.14 (1H, br-s), 5.71 (2H, s)

### Reference Example 63

### 2-[4-[2-[(4-Chlorobenzoyl) amino] ethyl] phenoxy]-2-methylpropanoic Acid [[[1-[[[(1,1-dimethylethoxy) carbonyl] amino] methyl] cyclopropyl] carbonyl] oxy]methyl Ester

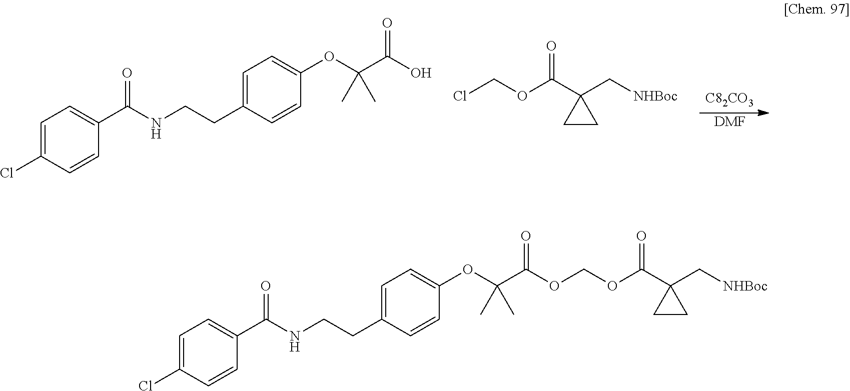

To a methanol solution of 200 mg (0.553 mmol) of bezafibrate, 90 mg (0.277 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 145 mg (0.553 mmol) of 1-[[[(1,1-dimethylethoxy) carbonyl] amino]methyl] cyclopropane carboxylic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 292 mg (90%) of the title compound.

¹H-NMR (CDCl₃, δ): 1.01 (2H, br-s), 1.21 (2H, br-s), 1.42 (9H, s), 1.60 (6H, s), 2.86 (2H, t, J=7 Hz), 3.26 (2H, d, J=7 Hz), 3.67 (2H, q, J=7 Hz), 5.12 (1H, br-s), 5.81 (2H, s), 6.10 (1H, br-s), 6.80 (2H, d, J=9 Hz), 7.10 (2H, d, J=9 Hz), 7.38 (2H, d, J=9 Hz), 7.62 (2H, d, J=9 Hz)

### Reference Example 64

### 2-[4-[2-[(4-Chlorobenzoyl) amino] ethyl] phenoxy]-2-methylpropanoic Acid [[[1-(aminomethyl) cyclopropyl] carbonyl] oxy] methyl Ester Hydrochloride

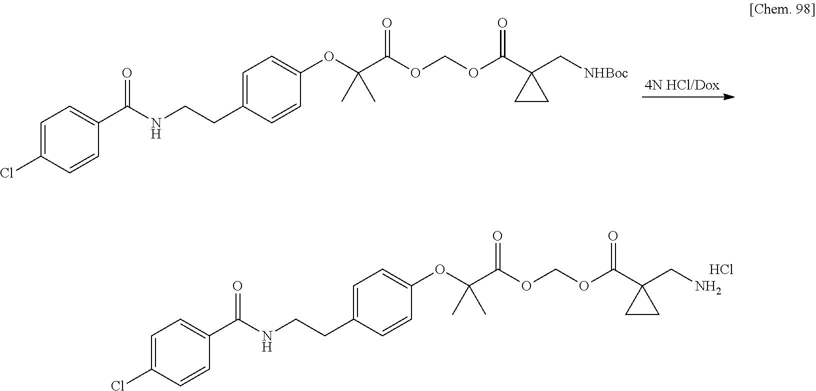 [SMILES: CC(C)(C)OC(=O)NCC1(C(=O)OCOC(=O)C(C)(C)Oc2ccc(CCNC(=O)c3ccc(Cl)cc3)cc2)CC1 ; CC(C)(Oc1ccc(CCNC(=O)c2ccc(Cl)cc2)cc1)C(=O)OCOC(=O)C1(CN)CC1 ; Cl]

To 289 mg (0.491 mmol) of 2-[4-[2-[(4-chlorobenzoyl) amino] ethyl]phenoxy]-2-methylpropanoic acid [[[1-[[[(1,1-dimethylethoxy) carbonyl] amino]methyl] cyclopropyl] carbonyl] oxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 183 mg (71%) of the title compound.

¹H-NMR (DMSO-d₆, δ): 1.18-1.25 (4H, m), 1.50 (6H, s), 2.78 (2H, t, J=7 Hz), 3.02 (2H, s), 3.44 (2H, q, J=7 Hz), 5.81 (2H, s), 6.73 (2H, d, J=9 Hz), 7.14 (2H, d, J=9 Hz), 7.53 (2H, d, J=9 Hz), 7.84 (2H, d, J=9 Hz), 8.07 (3H, br-s), 8.68 (1H, br)

### Example 24

### [1-[(Bezafibrate-methoxy) carbonyl] cyclopropyl] methyl] amino-chondroitin Sulfate Conjugate

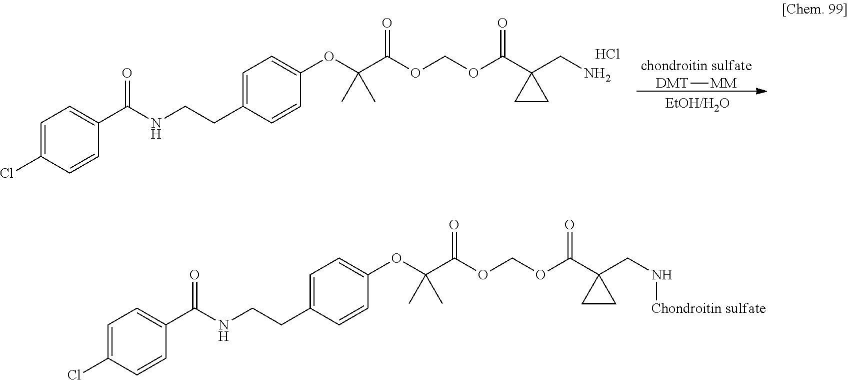

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 42 mg (0.080 mmol) of 2-[4-[2-[(4-chlorobenzoyl) amino]ethyl] phenoxy]-2-methylpropanoic acid [[[1-(aminomethyl) cyclopropyl] carbonyl]oxy] methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 4 mL) thereto until immediately before the reaction solution became cloudy. The reaction solution was added dropwise under stirring to 8 mL of 90% ethanol. To the mixed liquid, 7 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 217 mg of the title compound. Based on values of integral in ¹H-NMR, the introduction ratio of bezafibrate per unit of whole disaccharide (glucuronic acid) of chondroitin sulfate was 17%.

### Reference Example 65

### 2-[[[(1,1-Dimethylethoxy) carbonyl] amino] methyl]-3-ethylbutanoic acid 1-chloroethyl ester

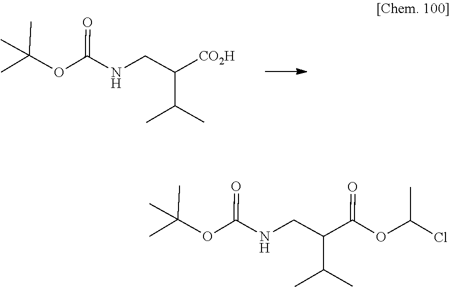 [SMILES: CC(C)C(CNC(=O)OC(C)(C)C)C(=O)O ; CC(Cl)OC(=O)C(CNC(=O)OC(C)(C)C)C(C)C]

Under cooling on ice, a methylene chloride solution of 3.58 g (22 mmol) of 1-chloroethyl chlorosulfonate was added dropwise to a water 5 mL-methylene chloride 5 mL mixed solution of 1.16 g (5.0 mmol) of 2-[[[(1,1-dimethylethoxy) carbonyl] amino]methyl]-3-methylbutanoic acid, 170 mg (0.5 mmol) of tetrabutylammonium hydrogen sulfate, and 1.68 g (20 mmol) of sodium hydrogen carbonate. The temperature of the reaction solution was returned to room temperature, and the reaction solution was stirred overnight. The methylene chloride layer of the reaction solution was isolated and washed with saturated saline. Thereafter, the organic layer was dried with anhydrous sodium sulfate, and the solvent was distilled off under reduced pressure. The residue was purified by silica gel column chromatography (10% ethyl acetate/hexane) to obtain 1.23 g (84%) of the title compound.

¹H-NMR (CDCl₃, δ): 0.97 (3H, d, J=7 Hz), 0.99 (3H, d, J=7 Hz), 1.43 (9H, s), 1.80 (3H, d, J=6 Hz), 1.91-2.07 (1H, m), 2.46-2.58 (1H, m), 3.18-3.52 (2H, m), 4.77 (1H, br-s), 6.53-6.62 (1H, m)

### Reference Example 66

### (2E)-3-[4-(1H-Imidazol-1-ylmethyl) phenyl]-2-propenoic Acid 1-[2-[[[(1,1-dimethylethoxy) carbonyl] amino] methyl]-3-methyl-1-oxobutoxy] ethyl Ester

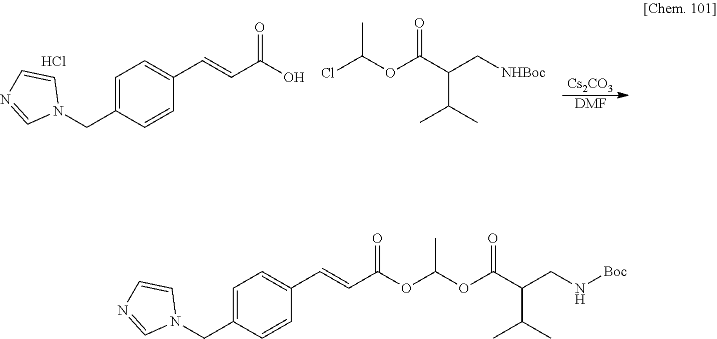

To a methanol solution of 170 mg (0.642 mmol) of ozagrel hydrochloride, 209 mg (0.642 mmol) of cesium carbonate was added, and the resulting was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 189 mg (0.642 mmol) of 2-[[[(1,1-dimethylethoxy) carbonyl] amino]methyl]-3-methylbutanoic acid 1-chloroethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 140 mg (45%) of the title compound.

¹H-NMR (CDCl₃, δ, mixture of diastereomers): 0.96-0.99 (6H, m), 1.39 (4.5H, s), 1.43 (4.5H, s), 1.566 (1.5H, d, J=6 Hz), 1.572 (1.5H, d, J=6 Hz), 1.92-1.99 (1H, m), 2.42 (0.5H, ddd, J=4, 8, 14 Hz), 2.46-2.51 (0.5H, m), 3.15 (0.5H, ddd, J=5, 10, 15 Hz), 3.23 (0.5H, ddd, J=6, 10, 14 Hz), 3.45-3.51 (1H, m), 4.92 (0.5H, br-s), 4.97 (0.5H, br-s), 5.15 (2H, s), 6.38 (0.5H, d, J=16 Hz), 6.41 (0.5H, d, J=16 Hz), 6.90 (1H, s), 6.99 (0.5H, q, J=6 Hz), 7.02 (0.5H, q, J=6 Hz), 7.11 (1H, s), 7.16 (2H, d, J=9 Hz), 7.51 (2H, d, J=9 Hz), 7.55 (1H, s), 7.70 (0.5H, d, J=16 Hz), 7.71 (0.5H, d, J=16 Hz)

### Reference Example 67

### (2E)-3-[4-(1H-Imidazol-1-ylmethyl) phenyl]-2-propenoic Acid 1-[2-(aminomethyl)-3-methyl-1-oxobutoxy] ethyl Ester Dihydrochloride

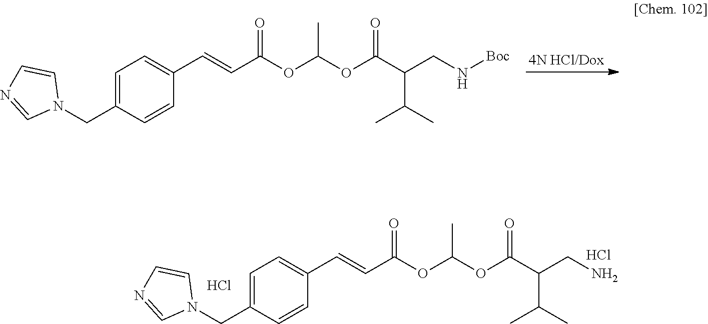 [SMILES: CC(OC(=O)/C=C/c1ccc(Cn2ccnc2)cc1)OC(=O)C(CNC(=O)OC(C)(C)C)C(C)C ; CC(OC(=O)/C=C/c1ccc(Cn2ccnc2)cc1)OC(=O)C(CN)C(C)C ; Cl ; Cl]

To 130 mg (0.268 mmol) of (2E)-3-[4-(1H-imidazol-1-ylmethyl) phenyl]-2-propenoic acid 1-[2-[[[(1,1-dimethylethoxy) carbonyl] amino] methyl]-3-methyl-1-oxobutoxy] ethyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 98 mg (80%) of the title compound.

¹H-NMR (DMSO-d₆, δ, mixture of diastereomers): 0.90-0.94 (6H, m), 1.54 (1.5H, d, J=7 Hz), 1.55 (1.5H, d, J=7 Hz), 1.98-2.05 (1H, m), 2.63-2.67 (1H, m), 2.89-2.96 (1H, m), 3.04-3.08 (1H, m), 5.47 (2H, s), 6.68 (1H, d, J=16 Hz), 6.93-6.96 (1H, m), 7.45 (2H, d, J=8 Hz), 7.68 (1H, s), 7.71 (0.5H, d, J=16 Hz), 7.72 (0.5H, d, J=16 Hz), 7.78 (1H, s), 7.80 (2H, d, J=Hz), 8.01 (1.5H, br-s), 8.04 (1.5H, br-s), 9.24 (1H, s), 14.56 (1H, br-s)

### Example 25

### [3-Methyl-2-[(1-ozagrel-ethoxy] carbonyl] butyl] amino-chondroitin Sulfate Conjugate

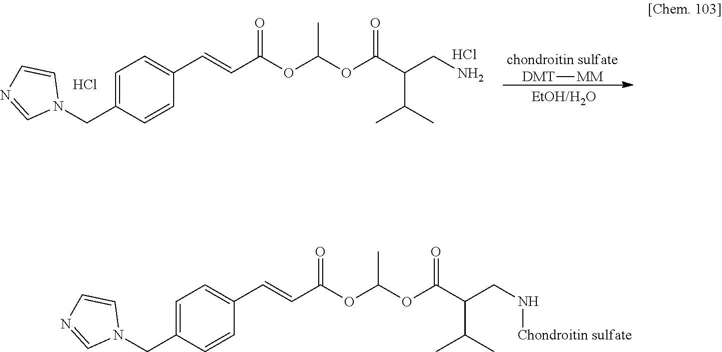

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 37 mg (0.080 mmol) of (2E)-3-[4-(H-imidazol-1-ylmethyl) phenyl]-2-propenoic acid 1-[2-(aminomethyl)-3-methyl-1-oxobutoxy] ethyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 13 mL of 94% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 185 mg of the title compound. Based on values of integral in ¹H-NMR, the introduction ratio of ozagrel per unit of whole disaccharide (glucuronic acid) of chondroitin sulfate was 6%.

### Reference Example 68

### 3-[[(1,1-Dimethylethoxy) carbonyl] amino]-2-methylpropanoic Acid Chloromethyl Ester

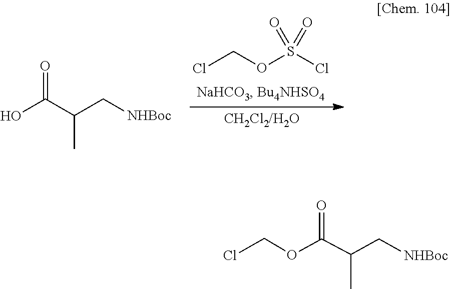 [SMILES: CC(CNC(=O)OC(C)(C)C)C(=O)O ; O=S(=O)(Cl)OCCl ; CC(CNC(=O)OC(C)(C)C)C(=O)OCCl]

Under cooling on ice, a methylene chloride 2 mL solution of 487 mg (2.95 mmol) of chloromethyl chlorosulfonate was added dropwise to a methylene chloride 2 mL-water 2 mL mixed solvent of 500 mg (2.46 mmol) of 3-[[(1,1-dimethylethoxy) carbonyl] amino]-2-methylpropanoic acid, 826 mg (9.84 mmol) of sodium hydrogen carbonate, and 83.5 mg (0.246 mmol) of tetrabutylammonium hydrogen sulfate. The temperature of the reaction solution was returned to room temperature, and the reaction solution was stirred overnight, and then extracted with diethyl ether. The obtained organic layer was washed with saturated saline, and then dried with anhydrous magnesium sulfate, and the solvent was distilled off under reduced pressure. The residue was purified by silica gel column chromatography to obtain 541 mg (87%) of the title compound.

¹H-NMR (CDCl₃, δ): 1.20 (3H, d, J=8 Hz), 1.43 (9H, s), 2.76-2.79 (1H, m), 3.25-3.31 (1H, m), 3.34-3.38 (1H, m), 4.88 (1H, br-s), 5.69 (1H, d, J=6 Hz), 5.75 (1H, d, J=6 Hz)

### Reference Example 69

### 2-[4-[2-[(4-Chlorobenzoyl) amino] ethyl] phenoxy]-2-methylpropanoic Acid [3-[[(1,1-dimethylethoxy) carbonyl] amino]-2-methyl-1-oxopropoxy] methyl Ester

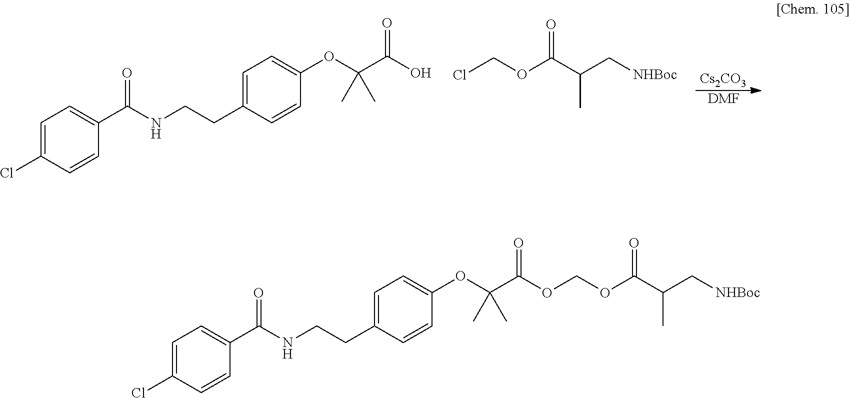

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 174 mg (0.691 mmol) of 3-[[(1,1-dimethylethoxy) carbonyl] amino]-2-methylpropanoic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 398 mg (quant.) of the title compound.

¹H-NMR (CDCl₃, δ): 1.13 (3H, d, J=8 Hz), 1.42 (9H, s), 1.60 (6H, s), 2.66-2.72 (1H, m), 2.86 (2H, t, J=7 Hz), 3.17-3.22 (1H, m), 3.27-3.30 (1H, m), 3.66 (2H, q, J=7 Hz), 4.87 (1H, br-s), 5.84 (1H, d, J=6 Hz), 5.86 (1H, d, J=6 Hz), 6.17 (1H, br-s), 6.81 (2H, d, J=9 Hz), 7.10 (2H, d, J=9 Hz), 7.38 (2H, d, J=9 Hz), 7.63 (2H, d, J=9 Hz)

### Reference Example 70

### 2-[4-[2-[(4-Chlorobenzoyl) amino] ethyl] phenoxy]-2-methylpropanoic Acid (3-amino-2-methyl-1-oxopropoxy) methyl Ester Hydrochloride

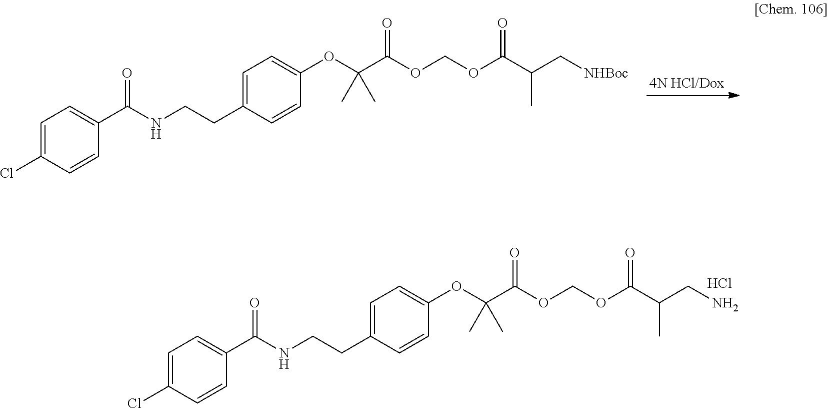 [SMILES: CC(CNC(=O)OC(C)(C)C)C(=O)OCOC(=O)C(C)(C)Oc1ccc(CCNC(=O)c2ccc(Cl)cc2)cc1 ; CC(CN)C(=O)OCOC(=O)C(C)(C)Oc1ccc(CCNC(=O)c2ccc(Cl)cc2)cc1 ; Cl]

To 395 mg (0.684 mmol) of 2-[4-[2-[(4-chlorobenzoyl) amino] ethyl] phenoxy]-2-methylpropanoic acid [3-[[(1,1-dimethylethoxy) carbonyl] amino]-2-methyl-1-oxopropoxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 309 mg (88%) of the title compound.

¹H-NMR (DMSO-d₆, δ): 1.13 (3H, d, J=7 Hz), 1.50 (6H, s), 2.78 (2H, t, J=8 Hz), 2.82-2.90 (2H, m), 3.05 (1H, dd, J=7, 12 Hz), 3.42-3.46 (2H, m), 5.80 (1H, d, J=6 Hz), 5.89 (1H, d, J=6 Hz), 6.75 (2H, d, J=9 Hz), 7.13 (2H, d, J=9 Hz), 7.53 (2H, d, J=9 Hz), 7.81 (3H, br-s), 7.83 (2H, d, J=9 Hz), 8.65 (1H, br)

### Example 26

### [3-(Bezafibrate-methoxy)-2-methyl-3-oxopropyl] amino-chondroitin Sulfate Conjugate

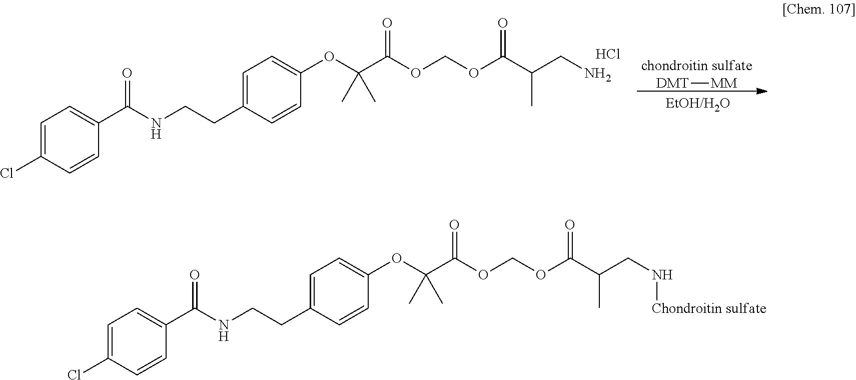

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 41 mg (0.080 mmol) of 2-[4-[2-[(4-chlorobenzoyl) amino]ethyl] phenoxy]-2-methylpropanoic acid (3-amino-2-methyl-1-oxopropoxy) methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 5 mL) thereto until immediately before the reaction solution became cloudy. The reaction solution was added dropwise under stirring to 8 mL of 90% ethanol. To the mixed liquid, 6 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 266 mg of the title compound. Based on values of integral in ¹H-NMR, the introduction ratio of bezafibrate per unit of whole disaccharide (glucuronic acid) of chondroitin sulfate was 15%.

### Reference Example 71

### 2-[[[(1,1-Dimethylethoxy) carbonyl] amino] methyl]-3,3-dimethylbutanoic Acid Chloromethyl Ester

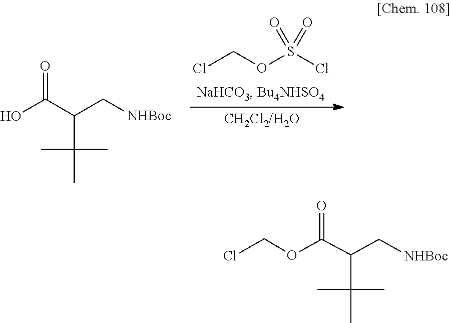 [SMILES: CC(C)(C)OC(=O)NCC(C(=O)O)C(C)(C)C ; O=S(=O)(Cl)OCCl ; CC(C)(C)OC(=O)NCC(C(=O)OCCl)C(C)(C)C]

Under cooling on ice, a methylene chloride 2 mL solution of 92.8 mg (0.563 mmol) of chloromethyl chlorosulfonate was added dropwise to a methylene chloride 2 mL-water 2 mL mixed solvent of 115 mg (0.469 mmol) of 2-[[[(1,1-dimethylethoxy) carbonyl] amino] methyl]-3,3-dimethylbutanoic acid, 157 mg (1.88 mmol) of sodium hydrogen carbonate, and 15.9 mg (0.0469 mmol)) of tetrabutylammonium hydrogen sulfate. The temperature of the reaction solution was returned to room temperature, and the reaction solution was stirred overnight, and then extracted with diethyl ether. The obtained organic layer was washed with saturated saline, and then dried with anhydrous magnesium sulfate, and the solvent was distilled off under reduced pressure. The residue was purified by silica gel column chromatography to obtain 133 mg (97%) of the title compound.

¹H-NMR (CDCl₃, δ): 1.02 (9H, s), 1.42 (9H, s), 2.63-2.64 (1H, m), 3.25 (1H, ddd, J=6, 11, 14 Hz), 3.53-3.54 (1H, m), 4.63 (1H, br-s), 5.69 (1H, d, J=6 Hz), 5.79 (1H, d, J=6 Hz)

### Reference Example 72

### (2E)-3-[4-(1H-Imidazol-1-ylmethyl) phenyl]-2-propenoic Acid [2-[[[(1,1-dimethylethoxy) carbonyl] amino] methyl]-3,3-[dimethyl-1-oxobutoxy] methyl Ester

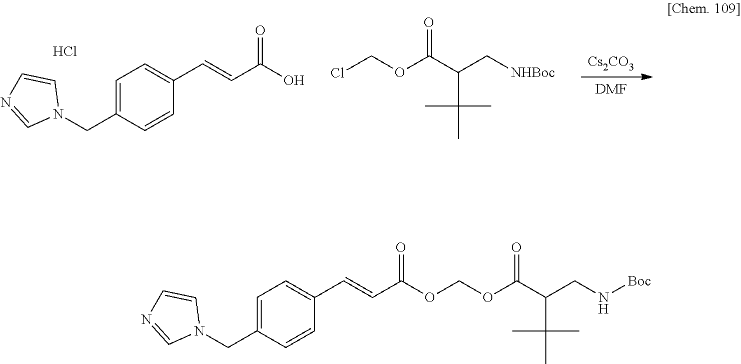

To a methanol solution of 120 mg (0.453 mmol) of ozagrel hydrochloride, 148 mg (0.453 mmol) of cesium carbonate was added, and the resulting was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 133 mg (0.453 mmol) of 2-[[[(1,1-dimethylethoxy) carbonyl] amino]methyl]-3,3-methylbutanoic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 220 mg (quant.) of the title compound.

¹H-NMR (CDCl₃, δ): 1.00 (9H, s), 1.39 (9H, s), 2.58-2.60 (1H, m), 3.22 (1H, ddd, J=6, 7, 9 Hz), 3.51-3.57 (1H, m), 4.69 (1H, br-s), 5.15 (2H, s), 5.88 (1H, d, J=6 Hz), 5.93 (1H, d, J=6 Hz), 6.43 (2H, d, J=16 Hz), 6.90 (1H, t, J=2 Hz), 7.11 (1H, s), 7.17 (1H, d, J=9 Hz), 7.51 (2H, d, J=9 Hz), 7.55 (1H, s), 7.74 (1H, d, J=16 Hz)

### Reference Example 73

### (2E)-3-[4-(1H-Imidazol-1-ylmethyl) phenyl]-2-propenoic Acid [2-(aminomethyl)-3,3-dimethyl-1-oxobutoxy] methyl Ester Dihydrochloride

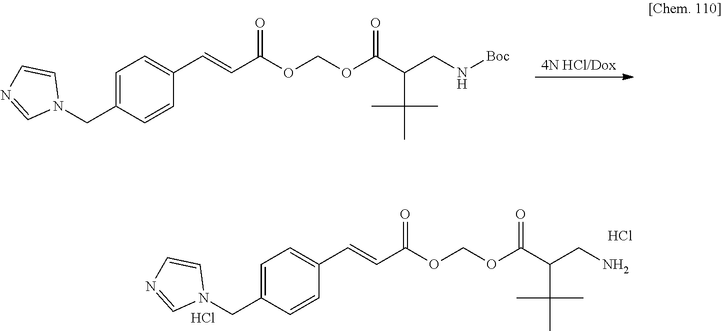 [SMILES: CC(C)(C)OC(=O)NCC(C(=O)OCOC(=O)/C=C/c1ccc(Cn2ccnc2)cc1)C(C)(C)C ; "4N HCl/Dox" ; CC(C)(C)C(CN)C(=O)OCOC(=O)/C=C/c1ccc(Cn2ccnc2)cc1 ; Cl ; Cl]

To 217 mg (0.447 mmol) of (2E)-3-[4-(1H-imidazol-1-ylmethyl) phenyl]-2-propenoic acid [2-[[[(1,1-dimethylethoxy) carbonyl] amino] methyl]-3,3-dimethyl-1-oxobutoxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 152 mg (75%) of the title compound.

¹H-NMR (DMSO-d₆, δ): 0.92 (9H, s), 2.54-2.55 (1H, m), 3.04 (2H, br-s), 5.47 (2H, s), 5.79 (1H, d, J=6 Hz), 5.94 (1H, d, J=6 Hz), 6.73 (1H, d, J=16 Hz), 7.46 (2H, d, J=8 Hz), 7.69 (1H, s), 7.75 (1H, d, J=16 Hz), 7.78 (1H, s), 7.81 (2H, d, J=8 Hz), 8.13 (3H, br-s), 14.65 (1H, br-s)

### Example 27

### [3,3-Dimethyl-2-[[(ozagrel-methoxy] carbonyl] butyl] amino-chondroitin Sulfate Conjugate

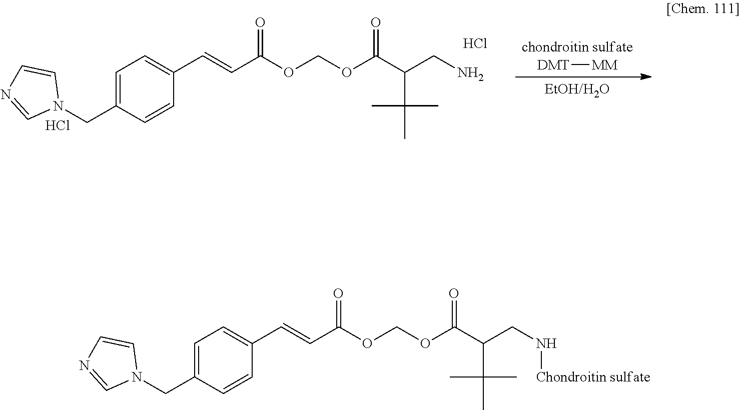

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 37 mg (0.080 mmol) of (2E)-3-[4-(1H-imidazol-1-ylmethyl) phenyl]-2-propenoic acid [2-(aminomethyl)-3,3-dimethyl-1-oxobutoxy] methyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 13 mL of 94% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 248 mg of the title compound. Based on values of integral in H-NMR, the introduction ratio of ozagrel per unit of whole disaccharide (glucuronic acid) of chondroitin sulfate was 7%.

### Reference Example 74

Trans-2-[[(1,1-dimethylethoxy) carbonyl] amino] cyclohexane Carboxylic Acid Chloromethyl Ester

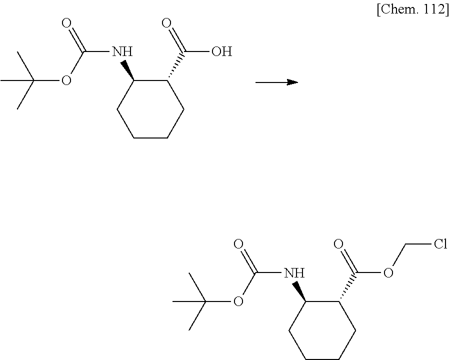 [SMILES: CC(C)(C)OC(=O)N[C@@H]1CCCC[C@H]1C(=O)O ; CC(C)(C)OC(=O)N[C@@H]1CCCC[C@H]1C(=O)OCCl]

Under cooling on ice, a methylene chloride solution of 367 mg (2.47 mmol) of chloromethyl chlorosulfonate was added dropwise to a water 5 mL-methylene chloride 6 mL mixed solution of 300 mg (1.23 mmol) of trans-2-[[(1,1-dimethylethoxy) carbonyl] amino] cyclohexane carboxylic acid, 41 mg (0.12 mmol) of tetrabutylammonium hydrogen sulfate, and 413 mg (4.92 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate. The solvent was condensed under reduced pressure, and the residue was purified by silica gel column chromatography (ethyl acetate/hexane) to obtain 311 mg (87%) of the title compound.

¹H-NMR (CDCl₃, δ): 1.17-1.27 (2H, m), 1.41 (9H, s), 1.57-1.66 (2H, m), 1.72-1.77 (2H, m), 1.92-2.05 (2H, m), 2.34 (1H, td, J=12, 4 Hz), 3.65-3.70 (1H, m), 4.48 (1H, br-s), 5.69 (2H, s)

### Reference Example 75

### 2-[4-[2-[(4-Chlorobenzoyl) amino] ethyl] phenoxy]-2-methylpropanoic Acid [[[trans-2-[[(1,1-dimethylethoxy) carbonyl] amino] cyclohexyl] carbonyl] oxy] methyl Ester

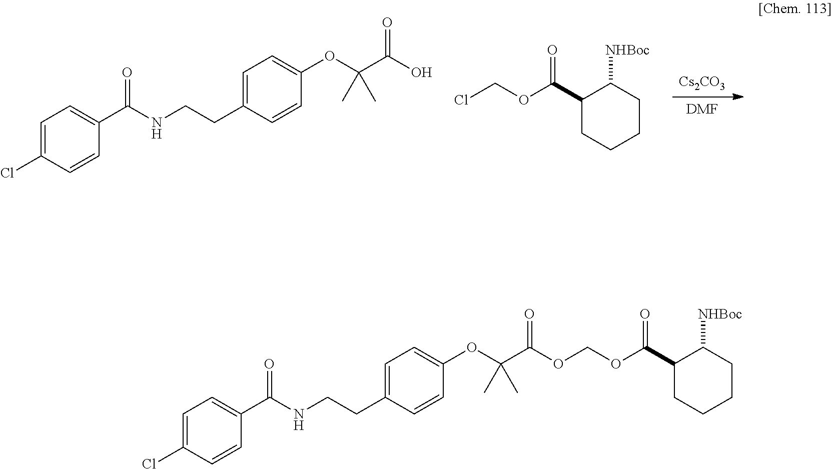

To a methanol solution of 64 mg (0.18 mmol) of bezafibrate, 29 mg (0.088 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 51 mg (0.18 mmol) of trans-2-[[(1,1-dimethylethoxy) carbonyl] amino]cyclohexane carboxylic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 108 mg (99%) of the title compound.

¹H-NMR (CDCl₃, δ): 1.13-1.27 (2H, m), 1.31-1.43 (10H, m), 1.51-1.62 (7H, m), 1.66-1.74 (2H, m), 1.83-1.86 (1H, m), 2.00-2.04 (1H, m), 2.27 (1H, dt, J=4, 11 Hz), 2.86 (2H, t, J=7 Hz), 3.62-3.69 (3H, m), 4.46 (1H, br-s), 5.81 (2H, s), 6.13 (1H, br-s), 6.83 (2H, d, J=9 Hz), 7.09 (2H, d, J=9 Hz), 7.38 (2H, d, J=9 Hz), 7.62 (2H, d, J=9 Hz)

### Reference Example 76

### 2-[4-[2-[(4-Chlorobenzoyl) amino] ethyl] phenoxy]-2-methylpropanoic Acid [[(trans-2-aminocyclohexyl) carbonyl] oxy] methyl Ester Hydrochloride

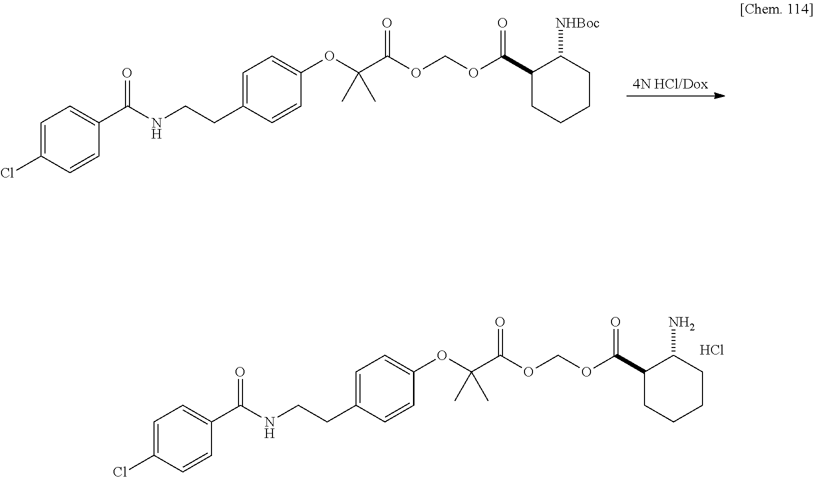

To 106 mg (0.172 mmol) of 2-[4-[2-[(4-chlorobenzoyl) amino] ethyl]phenoxy]-2-methylpropanoic acid [[[trans-2-[[(1,1-dimethylethoxy) carbonyl] amino]cyclohexyl] carbonyl] oxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 71 mg (75%) of the title compound.

¹H-NMR (DMSO-d₆, δ): 1.17-1.37 (4H, m), 1.50 (6H, s), 1.62-1.64 (1H, m), 1.69-1.71 (1H, m), 1.83-1.85 (1H, m), 1.94-1.96 (1H, m), 2.77 (2H, t, J=7 Hz), 3.19-3.28 (2H, m), 3.44 (2H, q, J=7 Hz), 5.79 (1H, d, J=6 Hz), 5.92 (1H, d, J=6 Hz), 6.75 (2H, d, J=9 Hz), 7.13 (2H, d, J=9 Hz), 7.53 (2H, d, J=9 Hz), 7.83 (2H, d, J=9 Hz), 7.96 (3H, br-s), 8.64 (1H, br)

### Example 28

### [trans-2-[(Bezafibrate-methoxy) carbonyl] cyclohexyl] amino-chondroitin Sulfate Conjugate

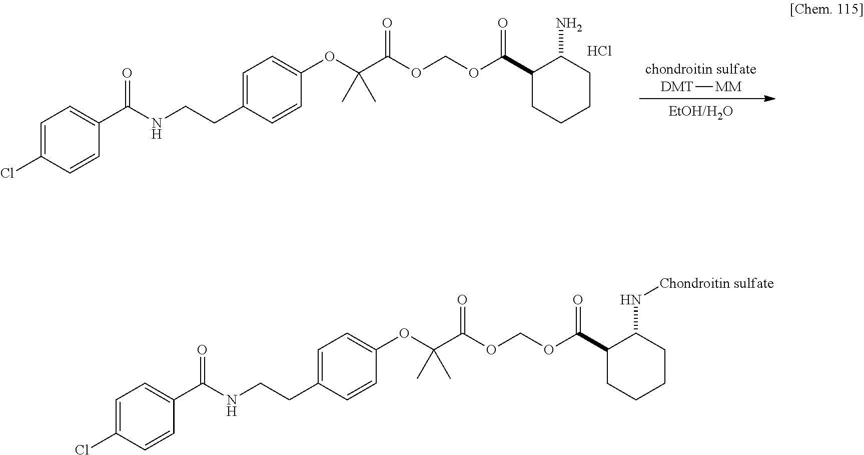

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 44 mg (0.080 mmol) of 2-[4-[2-[(4-chlorobenzoyl) amino]ethyl] phenoxy]-2-methylpropanoic acid [[(trans-2-aminocyclohexyl) carbonyl] oxy]methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 4 mL) thereto until immediately before the reaction solution became cloudy. The reaction solution was added dropwise under stirring to 8 mL of 90% ethanol. To the mixed liquid, 7 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 229 mg of the title compound. Based on values of integral in ¹H-NMR, the introduction ratio of bezafibrate per unit of whole disaccharide (glucuronic acid) of chondroitin sulfate was 6%.

### Reference Example 77

### 2-[[(1,1-Dimethylethoxy) carbonyl] amino]-2-ethylbutanoic Acid Chloromethyl Ester

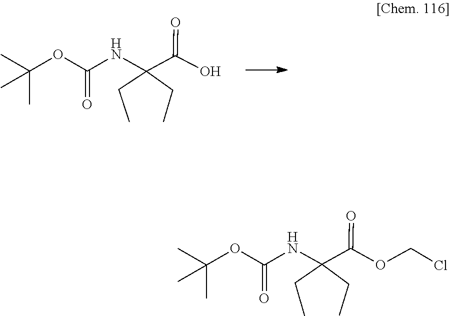

Under cooling on ice, a methylene chloride solution of 1036 mg (6.96 mmol) of 1-chloromethyl chlorosulfonate was added dropwise to a water 15 mL-methylene chloride 15 mL mixed solution of 804 mg (3.48 mmol) of 2-[[(1,1-dimethylethoxy) carbonyl] amino]-2-ethylbutanoic acid, 118 mg (0.35 mmol) of tetrabutylammonium hydrogen sulfate, and 1169 mg (13.92 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate. The solvent was condensed under reduced pressure, and the residue was purified by silica gel column chromatography (ethyl acetate/hexane) to obtain 882 mg (91%) of the title compound.

¹H-NMR (CDCl₃, δ): 0.81 (6H, t, J=8 Hz), 1.44 (9H, s), 1.79-1.87 (2H, m), 2.22 (2H, br-s), 5.28 (1H, br-s), 5.77 (2H, s)

### Reference Example 78

### 2-[4-[2-[(4-Chlorobenzoyl) amino] ethyl] phenoxy]-2-methylpropanoic Acid [2-[[(1,1-dimethylethoxy) carbonyl] amino]-2-ethyl-1-oxobutoxy] methyl Ester

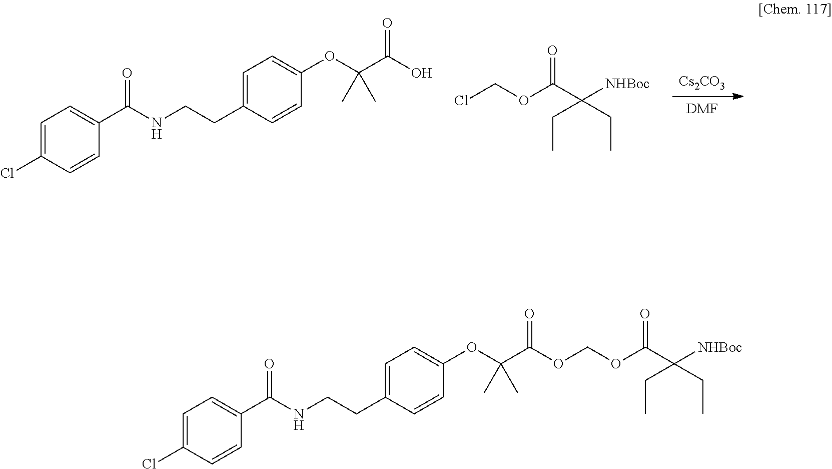

To a methanol solution of 250 mg (0.691 mmol) of bezafibrate, 112 mg (0.345 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 161 mg (0.576 mmol) of 2-[[(1,1-dimethylethoxy) carbonyl] amino]-2-ethylbutanoic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 295 mg (85%) of the title compound.

¹H-NMR (CDCl₃, δ): 0.75 (6H, t, J=8 Hz), 1.43 (9H, s), 1.58 (6H, s), 1.73-1.80 (2H, m), 2.21 (2H, br-s), 2.87 (2H, t, J=7 Hz), 3.67 (2H, q, J=7 Hz), 5.30 (1H, br-s), 5.89 (2H, s), 6.08 (1H, br-s), 6.83 (2H, d, J=9 Hz), 7.10 (2H, d, J=9 Hz), 7.38 (2H, d, J=9 Hz), 7.62 (2H, d, J=9 Hz)

### Reference Example 79

### 2-[4-[2-[(4-chlorobenzoyl) amino] ethyl] phenoxy]-2-methylpropanoic Acid (2-amino-2-ethyl-1-oxobutoxy) methyl Ester Hydrochloride

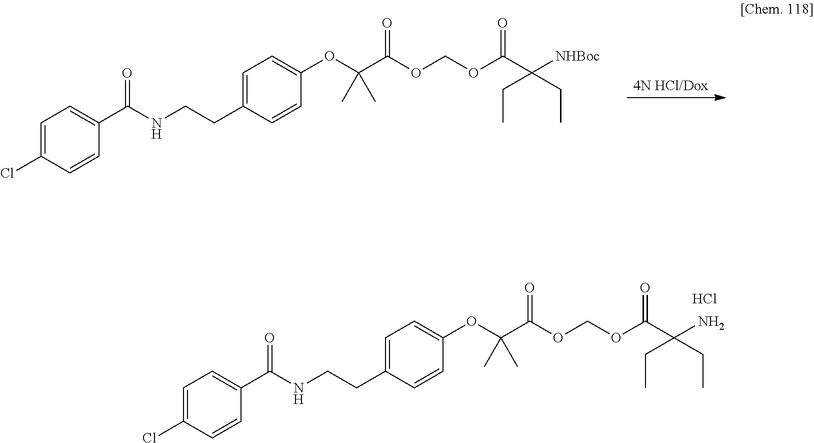 [SMILES: CCC(CC)(NC(=O)OC(C)(C)C)C(=O)OCOC(=O)C(C)(C)Oc1ccc(CCNC(=O)c2ccc(Cl)cc2)cc1 ; "4N HCl/Dox" ; CCC(N)(CC)C(=O)OCOC(=O)C(C)(C)Oc1ccc(CCNC(=O)c2ccc(Cl)cc2)cc1 ; Cl]

To 290 mg (0.479 mmol) of 2-[4-[2-[(4-chlorobenzoyl) amino] ethyl]phenoxy]-2-methylpropanoic acid [2-[[(1,1-dimethylethoxy) carbonyl] amino]-2-ethyl-1-oxobutoxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 229 mg (88%) of the title compound.

¹H-NMR (DMSO-d₆, δ): 0.87 (6H, t, J=8 Hz), 1.50 (6H, s), 1.83 (4H, q, J=8 Hz), 2.78 (2H, t, J=8 Hz), 3.42-3.46 (2H, m), 5.97 (2H, s), 6.76 (2H, d, J=9 Hz), 7.14 (2H, d, J=9 Hz), 7.53 (2H, d, J=9 Hz), 7.84 (2H, d, J=9 Hz), 8.61 (3H, br-s), 8.66 (1H, t, J=6 Hz)

### Example 29

### [1-[(Bezafibrate-methoxy) carbonyl]-1-ethylpropyl] amino-chondroitin Sulfate Conjugate

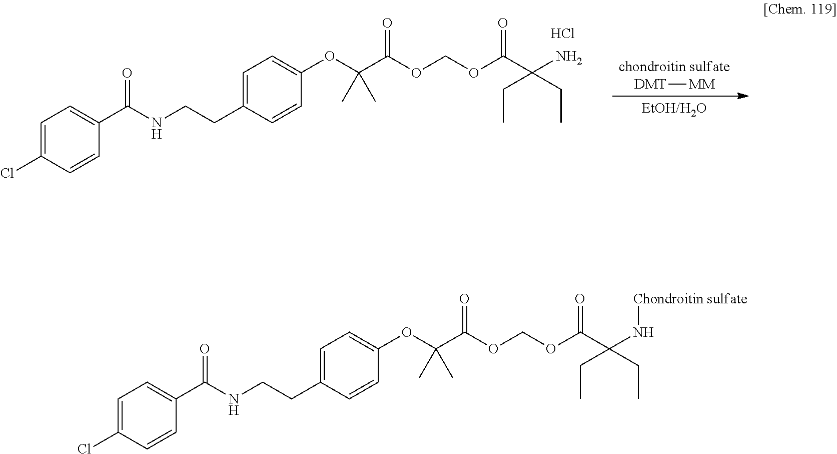

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 43 mg (0.080 mmol) of 2-[4-[2-[(4-chlorobenzoyl) amino]ethyl] phenoxy]-2-methylpropanoic acid (2-amino-2-ethyl-1-oxobutoxy) methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 8 mL of 90% ethanol. To the mixed liquid, 9 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 201 mg of the title compound. Based on values of integral in ¹H-NMR, the introduction ratio of bezafibrate per unit of whole disaccharide (glucuronic acid) of chondroitin sulfate was 0.5%.

### Reference Example 80

### 2-[[[(1,1-Dimethylethoxy) carbonyl] amino] methyl] butanoic Acid Chloromethyl Ester

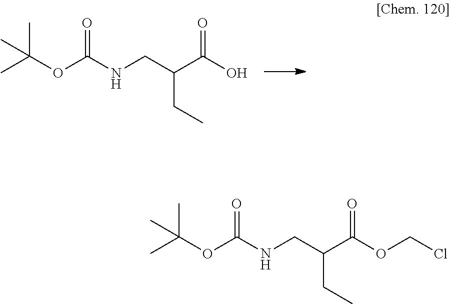

Under cooling on ice, a methylene chloride solution of 2.65 g (17.77 mmol) of 1-chloromethyl chlorosulfonate was added dropwise to a water 20 mL-methylene chloride 20 mL mixed solution of 1.93 mg (8.88 mmol) of 2-[[[(1,1-dimethylethoxy) carbonyl] amino] methyl] butanoic acid, 302 mg (0.89 mmol) of tetrabutylammonium hydrogen sulfate, and 2.98 g (35.54 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate. The solvent was condensed under reduced pressure, and the residue was purified by silica gel column chromatography (ethyl acetate/hexane) to obtain 1.97 mg (84%) of the title compound.

¹H-NMR (CDCl₃, δ): 0.96 (3/2H, t, J=8 Hz), 0.97 (3/2H, t, J=8 Hz), 1.43 (9H, s), 1.57-1.74 (2H, m), 2.65 (1H, br-s), 3.27-3.31 (1H, m), 3.39-3.42 (1H, m), 4.83 (1H, br-s), 5.69-5.78 (2H, m)

### Reference Example 81

### 2-[4-[2-[(4-Chlorobenzoyl) amino] ethyl] phenoxy]-2-methylpropanoic Acid [2-[[[(1,1-dimethylethoxy) carbonyl] amino] methyl]-1-oxobutoxy] methyl Ester

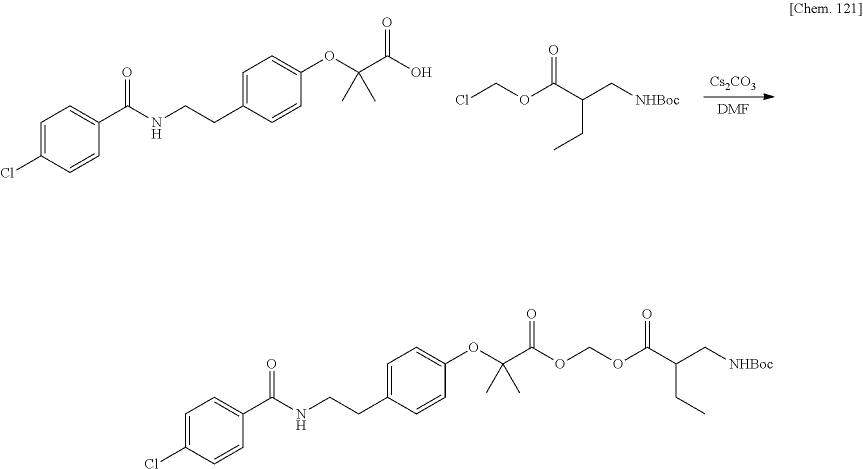

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 220 mg (0.829 mmol) of 2-[[[(1,1-dimethylethoxy) carbonyl] amino]methyl] butanoic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 482 mg (98%) of the title compound.

¹H-NMR (CDCl₃, δ): 0.90 (3H, t, J=8 Hz), 1.42 (9H, s), 1.50-1.66 (8H, m), 2.53-2.59 (1H, m), 2.86 (2H, t, J=7 Hz), 3.19 (1H, ddd, J=6, 9, 14 Hz), 3.31-3.36 (1H, m), 3.66 (2H, q, J=7 Hz), 4.80 (1H, br-s), 5.84 (1H, d, J=6 Hz), 5.87 (1H, d, J=6 Hz), 6.17 (1H, br-s), 6.82 (2H, d, J=9 Hz), 7.10 (2H, d, J=9 Hz), 7.38 (2H, d, J=9 Hz), 7.63 (2H, d, J=9 Hz)

### Reference Example 82

### 2-[4-[2-[(4-Chlorobenzoyl) amino] ethyl] phenoxy]-2-methylpropanoic Acid [2-(aminomethyl)-1-oxobutoxy] methyl Ester Hydrochloride

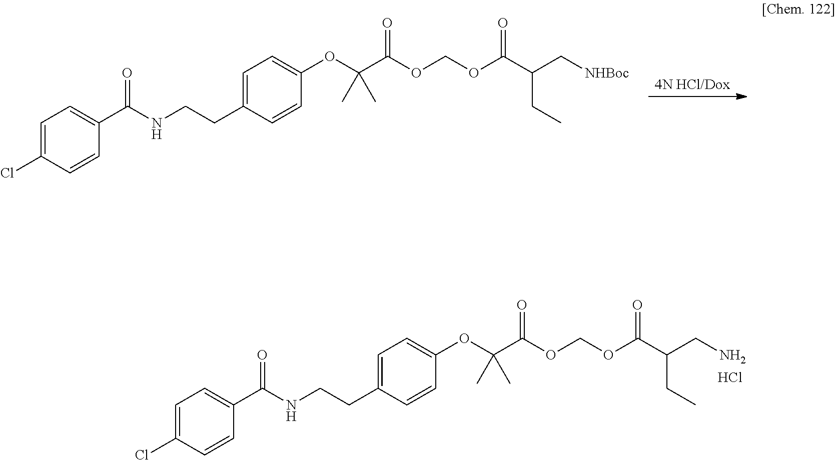 [SMILES: CCC(CNC(=O)OC(C)(C)C)C(=O)OCOC(=O)C(C)(C)Oc1ccc(CCNC(=O)c2ccc(Cl)cc2)cc1 ; "4N HCl/Dox" ; CCC(CN)C(=O)OCOC(=O)C(C)(C)Oc1ccc(CCNC(=O)c2ccc(Cl)cc2)cc1 ; Cl]

To 448 mg (0.758 mmol) of 2-[4-[2-[(4-chlorobenzoyl) amino] ethyl] phenoxy]-2-methylpropanoic acid [2-[[[(1,1-dimethylethoxy) carbonyl] amino]methyl]-1-oxobutoxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 397 mg (99%) of the title compound.

¹H-NMR (DMSO-d₆, δ): 0.80 (3H, t, J=8 Hz), 1.49 (6H, s), 1.52-1.67 (2H, m), 2.74-2.80 (3H, m), 2.91 (1H, dd, J=6, 13 Hz), 3.04 (1H, dd, J=8, 13 Hz), 3.42-3.46 (2H, m), 5.79 (1H, d, J=6 Hz), 5.92 (1H, d, J=6 Hz), 6.76 (2H, d, J=9 Hz), 7.14 (2H, d, J=9 Hz), 7.53 (2H, d, J=9 Hz), 7.85 (2H, d, J=9 Hz), 8.15 (3H, br-s), 8.69 (1H, t, J=6 Hz)

### Example 30

### [2-[(Bezafibrate-methoxy) carbonyl] butyl] amino-chondroitin Sulfate Conjugate

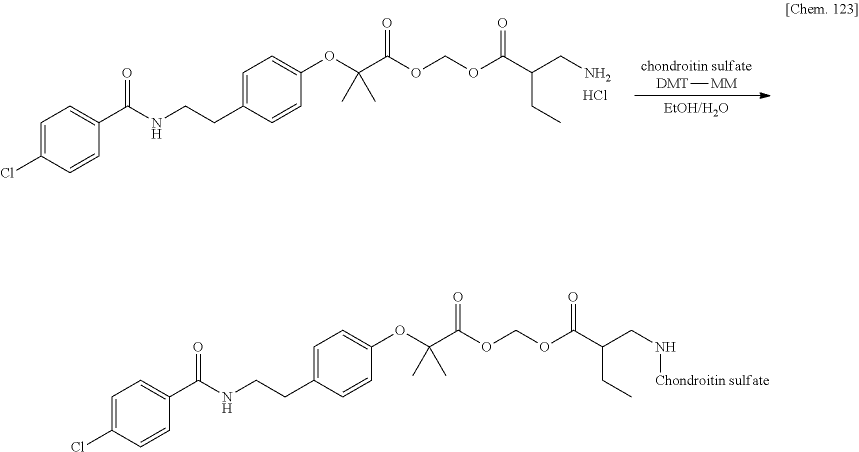

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 42 mg (0.080 mmol) of 2-[4-[2-[(4-chlorobenzoyl) amino]ethyl] phenoxy]-2-methylpropanoic acid [2-(aminomethyl)-1-oxobutoxy] methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 4 mL) thereto until immediately before the reaction solution became cloudy. The reaction solution was added dropwise under stirring to 8 mL of 90% ethanol. To the mixed liquid, 7 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 210 mg of the title compound. Based on values of integral in ¹H-NMR, the introduction ratio of bezafibrate per unit of whole disaccharide (glucuronic acid) of chondroitin sulfate was 15%.

### Reference Example 83

### Carbonic Acid Chloromethyl 2-[[(1,1-dimethylethoxy) carbonyl] amino] ethyl Ester

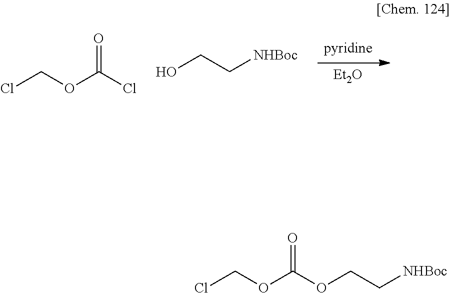 [SMILES: "Et2O" ; O=C(Cl)OCCl ; CC(C)(C)OC(=O)NCCO ; "pyridine" ; CC(C)(C)OC(=O)NCCOC(=O)OCCl]

To a diethyl ether (30 mL) solution of 2.00 g (12.4 mmol) of N-(2-hydroxyethyl) carbamic acid 1,1-dimethylethyl ester, 1.32 mL (14.9 mmol) of methyl chloroformate was added at −15° C. Subsequently, a diethyl ether (15 mL) solution of 1.20 mL (14.9 mmol) of pyridine in was added dropwise thereto. The temperature of the reaction solution was returned to room temperature, and the reaction solution was stirred for six hours. Thereafter, water was added to the reaction solution, and the resulting mixture was extracted with diethyl ether. The obtained organic layer was washed with a saturated ammonium chloride aqueous solution, saturated sodium bicarbonate water, and saturated saline, and then dried with anhydrous magnesium sulfate, and the solvent was distilled off under reduced pressure. The residue was purified by silica gel column chromatography to obtain 2.63 g (84%) of the title compound.

¹H-NMR (CDCl₃, δ): 1.45 (9H, s), 3.44-3.45 (2H, m), 4.29 (2H, t, J=5 Hz), 4.82 (1H, br-s), 5.74 (2H, s)

### Reference Example 84

### 2-[4-[2-[(4-Chlorobenzoyl) amino] ethyl] phenoxy]-2-methylpropanoic Acid [[[[2-[[(1,1-dimethylethoxy) carbonyl] amino] ethoxy] carbonyl] oxy] methyl Ester

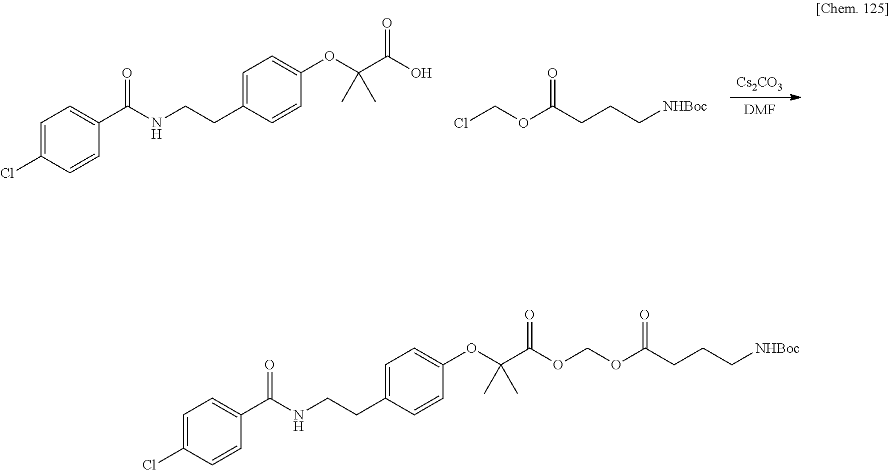

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 210 mg (0.829 mmol) of carbonic acid chloromethyl 2-[[(1,1-dimethylethoxy) carbonyl] amino] ethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 381 mg (79%) of the title compound.

¹H-NMR (CDCl₃, δ): 1.44 (9H, s), 1.61 (6H, s), 2.86 (2H, t, J=7 Hz), 3.37-3.40 (2H, m), 3.66 (2H, q, J=7 Hz), 4.18 (2H, t, J=6 Hz), 4.88 (1H, br-s), 5.83 (2H, s), 6.19 (1H, br-s), 6.82 (2H, d, J=9 Hz), 7.09 (2H, d, J=9 Hz), 7.38 (2H, d, J=9 Hz), 7.63 (2H, d, J=9 Hz)

### Reference Example 85

### 2-[4-[2-[(4-Chlorobenzoyl) amino] ethyl] phenoxy]-2-methylpropanoic Acid [[(2-aminoethoxy) carbonyl] oxy] methyl Ester Hydrochloride

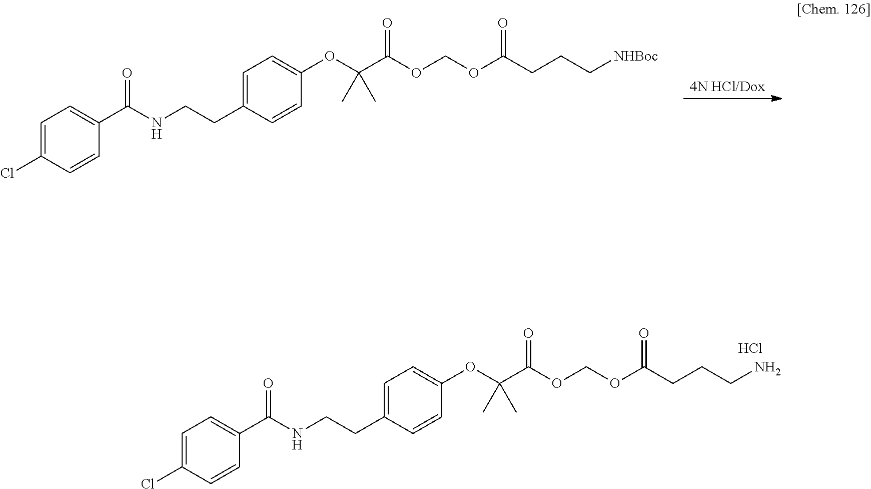 [SMILES: CC(C)(C)OC(=O)NCCCC(=O)OCOC(=O)C(C)(C)Oc1ccc(CCNC(=O)c2ccc(Cl)cc2)cc1 ; "4N HCl/Dox" ; CC(C)(Oc1ccc(CCNC(=O)c2ccc(Cl)cc2)cc1)C(=O)OCOC(=O)CCCN ; Cl]

To 343 mg (0.592 mmol) of 2-[4-[2-[(4-chlorobenzoyl) amino] ethyl]phenoxy]-2-methylpropanoic acid [[[[2-[[(1,1-dimethylethoxy) carbonyl] amino]ethoxy] carbonyl] oxy] methyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 292 mg (96%) of the title compound.

¹H-NMR (DMSO-d₆, δ): 1.51 (6H, s), 2.79 (2H, t, J=8 Hz), 3.13 (2H, br-s), 3.42-3.46 (2H, m), 4.33 (2H, t, J=6 Hz), 5.84 (2H, s), 6.76 (2H, d, J=9 Hz), 7.14 (2H, d, J=9 Hz), 7.53 (2H, d, J=9 Hz), 7.85 (2H, d, J=9 Hz), 8.21 (3H, br-s), 8.70 (1H, t, J=6 Hz)

### Example 31

### [2-[[(Bezafibrate-methoxy) carbonyl] oxy] ethyl] amino-chondroitin Sulfate Conjugate

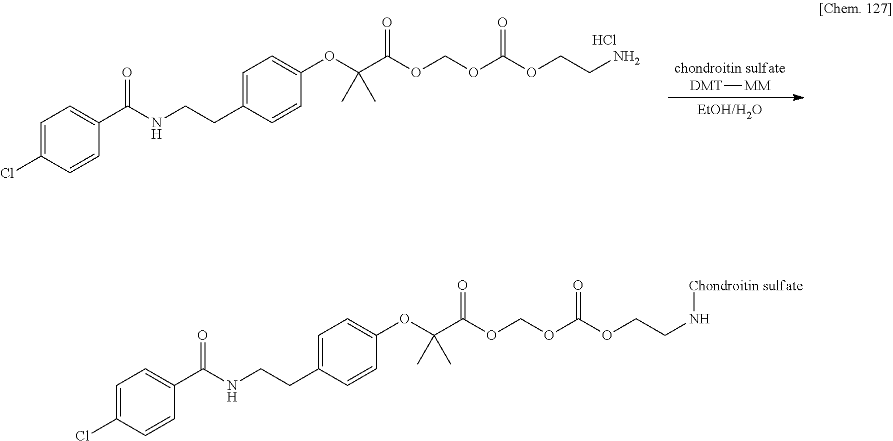

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 41 mg (0.080 mmol) of 2-[4-[2-[(4-chlorobenzoyl) amino]ethyl] phenoxy]-2-methylpropanoic acid [[(2-aminoethoxy) carbonyl] oxy] methyl Ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 101 mg of the title compound. Based on values of integral in ¹H-NMR, the introduction ratio of bezafibrate per unit of whole disaccharide (glucuronic acid) of chondroitin sulfate was 15%.

### Reference Example 86

### Carbonic Acid 1-chloroethyl 2-[[(1,1-dimethylethoxy) carbonyl] amino] ethyl Ester

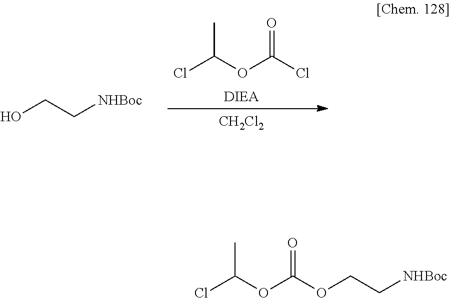

To a diethyl ether (20 mL) solution of 1.50 g (9.31 mmol) of 2-[[(1,1-dimethylethoxy) carbonyl] amino] ethanol, 1.22 mL (11.2 mmol) of 1-chloroethyl chloroformate was added at −15° C. Subsequently, a diethyl ether (10 mL) solution of 11.2 mL (14.0 mmol) of pyridine was added dropwise thereto. The temperature of the reaction solution was returned to room temperature, and the reaction solution was stirred overnight. Thereafter, water was added to the reaction solution, and the resulting mixture was extracted with diethyl ether. The obtained organic layer was washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to obtain 2.18 g (88%) of the title compound.

¹H-NMR (CDCl₃, δ): 1.45 (9H, s), 1.84 (3H, d, J=6 Hz), 3.44-3.45 (2H, m), 4.27 (2H, t, J=5 Hz), 4.82 (1H, br-s), 6.43 (1H, q, J=6 Hz)

### Reference Example 87

### 2-[4-[2-[(4-Chlorobenzoyl) amino] ethyl] phenoxy]-2-methylpropanoic Acid 1-[[[2-[[(1,1-dimethylethoxy) carbonyl] amino] ethoxy] carbonyl] oxy] ethyl Ester

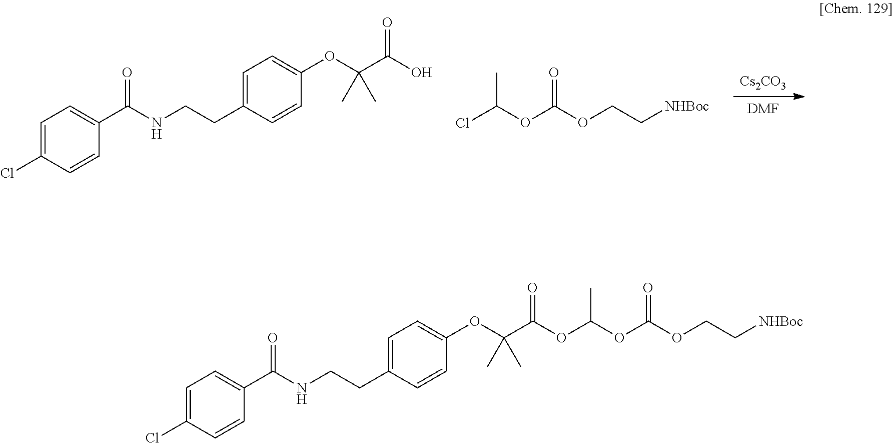

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 222 mg (0.829 mmol) of carbonic acid 1-chloroethyl 2-[[(1,1-dimethylethoxy) carbonyl] amino] ethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 352 mg (72%) of the title compound.

¹H-NMR (CDCl₃, δ): 1.43 (9H, s), 1.51 (3H, d, J=6 Hz), 1.59 (3H, s), 1.60 (3H, s), 2.86 (2H, t, J=7 Hz), 3.38-3.40 (2H, m), 3.64-3.68 (2H, m), 4.18 (2H, q, J=6 Hz), 4.87 (1H, br-s), 6.15 (1H, br-s), 6.79-6.83 (3H, m), 7.09 (2H, d, J=9 Hz), 7.38 (2H, d, J=9 Hz), 7.62 (2H, d, J=9 Hz)

### Reference Example 88

### 2-[4-[2-[(4-Chlorobenzoyl) amino] ethyl] phenoxy]-2-methylpropanoic Acid 1-[[(2-aminoethoxy) carbonyl] oxy] ethyl Ester Hydrochloride

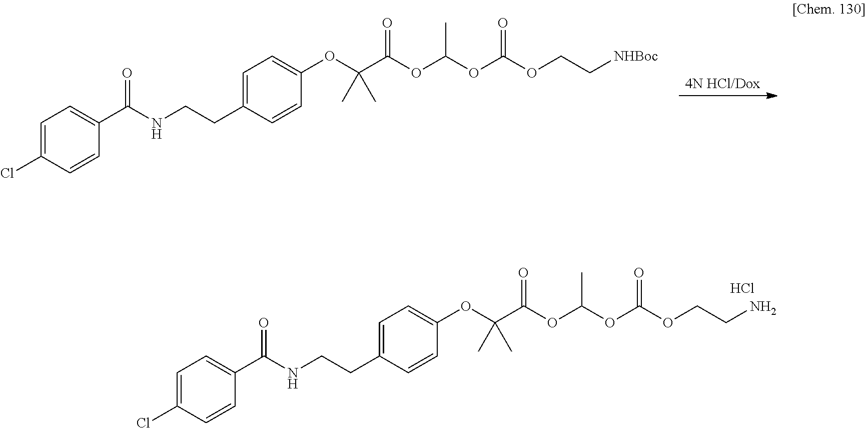 [SMILES: CC(OC(=O)OCCNC(=O)OC(C)(C)C)OC(=O)C(C)(C)Oc1ccc(CCNC(=O)c2ccc(Cl)cc2)cc1 ; "4N HCl/Dox" ; CC(OC(=O)OCCN)OC(=O)C(C)(C)Oc1ccc(CCNC(=O)c2ccc(Cl)cc2)cc1 ; Cl]

To 350 mg (0.590 mmol) of 2-[4-[2-[(4-chlorobenzoyl) amino] ethyl]phenoxy]-2-methylpropanoic acid 1-[[[[2-[[(1,1-dimethylethoxy) carbonyl] amino]ethoxy] carbonyl] oxy] ethyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 168 mg (58%) of the title compound.

¹H-NMR (DMSO-d₆, δ): 1.44 (3H, d, J=6 Hz), 1.49 (3H, s), 1.50 (3H, s), 2.78 (2H, t, J=8 Hz), 3.12-3.14 (2H, m), 3.41-3.45 (2H, m), 4.27-4.38 (2H, m), 6.73-6.77 (3H, m), 7.14 (2H, d, J=9 Hz), 7.53 (2H, d, J=9 Hz), 7.84 (2H, d, J=9 Hz), 8.13 (3H, br-s), 8.67 (1H, t, J=6 Hz)

### Example 32

### [2-[[(1-Bezafibrate-ethoxy) carbonyl] oxy] ethyl] amino-chondroitin Sulfate Conjugate

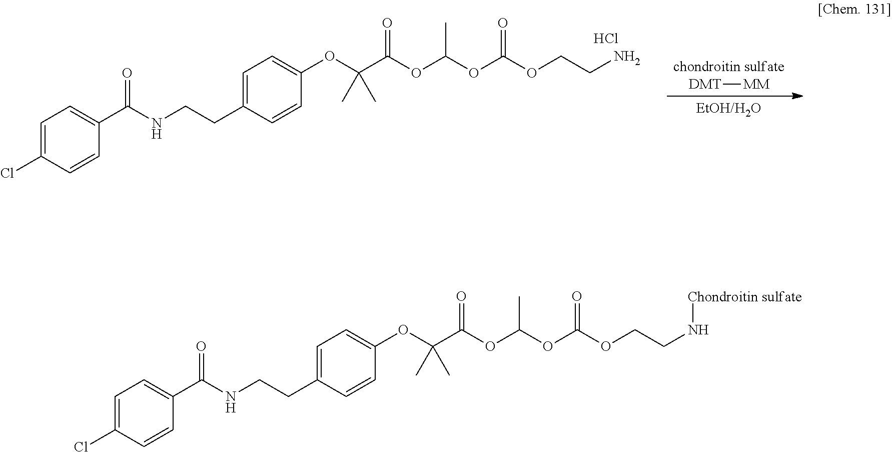

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 42 mg (0.080 mmol) of 2-[4-[2-[(4-chlorobenzoyl) amino]ethyl] phenoxy]-2-methylpropanoic acid 1-[[(2-aminoethoxy) carbonyl] oxy] ethyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 4 mL) thereto until immediately before the reaction solution became cloudy. The reaction solution was added dropwise under stirring to 8 mL of 90% ethanol. To the mixed liquid, 7 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 253 mg of the title compound. Based on values of integral in ¹H-NMR, the introduction ratio of bezafibrate per unit of whole disaccharide (glucuronic acid) of chondroitin sulfate was 18%.

### Reference Example 89

### 5-(Aminosulfonyl)-4-chloro-2-[(2-furanylmethyl) amino] benzoic Acid 1-[[[2-[[(1,1-dimethylethoxy) carbonyl] amino] ethoxy] carbonyl] oxy] ethyl Ester

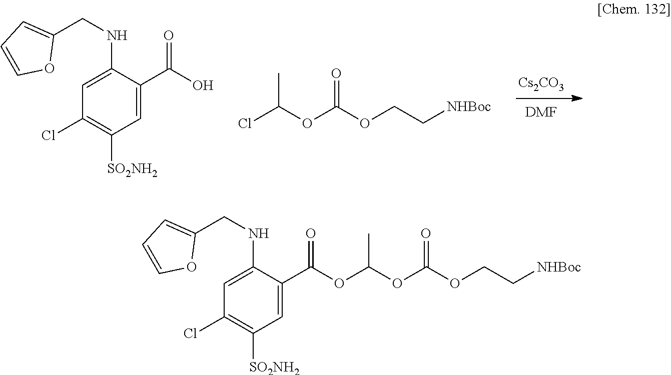

To a methanol solution of 300 mg (0.907 mmol) of furosemide, 148 mg (0.454 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 242 mg (0.907 mmol) of carbonic acid 1-chloroethyl 2-[[(1,1-dimethylethoxy) carbonyl] amino] ethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 93 mg (18%) of the title compound.

¹H-NMR (CDCl₃, δ, mixture of rotamers): 1.42 (9H, s), 1.65 (3H, d, J=6 Hz), 3.41-3.42 (2H, m), 4.23 (2H, br-s), 4.45 (2H, d, J=6 Hz), 4.88-5.01 (2.5H, m), 5.75 (0.5H, br-s), 6.28 (1H, dd, J=1, 3 Hz), 6.35 (1H, dd, J=2, 3 Hz), 6.90 (1H, s), 6.96-6.97 (1H, m), 7.40 (1H, dd, J=1, 2 Hz), 8.55 (1H, br), 8.60 (1H, s)

### Reference Example 90

### 5-(Aminosulfonyl)-4-chloro-2-[(2-furanylmethyl) amino] benzoic Acid 2-[[(2-aminoethoxy) carbonyl] oxy] ethyl Ester Dihydrochloride

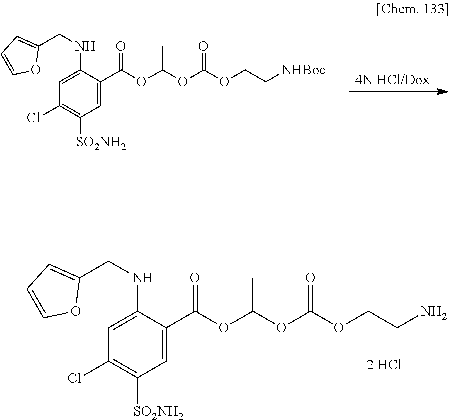 [SMILES: CC(OC(=O)OCCNC(=O)OC(C)(C)C)OC(=O)c1cc(S(N)(=O)=O)c(Cl)cc1NCc1ccco1 ; "4N HCl/Dox" ; CC(OC(=O)OCCN)OC(=O)c1cc(S(N)(=O)=O)c(Cl)cc1NCc1ccco1]

To 75 mg (0.147 mmol) of 5-(aminosulfonyl)-4-chloro-2-[(2-furanylmethyl) amino] benzoic acid 1-[[[2-[[(1,1-dimethylethoxy) carbonyl] amino] ethoxy] carbonyl]oxy] ethyl ester, 2 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and the precipitated solid was collected by filtration to obtain 33 mg (51%) of the title compound.

¹H-NMR (DMSO-d₆, δ): 1.60 (3H, d, J=5 Hz), 3.12-3.13 (2H, m), 4.27-4.35 (2H, m), 4.63 (2H, d, J=6 Hz), 6.39 (1H, dd, J=1, 3 Hz), 6.43 (1H, dd, J=2, 3 Hz), 6.92 (1H, q, J=5 Hz), 7.16 (1H, s), 7.39 (2H, s), 7.63 (1H, dd, J=1, 2 Hz), 8.06 (3H, br), 8.36 (1H, s), 8.39 (1H, t, J=6 Hz)

### Example 33

### [2-[[(1-Furosemide-ethoxy) carbonyl] oxy] ethyl] amino-chondroitin Sulfate Conjugate

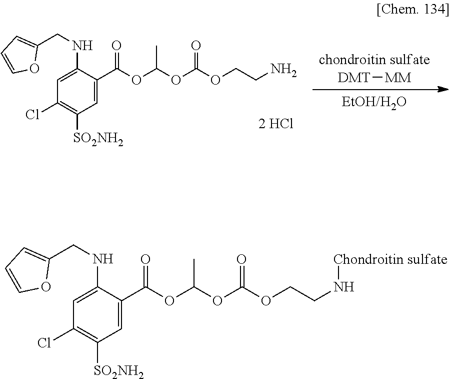

To 2.0 g (0.20 mmol) of a 5% sodium chondroitin sulfate aqueous solution, 1 mL of ethanol was added dropwise under stirring. To the mixed liquid, an ethanol 1.1 mL-water 0.5 mL solution of 20 mg (0.040 mmol) of 5-(aminosulfonyl)-4-chloro-2-[(2-furanylmethyl) amino] benzoic acid 2-[[(2-aminoethoxy) carbonyl] oxy] ethyl ester dihydrochloride was added. Subsequently, an ethanol 0.4 mL solution of 11 mg (0.040 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 50 μL of a 20% sodium chloride aqueous solution was added, and ethanol was further added dropwise (about 1.5 mL) until immediately before the reaction solution became cloudy. The reaction solution was added dropwise under stirring to 8 mL of 90% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 120 mg of the title compound. Based on values of integral in ¹H-NMR, the introduction ratio of furosemide per unit of whole disaccharide (glucuronic acid) of chondroitin sulfate was 13%.

### Reference Example 91

### Carbonic Acid 1-chloroethyl 2-[[(1,1-dimethylethoxy) carbonyl] amino]-1-methylethyl Ester

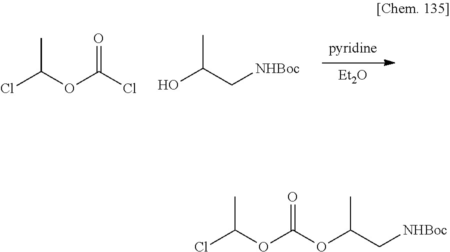

To a diethyl ether (15 mL) solution of 1.78 g (10.2 mmol) of N-(2-hydroxypropyl) carbamic acid 1,1-dimethylethyl ester, 1.33 mL (12.2 mmol) of 1-chloroethyl chloroformate was added at −15° C. Subsequently, a diethyl ether (10 mL) solution of 1.23 mL (15.2 mmol) of pyridine was added dropwise thereto. The temperature of the reaction solution was returned to room temperature, and the reaction solution was stirred for six hours. Thereafter, water was added to the reaction solution, and the resulting mixture was extracted with diethyl ether. The obtained organic layer was washed with a saturated ammonium chloride aqueous solution, saturated sodium bicarbonate water, and saturated saline, and then dried with anhydrous magnesium sulfate, and the solvent was distilled off under reduced pressure. The residue was purified by silica gel column chromatography to obtain 2.25 g (79%) of the title compound.

¹H-NMR (CDCl₃, δ, mixture of diastereomers): 1.31 (1.5H, d, J=6 Hz), 1.32 (1.5H, d, J=6 Hz), 1.44 (9H, s), 1.83 (1.5H, d, J=6 Hz), 1.84 (1.5H, d, J=6 Hz), 3.22-3.36 (1H, m), 3.41-3.43 (1H, m), 4.80 (1H, br-s), 4.87-4.91 (1H, m), 6.43 (1H, q, J=6 Hz)

### Reference Example 92

### 2-[4-[2-[(4-Chlorobenzoyl) amino] ethyl] phenoxy]-2-methylpropanoic Acid 1-[[[2-[[(1,1-dimethylethoxy) carbonyl] amino]-1-methylethoxy] carbonyl] oxy] ethyl Ester

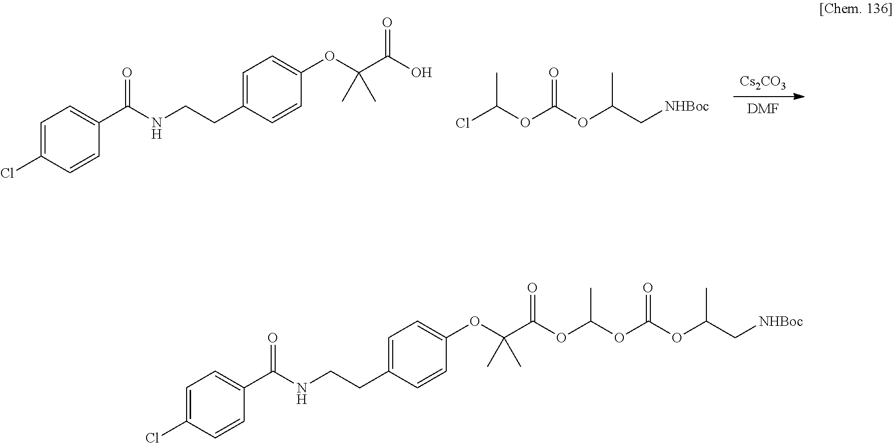

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 233 mg (0.829 mmol) of carbonic acid 1-chloroethyl 2-[[(1,1-dimethylethoxy) carbonyl] amino]-1-methylethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 478 mg (95%) of the title compound.

¹H-NMR (DMSO-d₆, δ, mixture of diastereomers): 1.12 (1.5H, d, J=7 Hz), 1.17 (1.5H, d, J=7 Hz), 1.35 (4.5H, s), 1.37 (4.5H, s), 1.40 (1.5H, d, J=6 Hz), 1.42 (1.5H, d, J=5 Hz), 1.47 (1.5H, s), 1.49 (4.5H, s), 2.77 (2H, t, J=8 Hz), 3.01-3.17 (2H, m), 3.40-3.46 (2H, m), 4.66-4.72 (1H, m), 6.68-6.72 (1H, m), 6.74-6.77 (2H, m), 6.99-7.03 (1H, m), 7.11-7.14 (2H, m), 7.52 (2H, d, J=9 Hz), 7.82 (2H, d, J=9 Hz), 8.60 (1H, t, J=6 Hz)

### Reference Example 93

### 2-[4-[2-[(4-Chlorobenzoyl) amino] ethyl] phenoxy]-2-methylpropanoic Acid 1-[[(2-amino-1-methylethoxy) carbonyl] oxy] ethyl Ester Hydrochloride

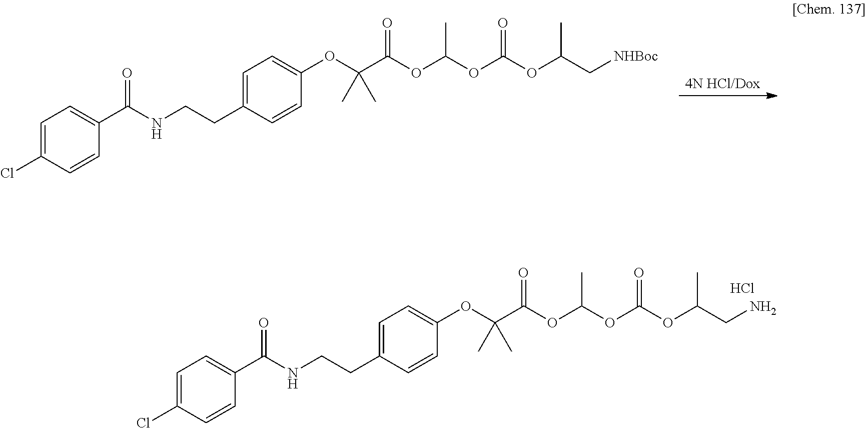 [SMILES: CC(CNC(=O)OC(C)(C)C)OC(=O)OC(C)OC(=O)C(C)(C)Oc1ccc(CCNC(=O)c2ccc(Cl)cc2)cc1 ; "4N HCl/Dox" ; CC(CN)OC(=O)OC(C)OC(=O)C(C)(C)Oc1ccc(CCNC(=O)c2ccc(Cl)cc2)cc1 ; Cl]

To 460 mg (0.758 mmol) of 2-[4-[2-[(4-chlorobenzoyl) amino] ethyl]phenoxy]-2-methylpropanoic acid 1-[[[[2-[[(1,1-dimethylethoxy) carbonyl] amino]-1-methylethoxy] carbonyl] oxy] ethyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 232 mg (56%) of the title compound.

¹H-NMR (DMSO-d₆, δ, mixture of diastereomers): 1.23 (1.5H, d, J=6 Hz), 1.29 (1.5H, d, J=6 Hz), 1.42 (1.5H, d, J=6 Hz), 1.45 (1.5H, d, J=6 Hz), 1.48 (1.5H, s), 1.50 (4.5H, s), 2.76-2.80 (2H, m), 2.97-3.08 (2H, m), 3.42-3.46 (2H, m), 4.88-4.92 (1H, m), 6.72-6.77 (3H, m), 7.13 (1H, d, J=9 Hz), 7.14 (1H, d, J=9 Hz), 7.53 (2H, d, J=9 Hz), 7.84 (2H, d, J=9 Hz), 8.18 (3H, br-s), 8.66-8.68 (1H, m)

### Example 34

### [2-[[(1-Bezafibrate-ethoxy) carbonyl] oxy] propyl] amino-chondroitin Sulfate Conjugate

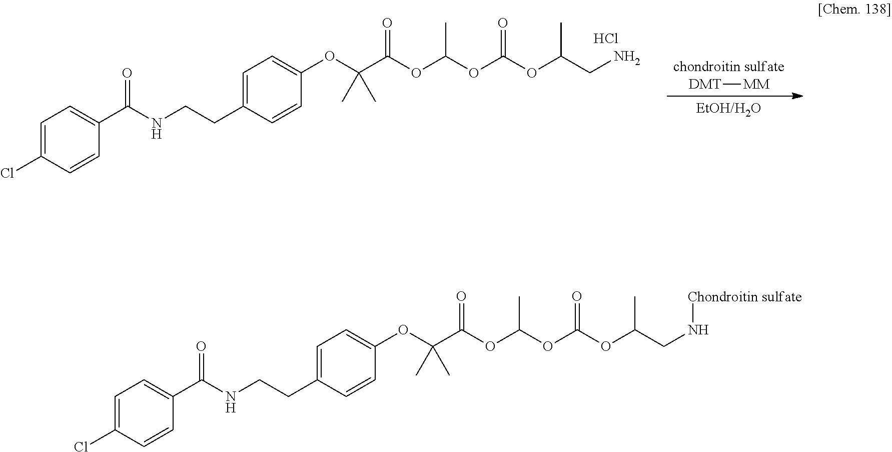

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 43 mg (0.080 mmol) of 2-[4-[2-[(4-chlorobenzoyl) amino]ethyl] phenoxy]-2-methylpropanoic acid 1-[[(2-amino-1-methylethoxy) carbonyl] oxy]ethyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 194 mg of the title compound. Based on values of integral in ¹H-NMR, the introduction ratio of bezafibrate per unit of whole disaccharide (glucuronic acid) of chondroitin sulfate was 16%.

### Reference Example 94

### N-(2-Hydroxy-2-methylpropyl) carbamic Acid 1,1-dimethylethyl Ester

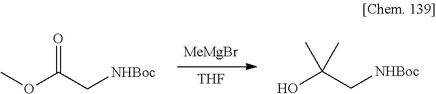

To a THE (15 mL) solution of 1.47 g (7.77 mmol) of N-Boc-glycine methyl ester, 31 mL (31.1 mmol) of a 1 M solution of methylmagnesium bromide in THE was added dropwise at −15° C. The temperature of the reaction solution was returned to 0° C., and the reaction solution was stirred for six hours. Thereafter, to the reaction solution, water was added, and subsequently 10% potassium hydrogen sulfate was added. The resulting mixture was extracted with ethyl acetate. The obtained organic layer was washed with saturated sodium bicarbonate water and saturated saline, and then dried with anhydrous magnesium sulfate, and the solvent was distilled off under reduced pressure to obtain 1.52 g (quant.) of the title compound.

¹H-NMR (CDCl₃, δ): 1.21 (6H, s), 1.45 (9H, s), 2.19 (1H, br-s), 3.12 (2H, d, J=6 Hz), 4.93 (1H, br-s)

### Reference Example 95

Carbonic Acid 1-chloroethyl 2-[[(1,1-dimethylethoxy) carbonyl] amino]-1,1-dimethylethyl Ester

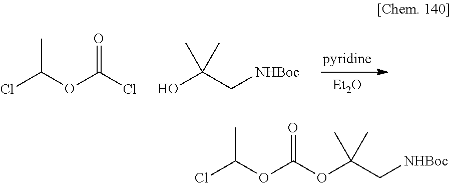

To a diethyl ether (9 mL) solution of 600 mg (3.17 mmol) of N-(2-hydroxy-2-methylpropyl) carbamic acid 1,1-dimethylethyl ester, 0.415 mL (3.80 mmol)) of 1-chloroethyl chloroformate was added at −15° C. Subsequently, a diethyl ether (3 mL) solution of 0.383 mL (4.76 mmol) of pyridine was added dropwise thereto. The temperature of the reaction solution was returned to room temperature, and the reaction solution was stirred overnight. Thereafter, water was added to the reaction solution, and the resulting mixture was extracted with diethyl ether. The obtained organic layer was washed with a saturated ammonium chloride aqueous solution, saturated sodium bicarbonate water, and saturated saline, and then dried with anhydrous magnesium sulfate, and the solvent was distilled off under reduced pressure. The residue was purified by silica gel column chromatography to obtain 703 mg (75%) of the title compound.

¹H-NMR (CDCl₃, δ): 1.45 (9H, s), 1.50 (6H, s), 1.82 (3H, d, J=6 Hz), 3.37 (1H, dd, J=7, 10 Hz), 3.42 (1H, dd, J=7, 10 Hz), 4.93 (1H, br-s), 6.38 (1H, q, J=6 Hz)

### Reference Example 96

### 2-[4-[2-[(4-Chlorobenzoyl) amino] ethyl] phenoxy]-2-methylpropanoic Acid 1-[[[2-[[(1,1-dimethylethoxy) carbonyl] amino]-1, 1-dimethylethoxy] carbonyl] oxy]ethyl Ester

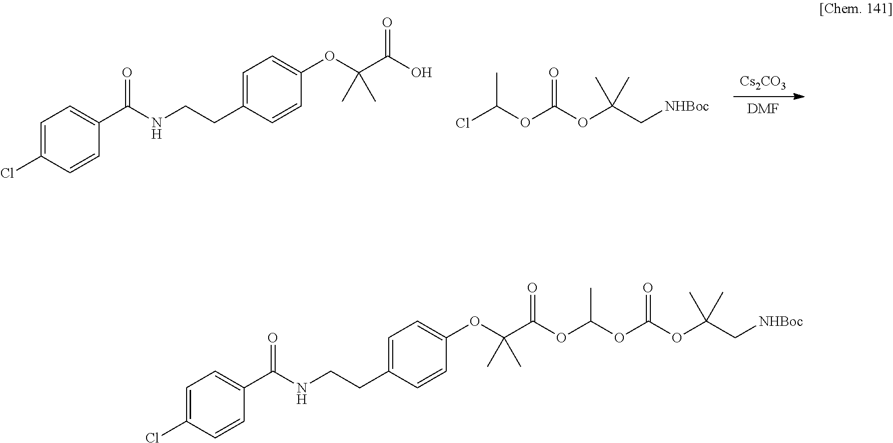

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 245 mg (0.829 mmol) of carbonic acid 1-chloroethyl 2-[[(1,1-dimethylethoxy) carbonyl] amino]-1,1-dimethylethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 453 mg (88%) of the title compound.

¹H-NMR (CDCl₃, δ): 1.43-1.44 (12H, m), 1.49 (3H, d, J=6 Hz), 1.58 (3H, s), 1.59 (3H, s), 1.60 (3H, s), 2.86 (2H, t, J=7 Hz), 3.37 (2H, d, J=6 Hz), 3.64-3.68 (2H, m), 4.95 (1H, t, J=6 Hz), 6.11 (1H, br-s), 6.77 (1H, q, J=6 Hz), 6.83 (2H, d, J=9 Hz), 7.09 (2H, d, J=9 Hz), 7.38 (2H, d, J=9 Hz), 7.62 (2H, d, J=9 Hz)

### Reference Example 97

### 2-[4-[2-[(4-Chlorobenzoyl) amino] ethyl] phenoxy]-2-methylpropanoic Acid 1-[[(2-amino-1,1-dimethylethoxy) carbonyl] oxy] ethyl Ester Hydrochloride

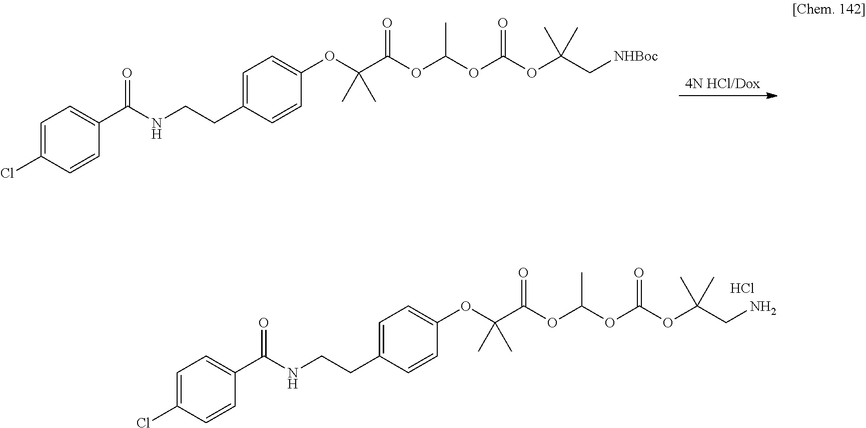 [SMILES: CC(OC(=O)OC(C)(C)CNC(=O)OC(C)(C)C)OC(=O)C(C)(C)Oc1ccc(CCNC(=O)c2ccc(Cl)cc2)cc1 ; "4N HCl/Dox" ; CC(OC(=O)OC(C)(C)CN)OC(=O)C(C)(C)Oc1ccc(CCNC(=O)c2ccc(Cl)cc2)cc1 ; Cl]

To 440 mg (0.708 mmol) of 2-[4-[2-[(4-chlorobenzoyl) amino] ethyl]phenoxy]-2-methylpropanoic acid 1-[[[[2-[[(1,1-dimethylethoxy) carbonyl] amino]-1,1-dimethylethoxy] carbonyl] oxy] ethyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 312 mg (79%) of the title compound.

¹H-NMR (DMSO-d₆, δ): 1.41 (3H, d, J=6 Hz), 1.46 (3H, s), 1.48 (3H, s), 1.489 (3H, s), 1.494 (3H, s), 2.78 (2H, t, J=8 Hz), 3.08 (1H, br-d, J=13 Hz), 3.19 (1H, br-d, 13 Hz), 3.42-3.46 (2H, m), 6.71 (1H, q, J=6 Hz), 6.75 (2H, d, J=9 Hz), 7.13 (2H, d, J=9 Hz), 7.53 (2H, d, J=9 Hz), 7.85 (2H, d, J=9 Hz), 8.31 (3H, br-s), 8.69 (1H, t, J=6 Hz)

### Example 35

### [2-[[(1-Bezafibrate-ethoxy) carbonyl] oxy]-2-methylpropyl] amino-chondroitin Sulfate Conjugate

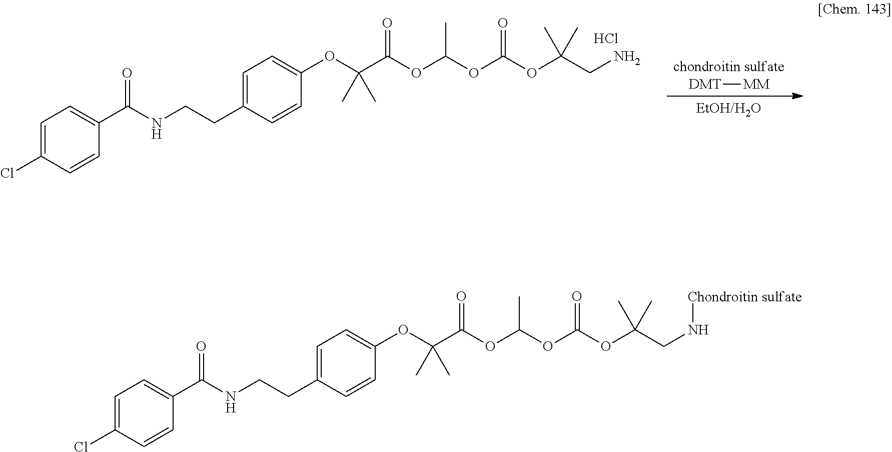

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 44 mg (0.080 mmol) of 2-[4-[2-[(4-chlorobenzoyl) amino]ethyl] phenoxy]-2-methylpropanoic acid 1-[[(2-amino-1,1-dimethylethoxy) carbonyl]oxy] ethyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 8 mL of 90% ethanol. To the mixed liquid, 9 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 190 mg of the title compound. Based on values of integral in ¹H-NMR, the introduction ratio of bezafibrate per unit of whole disaccharide (glucuronic acid) of chondroitin sulfate was 4%.

### Reference Example 98

### N-(2-Hydroxy-2-phenylethyl) carbamic Acid 1,1-dimethylethyl Ester

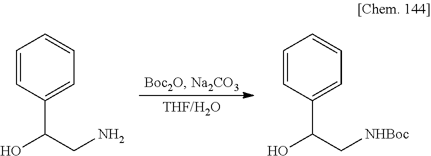

To a water (30 mL)-THF (30 mL) mixed solution of 2.00 g (14.6 mmol) of 2-amino-1-phenylethanol and 3.55 g (33.5 mmol) of sodium carbonate, under cooling on ice, a THF (30 mL) solution of 3.50 g (16.0 mmol) of tert-butyl dicarbonate was added, and the resulting mixture was stirred at room temperature for three hours. Water was added to the reaction solution, and the resulting mixture was extracted with ethyl acetate. The obtained organic layer was washed with saturated sodium bicarbonate water and saturated saline, and then dried with anhydrous magnesium sulfate, and the solvent was distilled off under reduced pressure. The obtained residue was recrystallized from an ethyl acetate-hexane mixed solvent to obtain 3.42 g (99%) of the title compound.

¹H-NMR (CDCl₃, δ): 1.45 (9H, s), 3.10 (1H, br-s), 3.26 (1H, ddd, J=6, 8, 14 Hz), 3.47-3.51 (1H, m), 4.82-4.83 (1H, m), 4.95 (1H, br-s), 7.17-7.37 (5H, m)

### Reference Example 99

### Carbonic Acid 1-chloroethyl 2-[[(1,1-dimethylethoxy) carbonyl] amino]-1-phenylethyl Ester

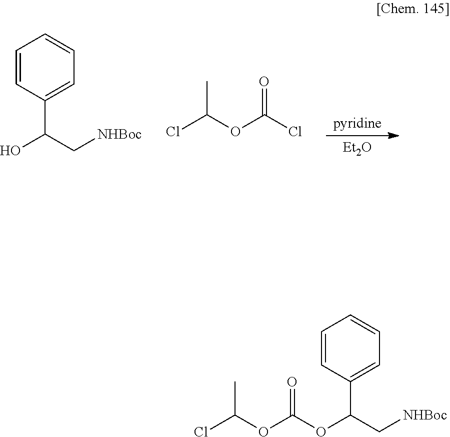

To a diethyl ether (15 mL) solution of 1.00 g (4.21 mmol) of N-(2-hydroxy-2-phenylethyl) carbamic acid 1,1-dimethylethyl ester, 0.552 mL (5.06 mmol) of 1-chloroethyl chloroformate was added at −15° C. Subsequently, a diethyl ether (5 mL) solution of 0.509 mL (6.32 mmol) of pyridine was added dropwise thereto. The temperature of the reaction solution was returned to room temperature, and the reaction solution was stirred overnight. Thereafter, water was added to the reaction solution, and the resulting mixture was extracted with diethyl ether. The obtained organic layer was washed with a saturated ammonium chloride aqueous solution, saturated sodium bicarbonate water, and saturated saline, and then dried with anhydrous magnesium sulfate, and the solvent was distilled off under reduced pressure. The residue was purified by silica gel column chromatography to obtain 1.09 g (75%) of the title compound as a 1:2 diastereomeric mixture.

¹H-NMR (CDCl₃, δ, mixture of diastereomers): 1.43 (9H, s), 1.80 (1H, d, J=6 Hz), 1.84 (2H, d, J=6 Hz), 3.43-3.48 (1H, m), 3.59-3.64 (1H, m), 4.79 (2/3H, br-s), 4.83 (1/3H, br-s), 5.71-5.73 (1H, m), 6.36 (1/3H, q, J=6 Hz), 6.40 (2/3H, q, J=6 Hz), 7.32-7.39 (5H, m)

### Reference Example 100

### 2-[4-[2-[(4-Chlorobenzoyl) amino] ethyl] phenoxy]-2-methylpropanoic Acid 1-[[[2-[[(1,1-dimethylethoxy) carbonyl] amino]-1-phenylethoxy] carbonyl] oxy] ethyl Ester

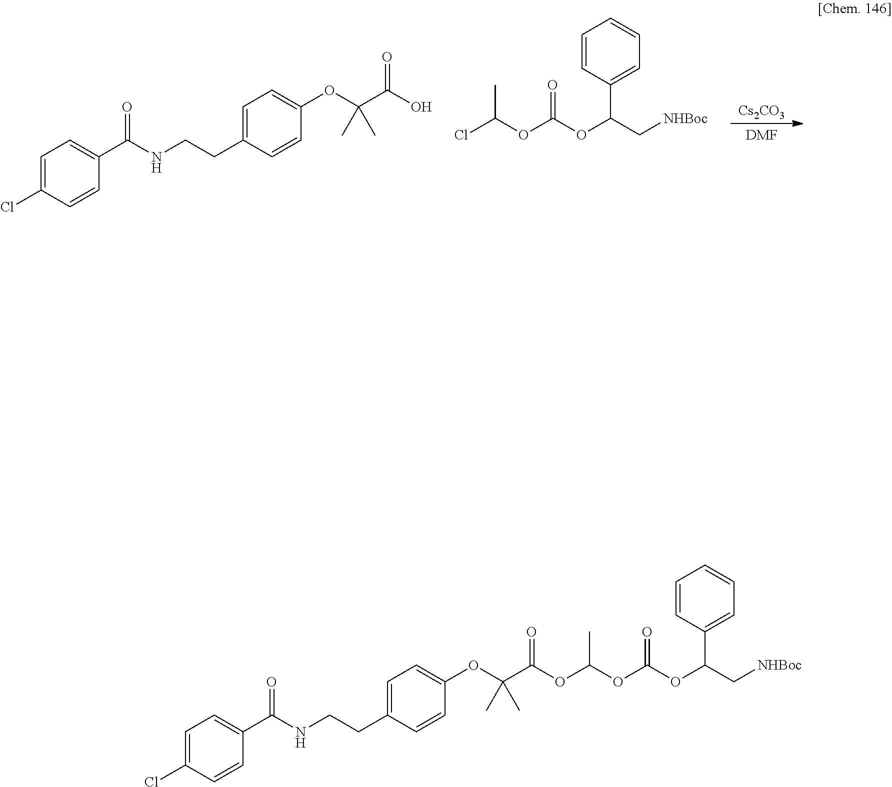

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 285 mg (0.829 mmol) of carbonic acid 1-chloroethyl 2-[[(1,1-dimethylethoxy) carbonyl] amino]-1-phenylethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 285 mg (55%) of the title compound as a 1:2 diastereomeric mixture.

¹H-NMR (CDCl₃, δ, mixture of diastereomers): 1.40 (6H, s), 1.43 (3H, s), 1.47 (2H, d, J=6 Hz), 1.50 (1H, d, J=6 Hz), 1.58 (6H, s), 2.82 (2/3H, t, J=7 Hz), 2.87 (4/3H, t, J=7 Hz), 3.41-3.55 (2H, m), 3.62-3.71 (2H, m), 4.78 (2/3H, br-s), 4.83 (1/3H, br-s), 5.63-5.66 (1H, m), 6.05 (1/3H, br-s), 6.18 (2/3H, br-s), 6.75-6.84 (3H, m), 7.00 (2/3H, d, J=9 Hz), 7.11 (4/3H, d, J=9 Hz), 7.31-7.38 (7H, m), 7.59-7.62 (2H, m)

### Reference Example 101

### 2-[4-[2-[(4-Chlorobenzoyl) amino] ethyl] phenoxy]-2-methylpropanoic Acid 1-[[(2-amino-1-phenylethoxy) carbonyl] oxy] ethyl Ester Hydrochloride

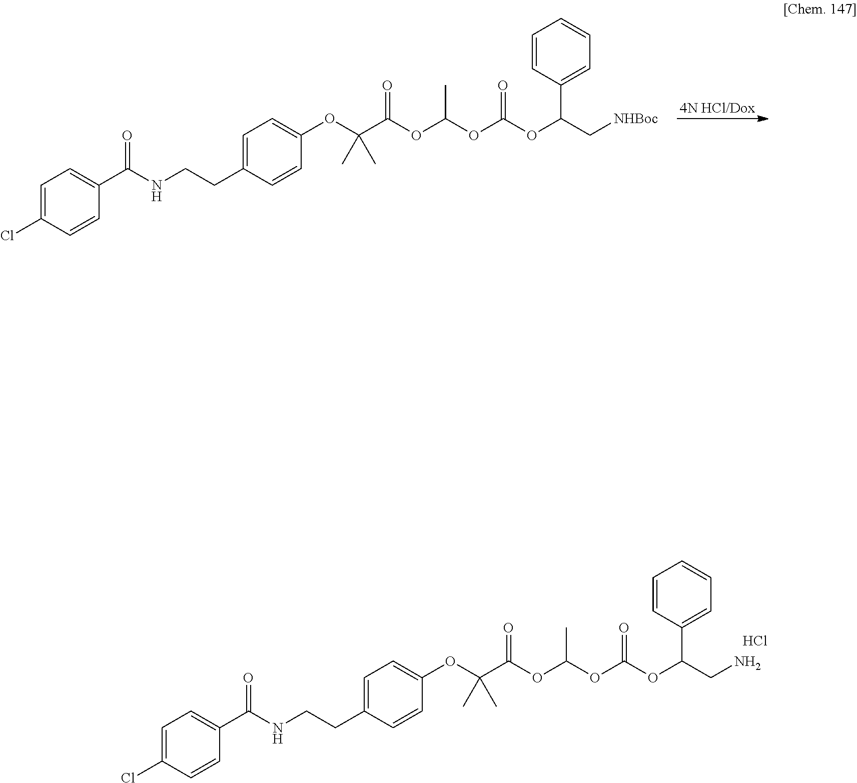 [SMILES: CC(OC(=O)OC(CNC(=O)OC(C)(C)C)c1ccccc1)OC(=O)C(C)(C)Oc1ccc(CCNC(=O)c2ccc(Cl)cc2)cc1 ; "4N HCl/Dox" ; CC(OC(=O)OC(CN)c1ccccc1)OC(=O)C(C)(C)Oc1ccc(CCNC(=O)c2ccc(Cl)cc2)cc1 ; Cl]

To 250 mg (0.374 mmol) of 2-[4-[2-[(4-chlorobenzoyl) amino] ethyl]phenoxy]-2-methylpropanoic acid 1-[[[[2-[[(1,1-dimethylethoxy) carbonyl] amino]-1-phenylethoxy] carbonyl] oxy] ethyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 109 mg (48%) of the title compound as a 1:2 diastereomer mixture.

¹H-NMR (DMSO-d₆, δ, mixture of diastereomers): 1.29 (1H, s), 1.31 (1H, s), 1.38 (2H, d, J=6 Hz), 1.45 (1H, d, J=6 Hz), 1.48 (2H, s), 1.49 (2H, s), 2.75-2.80 (2H, m), 3.20-3.24 (1H, m), 3.38-3.46 (3H, m), 5.78-5.83 (1H, m), 6.69-6.76 (3H, m), 7.04 (1/3H, d, J=9 Hz), 7.14 (4/3H, d, J=9 Hz), 7.37-7.45 (5H, m), 7.53 (2H, d, J=9 Hz), 7.82-7.85 (2H, m), 8.33 (3H, br-s), 8.65 (1H, t, J=6 Hz)

### Example 36

### [2-[[(1-Bezafibrate-ethoxy) carbonyl] oxy]-2-phenylethyl] amino-chondroitin Sulfate Conjugate

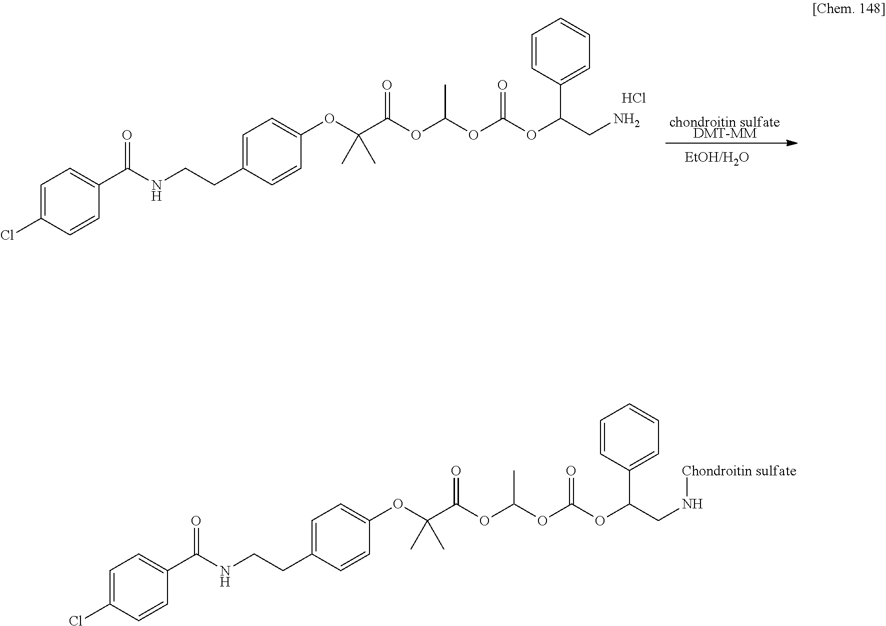

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 48 mg (0.080 mmol) of 2-[4-[2-[(4-chlorobenzoyl) amino]ethyl] phenoxy]-2-methylpropanoic acid 1-[[(2-amino-1-phenylethoxy) carbonyl] oxy]ethyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 4 mL) thereto until immediately before the reaction solution became cloudy. The reaction solution was added dropwise under stirring to 8 mL of 90% ethanol. To the mixed liquid, 7 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 192 mg of the title compound. Based on values of integral in H-NMR, the introduction ratio of bezafibrate per unit of whole disaccharide (glucuronic acid) of chondroitin sulfate was 13%.

### Reference Example 102

### N-(2-Hydroxybutyl) carbamic Acid 1,1-dimethylethyl Ester

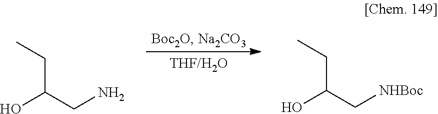

To a water (50 mL)-THF (50 mL) mixed solution of 4.46 g (50.0 mmol) of 1-amino-2-butanol and 12.2 g (115 mmol) of sodium carbonate, under cooling on ice, a THF (50 mL) solution of 12.0 g (55.0 mmol) of tert-butyl dicarbonate was added, and the resulting mixture was stirred at room temperature for three hours. Water was added to the reaction solution, and the resulting mixture was extracted with ethyl acetate. The obtained organic layer was washed with saturated sodium bicarbonate water and saturated saline, and then dried with anhydrous magnesium sulfate, and the solvent was distilled off under reduced pressure to obtain 9.62 g (quant.) of the title compound.

¹H-NMR (CDCl₃, δ): 0.96 (3H, t, J=8 Hz), 1.45-1.50 (1H, m), 2.28 (1H, br-s), 2.99-3.04 (1H, m), 3.30-3.33 (1H, m), 3.61-3.62 (1H, m), 4.92 (1H, br-s)

### Reference Example 103

### Carbonic Acid 1-chloroethyl 1-[[[(1,1-dimethylethoxy) carbonyl] amino]methyl] propyl Ester

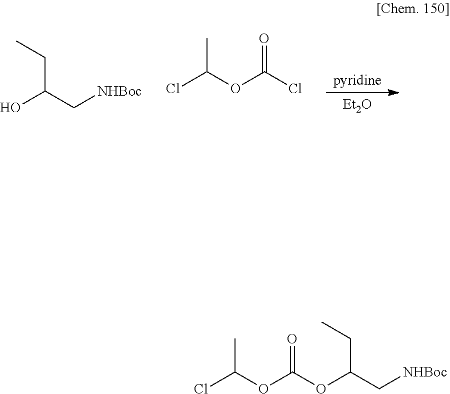

To a diethyl ether (30 mL) solution of 2.84 g (15.0 mmol) of N-(2-hydroxybutyl) carbamic acid 1,1-dimethylethyl ester, 1.96 mL (18.0 mmol) of 1-chloroethyl chloroformate was added at −15° C. Subsequently, a diethyl ether (10 mL) solution of 1.81 mL (22.5 mmol) of pyridine was added dropwise thereto. The temperature of the reaction solution was returned to room temperature, and the reaction solution was stirred overnight. Thereafter, water was added to the reaction solution, and the resulting mixture was extracted with diethyl ether. The obtained organic layer was washed with a saturated ammonium chloride aqueous solution, saturated sodium bicarbonate water, and saturated saline, and then dried with anhydrous magnesium sulfate, and the solvent was distilled off under reduced pressure. The residue was purified by silica gel column chromatography to obtain 4.12 g (93%) of the title compound as a 1:2 diastereomeric mixture.

¹H-NMR (CDCl₃, δ, mixture of diastereomers): 0.95-0.99 (3H, m), 1.44 (9H, s), 1.62-1.72 (2H, m), 1.83-1.85 (3H. m), 3.23-3.28 (1H, m), 3.43-3.49 (1H, m), 4.73-4.78 (2H, m), 6.43 (1H, q, J=6 Hz)

### Reference Example 104

### 2-[4-[2-[(4-Chlorobenzoyl) amino] ethyl] phenoxy]-2-methylpropanoic Acid 1-[[[1-[[[(1,1-dimethylethoxy) carbonyl] amino] methyl] propoxy] carbonyl] oxy] ethyl Ester

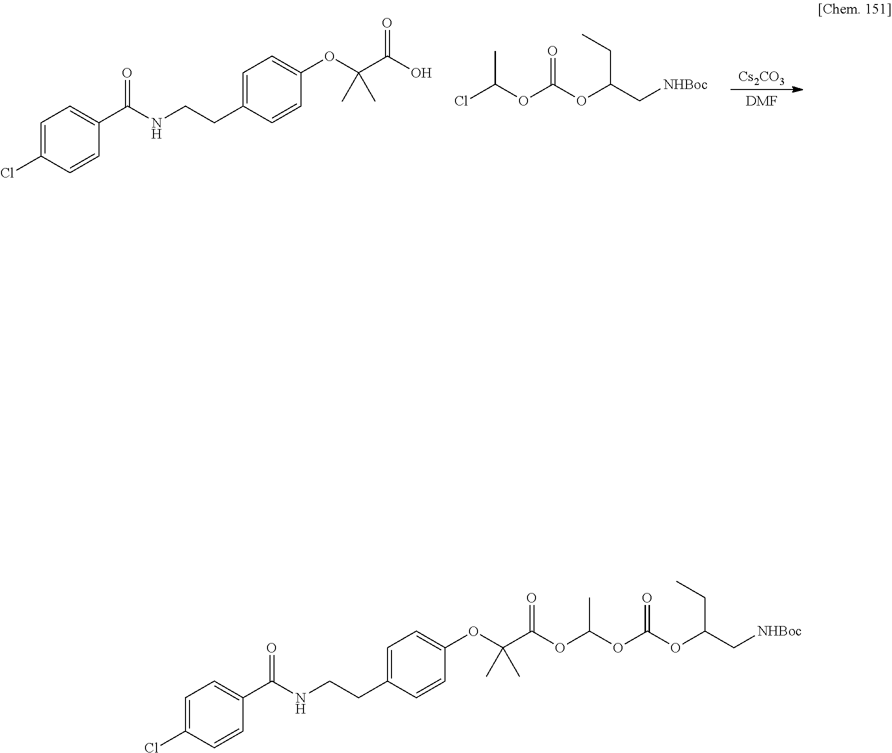

To a methanol solution of 400 mg (1.11 mmol) of bezafibrate, 180 mg (0.553 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 327 mg (1.11 mmol) of carbonic acid 1-chloroethyl 1-[[[(1,1-dimethylethoxy) carbonyl] amino] methyl] propyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 592 mg (86%) of the title compound as a 1:2 diastereomeric mixture.

¹H-NMR (CDCl₃, δ, mixture of diastereomers): 0.91 (1H, t, J=8 Hz), 0.94 (2H, t, J=8 Hz), 1.41 (6H, s), 1.43 (3H, s), 1.51 (3H, d, J=6 Hz), 1.58-1.67 (8H, m), 2.86 (2H, t, J=7 Hz), 3.18-3.24 (1H, m), 3.36-3.41 (1H, m), 3.62-3.70 (2H, m), 4.65-4.70 (1H, m), 4.78 (1H, br-s), 6.11 (1/3H, br-s), 6.19 (2/3H, br-s), 6.78-6.83 (3H, m), 7.08-7.10 (2H, m), 7.37 (2H, d, J=9 Hz), 7.61-7.64 (2H, m)

### Reference Example 105

### 2-[4-[2-[(4-Chlorobenzoyl) amino] ethyl] phenoxy]-2-methylpropanoic Acid 1-[[[1-(aminomethyl) propoxy] carbonyl] oxy] ethyl Ester Hydrochloride

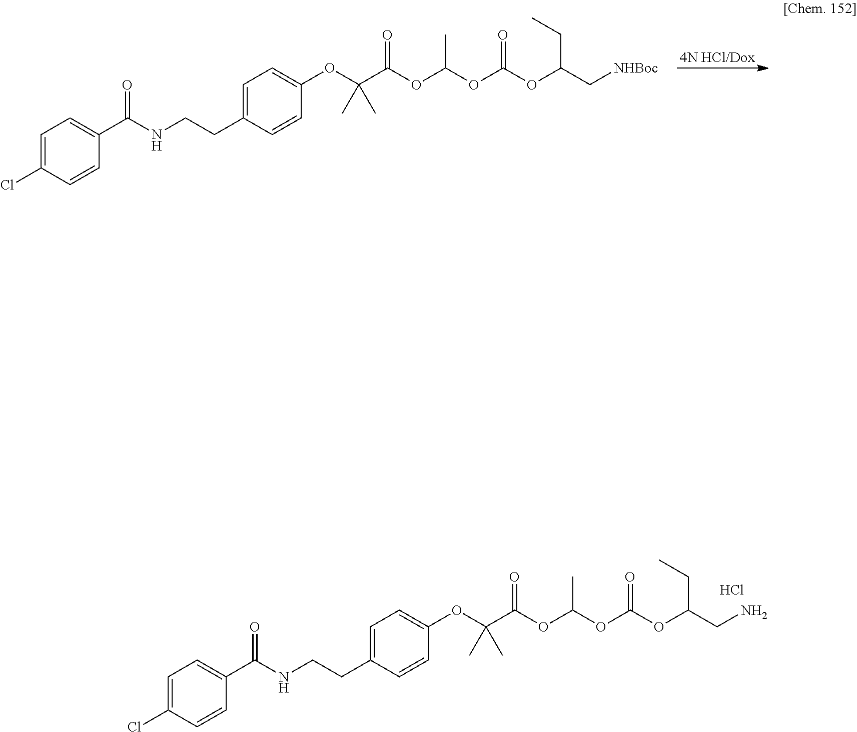 [SMILES: CCC(CNC(=O)OC(C)(C)C)OC(=O)OC(C)OC(=O)C(C)(C)Oc1ccc(CCNC(=O)c2ccc(Cl)cc2)cc1 ; "4N HCl/Dox" ; CCC(CN)OC(=O)OC(C)OC(=O)C(C)(C)Oc1ccc(CCNC(=O)c2ccc(Cl)cc2)cc1 ; Cl]

To 520 mg (0.837 mmol) of 2-[4-[2-[(4-chlorobenzoyl) amino] ethyl]phenoxy]-2-methylpropanoic acid 1-[[[[1-[[[(1,1-dimethylethoxy) carbonyl] amino]methyl]-propoxy] carbonyl] oxy] ethyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 247 mg (60%) of the title compound as a 1:2 diastereomer mixture.

¹H-NMR (DMSO-d₆, δ, mixture of diastereomers): 0.81 (1H, t, J=8 Hz), 0.86 (2H, t, J=8 Hz), 1.42-1.50 (9H, m), 1.53-1.73 (2H, m), 2.76-2.80 (2H, m), 3.02-3.06 (2H, m), 3.42-3.46 (2H, m), 4.77-4.84 (1H, m), 6.72-6.78 (3H, m), 7.12-7.15 (2H, m), 7.53 (2H, d, J=9 Hz), 7.84 (2H, d, J=9 Hz), 8.22 (3H, br-s), 8.67-8.69 (1H, m)

### Example 37

### [2-[[(1-Bezafibrate-ethoxy) carbonyl] oxy] butyl] amino-chondroitin Sulfate Conjugate

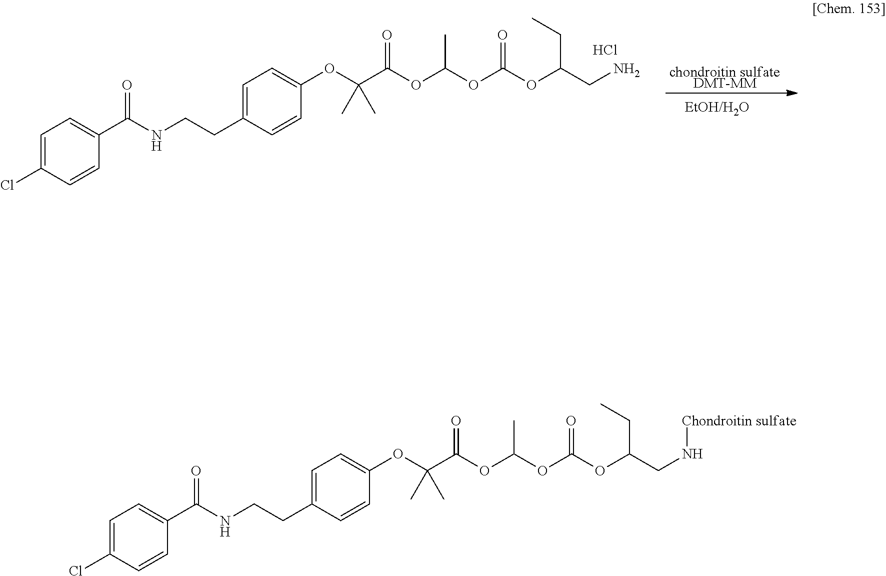

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 44 mg (0.080 mmol) of 2-[4-[2-[(4-chlorobenzoyl) amino]ethyl] phenoxy]-2-methylpropanoic acid 1-[[[1-(aminomethyl) propoxy] carbonyl] oxy]ethyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 4 mL) thereto until immediately before the reaction solution became cloudy. The reaction solution was added dropwise under stirring to 8 mL of 90% ethanol. To the mixed liquid, 7 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 206 mg of the title compound. Based on values of integral in H-NMR, the introduction ratio of bezafibrate per unit of whole disaccharide (glucuronic acid) of chondroitin sulfate was 13%.

### Reference Example 106

### N-(2-Hydroxy-3-methylbutyl) carbamic Acid 1,1-dimethylethyl Ester

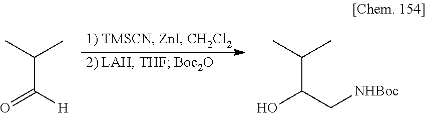

To a methylene chloride (30 mL) solution of 2.00 g (27.7 mmol) of isobutyraldehyde and 3.81 mL (30.5 mmol) of TMSCN, 8.9 mg (0.028 mmol) of zinc iodide was added under cooling on ice. The temperature of the reaction solution was returned to room temperature, and the reaction solution was stirred for one hour. Thereafter, the solvent was distilled off under reduced pressure. To the residue, THF (15 mL) and 5 M hydrochloric acid (30 mL) were added, and the resulting mixture was stirred for two hours. Sodium chloride was added to the reaction solution, and the resulting mixture was extracted with ethyl acetate. The obtained organic layer was dried with anhydrous magnesium sulfate, and the solvent was distilled off under reduced pressure. The residue was added dropwise to a THE (60 mL) suspension of 3.18 g (83.2 mmol) of LiAlH₄, and the resulting mixture was stirred for one hour while being heated under reflux. A saturated sodium sulfate aqueous solution was added to the reaction solution under cooling on ice, and then a THF (10 mL) solution of 9.08 g (41.6 mmol) of Boc₂O was added. The temperature of the reaction solution was returned to room temperature, and the reaction solution was stirred overnight. Thereafter, the reaction solution was filtered, and the solvent was distilled off under reduced pressure. The residue was dissolved in ethyl acetate, washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to obtain 4.01 g (71%) of the title compound.

¹H-NMR (CDCl₃, δ, mixture of rotamers): 0.92-0.97 (6H, m), 1.445 (4.5H, s), 1.448 (4.5H, s), 1.63-1.72 (1H, m), 2.29-2.36 (1H, m), 3.04-3.05 (1H, m), 3.32-3.36 (1H, m), 3.40-3.41 (1H, m), 4.92 (1H, br-s)

### Reference Example 107

Carbonic Acid 1-chloroethyl 1-[[[(1,1-dimethylethoxy) carbonyl] amino]methyl]-2-methyl Propyl Ester

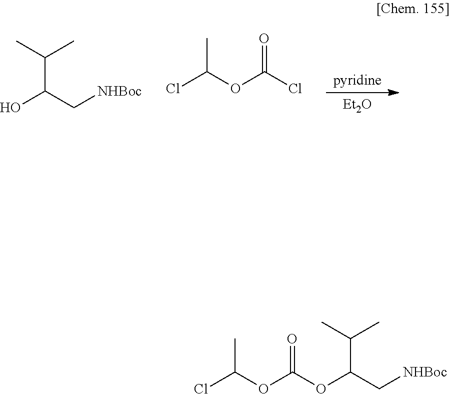

To a diethyl ether (30 mL) solution of 1.56 g (7.67 mmol) of N-(2-hydroxy-3-methylbutyl) carbamic acid 1,1-dimethylethyl ester, 1.01 mL (9.21 mmol) of 1-chloroethyl chloroformate was added at −15° C. Subsequently, a diethyl ether (10 mL) solution of 0.927 mL (11.5 mmol) of pyridine was added dropwise thereto. The temperature of the reaction solution was returned to room temperature, and the reaction solution was stirred overnight. Thereafter, water was added to the reaction solution, and the resulting mixture was extracted with diethyl ether. The obtained organic layer was washed with a saturated ammonium chloride aqueous solution, saturated sodium bicarbonate water, and saturated saline, and then dried with anhydrous magnesium sulfate, and the solvent was distilled off under reduced pressure. The residue was purified by silica gel column chromatography to obtain 2.37 g (quant.) of the title compound as a 1:1 diastereomeric mixture.

¹H-NMR (CDCl₃, δ, mixture of diastereomers): 0.97-0.99 (6H, m), 1.44 (9H, s), 1.840 (1.5H, d, J=6 Hz), 1.844 (1.5H, d, J=6 Hz), 1.91-1.99 (1H, m), 3.21-3.29 (1H, m), 3.45-3.49 (1H, m), 4.64-4.73 (2H, m), 6.43 (1H, q, J=6 Hz)

### Reference Example 108

### 2-[4-[2-[(4-Chlorobenzoyl) amino] ethyl] phenoxy]-2-methylpropanoic Acid 1-[[[1-[[[(1,1-dimethylethoxy) carbonyl] amino] methyl]-2-methyl propoxy] carbonyl]oxy] ethyl Ester

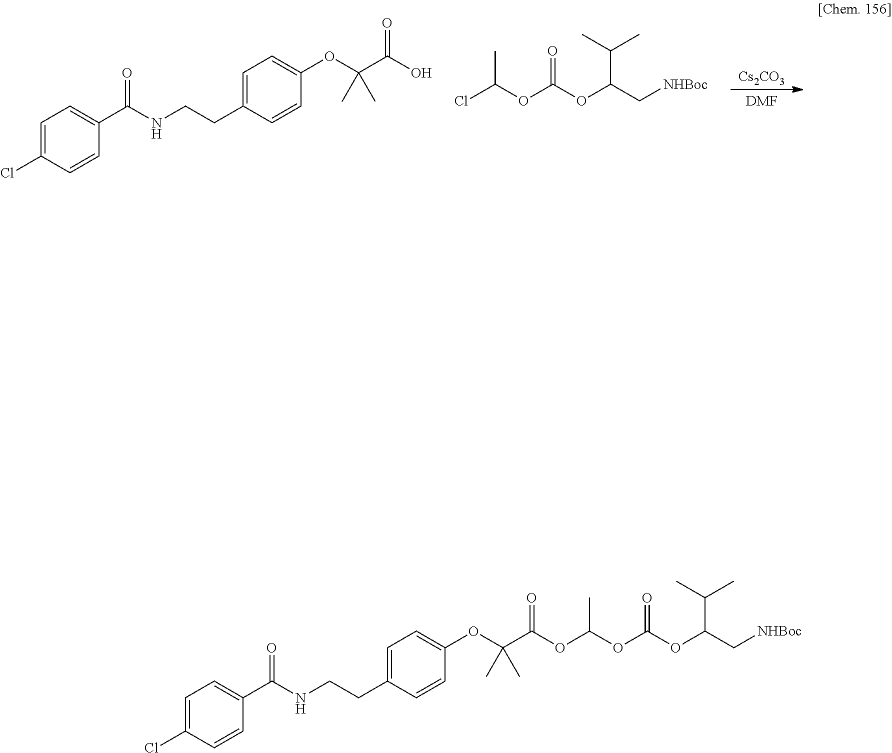

To a methanol solution of 400 mg (1.11 mmol) of bezafibrate, 180 mg (0.553 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 342 mg (1.11 mmol) of carbonic acid 1-chloroethyl 1-[[[(1,1-dimethylethoxy) carbonyl] amino] methyl]-2-methyl propyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 501 mg (71%) of the title compound as a 1:1 diastereomeric mixture.

¹H-NMR (CDCl₃, δ, mixture of diastereomers): 0.92 (1.5H, d, J=7 Hz), 0.93 (1.5H, d, J=7 Hz), 0.95 (1.5H, d, J=7 Hz), 0.96 (1.5H, d, J=7 Hz), 1.40 (4.5H, s), 1.43 (4.5H, s), 1.51 (1.5H, d, J=6 Hz), 1.52 (1.5H, d, J=6 Hz), 1.57-1.59 (3H, m), 1.86-1.95 (1H, m), 2.86 (2H, t, J=7 Hz), 3.17-3.25 (1H, m), 3.40-3.46 (1H, m), 3.62-3.70 (2H, m), 4.56-4.61 (1H, m), 4.76 (1H, br-s), 6.14 (0.5H, br-s), 6.23 (0.5H, br-s), 6.78-6.84 (3H, m), 7.08 (1H, d, J=9 Hz), 7.09 (1H, d, J=9 Hz), 7.37 (2H, d, J=9 Hz), 7.62 (1H, d, J=9 Hz), 7.63 (1H, d, J=9 Hz)

### Reference Example 109

### 2-[4-2-[(4-Chlorobenzoyl) amino] ethyl] phenoxy]-2-methylpropanoic Acid 1-[[[1-(aminomethyl)-2-methylpropoxy] carbonyl] oxy] ethyl Ester Hydrochloride

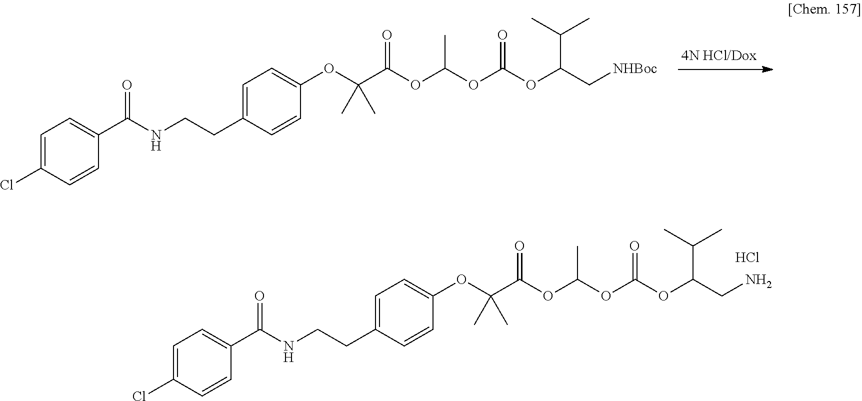 [SMILES: CC(OC(=O)OC(CNC(=O)OC(C)(C)C)C(C)C)OC(=O)C(C)(C)Oc1ccc(CCNC(=O)c2ccc(Cl)cc2)cc1 ; CC(OC(=O)OC(CN)C(C)C)OC(=O)C(C)(C)Oc1ccc(CCNC(=O)c2ccc(Cl)cc2)cc1 ; Cl]

To 500 mg (0.787 mmol) of 2-[4-[2-[(4-chlorobenzoyl) amino] ethyl]phenoxy]-2-methylpropanoic acid 1-[[[[1-[[[(1,1-dimethylethoxy) carbonyl] amino]methyl]-2-methylpropoxy] carbonyl] oxy] ethyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 282 mg (63%) of the title compound as a 1:1 diastereomer mixture.

¹H-NMR (DMSO-d₆, δ, mixture of diastereomers): 0.83 (1.5H, d, J=7 Hz), 0.84 (1.5H, d, J=7 Hz), 0.89 (3H, d, J=7 Hz), 1.42-1.51 (9H, m), 1.88-2.03 (1H, m), 2.76-2.80 (2H, m), 3.01-3.09 (2H, m), 3.42-3.46 (2H, m), 4.71-4.75 (1H, m), 6.74-6.78 (3H, m), 7.12 (1H, d, J=9 Hz), 7.14 (1H, d, J=9 Hz), 7.53 (2H, d, J=9 Hz), 7.84 (2H, d, J=9 Hz), 8.18 (3H, br-s), 8.66 (1H, t, J=6 Hz)

### Example 38

### [2-[[(1-Bezafibrate-ethoxy) carbonyl] oxy]-3-methylbutyl] amino-chondroitin Sulfate Conjugate

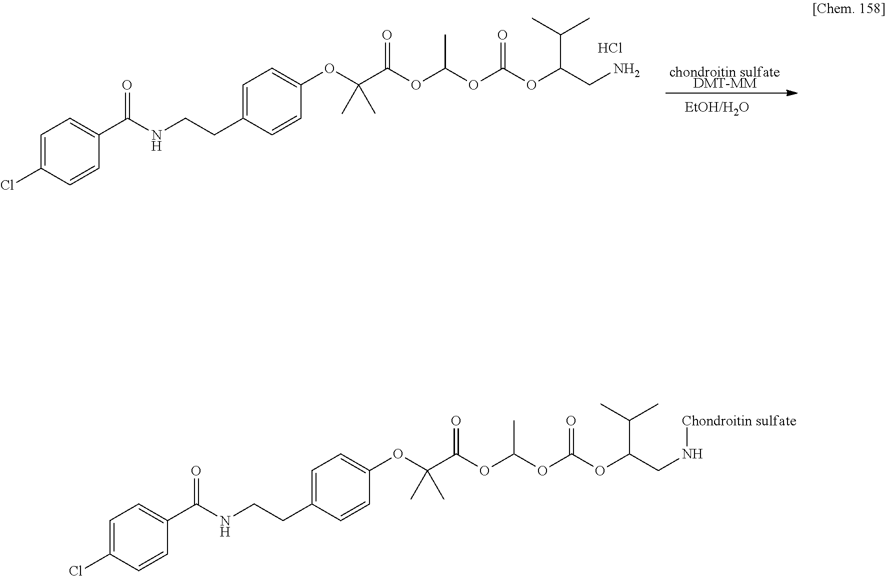

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 45 mg (0.080 mmol) of 2-[4-[2-[(4-chlorobenzoyl) amino]ethyl] phenoxy]-2-methylpropanoic acid 1-[[[1-(aminomethyl)-2-methyl propoxy]carbonyl] oxy] ethyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 205 mg of the title compound. Based on values of integral in ¹H-NMR, the introduction ratio of bezafibrate per unit of whole disaccharide (glucuronic acid) of chondroitin sulfate was 10%.

### Reference Example 110

### N-(2-Hydroxy-3,3-dimethylbutyl) carbamic Acid 1,1-dimethylethyl Ester

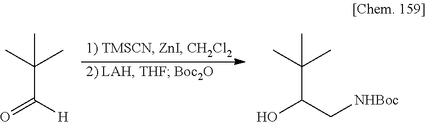

To a methylene chloride (30 mL) solution of 2.58 g (30.0 mmol) of pivalaldehyde and 3.27 mL (33.0 mmol) of TMSCN, 9.6 mg (0.030 mmol) of zinc iodide was added under cooling on ice. The temperature of the reaction solution was returned to room temperature, and the reaction solution was stirred for one hour. Thereafter, the solvent was distilled off under reduced pressure. To the residue, THE (15 mL) and 5 M hydrochloric acid (30 mL) were added, and the resulting mixture was stirred for two hours. Sodium chloride was added to the reaction solution, and the resulting mixture was extracted with ethyl acetate. The obtained organic layer was dried with anhydrous magnesium sulfate, and the solvent was distilled off under reduced pressure. The residue was added dropwise to a THE (60 mL) suspension of 3.42 g (90.0 mmol) of LiAlH₄, and the resulting mixture was stirred for one hour while being heated under reflux. A saturated sodium sulfate aqueous solution was added to the reaction solution under cooling on ice, and then THF (50 mL) and a THE (10 mL) solution of 8.51 g (39.0 mmol) of Boc₂O were added. The temperature of the reaction solution was returned to room temperature, and the reaction solution was stirred overnight. Thereafter, the reaction solution was filtered, and the solvent was distilled off under reduced pressure. The residue was dissolved in ethyl acetate, washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to obtain 4.48 g (69%) of the title compound.

¹H-NMR (CDCl₃, δ): 0.93 (9H, s), 1.45 (9H, s), 2.36 (1H, br-s), 2.94-2.99 (1H, m), 3.32 (1H, ddd, J=3, 5, 10 Hz), 3.40 (1H, ddd, J=3, 8, 14), 4.93 (1H, br-s)

### Reference Example 111

### Carbonic Acid 1-chloroethyl 1-[[[(1,1-dimethylethoxy) carbonyl] amino]methyl]-2,2-dimethyl Propyl Ester

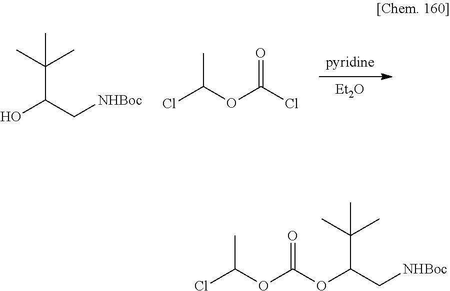

To a diethyl ether (25 mL) solution of 1.30 g (5.98 mmol) of N-(2-hydroxy-3,3-dimethylbutyl) carbamic acid 1,1-dimethylethyl ester, 0.783 mL (7.18 mmol) of 1-chloroethyl chloroformate was added at −15° C. Subsequently, a diethyl ether (5 mL) solution of 0.723 mL (8.97 mmol) of pyridine was added dropwise thereto. The temperature of the reaction solution was returned to room temperature, and the reaction solution was stirred overnight. Thereafter, water was added to the reaction solution, and the resulting mixture was extracted with diethyl ether. The obtained organic layer was washed with a saturated ammonium chloride aqueous solution, saturated sodium bicarbonate water, and saturated saline, and then dried with anhydrous magnesium sulfate, and the solvent was distilled off under reduced pressure. The residue was purified by silica gel column chromatography to obtain 1.83 g (94%) of the title compound as a 1:2 diastereomeric mixture.

¹H-NMR (CDCl₃, δ, mixture of diastereomers): 0.977 (6H, s), 0.979 (3H, s), 1.43 (9H, s), 1.84 (1H, d, J=6 Hz), 1.85 (2H, d, J=6 Hz), 3.10-3.18 (1H, m), 3.53-3.59 (1H, m), 4.65-4.72 (2H, m), 6.41-6.45 (1H, m)

### Reference Example 112

### 2-[4-[2-[(4-Chlorobenzoyl) amino] ethyl] phenoxy]-2-methylpropanoic Acid 1-[[[1-[[[(1,1-dimethylethoxy) carbonyl] amino] methyl]-2,2-dimethyl propoxy]carbonyl] oxy] ethyl Ester

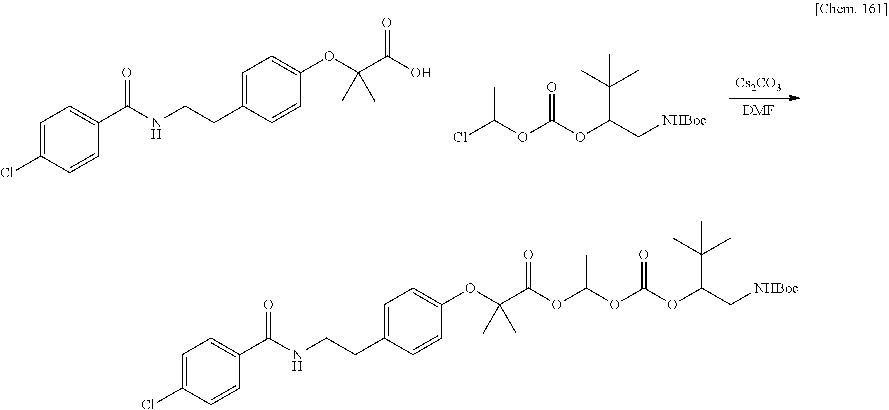

To a methanol solution of 400 mg (1.11 mmol) of bezafibrate, 180 mg (0.553 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 358 mg (1.11 mmol) of carbonic acid 1-chloroethyl 1-[[[(1,1-dimethylethoxy) carbonyl] amino] methyl]-2,2-dimethyl propyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 642 mg (90%) of the title compound as a 1:2 diastereomeric mixture.

¹H-NMR (CDCl₃, δ, mixture of diastereomers): 0.93 (3H, s), 0.96 (6H, s), 1.39 (6H, s), 1.43 (3H, s), 1.51-1.52 (3H, m), 1.56-1.59 (6H, m), 2.86 (2H, t, J=7 Hz), 3.03-3.16 (1H, m), 3.52-3.70 (3H, m), 4.58-4.62 (1H, m), 4.74 (1/3H, br-s), 4.79 (2/3H, br-s), 6.13 (1/3H, br-s), 6.21 (2/3H, br-s), 6.78 (1H, q, J=6 Hz), 6.83 (2H, d, J=9 Hz), 7.08-7.10 (2H, m), 7.37 (2H, d, J=9 Hz), 7.62 (2/3H, d, J=9 Hz), 7.63 (4/3H, d, J=9 Hz)

### Reference Example 113

### 2-[4-[2-[(4-Chlorobenzoyl) amino] ethyl] phenoxy]-2-methylpropanoic Acid 1-[[[1-(aminomethyl)-2,2-dimethylpropoxy] carbonyl] oxy] ethyl Ester Hydrochloride

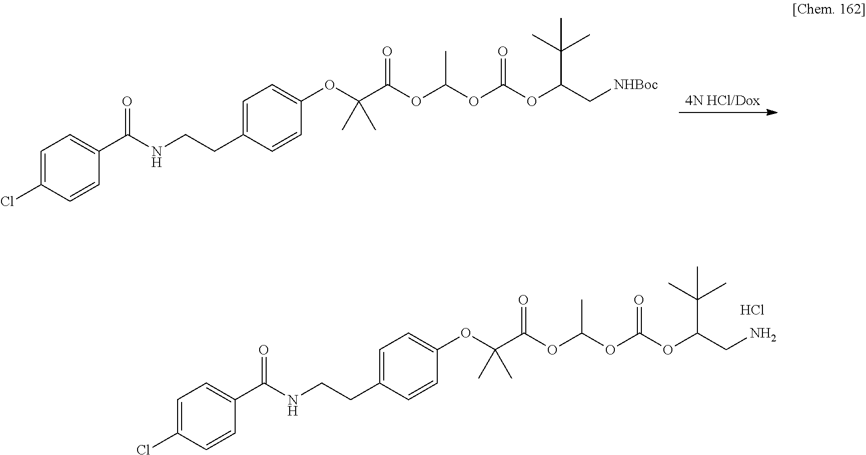 [SMILES: CC(OC(=O)OC(CNC(=O)OC(C)(C)C)C(C)(C)C)OC(=O)C(C)(C)Oc1ccc(CCNC(=O)c2ccc(Cl)cc2)cc1 ; "4N HCl/Dox" ; CC(OC(=O)OC(CN)C(C)(C)C)OC(=O)C(C)(C)Oc1ccc(CCNC(=O)c2ccc(Cl)cc2)cc1 ; Cl]

To 642 mg (0.989 mmol) of 2-[4-[2-[(4-chlorobenzoyl) amino] ethyl]phenoxy]-2-methylpropanoic acid 1-[[[[1-[[[(1,1-dimethylethoxy) carbonyl] amino]methyl]-2,2-dimethylpropoxy] carbonyl] oxy] ethyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 481 mg (83%) of the title compound as a 1:2 diastereomer mixture.

¹H-NMR (DMSO-d₆, δ, mixture of diastereomers): 0.86 (3H, s), 0.90 (6H, s), 1.43-1.52 (9H, m), 2.76-2.80 (2H, m), 2.91-2.96 (1H, m), 3.16-3.19 (1H, m), 3.43-3.47 (2H, m), 4.70 (1/3H, dd, J=1, 10 Hz), 4.73 (2/3H, dd, J=2, 10 Hz), 6.73 (1H, q, J=6 Hz), 6.77 (2H, d, J=9 Hz), 7.12 (2/3H, d, J=9 Hz), 7.15 (4/3H, d, J=9 Hz), 7.52 (2H, d, J=9 Hz), 7.85 (2H, d, J=9 Hz), 8.27 (3H, br-s), 8.69 (1H, t, J=6 Hz)

### Example 39

### [2-[[(1-Bezafibrate-ethoxy) carbonyl] oxy]-3,3-dimethylbutyl] amino-chondroitin Sulfate Conjugate

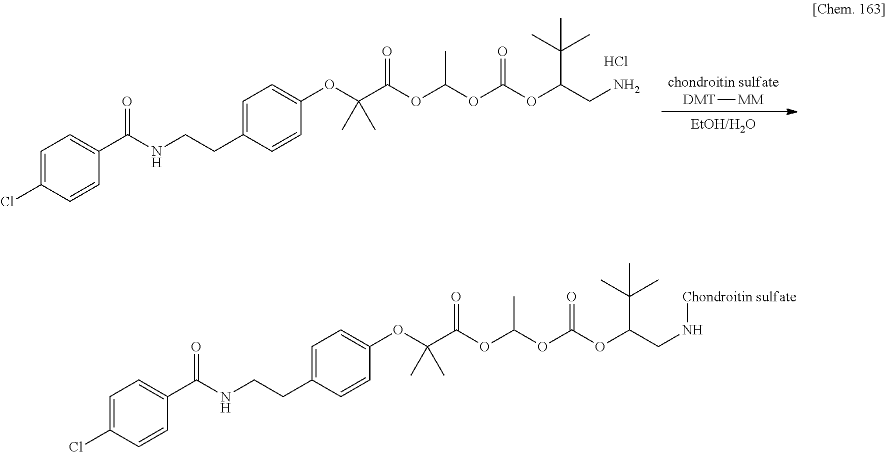

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 47 mg (0.080 mmol) of 2-[4-[2-[(4-chlorobenzoyl) amino]ethyl] phenoxy]-2-methylpropanoic acid 1-[[[1-(aminomethyl)-2,2-dimethyl propoxy]carbonyl] oxy] ethyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 252 mg of the title compound. Based on values of integral in ¹H-NMR, the introduction ratio of bezafibrate per unit of whole disaccharide (glucuronic acid) of chondroitin sulfate was 6%.

### Reference Example 114

### Carbonic Acid 1-chloro-2-methylpropyl 1-[[[(1,1-dimethylethoxy) carbonyl]amino] methyl]-2,2-dimethyl Propyl Ester

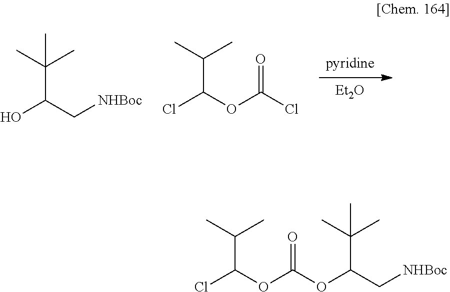

To a diethyl ether (25 mL) solution of 1.00 g (4.60 mmol) of N-(2-hydroxy-3,3-dimethylbutyl) carbamic acid 1,1-dimethylethyl ester, 0.739 mL (5.06 mmol) of 1-chloro-2-methylpropyl chloroformate was added at −15° C. Subsequently, a diethyl ether (5 mL) solution of 0.556 mL (6.90 mmol) of pyridine was added dropwise thereto. The temperature of the reaction solution was returned to room temperature, and the reaction solution was stirred overnight. Thereafter, water was added to the reaction solution, and the resulting mixture was extracted with diethyl ether. The obtained organic layer was washed with a saturated ammonium chloride aqueous solution, saturated sodium bicarbonate water, and saturated saline, and then dried with anhydrous magnesium sulfate, and the solvent was distilled off under reduced pressure. The residue was purified by silica gel column chromatography to obtain 1.50 g (92%) of the title compound as a 1:2 diastereomeric mixture.

¹H-NMR (CDCl₃, δ, mixture of diastereomers): 0.97 (3H, s), 0.98 (6H, s), 1.06-1.10 (6H, m), 1.42 (6H, s), 1.44 (3H, s), 2.19-2.25 (1H, m), 3.09-3.19 (1H, m), 3.53-3.58 (1H, m), 4.66-4.72 (2H, m), 6.19 (1H, d, J=5 Hz)

### Reference Example 115

### 2-[4-[2-[(4-Chlorobenzoyl) amino] ethyl] phenoxy]-2-methylpropanoic Acid 1-[[[1-[[[(1,1-dimethylethoxy) carbonyl] amino] methyl]-2,2-dimethyl propoxy]carbonyl] oxy]-2-methylpropyl Ester

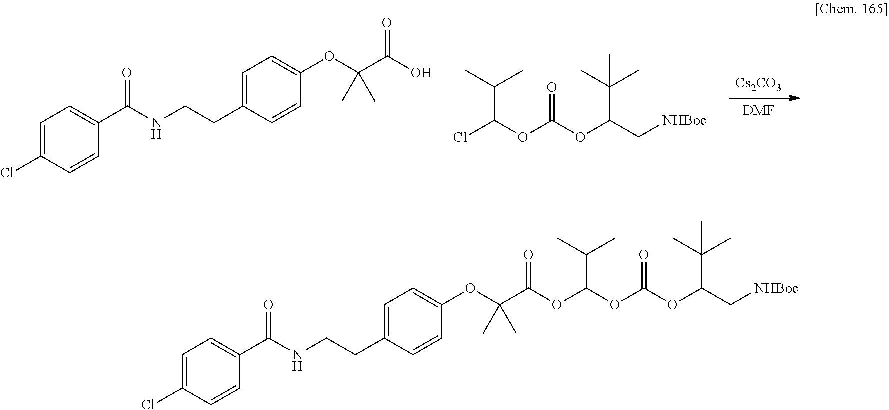

To a methanol solution of 400 mg (1.11 mmol) of bezafibrate, 180 mg (0.553 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 389 mg (1.11 mmol) of carbonic acid 1-chloro-2-methylpropyl 1-[[[(1,1-dimethylethoxy) carbonyl] amino] methyl]-2,2-dimethyl propyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 123 mg (16%) of the title compound as a 1:5 diastereomeric mixture.

¹H-NMR (CDCl₃, δ, major diastereomer): 0.91-0.96 (15H, m), 1.39 (9H, s), 1.59-1.61 (6H, m), 2.03-2.09 (1H, m), 2.85 (2H, t, J=7 Hz), 3.01-3.07 (1H, m), 3.52-3.71 (3H, m), 4.60 (1H, dd, J=2, 10 Hz), 4.81 (1H, br-s), 6.18 (1H, br-s), 6.50 (1H, d, J=5 Hz), 6.83 (2H, d, J=9 Hz), 7.09 (2H, d, J=9 Hz), 7.38 (2H, d, J=9 Hz), 7.63 (2H, d, J=9 Hz)

### Reference Example 116

### 2-[4-[2-[(4-Chlorobenzoyl) amino] ethyl] phenoxy]-2-methylpropanoic Acid 1-[[[1-(aminomethyl)-2,2-dimethylpropoxy] carbonyl] oxy]-2-methylpropyl Ester Hydrochloride

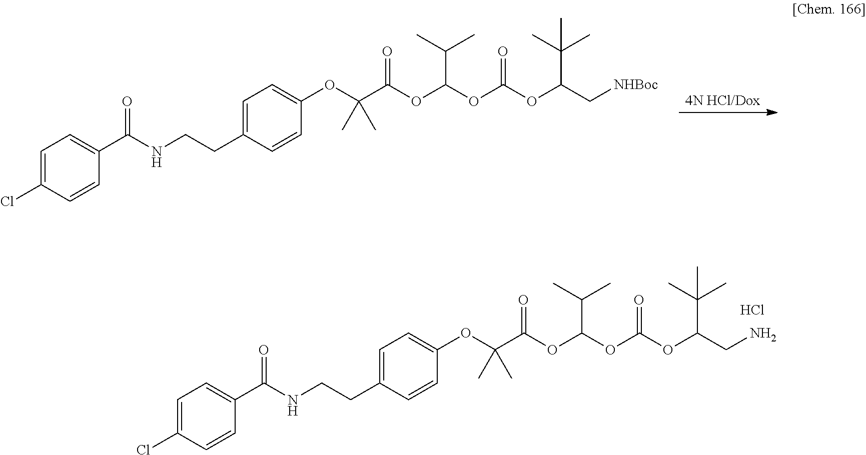 [SMILES: CC(C)C(OC(=O)OC(CNC(=O)OC(C)(C)C)C(C)(C)C)OC(=O)C(C)(C)Oc1ccc(CCNC(=O)c2ccc(Cl)cc2)cc1 ; "4N HCl/Dox" ; CC(C)C(OC(=O)OC(CN)C(C)(C)C)OC(=O)C(C)(C)Oc1ccc(CCNC(=O)c2ccc(Cl)cc2)cc1 ; Cl]

To 120 mg (0.177 mmol) of 2-[4-[2-[(4-chlorobenzoyl) amino] ethyl]phenoxy]-2-methylpropanoic acid 1-[[[[l-[[[(1,1-dimethylethoxy) carbonyl] amino]methyl]-2,2-dimethylpropoxy] carbonyl] oxy]-2-methylpropyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 93 mg (86%) of the title compound as a 1:5 diastereomer mixture.

¹H-NMR (DMSO-d₆, δ, major diastereomer): 0.80 (3H, d, J=7 Hz), 0.85 (3H, d, J=7 Hz), 0.90 (9H, s), 1.54 (6H, s), 1.99-2.06 (1H, m), 2.77 (2H, t, J=8 Hz), 2.94-2.98 (1H, m), 3.17-3.20 (1H, m), 3.41-3.46 (2H, m), 4.71 (1H, d, J=10 Hz), 6.53 (1H, d, J=5 Hz), 6.77 (2H, d, J=9 Hz), 7.14 (2H, d, J=9 Hz), 7.53 (2H, d, J=9 Hz), 7.83 (2H, d, J=9 Hz), 8.12 (3H, br-s), 8.64 (1H, br-s)

### Example 40

### [2-[[(1-Bezafibrate-2-methylpropoxy) carbonyl] oxy]-3,3-dimethylbutyl] amino-chondroitin Sulfate Conjugate

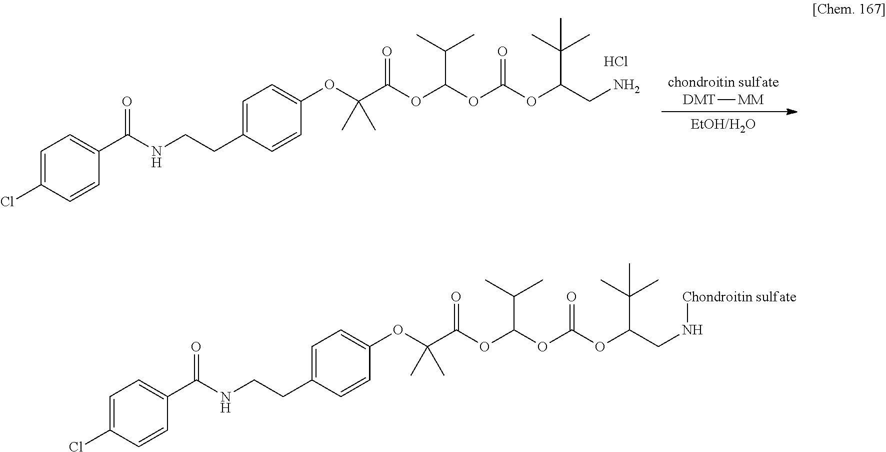

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 49 mg (0.080 mmol) of 2-[4-[2-[(4-chlorobenzoyl) amino]ethyl] phenoxy]-2-methylpropanoic acid 1-[[[1-(aminomethyl)-2,2-dimethyl propoxy]carbonyl] oxy]-2-methylpropyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and 4 mL of ethanol was added dropwise thereto. The reaction solution was added dropwise under stirring to 8 mL of 90% ethanol. To the mixed liquid, 7 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 201 mg of the title compound. Based on values of integral in ¹H-NMR, the introduction ratio of bezafibrate per unit of whole disaccharide (glucuronic acid) of chondroitin sulfate was 10%.

### Reference Example 117

### N-(2-Cyclohexyl-2-hydroxyethyl) carbamic Acid 1,1-dimethylethyl Ester

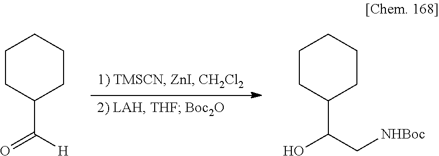

To a methylene chloride (30 mL) solution of 3.07 g (30.0 mmol) of cyclohexanecarboxy aldehyde and 3.27 mL (33.0 mmol) of TMSCN, 9.6 mg (0.030 mmol) of zinc iodide was added under cooling on ice. The temperature of the reaction solution was returned to room temperature, and the reaction solution was stirred for one hour. Thereafter, the solvent was distilled off under reduced pressure. To the residue, THF (15 mL) and 5 M hydrochloric acid (30 mL) were added, and the resulting mixture was stirred for two hours. Sodium chloride was added to the reaction solution, and the resulting mixture was extracted with ethyl acetate. The obtained organic layer was dried with anhydrous magnesium sulfate, and the solvent was distilled off under reduced pressure. The residue was added dropwise to a THE (60 mL) suspension of 3.42 g (90.0 mmol) of LiAlH₄, and the resulting mixture was stirred for one hour while being heated under reflux. A saturated sodium sulfate aqueous solution was added to the reaction solution under cooling on ice, and then THE (50 mL) and a THE (10 mL) solution of 8.51 g (39.0 mmol) of Boc₂O were added. The temperature of the reaction solution was returned to room temperature, and the reaction solution was stirred overnight. Thereafter, the reaction solution was filtered, and the solvent was distilled off under reduced pressure. The residue was dissolved in ethyl acetate, washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to obtain 5.98 g (82%) of the title compound.

¹H-NMR (CDCl₃, δ): 0.90-1.40 (7H, m), 1.47 (9H, s), 1.66-1.68 (1H, m), 1.73-1.78 (2H, m), 1.84-1.87 (1H, m), 2.29 (1H, br-s), 3.02-3.08 (1H, m), 3.33-3.45 (2H, m), 4.93 (1H, br-s)

### Reference Example 118

### Carbonic Acid 1-chloroethyl 1-cyclohexyl-2-[[(1,1-dimethylethoxy) carbonyl]amino] ethyl] ester

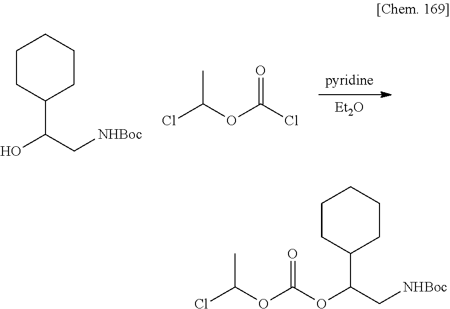

To a diethyl ether (25 mL) solution of 1.50 g (6.16 mmol) of N-(2-cyclohexyl-2-hydroxyethyl) carbamic acid 1,1-dimethylethyl ester, 0.807 mL (7.40 mmol)) of 1-chloroethyl chloroformate was added at −15° C. Subsequently, a diethyl ether (5 mL) solution of 0.745 mL (9.25 mmol) of pyridine was added dropwise thereto. The temperature of the reaction solution was returned to room temperature, and the reaction solution was stirred overnight. Thereafter, water was added to the reaction solution, and the resulting mixture was extracted with diethyl ether. The obtained organic layer was washed with a saturated ammonium chloride aqueous solution, saturated sodium bicarbonate water, and saturated saline, and then dried with anhydrous magnesium sulfate, and the solvent was distilled off under reduced pressure. The residue was purified by silica gel column chromatography to obtain 2.03 g (94%) of the title compound as a 1:1 diastereomeric mixture.

¹H-NMR (CDCl₃, δ, mixture of diastereomers): 1.01-1.27 (5H, m), 1.44 (9H, s), 1.58-1.77 (6H, m), 1.838 (1.5H, d, J=6 Hz), 1.841 (1.5H, d, J=6 Hz), 3.23-3.31 (1H, m), 3.27-3.49 (1H, m), 4.64-4.72 (2H, m), 6.41-6.45 (1H, m)

### Reference Example 119

### 2-[4-[2-[(4-Chlorobenzoyl) amino] ethyl] phenoxy]-2-methylpropanoic Acid 1-[[[1-cyclohexyl-2-[[(1,1-dimethylethoxy) carbonyl] amino] ethoxy] carbonyl] oxy]ethyl Ester

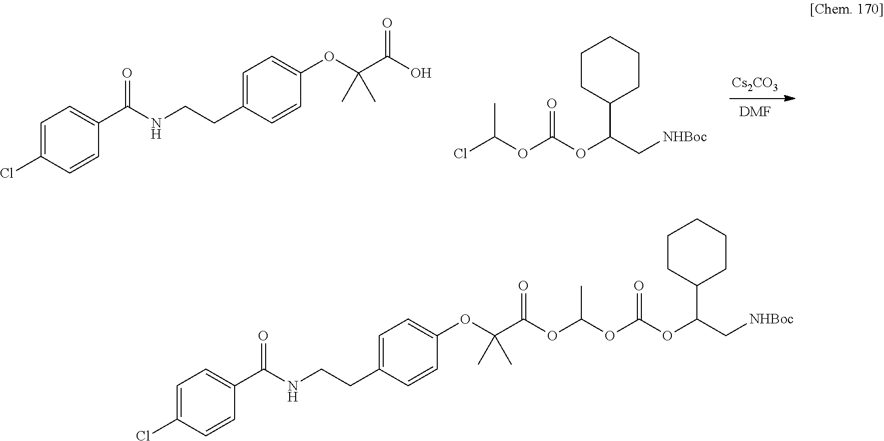

To a methanol solution of 400 mg (1.11 mmol) of bezafibrate, 180 mg (0.553 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 386 mg (1.11 mmol) of carbonic acid 1-chloroethyl 1-cyclohexyl-2-[[(1,1-dimethylethoxy) carbonyl] amino] ethyl] ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 450 mg (60%) of the title compound as a 1:1 diastereomeric mixture.

¹H-NMR (CDCl₃, δ, mixture of diastereomers): 1.01-1.23 (5H, m), 1.40 (4.5H, s), 1.43 (4.5H, s), 1.51-1.52 (3H, m), 1.57-1.73 (12H, m), 2.86 (2H, t, J=7 Hz), 3.20-3.27 (1H, m), 3.34-3.48 (1H, m), 3.62-3.72 (2H, m), 4.56-4.61 (1H, m), 4.74 (1H, br-s), 6.07 (0.5H, br-s), 6.17 (0.5H, br-s), 6.77-6.84 (3H, m), 7.09 (2H, d, J=9 Hz), 7.376 (1H, d, J=9 Hz), 7.279 (1H, d, J=9 Hz), 7.62 (1H, d, J=9 Hz), 7.63 (1H, d, J=9 Hz)

### Reference Example 120

### 2-[4-[2-[(4-Chlorobenzoyl) amino] ethyl] phenoxy]-2-methylpropanoic Acid 1-[[(2-amino-1-cyclohexylethoxy) carbonyl] oxy] ethyl Ester Hydrochloride

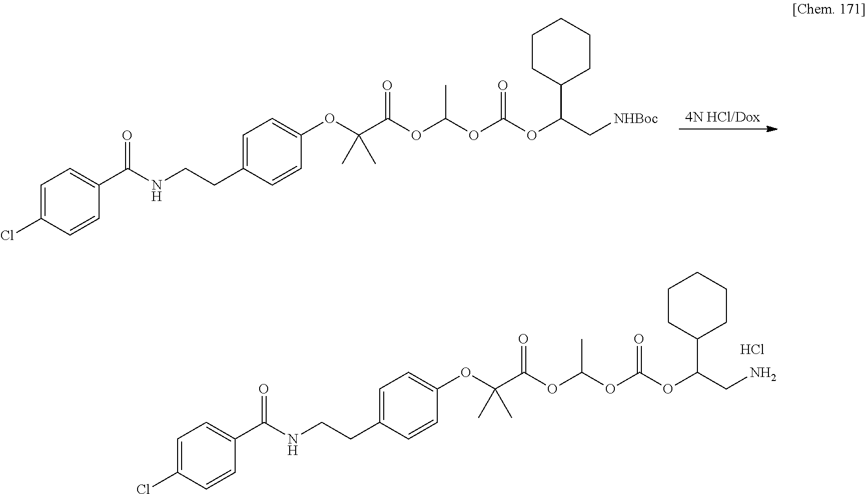 [SMILES: CC(OC(=O)OC(CNC(=O)OC(C)(C)C)C1CCCCC1)OC(=O)C(C)(C)Oc1ccc(CCNC(=O)c2ccc(Cl)cc2)cc1 ; "4N HCl/Dox" ; CC(OC(=O)OC(CN)C1CCCCC1)OC(=O)C(C)(C)Oc1ccc(CCNC(=O)c2ccc(Cl)cc2)cc1 ; Cl]

To 430 mg (0.637 mmol) of 2-[4-[2-[(4-chlorobenzoyl) amino] ethyl] phenoxy]-2-methylpropanoic acid 1-[[[1-cyclohexyl-2-[[(1,1-dimethylethoxy) carbonyl]amino] ethoxy] carbonyl] oxy] ethyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 345 mg (89%) of the title compound as a 1:1 diastereomer mixture.

¹H-NMR (DMSO-d₆, δ, mixture of diastereomers): 0.91-1.20 (5H, m), 1.43 (1.5H, d, J=6 Hz), 1.46-1.51 (7.5H, m), 1.56-1.72 (6H, m), 2.77-2.80 (2H, m), 3.02-3.10 (2H, m), 3.42-3.45 (1H, m), 4.72-4.73 (1H, m), 6.71-6.77 (3H, m), 7.12-7.15 (2H, m), 7.53 (2H, d, J=9 Hz), 7.84 (2H, d, J=9 Hz), 8.19 (3H, br-s), 8.68 (1H, br-s)

### Example 41

### [2-[[(1-Bezafibrate-ethoxy) carbonyl] oxy]-2-cyclohexylethyl] amino-chondroitin Sulfate Conjugate

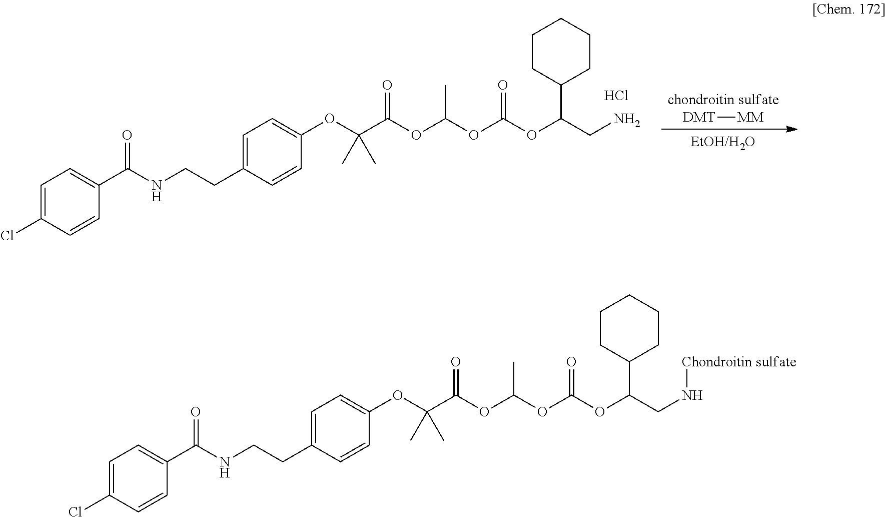

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 49 mg (0.080 mmol) of 2-[4-[2-[(4-chlorobenzoyl) amino]ethyl] phenoxy]-2-methylpropanoic acid 1-[[(2-amino-1-cyclohexylethoxy) carbonyl]oxy] ethyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 202 mg of the title compound. Based on values of integral in ¹H-NMR, the introduction ratio of bezafibrate per unit of whole disaccharide (glucuronic acid) of chondroitin sulfate was 9%.

### Reference Example 121

### N-(2-Hydroxy-1-methylethyl) carbamic Acid 1,1-dimethylethyl Ester

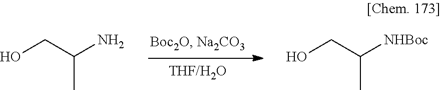

To a water (50 mL)-THF (50 mL) mixed solution of 3.76 g (50.0 mmol) of 2-aminopropanol and 12.2 g (115 mmol) of sodium carbonate, under cooling on ice, a THF (50 mL) solution of 12.0 g (55.0 mmol) of tert-butyl dicarbonate was added, and the resulting mixture was stirred at room temperature for three hours. Water was added to the reaction solution, and the resulting mixture was extracted with ethyl acetate. The obtained organic layer was washed with saturated sodium bicarbonate water and saturated saline, and then dried with anhydrous magnesium sulfate, and the solvent was distilled off under reduced pressure. The obtained residue was recrystallized from a diethyl ether-hexane mixed solvent to obtain 8.17 g (93%) of the title compound.

¹H-NMR (CDCl₃, δ): 1.15 (3H, d, J=7 Hz), 1.45 (9H, s), 2.77 (1H, br-s), 3.48-3.52 (1H, m), 3.61-3.65 (1H, m), 3.76 (1H, br-s)

### Reference Example 122

### Carbonic Acid 1-chloroethyl 2-[[(1,1-dimethylethoxy) carbonyl] amino]propyl Ester

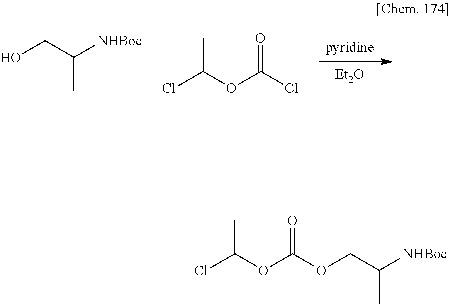

To a diethyl ether (30 mL) solution of 2.63 g (15.0 mmol) of N-(2-hydroxy-1-methylethyl) carbamic acid 1,1-dimethylethyl ester, 1.96 mL (18.0 mmol) of 1-chloroethyl chloroformate was added at −15° C. Subsequently, a diethyl ether (10 mL) solution of 1.81 mL (22.5 mmol) of pyridine was added dropwise thereto. The temperature of the reaction solution was returned to room temperature, and the reaction solution was stirred overnight. Thereafter, water was added to the reaction solution, and the resulting mixture was extracted with diethyl ether. The obtained organic layer was washed with a saturated ammonium chloride aqueous solution, saturated sodium bicarbonate water, and saturated saline, and then dried with anhydrous magnesium sulfate, and the solvent was distilled off under reduced pressure. The residue was purified by silica gel column chromatography to obtain 3.38 g (80%) of the title compound as a 1:1 diastereomeric mixture.

¹H-NMR (CDCl₃, δ, mixture of diastereomers): 1.197 (1.5H, d, J=7 Hz), 1.202 (1.5H, d, J=7 Hz), 1.44 (9H, s), 1.84 (3H, d, J=6 Hz), 3.99 (1H, br-s), 4.11-4.21 (2H, m), 4.59 (1H, br-s), 6.427 (0.5H, q, J=6 Hz), 6.430 (0.5H, q, J=6 Hz)

### Reference Example 123

### 2-[4-[2-[(4-Chlorobenzoyl) amino] ethyl] phenoxy]-2-methylpropanoic Acid 1-[[[2-[[(1,1-dimethylethoxy) carbonyl] amino] propoxy] carbonyl] oxy] ethyl Ester

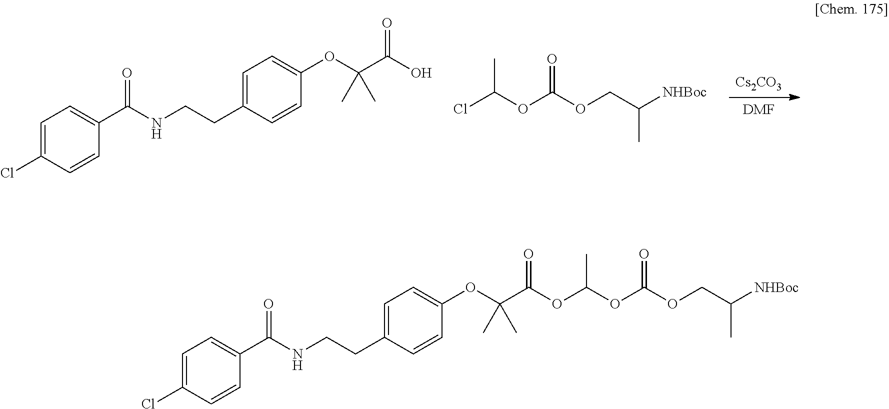

To a methanol solution of 400 mg (1.11 mmol) of bezafibrate, 180 mg (0.553 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 311 mg (1.11 mmol) of carbonic acid 1-chloroethyl 2-[[(1,1-dimethylethoxy) carbonyl] amino] propyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 483 mg (72%) of the title compound as a 1:1 diastereomeric mixture.

¹H-NMR (CDCl₃, 8, mixture of diastereomers): 1.160 (1.5H, d, J=8 Hz), 1.164 (1.5H, d, J=8 Hz), 1.43 (9H, s), 1.51 (3H, d, J=6 Hz), 1.59 (3H, s), 1.61 (3H, s), 2.86 (2H, t, J=7 Hz), 3.66 (2H, q, J=7 Hz), 3.95 (1H, br-s), 4.05-4.14 (2H, m), 4.63 (1H, br-s), 6.18 (1H, br-s), 6.79-6.84 (3H, m), 7.09 (2H, d, J=9 Hz), 7.37 (2H, d, J=9 Hz), 7.62 (1H, d, J=9 Hz), 7.63 (1H, d, J=9 Hz)

### Reference Example 124

### 2-[4-[2-[(4-Chlorobenzoyl) amino] ethyl] phenoxy]-2-methylpropanoic Acid 1-[[(2-aminopropoxy) carbonyl] oxy] ethyl Ester Hydrochloride

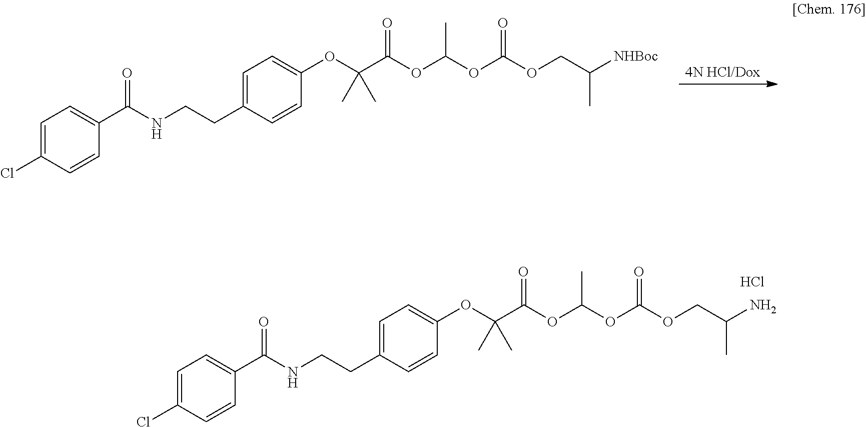 [SMILES: CC(COC(=O)OC(C)OC(=O)C(C)(C)Oc1ccc(CCNC(=O)c2ccc(Cl)cc2)cc1)NC(=O)OC(C)(C)C ; "4N HCl/Dox" ; CC(N)COC(=O)OC(C)OC(=O)C(C)(C)Oc1ccc(CCNC(=O)c2ccc(Cl)cc2)cc1 ; Cl]

To 413 mg (0.680 mmol) of 2-[4-[2-[(4-chlorobenzoyl) amino] ethyl]phenoxy]-2-methylpropanoic acid 1-[[[2-[[(1,1-dimethylethoxy) carbonyl] amino]propoxy] carbonyl] oxy] ethyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 130 mg (35%) of the title compound as a 1:1 diastereomer mixture.

¹H-NMR (DMSO-d₆, δ, mixture of diastereomers): 1.19-1.21 (3H, m), 1.44 (3H, d, J=6 Hz), 1.49 (3H, s), 1.50 (3H, s), 2.78 (2H, t, J=8 Hz), 3.42-3.45 (2H, m), 3.48-3.52 (1H, m), 4.16-4.30 (2H, m), 6.73-6.76 (3H, m), 7.14 (2H, d, J=9 Hz), 7.53 (2H, d, J=9 Hz), 7.83 (2H, d, J=9 Hz), 8.15 (3H, br-s), 8.66 (1H, t, J=6 Hz)

### Example 42

### [2-[[(1-Bezafibrate-ethoxy) carbonyl] oxy]-1-methylethyl] amino-chondroitin Sulfate Conjugate

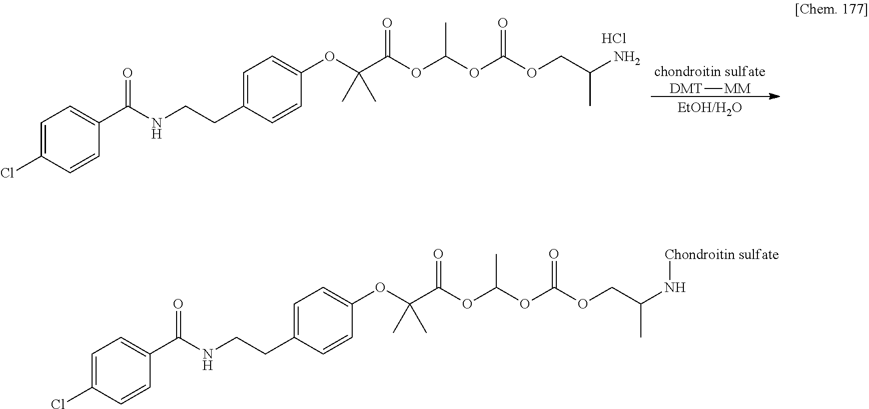

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 43 mg (0.080 mmol) of 2-[4-[2-[(4-chlorobenzoyl) amino]ethyl] phenoxy]-2-methylpropanoic acid 1-[[(2-aminopropoxy) carbonyl] oxy] ethyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 209 mg of the title compound. Based on values of integral in ¹H-NMR, the introduction ratio of bezafibrate per unit of whole disaccharide (glucuronic acid) of chondroitin sulfate was 11%.

### Reference Example 125

### Carbonic Acid 1-chloroethyl trans-2-[[(1,1-dimethylethoxy) carbonyl] amino]cyclohexyl Ester

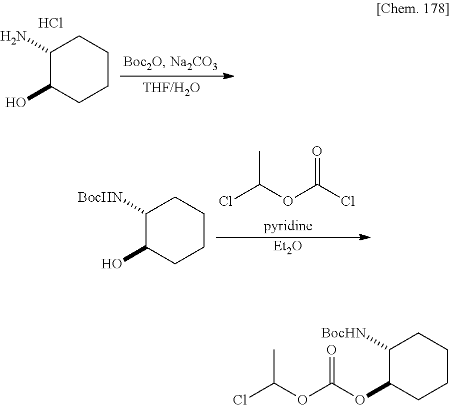

To a water (10 mL)-THF (10 mL) mixed solution of 1.00 g (6.60 mmol) of trans-2-aminocyclohexanol hydrochloride and 1.58 g (7.26 mmol) of sodium carbonate, under cooling on ice, a THE (10 mL) solution of 1.42 g (6.60 mmol) of tert-butyl dicarbonate was added, and the resulting mixture was stirred at room temperature for two hours. Water was added to the reaction solution, and the resulting mixture was extracted with ethyl acetate. The obtained organic layer was washed with saturated sodium bicarbonate water and saturated saline, and then dried with anhydrous magnesium sulfate, and the solvent was distilled off under reduced pressure. The obtained residue was dissolved in diethyl ether (20 mL), and 0.864 mL (7.91 mmol) of 1-chloroethyl chloroformate was added thereto at −15° C. Subsequently, a diethyl ether (10 mL) solution of 0.797 mL (9.89 mmol) of pyridine was added dropwise thereto. The temperature of the reaction solution was returned to room temperature, and the reaction solution was stirred overnight. Thereafter, water was added to the reaction solution, and the resulting mixture was extracted with diethyl ether. The obtained organic layer was washed with a saturated ammonium chloride aqueous solution, saturated sodium bicarbonate water, and saturated saline, and then dried with anhydrous magnesium sulfate, and the solvent was distilled off under reduced pressure. The residue was purified by silica gel column chromatography to obtain 1.73 g (82%) of the title compound as a 1:1 diastereomeric mixture.

¹H-NMR (CDCl₃, δ, mixture of diastereomers): 1.22-1.34 (3H, m), 1.42 (4.5H, s), 1.43 (4.5H, s), 1.48-1.57 (1H, m), 1.68-1.70 (1H, m), 1.76-1.79 (1H, m), 1.81 (1.5H, d, J=6 Hz), 1.82 (1.5H, d, J=6 Hz), 2.06-2.11 (2H, m), 3.61 (1H, br-s), 4.48-4.53 (1H, m), 4.57 (1H, br-s), 6.41 (1H, q, J=6 Hz)

### Reference Example 126

### 2-[4-[2-[(4-Chlorobenzoyl) amino] ethyl] phenoxy]-2-methylpropanoic Acid 1-[[[trans-2-[[(1,1-dimethylethoxy) carbonyl] amino] cyclohexyl] oxy] carbonyl] oxy]ethyl Ester

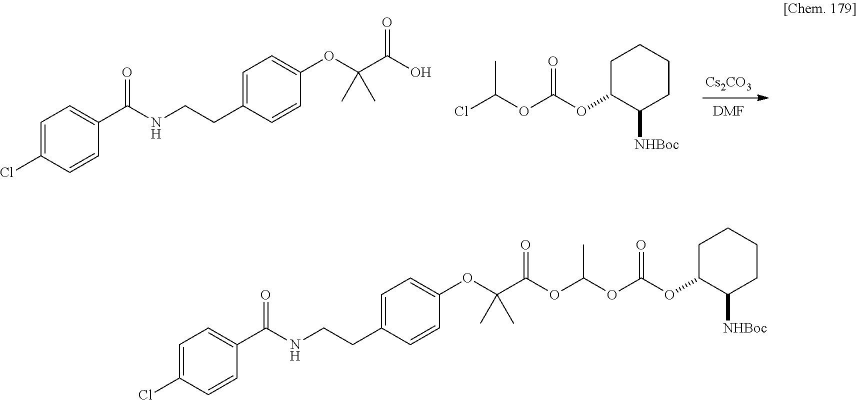

To a methanol solution of 400 mg (1.11 mmol) of bezafibrate, 180 mg (0.553 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 355 mg (1.11 mmol) of carbonic acid 1-chloroethyl trans-2-[[(1,1-dimethylethoxy) carbonyl] amino] cyclohexyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 334 mg (47%) of the title compound as a 1:1 diastereomeric mixture.

¹H-NMR (CDCl₃, δ, mixture of diastereomers): 1.15-1.36 (3H, m), 1.41 (4.5H, s), 1.42 (4.5H, s), 1.45-1.49 (4H, m), 1.57-1.60 (6H, m), 1.63-1.76 (2H, m), 1.97-2.09 (2H, m), 2.84-2.88 (2H, m), 3.57 (1H, br-s), 3.62-3.71 (2H, m), 4.38-3.45 (1H, m), 4.53 (1H, br-s), 6.11 (0.5H, br-s), 6.22 (0.5H, br-s), 6.79 (1H, q, J=6 Hz), 6.82 (1H, d, J=9 Hz), 6.83 (1H, d, J=9 Hz), 7.09 (1H, d, J=9 Hz), 7.10 (1H, d, J=9 Hz), 7.37 (1H, d, J=9 Hz), 7.38 (1H, d, J=9 Hz), 7.61-7.64 (2H, m)

### Reference Example 127

### 2-[4-[2-[(4-Chlorobenzoyl) amino] ethyl] phenoxy]-2-methylpropanoic Acid 1-[[[(trans-2-aminocyclohexyl) oxy] carbonyl] oxy] ethyl Ester Hydrochloride

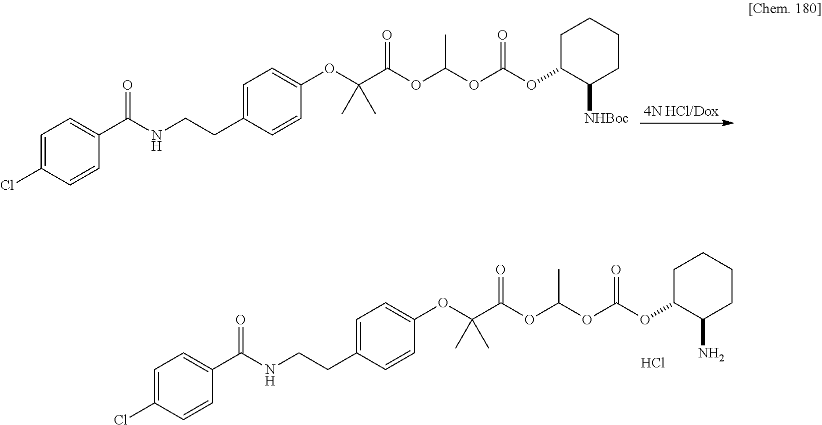

To 310 mg (0.479 mmol) of 2-[4-[2-[(4-chlorobenzoyl) amino] ethyl]phenoxy]-2-methylpropanoic acid 1-[[[trans-2-[[(1,1-dimethylethoxy) carbonyl] amino]cyclohexyl] oxy] carbonyl] oxy] ethyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 224 mg (80%) of the title compound as a 1:1 diastereomer mixture.

¹H-NMR (DMSO-d₆, 8, mixture of diastereomers): 1.25-1.51 (13H, m), 1.66-1.68 (2H, m), 1.91-2.09 (2H, m), 2.76-2.79 (2H, m), 3.15-3.21 (1H, m), 3.41-3.46 (2H, m), 4.50-4.56 (1H, m), 6.74-6.78 (3H, m), 6.11-6.16 (2H, m), 7.53 (2H, d, J=9 Hz), 7.83 (2H, d, J=9 Hz), 8.12 (3H, s), 8.64 (1H, t, J=6 Hz)

### Example 43

### [trans-2-[[(1-Bezafibrate-ethoxy) carbonyl] oxy] cyclohexyl] amino-chondroitin Sulfate Conjugate

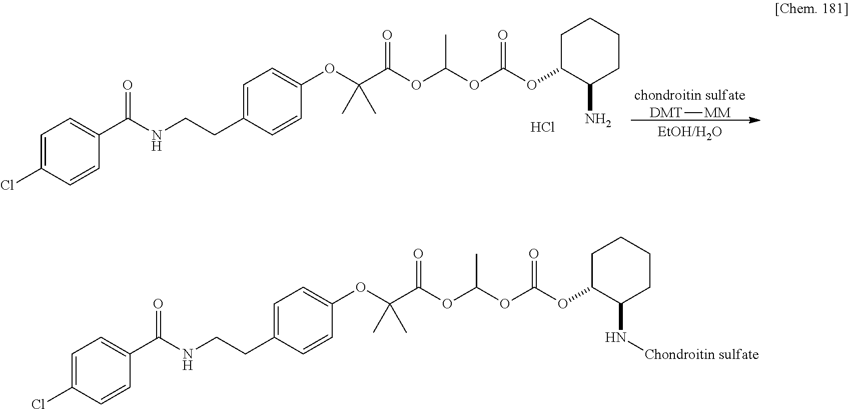

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 46 mg (0.080 mmol) of 2-[4-[2-[(4-chlorobenzoyl) amino]ethyl] phenoxy]-2-methylpropanoic acid 1-[[[(trans-2-aminocyclohexyl) oxy] carbonyl]oxy] ethyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 218 mg of the title compound. Based on values of integral in ¹H-NMR, the introduction ratio of bezafibrate per unit of whole disaccharide (glucuronic acid) of chondroitin sulfate was 10%.

### Reference Example 128

### N-[2-[[(1,1-Dimethylethoxy) carbonyl] amino] ethyl] carbamic Acid Chloromethyl Ester

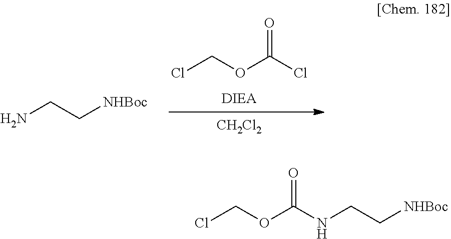

To a methylene chloride solution of 1.00 g (6.24 mmol) of N-([(1,1-dimethylethoxy) carbonyl])-ethylenediamine and 1.21 mL (9.36 mmol) of N,N-diisopropylethylamine, a methylene chloride solution of 0.666 mL (7.49 mmol) of chloromethyl chloroformate was added dropwise under cooling on ice. After 15 minutes, water was added to the reaction solution, and the resulting mixture was extracted with diethyl ether. The obtained organic layer was washed with a 5% potassium hydrogen sulfate aqueous solution, saturated sodium bicarbonate water, and saturated saline, and then dried with anhydrous magnesium sulfate, and the solvent was distilled off under reduced pressure. The residue was recrystallized from a hexane-chloroform mixed solvent to obtain 1.27 g (80%) of the title compound.

¹H-NMR (CDCl₃, δ): 1.44 (9H, s), 3.28-3.30 (2H, m), 3.33-3.36 (2H, m), 4.81 (1H, br-s), 5.50 (1H, br-s), 5.74 (2H, s)

### Reference Example 129

### 2-[4-[2-[(4-Chlorobenzoyl) amino] ethyl] phenoxy]-2-methylpropanoic Acid [[[[2-[[(1,1-dimethylethoxy) carbonyl] amino] ethyl] amino] carbonyl] oxy] methyl Ester

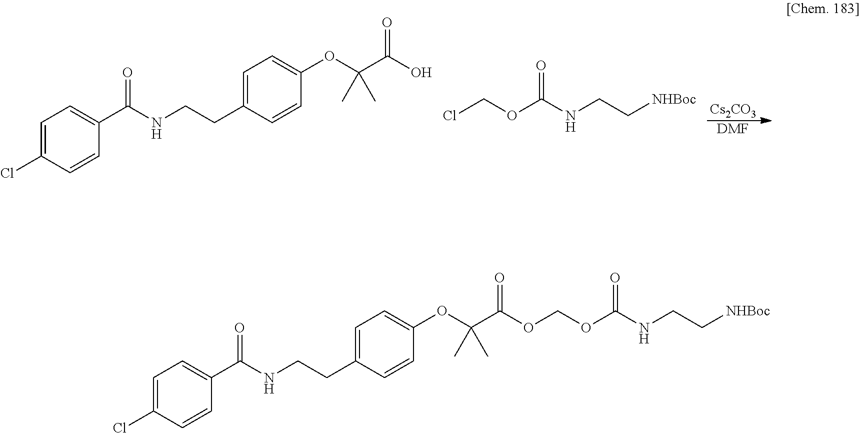

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 209 mg (0.829 mmol) of N-[2-[[(1,1-dimethylethoxy) carbonyl] amino]ethyl] carbamic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was recrystallized from a hexane-chloroform mixed solvent to obtain 323 mg (67%) of the title compound.

¹H-NMR (CDCl₃, δ): 1.43 (9H, s), 1.59 (6H, s), 2.86 (2H, t, J=7 Hz), 3.24-3.27 (4H, m), 3.67 (2H, q, J=7 Hz), 4.96 (1H, br-s), 5.43 (1H, br-s), 5.80 (2H, s), 6.18 (1H, br-s), 6.82 (2H, d, J=9 Hz), 7.09 (2H, d, J=9 Hz), 7.38 (2H, d, J=9 Hz), 7.62 (2H, d, J=9 Hz)

### Reference Example 130

### 2-[4-[2-[(4-Chlorobenzoyl) amino] ethyl] phenoxy]-2-methylpropanoic Acid [[[(2-aminoethyl) amino] carbonyl] oxy] methyl Ester Hydrochloride

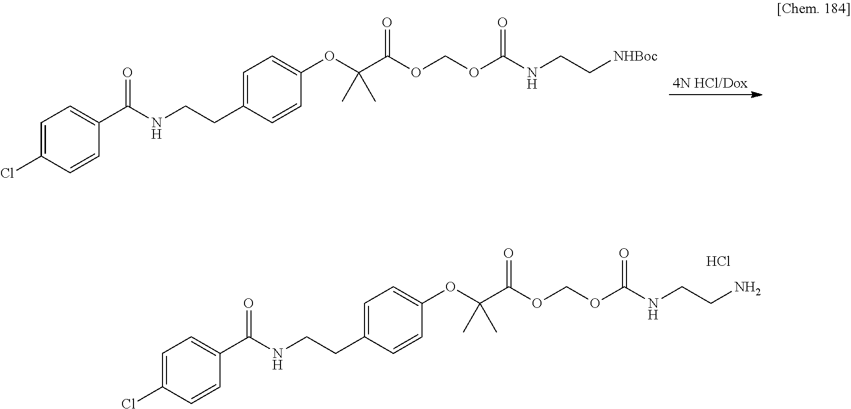 [SMILES: CC(C)(C)OC(=O)NCCNC(=O)OCOC(=O)C(C)(C)Oc1ccc(CCNC(=O)c2ccc(Cl)cc2)cc1 ; CC(C)(Oc1ccc(CCNC(=O)c2ccc(Cl)cc2)cc1)C(=O)OCOC(=O)NCCN ; Cl]

To 200 mg (0.346 mmol) of 2-[4-[2-[(4-chlorobenzoyl) amino] ethyl] phenoxy]-2-methylpropanoic acid [[[[2-[[(1,1-dimethylethoxy) carbonyl] amino] ethyl]amino] carbonyl] oxy] methyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 153 mg (86%) of the title compound.

¹H-NMR (DMSO-d₆, δ): 1.49 (6H, s), 2.79 (2H, t, J=8 Hz), 2.86 (2H, q, J=6 Hz), 3.26 (2H, q, J=6 Hz), 3.42-3.47 (2H, m), 5.76 (2H, s), 6.76 (2H, d, J=9 Hz), 7.14 (2H, d, J=9 Hz), 7.54 (2H, d, J=9 Hz), 7.70 (1H, br-s), 7.85 (2H, d, J=9 Hz), 7.92 (3H, br-s), 8.70 (1H, br-s)

### Example 44

### [2-[[(Bezafibrate-methoxy) carbonyl] amino] ethyl] amino-chondroitin Sulfate Conjugate

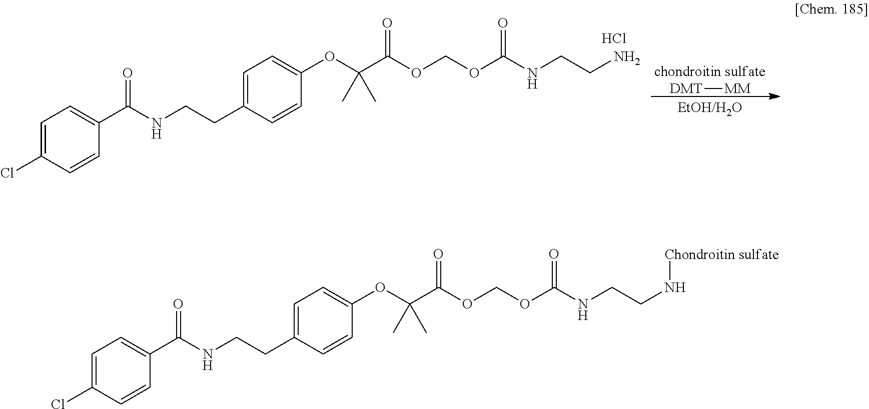

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 41 mg (0.080 mmol) of 2-[4-[2-[(4-chlorobenzoyl) amino]ethyl] phenoxy]-2-methylpropanoic acid [[[(2-aminoethyl) amino] carbonyl] oxy]methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 222 mg of the title compound. Based on values of integral in H-NMR, the introduction ratio of bezafibrate per unit of whole disaccharide (glucuronic acid) of chondroitin sulfate was 17%.

### Reference Example 131

### N-[2-[[(1,1-Dimethylethoxy) carbonyl] amino] ethyl] carbamic acid 1-chloroethyl Ester

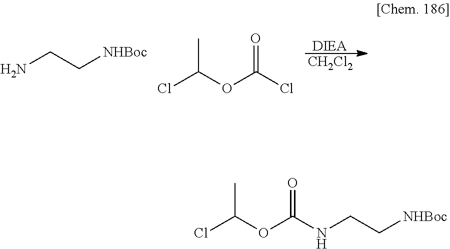

To a methylene chloride solution of 1.50 g (9.36 mmol) of N-[(1,1-dimethylethoxy) carbonyl]-ethylenediamine and 2.45 mL (14.0 mmol) of N,N-diisopropylethylamine, a methylene chloride solution of 1.23 mL (11.2 mmol) of 1-chloroethyl chloroformate was added dropwise under cooling on ice. After 30 minutes, a saturated ammonium chloride aqueous solution was added to the reaction solution, and the resulting mixture was extracted with ethyl acetate. The obtained organic layer was washed with a 5% potassium hydrogen sulfate aqueous solution, saturated sodium bicarbonate water, and saturated saline, and then dried with anhydrous magnesium sulfate, and the solvent was distilled off under reduced pressure. The obtained residue was recrystallized from an ethyl acetate-hexane mixed solvent to obtain 1.76 g (67%) of the title compound.

¹H-NMR (CDCl₃, δ): 1.45 (9H, s), 1.78 (3H, d, J=6 Hz), 3.27-3.36 (4H, m), 4.81 (1H, br-s), 5.40 (1H, br-s), 6.56 (1H, q, J=6 Hz)

### Reference Example 132

### 2-[4-[2-[(4-Chlorobenzoyl) amino] ethyl] phenoxy]-2-methylpropanoic Acid 1-[[[[2-[[(1,1-dimethylethoxy) carbonyl] amino] ethyl] amino] carbonyl] oxy] ethyl Ester

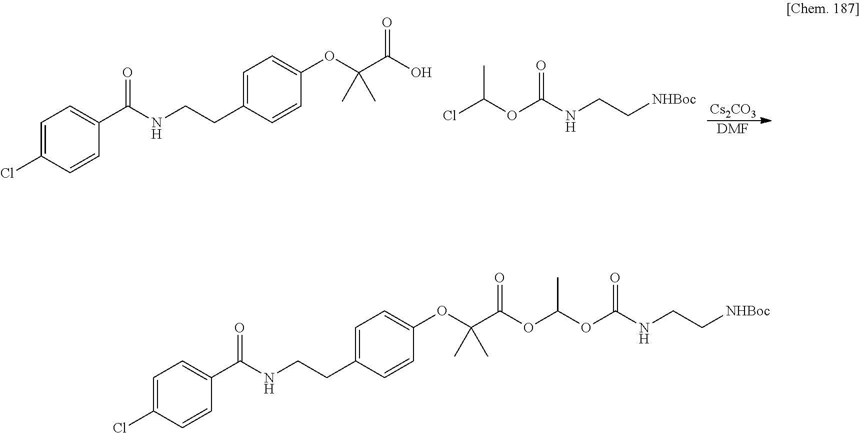

To a methanol solution of 400 mg (1.11 mmol) of bezafibrate, 185 mg (0.553 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 295 mg (1.11 mmol) of N-[2-[[(1,1-dimethylethoxy) carbonyl] amino] ethyl]carbamic acid 1-chloroethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 432 mg (66%) of the title compound.

¹H-NMR (CDCl₃, δ): 1.42-1.44 (12H, m), 1.575 (3H, s), 1.581 (3H, s), 2.85 (2H, t, J=7 Hz), 3.22-3.28 (4H, m), 3.59-3.73 (2H, m), 4.93 (1H, br-s), 5.33 (1H, br-s), 6.23 (1H, br-s), 6.82 (2H, d, J=9 Hz), 6.88 (1H, q, J=6 Hz), 7.08 (2H, d, J=9 Hz), 7.37 (2H, d, J=9 Hz), 7.62 (2H, d, J=9 Hz)

### Reference Example 133

### N-[2-[[(1,1-Dimethylethoxy) carbonyl] amino] ethyl]-N-methylcarbamic Acid Chloromethyl Ester

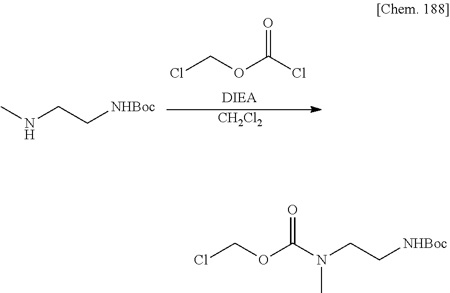

To a methylene chloride solution of 1.00 g (5.74 mmol) of N-([(1,1-dimethylethoxy) carbonyl])-N′-methylethylenediamine and 1.50 mL (8.61 mmol) of N, N-diisopropylethylamine, a methylene chloride solution of 0.612 mL (6.89 mmol) of chloromethyl chloroformate was added dropwise under cooling on ice. After 15 minutes, water was added to the reaction solution, and the resulting mixture was extracted with diethyl ether. The obtained organic layer was washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to obtain 1.47 g (96%) of the title compound.

¹H-NMR (CDCl₃, δ, mixture of rotamers): 1.44 (9H, s), 2.98 (1.5H, s), 3.00 (1.5H, s), 3.27-3.33 (2H, m), 3.39-3.47 (2H, m), 4.67 (0.5H, br-s), 4.79 (0.5H, br-s), 5.78 (1H, s), 5.79 (1H, s)

### Reference Example 134

### 2-[4-[2-[(4-Chlorobenzoyl) amino] ethyl] phenoxy]-2-methylpropanoic Acid [[[[2-[[(1,1-dimethylethoxy) carbonyl] amino] ethyl] methylamino] carbonyl] oxy]methyl Ester

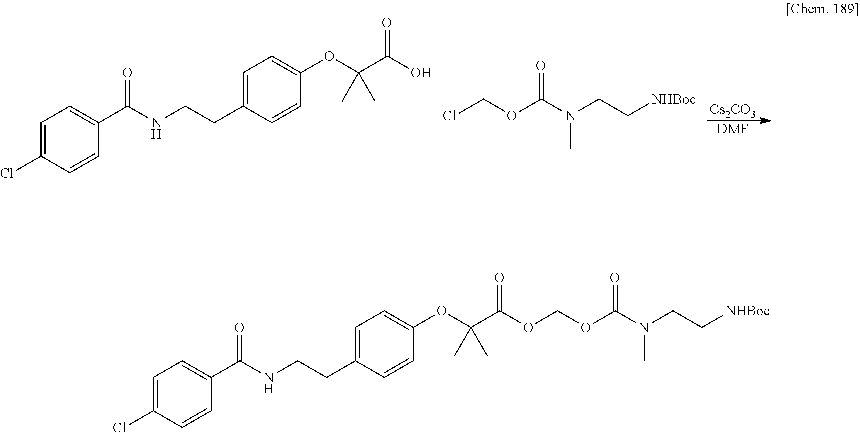

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 221 mg (0.829 mmol) of N-[2-[[(1,1-dimethylethoxy) carbonyl] amino]ethyl]-N-methylcarbamic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 316 mg (64%) of the title compound.

¹H-NMR (CDCl₃, δ, mixture of rotamers): 1.41 (4.5H, s), 1.43 (4.5H, s), 1.60 (6H, s), 2.86 (2H, t, J=7 Hz), 2.91 (1.5H, s), 2.93 (1.5H, s), 3.17-3.18 (1H, m), 3.24-3.25 (1H, m), 3.29-3.30 (1H, m), 3.35-3.37 (1H, m), 3.66 (2H, q, J=7 Hz), 4.61 (0.5H, br-s), 4.80 (0.5H, s), 5.84 (2H, s), 6.21 (1H, br-s), 6.82 (2H, d, J=9 Hz), 7.08 (2H, d, J=9 Hz), 7.38 (2H, d, J=9 Hz), 7.63 (2H, d, J=9 Hz)

### Reference Example 135

### 2-[4-[2-[(4-Chlorobenzoyl) amino] ethyl] phenoxy]-2-methylpropanoic Acid [[[(2-aminoethyl) methylamino] carbonyl] oxy] methyl Ester Hydrochloride

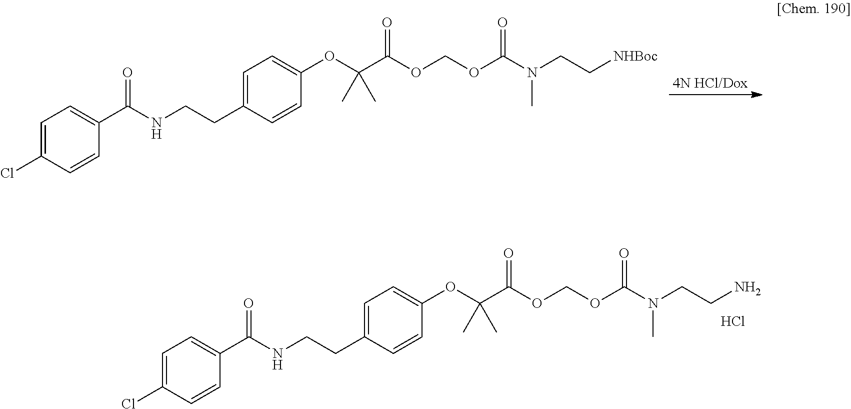 [SMILES: CN(CCNC(=O)OC(C)(C)C)C(=O)OCOC(=O)C(C)(C)Oc1ccc(CCNC(=O)c2ccc(Cl)cc2)cc1 ; CN(CCN)C(=O)OCOC(=O)C(C)(C)Oc1ccc(CCNC(=O)c2ccc(Cl)cc2)cc1 ; Cl]

To 210 mg (0.355 mmol) of 2-[4-[2-[(4-chlorobenzoyl) amino] ethyl]phenoxy]-2-methylpropanoic acid [[[[2-[[(1,1-dimethylethoxy) carbonyl] amino] ethyl]methylamino] carbonyl] oxy] methyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 137 mg (78%) of the title compound.

¹H-NMR (DMSO-d₆, δ): 1.50 (6H, s), 2.78 (2H, t, J=8 Hz), 2.86 (3H, s), 2.91-2.95 (2H, m), 3.42-3.45 (4H, m), 5.78-5.79 (2H, m), 6.75 (2H, d, J=9 Hz), 7.12 (2H, d, J=9 Hz), 7.53 (2H, d, J=9 Hz), 7.83-7.85 (2H, m), 7.95 (3H, br-s), 8.67 (1H, br-s)

### Example 45

### [2-[[(Bezafibrate-methoxy) carbonyl] methylamino] ethyl] amino-chondroitin Sulfate Conjugate

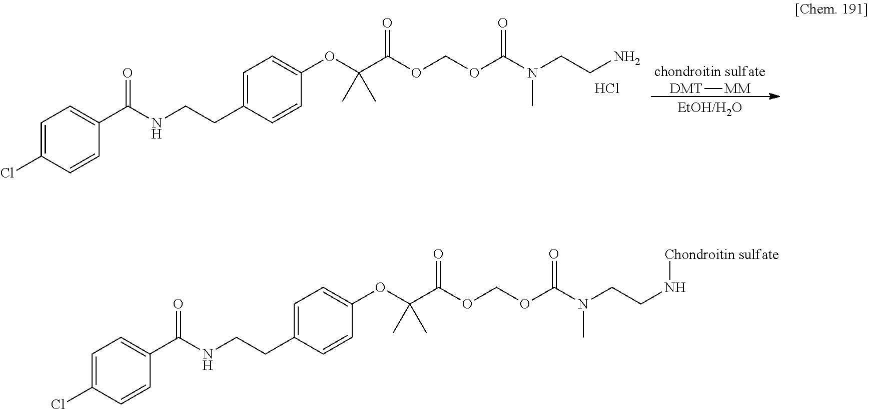

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 42 mg (0.080 mmol) of 2-[4-[2-[(4-chlorobenzoyl) amino]ethyl] phenoxy]-2-methylpropanoic acid [[[(2-aminoethyl) methylamino] carbonyl]oxy] methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 5 mL) thereto until immediately before the reaction solution became cloudy. The reaction solution was added dropwise under stirring to 8 mL of 90% ethanol. To the mixed liquid, 6 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 253 mg of the title compound. Based on values of integral in ¹H-NMR, the introduction ratio of bezafibrate per unit of whole disaccharide (glucuronic acid) of chondroitin sulfate was 16%.

### Reference Example 136

### N-[2-[[(1,1-Dimethylethoxy) carbonyl] amino] ethyl]-N-methylcarbamic acid 1-chloroethyl Ester

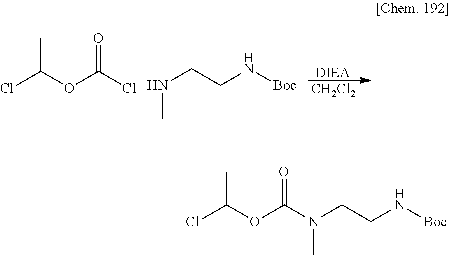

To a methylene chloride solution of 2.00 g (11.5 mmol) of N-([(1,1-dimethylethoxy) carbonyl])-N′-methylethylenediamine and 2.34 mL (13.8 mmol) of N,N-diisopropylethylamine, a methylene chloride solution of 1.38 mL (12.6 mmol) of 1-chloroethyl chloroformate was added dropwise under cooling on ice. After 15 minutes, a saturated ammonium chloride aqueous solution was added to the reaction solution, and the resulting mixture was extracted with diethyl ether. The obtained organic layer was washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to obtain 2.14 g (66%) of the title compound.

¹H-NMR (CDCl₃, δ, mixture of diastereomers): 1.43 (9H, s), 1.81 (3H, d, J=6 Hz), 2.97 (1.5H, s), 2.99 (1.5H, s), 3.30-3.50 (4H, m), 4.67 (0.5H, br-s), 4.79 (0.5H, br-s), 6.56 (0.5H, q, J=6 Hz), 6.61 (0.5H, q, J=6 Hz)

### Reference Example 137

### 2-[4-[2-[(4-Chlorobenzoyl) amino] ethyl] phenoxy]-2-methylpropanoic Acid 1-[[[[2-[[(11-dimethylethoxy) carbonyl] amino] ethyl] methylamino] carbonyl] oxy]ethyl Ester

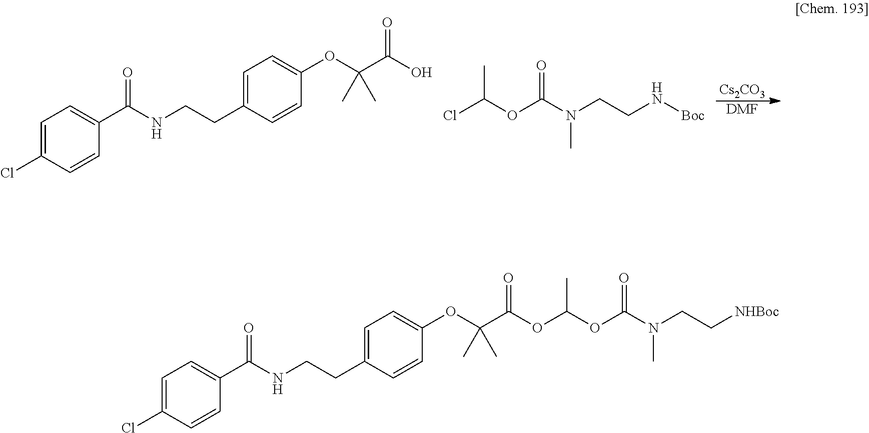

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 232 mg (0.829 mmol) of N-[2-[[(1,1-dimethylethoxy) carbonyl] amino]ethyl]-N-methylcarbamic acid 1-chloroethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 305 mg (62%) of the title compound.

¹H-NMR (CDCl₃, δ): 1.42 (9H, s), 1.49 (3H, d, J=6 Hz), 1.58 (3H, s), 1.59 (3H, s), 2.86 (2H, t, J=7 Hz), 2.89 (3H, s), 3.12-3.40 (4H, m), 3.62-3.73 (2H, m), 4.57 (0.5H, br-s), 4.82 (0.5H, br-s), 6.12 (0.5H, br-s), 6.19 (0.5H, br-s), 6.83-6.87 (3H, m), 7.09 (2H, d, J=8 Hz), 7.38 (2H, d, J=8 Hz), 7.62 (2H, d, J=8 Hz)

### Reference Example 138

### 2-[4-[2-[(4-Chlorobenzoyl) amino] ethyl] phenoxy]-2-methylpropanoic Acid 1-[[[(2-aminoethyl) methylamino] carbonyl] oxy] ethyl Ester Hydrochloride

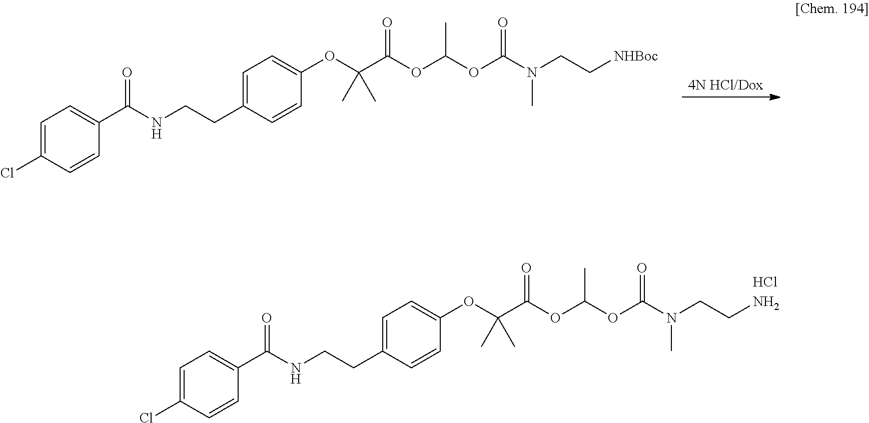 [SMILES: CC(OC(=O)N(C)CCNC(=O)OC(C)(C)C)OC(=O)C(C)(C)Oc1ccc(CCNC(=O)c2ccc(Cl)cc2)cc1 ; "4N HCl/Dox" ; CC(OC(=O)N(C)CCN)OC(=O)C(C)(C)Oc1ccc(CCNC(=O)c2ccc(Cl)cc2)cc1 ; Cl]

To 300 mg (0.495 mmol) of 2-[4-[2-[(4-chlorobenzoyl) amino] ethyl]phenoxy]-2-methylpropanoic acid 1-[[[[2-[[(1,1-dimethylethoxy) carbonyl] amino]ethyl] methylamino] carbonyl] oxy] methyl ester, 3 mL of a 4 N hydrochloric acid/dioxane solution was added, and the resulting mixture was allowed to stand for one hour. The solvent was distilled off under reduced pressure, and then hexane/ethyl acetate (1/1) was added to the residue. The resulting mixture was stirred for three hours. The precipitated crystals were collected by filtration to obtain 149 mg (56%) of the title compound.

¹H-NMR (DMSO-d₆, δ): 1.42 (3H, d, J=5 Hz), 1.48 (6H, s), 2.58 (211, br-s), 2.75-2.79 (2H, m), 2.85-2.94 (2H, m), 3.16 (3H, s), 3.41-3.46 (2H, m), 6.76 (2H, d, J=9 Hz), 7.12 (2H, d, J=9 Hz), 7.53 (2H, d, J=9 Hz), 7.83-7.85 (2H, m), 8.01 (1H, br) 8.35 (3H, br-s), 8.64-8.69 (1H, m)

### Reference Example 139

### 5-(Aminosulfonyl)-4-chloro-2-[(2-furanylmethyl) amino] benzoic Acid 1-[[[[[[2-[[(1,1-dimethylethoxy) carbonyl] amino] ethyl] methylamino] carbonyl] oxy]ethyl Ester

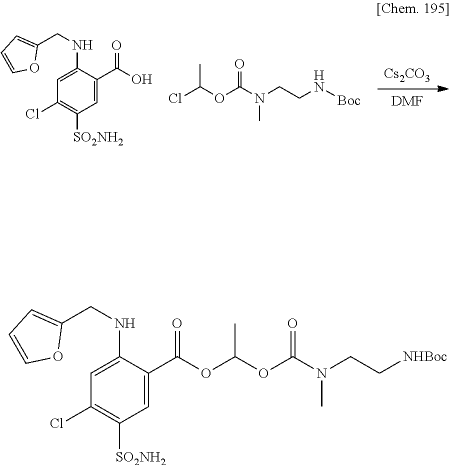

To a methanol solution of 300 mg (0.907 mmol) of furosemide, 147 mg (0.454 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 254 mg (0.907 mmol) of N-[2-[[(1,1-dimethylethoxy) carbonyl] amino]ethyl]-N-methylcarbamic acid 1-chloroethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 212 mg (41%) of the title compound.

¹H-NMR (CDCl₃, δ, mixture of diastereomers): 1.38 (4.5H, s), 1.40 (4.5H, s), 1.61 (3H, d, J=5 Hz), 2.95 (1.5H, s), 2.99 (1.5H, s), 3.27-3.39 (4H, m), 4.43 (2H, d, J=6 Hz), 4.89-4.94 (1H, m), 5.13 (1H, br-s), 5.23 (1H, brs), 6.28 (1H, d, J=3 Hz), 6.34 (1H, dd, J=2, 3 Hz), 6.87 (1H, s), 6.99-7.04 (1H, m), 7.39 (1H, dd, J=1, 2 Hz), 8.50-8.56 (2H, m)

### Reference Example 140

### 5-(Aminosulfonyl)-4-chloro-2-[(2-furanylmethyl) amino] benzoic Acid 1-[[[(2-aminoethyl) methylamino] carbonyl] oxy] ethyl Ester Dihydrochloride

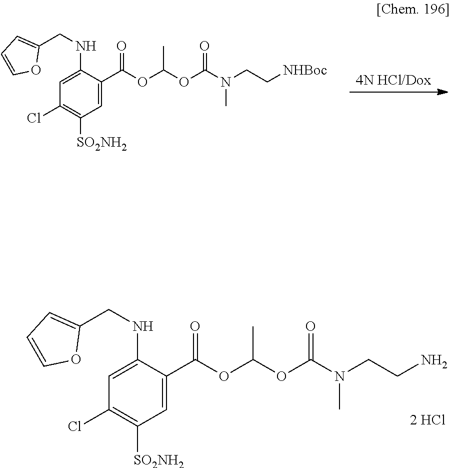 [SMILES: CC(OC(=O)c1cc(S(N)(=O)=O)c(Cl)cc1NCc1ccco1)OC(=O)N(C)CCNC(=O)OC(C)(C)C ; "4N HCl/Dox" ; CC(OC(=O)c1cc(S(N)(=O)=O)c(Cl)cc1NCc1ccco1)OC(=O)N(C)CCN]

To 200 mg (0.330 mmol) of 5-(aminosulfonyl)-4-chloro-2-[(2-furanylmethyl) amino] benzoic acid 1-[[[[[[2-[[(1,1-dimethylethoxy) carbonyl] amino] ethyl]methylamino] carbonyl] oxy] ethyl ester, 4 mL of a 4 N hydrochloric acid/dioxane solution was added, and the resulting mixture was stirred for one hour. The solvent was distilled off under reduced pressure, and the residue was dried using a vacuum pump overnight. Ethyl acetate was added to the residue, and then the precipitated solid was collected by filtration to obtain 94 mg (52%) of the title compound.

¹H-NMR (DMSO-d₆, δ): 1.57 (3H, d, J=6 Hz), 2.85-2.94 (5H, m), 3.34-3.39 (1H, m), 3.53-3.60 (1H, m), 4.62 (2H, d, J=6 Hz), 6.38 (1H, d, J=3 Hz), 6.43 (1H, dd, J=2, 3 Hz), 6.90 (1H, q, J=6 Hz), 7.14 (1H, s), 7.38 (2H, s), 7.63 (1H, d, J=2 Hz), 7.91 (3H, br-s), 8.35 (1H, s), 8.39-8.42 (1H, m)

### Example 46

### [2-[[(1-Furosemide-ethoxy) carbonyl] methylamino] ethyl] amino-chondroitin Sulfate Conjugate

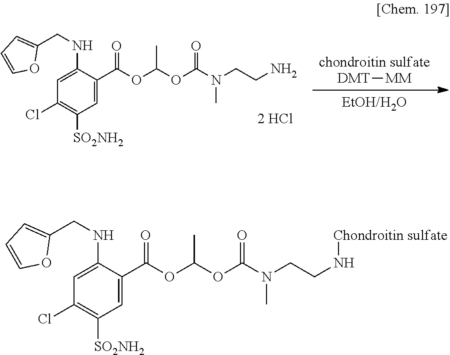

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 44 mg (0.080 mmol) of 5-(aminosulfonyl)-4-chloro-2-[(2-furanylmethyl) amino] benzoic acid 1-[[[(2-aminoethyl) methylamino] carbonyl] oxy]ethyl ester dihydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 160 mg of the title compound. Based on values of integral in ¹H-NMR, the introduction ratio of furosemide per unit of whole disaccharide (glucuronic acid) of chondroitin sulfate was 15%.

### Reference Example 141

### N-[2-[[(1,1-Dimethylethoxy) carbonyl] amino] ethyl]-N-methylcarbamic Acid 1-chloro-2-methylpropyl Ester

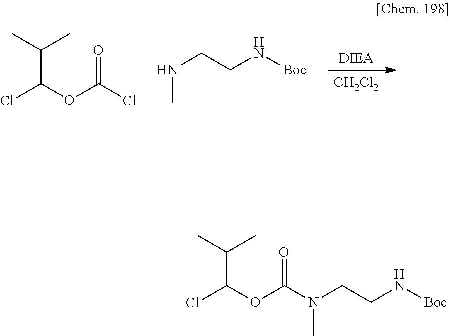

To a methylene chloride solution of 800 mg (4.68 mmol) of N-([(1,1-dimethylethoxy) carbonyl])-N′-methylethylenediamine and 978 μL (4.68 mmol) of N,N-diisopropylethylamine, a methylene chloride solution of 815 mg (5.61 mmol) of 1-chloro-2-methylpropyl chloroformate was added dropwise under cooling on ice. After 15 minutes, saturated sodium bicarbonate water was added to the reaction solution, and the resulting mixture was extracted with diethyl ether. The obtained organic layer was washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to obtain 1.25 g (87%) of the title compound.

¹H-NMR (CDCl₃, δ): 1.07 (3H, d, J=7 Hz), 1.08 (3H, d, J=7 Hz), 1.43 (9H, s), 2.16-2.22 (1H, m), 2.97 (1.5H, s), 3.00 (1.5H, s), 3.29-3.47 (4H, m), 4.68 (0.5H, br-s), 4.77 (0.5H, br-s), 6.32 (0.5H, d, J=5 Hz), 6.37 (0.5H, d, J=4 Hz)

### Reference Example 142

### 2-[4-[2-[(4-Chlorobenzoyl) amino] ethyl] phenoxy]-2-methylpropanoic Acid 1-[[[[2-[[(1,1-dimethylethoxy) carbonyl] amino] ethyl] methylamino] carbonyl] oxy]-2-methylpropyl Ester

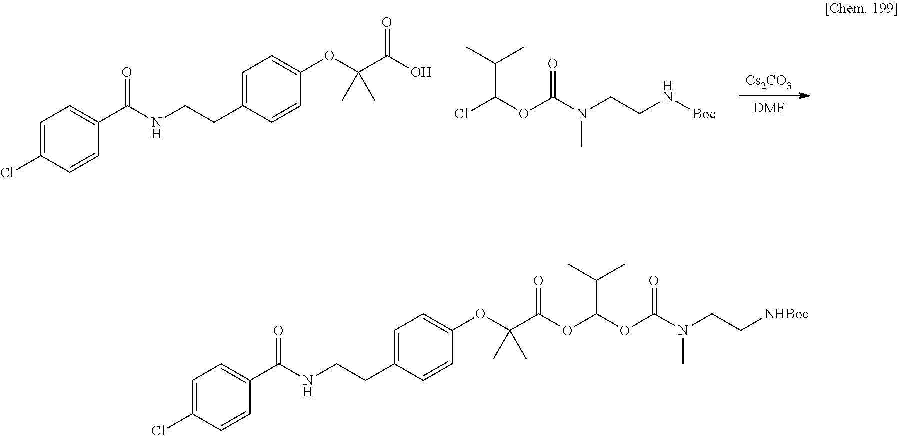

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 256 mg (0.829 mmol) of N-[2-[[(1,1-dimethylethoxy) carbonyl] amino]ethyl]-N-methylcarbamic acid 1-chloro-2-methylpropyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 168 mg (32%) of the title compound.

¹H-NMR (CDCl₃, δ, mixture of rotamers): 0.90-0.92 (6H, m), 1.42 (9H, s), 1.58 (3H, s), 1.61 (3H, s), 2.84-2.89 (5H, m) 2.10-3.38 (4H, m), 3.63-3.71 (2H, m), 4.56 (0.5H, br-s), 4.86 (0.5H, br-s), 6.12 (0.5H, br-s), 6.20 (0.5H, br-s), 6.61 (0.5H, d, J=5 Hz), 6.68 (0.5H, br-s), 6.82 (1H, d, J=8 Hz), 6.84 (1H, d, J=8 Hz), 7.08 (2H, d, J=8 Hz), 7.38 (2H, d, J=9 Hz), 7.62 (2H, d, J=9 Hz)

### Reference Example 143

### [2-[4-[2-[(4-Chlorobenzoyl) amino] ethyl] phenoxy]-2-methylpropanoic Acid 1-[[[(2-aminoethyl) methylamino] carbonyl] oxy]-2-methylpropyl Ester Hydrochloride

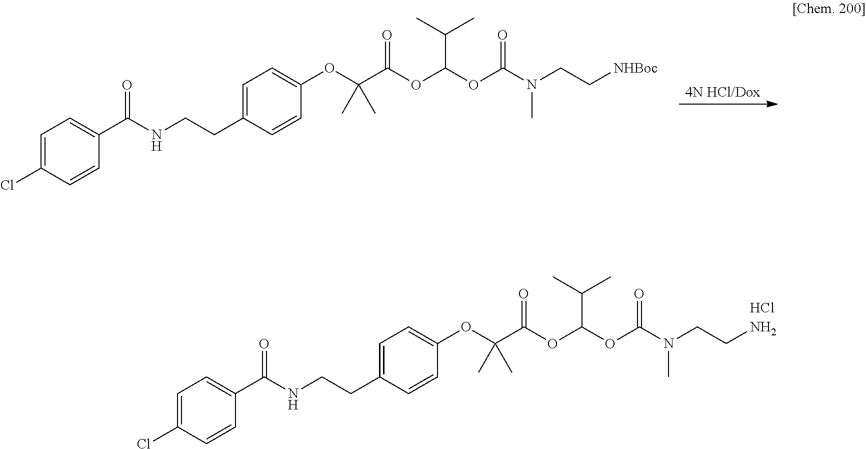 [SMILES: CC(C)C(OC(=O)N(C)CCNC(=O)OC(C)(C)C)OC(=O)C(C)(C)Oc1ccc(CCNC(=O)c2ccc(Cl)cc2)cc1 ; "4N HCl/Dox" ; CC(C)C(OC(=O)N(C)CCN)OC(=O)C(C)(C)Oc1ccc(CCNC(=O)c2ccc(Cl)cc2)cc1 ; Cl]

To 162 mg (0.255 mmol) of 2-[4-[2-[(4-chlorobenzoyl) amino] ethyl]phenoxy]-2-methylpropanoic acid 1-[[[[2-[[(1,1-dimethylethoxy) carbonyl] amino]ethyl] methylamino] carbonyl] oxy]-2-methylpropyl ester, 3 mL of a 4 N hydrochloric acid/dioxane solution was added, and the resulting mixture was allowed to stand for one hour. The solvent was distilled off under reduced pressure, and then ethyl acetate was added to the residue. The resulting mixture was stirred for three hours. The precipitated crystals were collected by filtration to obtain 112 mg (77%) of the title compound.

¹H-NMR (DMSO-d₆, δ, mixture of rotamers): 0.83-0.88 (6H, m), 1.48 (3H, s), 1.52 (3H, s), 1.97-2.00 (1H, m), 2.78 (2H, t, J=8 Hz), 2.85 (1.5H, s), 2.86 (1.5H, s), 2.91-2.94 (2H, m), 3.32-3.36 (1H, m), 3.41-3.45 (2H, m), 3.53-3.58 (1H, m), 6.51 (0.5H, d, J=4 Hz), 6.54 (0.5H, d, J=4 Hz), 6.73-6.77 (2H, m), 7.12 (2H, d, J=9 Hz), 7.53 (2H, d, J=9 Hz), 7.84 (2H, d, J=9 Hz), 7.99-8.02 (3H, m), 8.67 (1H, t, J=6 Hz)

### Example 47

### [2-[[(1-Bezafibrate-2-methylpropoxy) carbonyl] methylamino] ethyl] amino-chondroitin Sulfate Conjugate

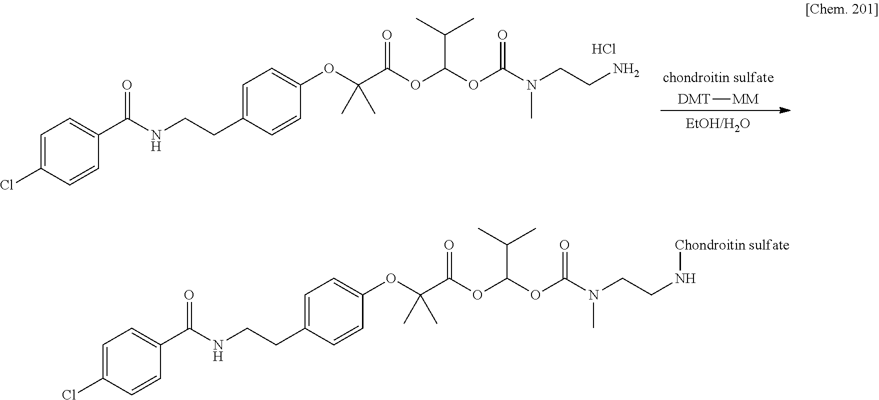

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 37 mg (0.080 mmol) of [2-[4-[2-[(4-chlorobenzoyl) amino]ethyl] phenoxy]-2-methylpropanoic acid 1-[[[(2-aminoethyl) methylamino] carbonyl]oxy]-2-methylpropyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 263 mg of the title compound. Based on values of integral in ¹H-NMR, the introduction ratio of bezafibrate per unit of whole disaccharide (glucuronic acid) of chondroitin sulfate was 11%.

### Reference Example 144

### N-[2-[[(1,1-Dimethylethoxy) carbonyl] amino] ethyl]-N-(1-methylethyl) carbamic Acid 1-chloro-2-methylpropyl Ester

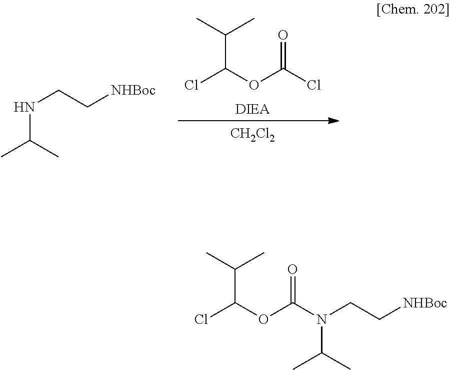

To a methylene chloride solution of 600 mg (2.97 mmol) of N-([(1,1-dimethylethoxy) carbonyl])-N′-(2-methylethyl) ethylenediamine and 0.620 mL (3.56 mmol) of N,N-diisopropylethylamine, a methylene chloride solution of 0.434 mL (2.97 mmol) of 1-chloro-2-methylpropyl chloroformate was added dropwise under cooling on ice. After three hours, water was added to the reaction solution, and the resulting mixture was extracted with diethyl ether. The obtained organic layer was washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to give 989 mg (99%) of the title compound.

¹H-NMR (CDCl₃, δ, mixture of rotamers): 1.07 (3H, d, J=7 Hz), 1.08 (3H, d, J=7 Hz), 1.20 (6H, d, J=7 Hz), 1.44 (9H, s), 2.17-2.23 (1H, m), 3.26-3.32 (4H, m), 4.13 (0.5H, br-s), 4.29 (0.5H, br-s), 4.73 (0.5H, br-s), 4.96 (0.5H, br-s), 6.39 (1H, br-d, J=12 Hz)

### Reference Example 145

### 2-[4-[2-[(4-Chlorobenzoyl) amino] ethyl] phenoxy]-2-methylpropanoic Acid 1-[[[[2-[[(1,1-dimethylethoxy) carbonyl] amino] ethyl] (1-methylethyl) amino]carbonyl] oxy]-2-methylpropyl Ester

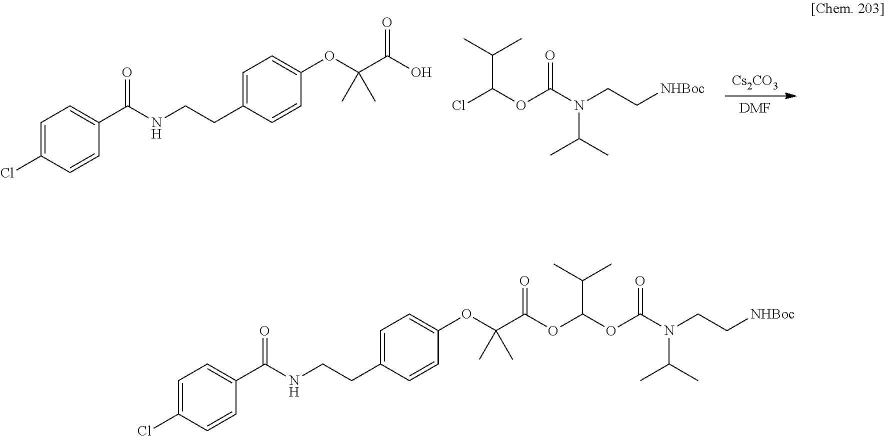

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 279 mg (0.829 mmol) of N-[2-[[(1,1-dimethylethoxy) carbonyl] amino]ethyl]-N-(1-methylethyl) carbamic acid 1-chloro-2-methylpropyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to give 363 mg (66%) of the title compound.

¹H-NMR (DMSO-d₆, δ, mixture of rotamers): 0.90-0.93 (6H, m), 1.09-1.12 (6H, m), 1.42 (9H, s), 1.60 (6H, s), 2.03-2.07 (1H, m), 2.85 (2H, t, J=7 Hz), 3.15-3.25 (4H, m), 3.65-3.66 (2H, m), 4.07-4.20 (1H, m), 4.63 (0.5H, br-s), 5.01 (0.5H, br-s), 6.15 (1H, br-s), 6.69 (1H, br-s), 6.83 (2H, d, J=9 Hz), 7.08 (2H, d, J=9 Hz), 7.38 (2H, d, J=9 Hz), 7.63 (2H, d, J=9 Hz)

### Reference Example 146

### 2-[4-[2-[(4-Chlorobenzoyl) amino] ethyl] phenoxy]-2-methylpropanoic Acid 1-[[(2-aminoethyl) (1-methylethyl) amino] carbonyl] oxy]-2-methylpropyl Ester Hydrochloride

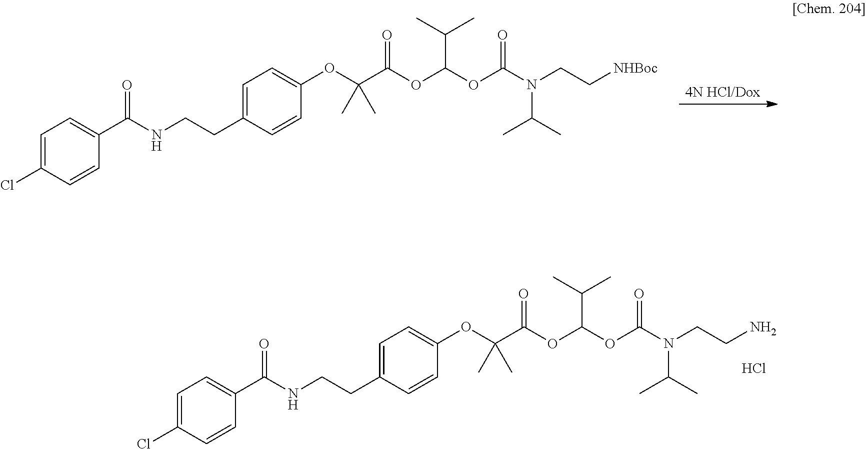 [SMILES: CC(C)C(OC(=O)N(CCNC(=O)OC(C)(C)C)C(C)C)OC(=O)C(C)(C)Oc1ccc(CCNC(=O)c2ccc(Cl)cc2)cc1 ; "4N HCl/Dox" ; CC(C)C(OC(=O)N(CCN)C(C)C)OC(=O)C(C)(C)Oc1ccc(CCNC(=O)c2ccc(Cl)cc2)cc1 ; Cl]

To 200 mg (0.302 mmol) of 2-[4-[2-[(4-chlorobenzoyl) amino] ethyl] phenoxy]-2-methylpropanoic acid 1-[[[[2-[[(1,1-dimethylethoxy) carbonyl] amino]ethyl] (1-methylethyl) amino] carbonyl] oxy]-2-methylpropyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 132 mg (73%) of the title compound.

¹H-NMR (CDCl₃, δ, mixture of rotamers): 0.85 (3H, d, J=7 Hz), 0.87 (3H, d, J=7 Hz), 1.02-1.10 (6H, m), 1.48 (3H, s), 1.51 (3H, s), 1.97-2.03 (1H, m), 2.77-2.88 (4H, m), 3.40-3.44 (4H, m), 4.01-4.17 (1H, m), 6.56 (1H, d, J=5 Hz), 6.73-6.77 (2H, m), 7.12 (2H, d, J=9 Hz), 7.53 (2H, d, J=9 Hz), 7.84 (2H, d, J=9 Hz), 7.98 (3H, br-s), 8.66 (1H, br-s)

### Example 48

### [2-[[(1-Bezafibrate-2-methylpropoxy) carbonyl] (1-methylethyl) amino] ethyl] amino-chondroitin Sulfate Conjugate

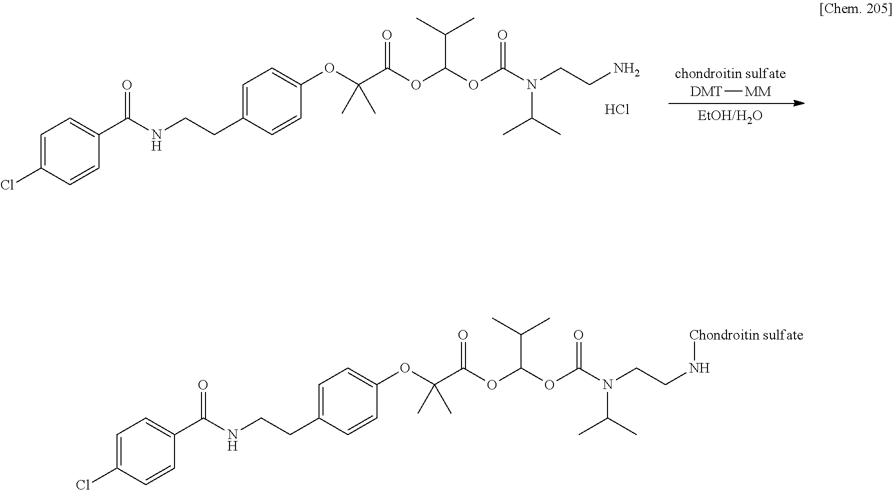

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 48 mg (0.080 mmol) of 2-[4-[2-[(4-chlorobenzoyl) amino]ethyl] phenoxy]-2-methylpropanoic acid 1-[[(2-aminoethyl) (1-methylethyl) amino]carbonyl] oxy]-2-methylpropyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 4 mL) thereto until immediately before the reaction solution became cloudy. The reaction solution was added dropwise under stirring to 8 mL of 90% ethanol. To the mixed liquid, 7 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 252 mg of the title compound. Based on values of integral in ¹H-NMR, the introduction ratio of bezafibrate per unit of whole disaccharide (glucuronic acid) of chondroitin sulfate was 13%.

### Reference Example 147

### N-[3-[[(1,1-Dimethylethoxy) carbonyl] amino] propyl]-N-methylcarbamic Acid Chloromethyl Ester

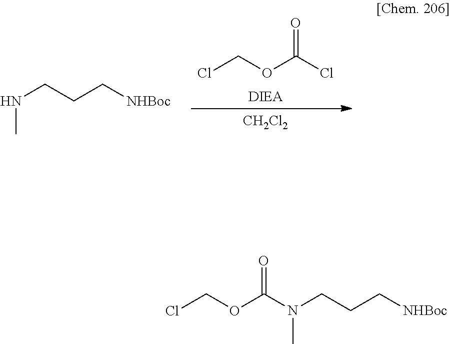

To a methylene chloride solution of 1.00 g (5.31 mmol) of N-([(1,1-dimethylethoxy) carbonyl])-N′-methylpropylenediamine and 1.39 mL (7.97 mmol) of N,N-diisopropylethylamine, a methylene chloride solution of 0.567 mL (6.37 mmol) of chloromethyl chloroformate was added dropwise under cooling on ice. After 15 minutes, water was added to the reaction solution, and the resulting mixture was extracted with diethyl ether. The obtained organic layer was washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to obtain 823 mg (61%) of the title compound.

¹H-NMR (CDCl₃, δ): 1.46 (9H, s), 1.68 (2H, br-s), 2.83 (3H, s), 3.19 (2H, br-s), 3.31 (2H, br-s), 5.75 (2H, s), 6.19 (1H, br-s)

### Reference Example 148

### 2-[4-[2-[(4-Chlorobenzoyl) amino] ethyl] phenoxy]-2-methylpropanoic Acid [[[[3-[[(1,1-dimethylethoxy) carbonyl] amino] propyl] methylamino] carbonyl] oxy]methyl Ester

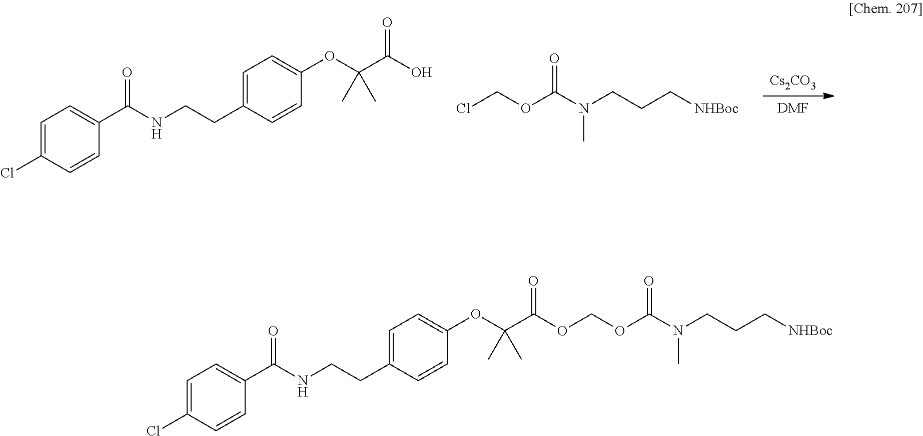

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 232 mg (0.829 mmol) of N-[3-[[(1,1-dimethylethoxy) carbonyl] amino]propyl]-N-methylcarbamic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 423 mg (84%) of the title compound.

¹H-NMR (CDCl₃, δ): 1.45 (9H, s), 1.59 (6H, s), 1.61-1.62 (2H, m), 2.81 (3H, s), 2.85 (2H, t, J=7 Hz), 3.12 (2H, br-s), 3.25-3.28 (2H, m), 3.66 (2H, q, J=7 Hz), 5.80 (2H, s), 6.00 (1H, br-s), 6.20 (1H, br-s), 6.83 (2H, d, J=9 Hz), 7.08 (2H, d, J=9 Hz), 7.37 (2H, d, J=9 Hz), 7.63 (2H, d, J=9 Hz)

### Reference Example 149

### 2-[4-[2-[(4-Chlorobenzoyl) amino] ethyl] phenoxy]-2-methylpropanoic Acid [[[(3-aminopropyl) methylamino] carbonyl] oxy] methyl Ester Hydrochloride

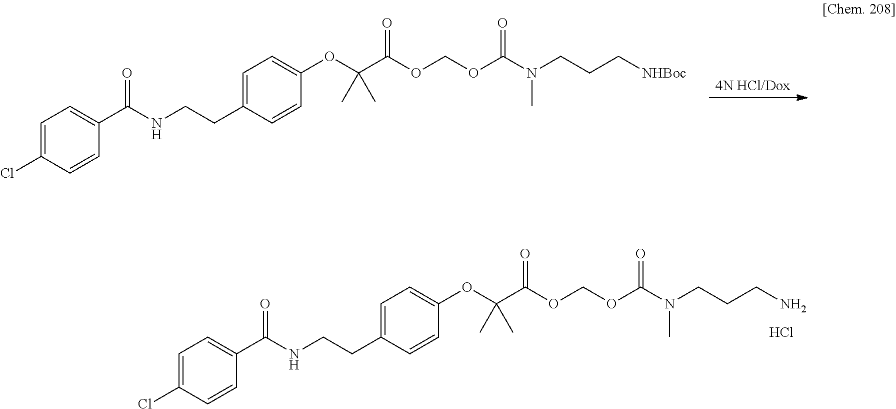 [SMILES: CN(CCCNC(=O)OC(C)(C)C)C(=O)OCOC(=O)C(C)(C)Oc1ccc(CCNC(=O)c2ccc(Cl)cc2)cc1 ; "4N HCl/Dox" ; CN(CCCN)C(=O)OCOC(=O)C(C)(C)Oc1ccc(CCNC(=O)c2ccc(Cl)cc2)cc1 ; Cl]

To 300 mg (0.495 mmol) of 2-[4-[2-[(4-chlorobenzoyl) amino] ethyl]phenoxy]-2-methylpropanoic acid [[[[3-[[(1,1-dimethylethoxy) carbonyl] amino]propyl] methylamino] carbonyl] oxy] methyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 218 mg (81%) of the title compound.

¹H-NMR (CD₃OD, δ): 1.56 (6H, s), 1.86 (2H, quin, J=8 Hz), 2.68 (3H, s), 2.88 (2H, t, J=8 Hz), 2.97 (2H, t, J=8 Hz), 2.23 (2H, t, J=8 Hz), 3.58 (2H, t, J=8 Hz), 5.83 (2H, s), 6.83 (2H, d, J=9 Hz), 7.17 (2H, d, J=9 Hz), 7.49 (2H, d, J=9 Hz), 7.77 (2H, d, J=9 Hz)

### Example 49

### [3-[[(Bezafibrate-methoxy) carbonyl] methylamino] propyl] amino-chondroitin Sulfate Conjugate

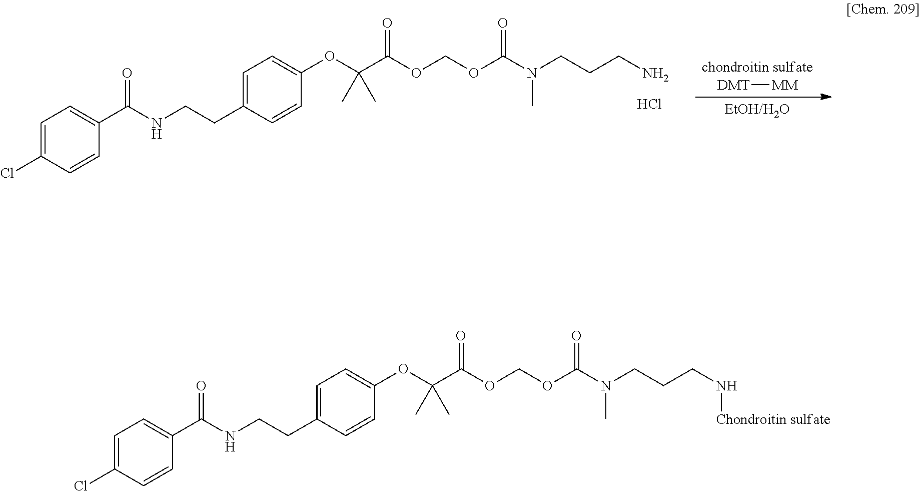

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 43 mg (0.080 mmol) of 2-[4-[2-[(4-chlorobenzoyl) amino]ethyl] phenoxy]-2-methylpropanoic acid [[[(3-aminopropyl) methylamino] carbonyl]oxy] methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 148 mg of the title compound. Based on values of integral in ¹H-NMR, the introduction ratio of bezafibrate per unit of whole disaccharide (glucuronic acid) of chondroitin sulfate was 5%.

### Reference Example 150

### N-[3-[[(1,1-Dimethylethoxy) carbonyl] amino] propyl]-N-methylcarbamic Acid 1-chloroethyl Ester

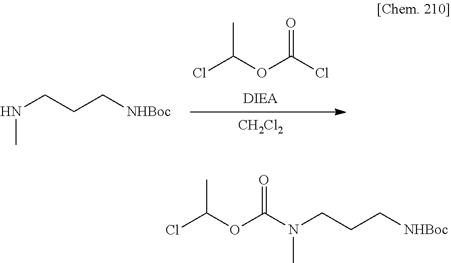

To a methylene chloride solution of 1.00 g (5.31 mmol) of N-(1,1-dimethylethoxy) carbonyl])-N′-methylpropylenediamine and 1.39 mL (7.97 mmol) of N,N-diisopropylethylamine, a methylene chloride solution of 0.696 mL (6.37 mmol) of 1-chloroethyl chloroformate was added dropwise under cooling on ice. After 15 minutes, water was added to the reaction solution, and the resulting mixture was extracted with diethyl ether. The obtained organic layer was washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to obtain 412 mg (26%) of the title compound.

¹H-NMR (CDCl₃, δ): 1.47 (9H, s), 1.66-1.72 (2H, m), 1.78 (3H, d, J=6 Hz), 2.82 (3H, s), 3.19-3.30 (4H, m), 6.04 (1H, br-s), 6.57 (1H, q, J=6 Hz)

### Reference Example 151

### 2-[4-[2-[(4-Chlorobenzoyl) amino] ethyl] phenoxy]-2-methylpropanoic Acid 1-[[[[3-[[(1,1-dimethylethoxy) carbonyl] amino] propyl] methylamino] carbonyl] oxy]ethyl Ester

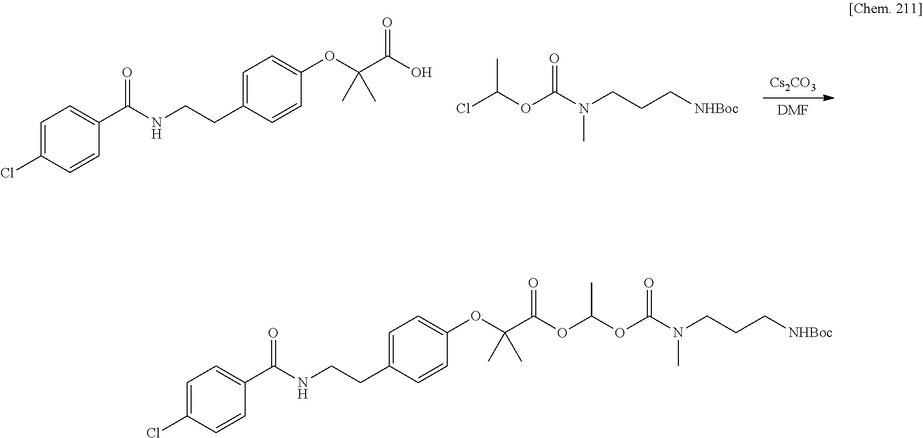

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 244 mg (0.829 mmol) of N-[3-[[(1,1-dimethylethoxy) carbonyl] amino]propyl]-N-methylcarbamic acid 1-chloroethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 273 mg (53%) of the title compound.

¹H-NMR (CDCl₃, δ): 1.44-1.50 (12H, m), 1.58-1.60 (8H, m), 2.80 (3H, s), 2.85 (2H, t, J=7 Hz), 3.10 (2H, br-s), 3.26 (2H, br-s), 3.60-3.71 (2H, m), 5.88 (1H, br-s), 6.19 (1H, br-s), 6.83-6.88 (3H, m), 7.08 (2H, d, J=9 Hz), 7.37 (2H, d, J=9 Hz), 7.63 (2H, d, J=9 Hz)

### Reference Example 152

### (3R)-3-[[(1,1-Dimetylethoxy) carbonyl] amino]-1-pyrrolidine Carboxylic Acid 1-chloroethyl Ester

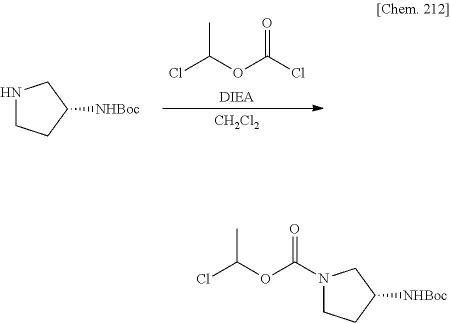

To a methylene chloride solution of 1.00 g (5.37 mmol) of (3R)-3-[[(1,1-dimetylethoxy) carbonyl] amino] pyrrolidine and 1.40 mL (8.05 mmol) of N,N-diisopropylethylamine, a methylene chloride solution of 921 mg (6.44 mmol) of 1-chloroethyl chloroformate was added dropwise under cooling on ice. After 15 minutes, water was added to the reaction solution, and the resulting mixture was extracted with diethyl ether. The obtained organic layer was washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to obtain 1.38 g (88%) of the title compound as a 1:1 diastereomeric mixture.

¹H-NMR (CDCl₃, δ, mixture of diastereomers): 1.45 (9H, s), 1.79-1.89 (4H, m), 2.11-2.19 (1H, m), 3.22-3.31 (1H, m), 3.41-3.59 (2H, m), 3.66-3.73 (1H, m), 4.22 (1H, br-s), 4.61 (0.5H, br-s), 4.64 (0.5H, br-s), 6.56-6.60 (1H, m)

### Reference Example 153

### 2-[4-[2-[(4-Chlorobenzoyl) amino] ethyl] phenoxy]-2-methylpropanoic Acid 1-[[[(3R)-3-[[(1,1-dimethylethoxy) carbonyl] amino]-1-pyrrolidinyl] carbonyl] oxy]ethyl Ester

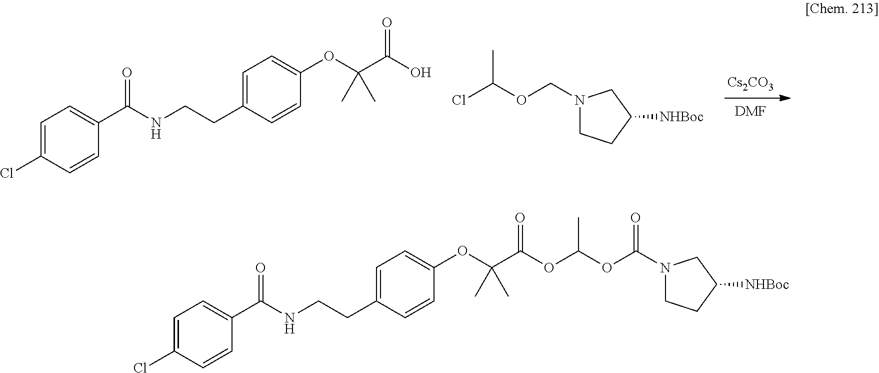

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 242 mg (0.829 mmol) of (3R)-3-[[(1,1-dimetylethoxy) carbonyl] amino]-1-pyrrolidine carboxylic acid 1-chloroethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 362 mg (71%) of the title compound.

¹H-NMR (CDCl₃, δ, mixture of diastereomers): 1.44 (9H, s), 1.48 (3H, d, J=6 Hz), 1.58 (3H, s), 1.59 (3H, s), 1.76-1.83 (1H, m), 2.07-2.14 (1H, m), 2.86 (2H, t, J=7 Hz), 3.10-3.21 (1H, m), 3.30-3.48 (2H, m), 3.57-3.73 (3H, m), 4.15 (1H, br-s), 4.61 (0.5H, br-s), 4.84 (0.5H, br-s), 6.22 (0.5H, br-s), 6.26 (0.5H, br-s), 6.83-6.90 (3H, m), 7.07 (2H, d, J=9 Hz), 7.38 (2H, d, J=9 Hz), 7.62-7.62 (2H, m)

### Reference Example 154

### 2-[4-[2-[(4-Chlorobenzoyl) amino] ethyl] phenoxy]-2-methylpropanoic Acid 1-[[[(3R)-3-amino-1-pyrrolidinyl] carbonyl] oxy] ethyl Ester Hydrochloride

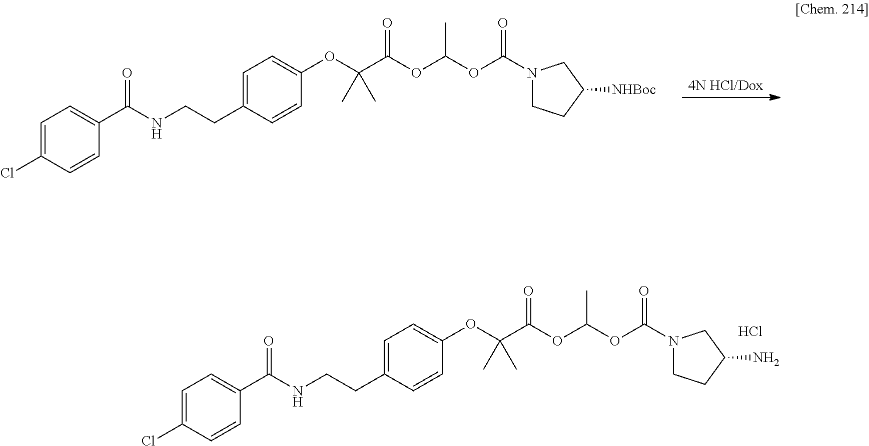 [SMILES: CC(OC(=O)N1CC[C@@H](NC(=O)OC(C)(C)C)C1)OC(=O)C(C)(C)Oc1ccc(CCNC(=O)c2ccc(Cl)cc2)cc1 ; "4N HCl/Dox" ; CC(OC(=O)N1CC[C@@H](N)C1)OC(=O)C(C)(C)Oc1ccc(CCNC(=O)c2ccc(Cl)cc2)cc1 ; Cl]

To 200 mg (0.324 mmol) of 2-[4-[2-[(4-chlorobenzoyl) amino] ethyl] phenoxy]-2-methylpropanoic acid 1-[[[(3R)-3-[[(1,1-dimethylethoxy) carbonyl] amino]-1-pyrrolidinyl] carbonyl] oxy] ethyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 167 mg (quant.) of the title compound.

¹H-NMR (DMSO-d₆, δ, mixture of diastereomers): 1.40-1.43 (3H, m), 1.47-1.49 (6H, m), 1.96-2.04 (1H, m), 2.13-2.29 (1H, m), 2.75-2.80 (2H, m), 3.25-3.72 (6H, m), 3.80 (1H, br-s), 6.71-6.77 (3H, m), 7.11-7.14 (2H, m), 7.53 (2H, d, J=9 Hz), 7.83 (1H, d, J=9 Hz), 7.84 (1H, d, J=9 Hz), 8.30 (3H, br-s), 8.67 (1H, br-s)

### Example 50

### [(3R)-1-[(1-Bezafibrate-ethoxy) carbonyl]-3-pyrrolidinyl] amino-chondroitin Sulfate Conjugate

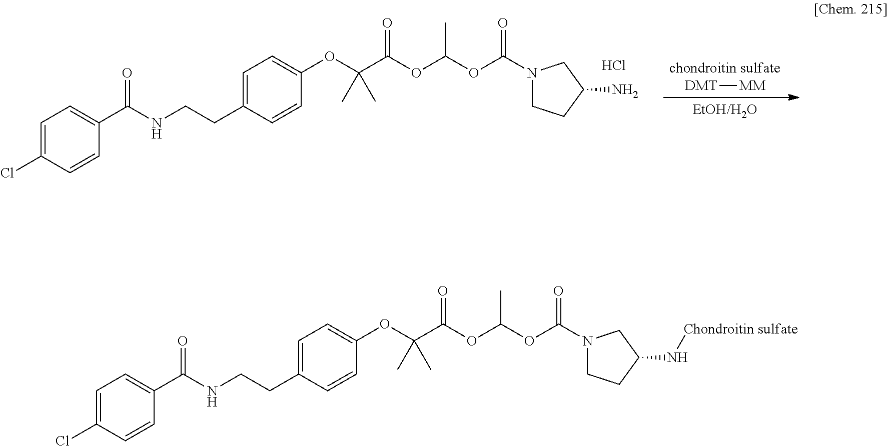

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 44 mg (0.080 mmol) of 2-[4-[2-[(4-chlorobenzoyl) amino]ethyl] phenoxy]-2-methylpropanoic acid 1-[[[(3R)-3-amino-1-pyrrolidinyl] carbonyl]oxy] ethyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 8 mL of 90% ethanol. To the mixed liquid, 9 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 236 mg of the title compound. Based on values of integral in ¹H-NMR, the introduction ratio of bezafibrate per unit of whole disaccharide (glucuronic acid) of chondroitin sulfate was 4%.

### Reference Example 155

### (3R)-3-[[(1,1-Dimetylethoxy) carbonyl] amino]-1-piperidine Carboxylic Acid Chloromethyl Ester

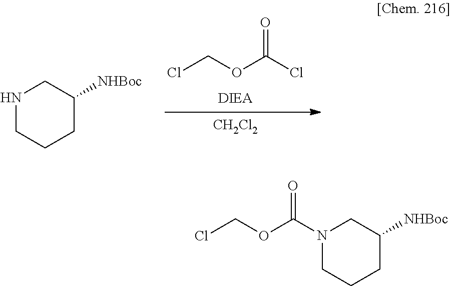

To a methylene chloride solution of 500 mg (2.50 mmol) of (3R)-3-[[(1,1-dimetylethoxy) carbonyl] amino] piperidine and 0.652 mL (3.75 mmol) of N,N-diisopropylethylamine, a methylene chloride solution of 386 mg (3.00 mmol) of chloromethyl chloroformate was added dropwise under cooling on ice. After 15 minutes, water was added to the reaction solution, and the resulting mixture was extracted with diethyl ether. The obtained organic layer was washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to obtain 630 mg (quant.) of the title compound.

¹H-NMR (CDCl₃, δ): 1.45-1.55 (1H, m), 1.69-1.73 (1H, m), 1.87-1.91 (1H, m), 3.22-3.35 (2H, m), 3.57-3.77 (3H, m), 4.54 (1H, br-s), 5.72-5.85 (2H, m)

### Reference Example 156

### 2-[4-[2-[(4-Chlorobenzoyl) amino] ethyl] phenoxy]-2-methylpropanoic Acid [[[(3R)-3-[[(1,1-dimethylethoxy) carbonyl] amino]-1-piperidinyl] carbonyl] oxy]methyl Ester

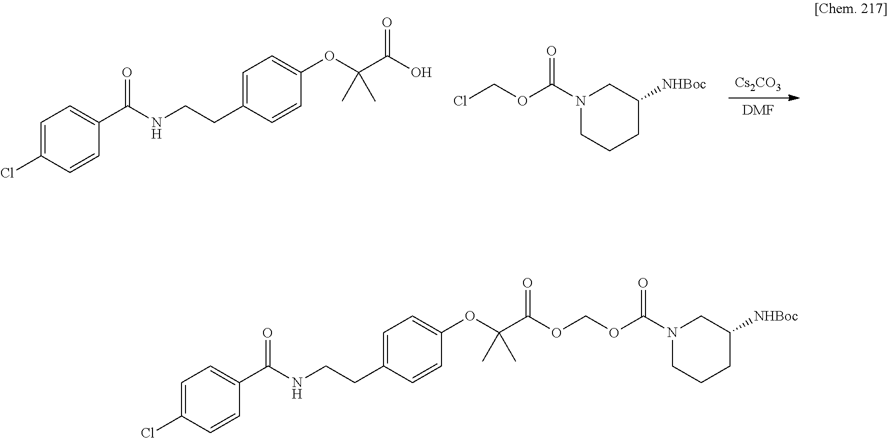

To a methanol solution of 300 mg (0.829 mmol) of bezafibrate, 135 mg (0.415 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 242 mg (0.829 mmol) of (3R)-3-[[(1,1-dimetylethoxy) carbonyl] amino]-1-pyrrolidine carboxylic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 512 mg (quant.) of the title compound.

¹H-NMR (CDCl₃, δ): 1.43-1.66 (18H, m), 1.88-1.91 (1H, m), 2.86 (2H, t, J=7 Hz), 3.02 (1H, br-s), 3.13 (1H, br-s), 3.59-3.69 (4H, m), 3.78-3.83 (1H, m), 4.63 (1H, br-s), 5.83 (1H, d, J=5 Hz), 5.87 (1H, d, J=5 Hz), 6.24 (1H, br-s), 6.82 (2H, d, J=9 Hz), 7.08 (2H, d, J=9 Hz), 7.37 (2H, d, J=9 Hz), 7.63 (2H, d, J=9 Hz)

### Reference Example 157

### 2-[4-[2-[(4-Chlorobenzoyl) amino] ethyl] phenoxy]-2-methylpropanoic Acid [[(3R)-(3-amino-1-piperidinyl) carbonyl] oxy] methyl Ester Hydrochloride

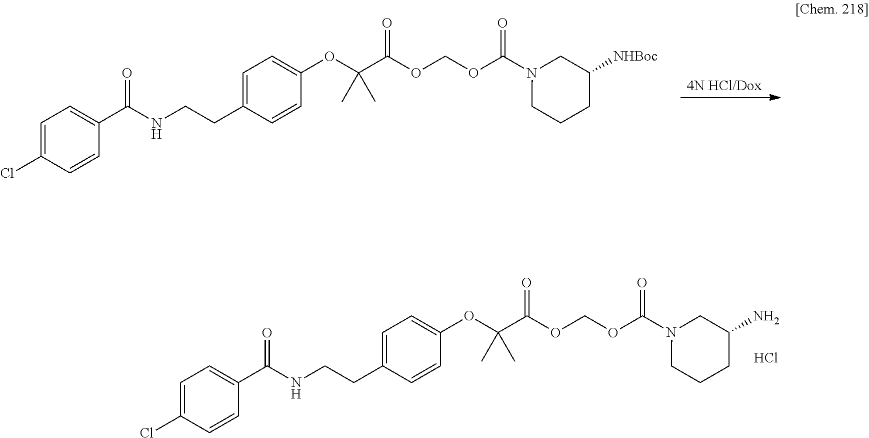 [SMILES: CC(C)(C)OC(=O)N[C@@H]1CCCN(C(=O)OCOC(=O)C(C)(C)Oc2ccc(CCNC(=O)c3ccc(Cl)cc3)cc2)C1 ; "4N HCl/Dox" ; CC(C)(Oc1ccc(CCNC(=O)c2ccc(Cl)cc2)cc1)C(=O)OCOC(=O)N1CCC[C@@H](N)C1 ; Cl]

To 300 mg (0.485 mmol) of 2-[4-[2-[(4-chlorobenzoyl) amino] ethyl]phenoxy]-2-methylpropanoic acid [[[(3R)-3-[[(1,1-dimethylethoxy) carbonyl] amino]-1-piperidinyl] carbonyl] oxy] methyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 242 mg (90%) of the title compound.

¹H-NMR (DMSO-d₆, δ, mixture of rotamers): 1.41-1.60 (8H, m), 1.71-1.74 (1H, m), 1.97-2.00 (1H, m), 2.79 (2H, t, J=8 Hz), 2.97-3.10 (3H, m), 3.43-3.47 (2H, m), 3.63-3.72 (1H, m), 3.98-4.00 (1H, m), 5.80 (2H, s), 6.75 (2H, d, J=9 Hz), 7.13 (2H, d, J=9 Hz), 7.54 (2H, d, J=9 Hz), 7.85 (2H, d, J=9 Hz), 8.17 (3H, br-s), 8.70 (1H, br-d, J=5 Hz)

### Example 51

### (3R)-[1-[(Bezafibrate-methoxy) carbonyl]-3-piperidinyl] amino-chondroitin Sulfate Conjugate

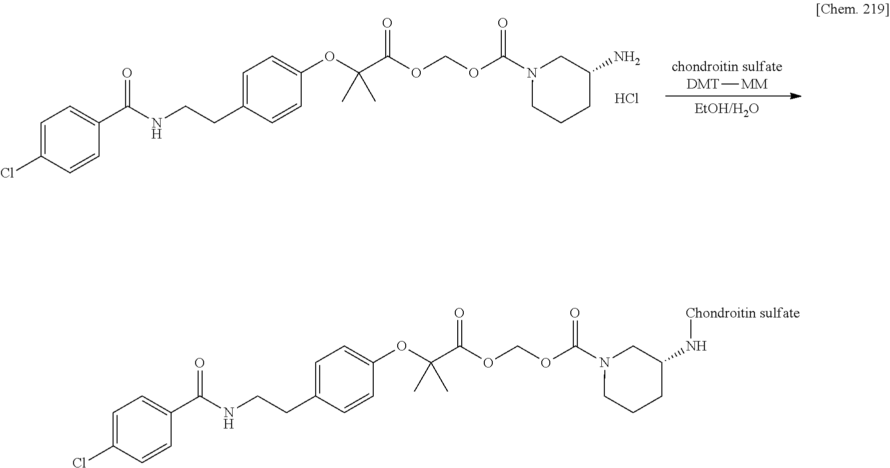

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 44 mg (0.080 mmol) of 2-[4-[2-[(4-chlorobenzoyl) amino]ethyl] phenoxy]-2-methylpropanoic acid [[(3R)-3-amino-1-piperidinyl] carbonyl] oxy]ethyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 3 mL) thereto until immediately before the reaction solution became cloudy. The reaction solution was added dropwise under stirring to 8 mL of 90% ethanol. To the mixed liquid, 8 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 218 mg of the title compound. Based on values of integral in ¹H-NMR, the introduction ratio of bezafibrate per unit of whole disaccharide (glucuronic acid) of chondroitin sulfate was 15%.

### Reference Example 158

### (1R,2S,4S)-2-[[(Phenylmethoxy) carbonyl] amino]-7-azabicyclo[2.2.1]heptane-7-carboxylic acid 9-fluorenylmethyl Ester

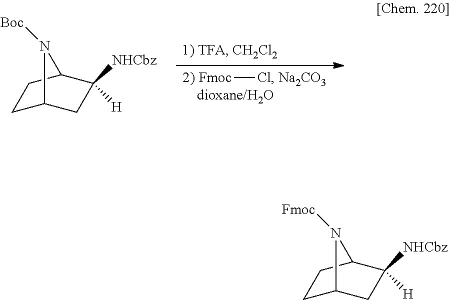

To a methylene chloride 4 mL solution of 300 mg (0.866 mmol) of (1R,2S,4S)-2-[[(phenylmethoxy) carbonyl] amino]-7-azabicyclo[2.2.1]heptane-7-carboxylic acid 1,1-dimethylethyl ester, 1 mL of trifluoroacetic acid was added under cooling on ice. The resulting mixture was stirred at room temperature for 30 minutes. Thereafter, 4 mL of toluene was added to the reaction solution, and the solvent was distilled off under reduced pressure. The residue was dissolved in a mixed solvent of 3 mL of 1,4-dioxane and 3 mL of water, and 275 mg (2.60 mmol) of sodium carbonate and 336 mg (1.30 mmol) of 9-fluorenylmethyl chloroformate were added thereto under cooling on ice. The resulting mixture was stirred at room temperature for one hour. Thereafter, water was added thereto, and the resulting mixture was extracted with diethyl ether. The obtained organic layer was washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to obtain 405 mg (quant.) of the title compound.

¹H-NMR (CDCl₃, δ, mixture of rotamers): 1.30-1.64 (5H, m), 1.90 (1H, dd, J=8, 13 Hz), 3.78 (1H, br-s), 4.15-4.19 (3H, m), 4.43 (1H, br-s), 4.49 (1H, br-s), 4.84 (1H, br-d, J=7 Hz), 5.04 (1H, d, J=13 Hz), 5.09 (1H, d, J=13 Hz), 7.26-7.40 (9H, m), 7.55-7.57 (2H, m), 7.73-7.76 (2H, m)

### Reference Example 159

### (1R,2S,4S)-2-[[(1,1-Dimethylethoxy) carbonyl] amino]-7-azabicyclo[2.2.1]heptane-7-carboxylic acid 9-fluorenylmethyl Ester

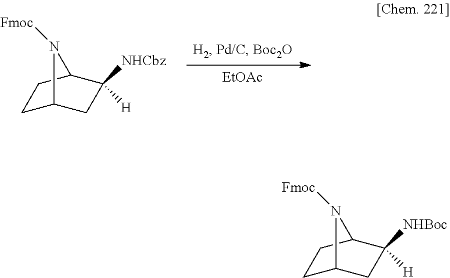

To an ethyl acetate 4 mL solution of 405 mg (0.864 mmol) of (1R,2S,4S)-2-[[(phenylmethoxy) carbonyl] amino]-7-azabicyclo[2.2.1]heptane-7-carboxylic acid 9-fluorenylmethyl ester, an ethyl acetate (4 mL) solution of 40 mg of Pd/C and 283 mg (1.30 mmol) of tert-butyl dicarbonate were added in an argon atmosphere. Thereafter, the reaction solution was stirred at room temperature for 1.5 hours in a hydrogen atmosphere, and then filtered, and the solvent of the filtrate was distilled off under reduced pressure. The residue was purified by silica gel column chromatography to obtain 320 mg (85%) of the title compound.

¹H-NMR (CDCl₃, δ): 1.30-1.63 (14H, m), 1.89 (1H, dd, J=8, 13 Hz), 3.72 (1H, br-s), 4.12-4.22 (3H, m), 4.45-4.46 (2H, m), 4.62 (1H, br-d, J=8 Hz), 7.32 (2H, dt, J=1, 8 Hz), 7.40 (2H, t, J=8 Hz), 7.59 (2H, d, J=8 Hz), 7.77 (2H, d, J=8 Hz)

### Reference Example 160

### (1R,2S,4S)-2-[[(1,1-Dimethylethoxy) carbonyl] amino]-7-azabicyclo[2.2.1]heptane-7-carboxylic Acid Chloromethyl Ester

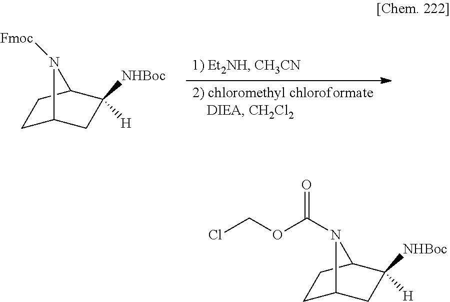

To an acetonitrile 2 mL solution of 310 mg (0.662 mmol) of (1R,2S,4S)-2-[[(1,1-dimethylethoxy) carbonyl] amino]-7-azabicyclo[2.2.1]heptane-7-carboxylic acid 9-fluorenylmethyl ester, 0.5 mL of diethylamine was added under cooling on ice. The resulting mixture was stirred at room temperature for 45 minutes. Thereafter, chloroform was added to the reaction solution, and the solvent was distilled off under reduced pressure. The residue was dissolved in a methylene chloride 4 mL solvent, and 0.173 mL (0.992 mmol) of N,N-diisopropylethylamine and 102 mg (0.794 mmol) of chloromethyl chloride carbonate were added under cooling on ice. The resulting mixture was stirred for 1.5 hours under cooling on ice. Thereafter, water was added thereto, and the resulting mixture was extracted with diethyl ether. The obtained organic layer was washed with a 5% potassium hydrogen sulfate aqueous solution, saturated sodium bicarbonate water, and saturated saline, and then dried with anhydrous magnesium sulfate, and the solvent was distilled off under reduced pressure. The residue was purified by silica gel column chromatography to obtain 178 mg (88%) of the title compound.

¹H-NMR (CDCl₃, δ, mixture of rotamers): 1.38-1.50 (12H, m), 1.73-1.77 (2H, m), 1.97 (1H, dd, J=9, 13 Hz), 3.76 (1H, br-s), 4.26 (1H, d, J=4 Hz), 4.37 (1H, br-s), 4.56 (1H, br-s), 5.73 (1H, br-s), 5.78 (1H, br-s)

### Reference Example 161

### 2-[4-[2-[(4-Chlorobenzoyl) amino] ethyl] phenoxy]-2-methylpropanoic Acid [[[[(1R,2S,4S)-2-[[(1,1-dimethylethoxy) carbonyl] amino]-7-azabicyclo[2.2.1]heptan-7-yl] carbonyl] oxy] methyl Ester

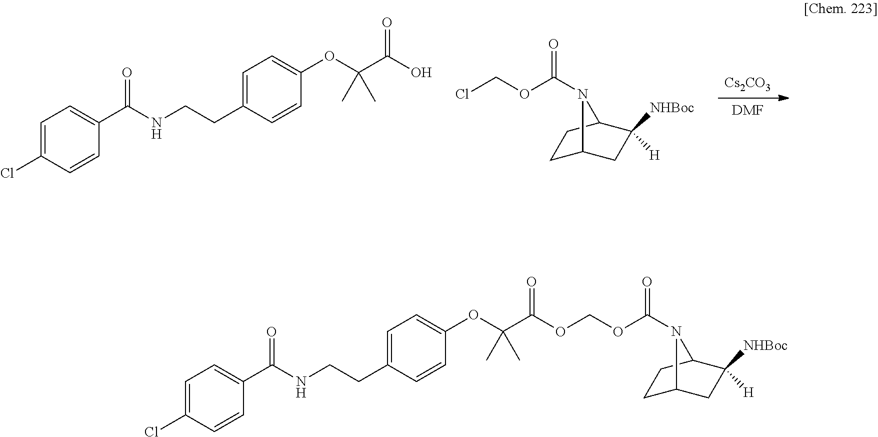

To a methanol solution of 250 mg (0.691 mmol) of bezafibrate, 112 mg (0.345 mmol) of cesium carbonate was added, and the resulting mixture was stirred at room temperature for 30 minutes. The solvent of the mixed suspension was distilled off under reduced pressure, and the residue was dried using a vacuum pump overnight. The residue was dissolved in N,N-dimethylformamide, and an N,N-dimethylformamide solution of 175 mg (0.576 mmol) of (1R,2S,4S)-2-[[(1,1-dimethylethoxy) carbonyl]amino]-7-azabicyclo[2.2.1]heptane-7-carboxylic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 371 mg (quant.) of the title compound.

¹H-NMR (CDCl₃, δ): 1.32-1.46 (12H, m), 1.59 (6H, s), 1.63-1.64 (2H, m), 1.91 (1H, dd, J=8, 13 Hz), 2.86 (2H, t, J=7 Hz), 3.66 (2H, q, J=7 Hz), 3.71 (1H, br-s), 4.17 (1H, br-s), 4.28 (1H, br-s), 4.57 (1H, br-s), 5.82 (2H, s), 6.24 (1H, br-s), 6.83 (2H, d, J=9 Hz), 7.09 (2H, d, J=9 Hz), 7.38 (2H, d, J=9 Hz), 7.63 (2H, d, J=9 Hz)

### Reference Example 162

### 2-[4-[2-[(4-Chlorobenzoyl) amino] ethyl] phenoxy]-2-methylpropanoic Acid [[[(1R,2S,4S)-2-amino-7-azabicyclo[2.2.1]heptan-7-yl] carbonyl] oxy] methyl Ester Hydrochloride

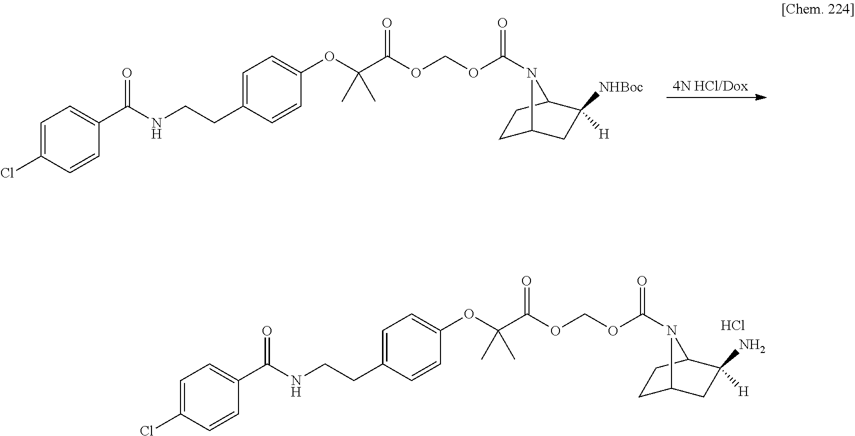 [SMILES: CC(C)(C)OC(=O)N[C@H]1CC2CCC1N2C(=O)OCOC(=O)C(C)(C)Oc1ccc(CCNC(=O)c2ccc(Cl)cc2)cc1 ; "4N HCl/Dox" ; CC(C)(Oc1ccc(CCNC(=O)c2ccc(Cl)cc2)cc1)C(=O)OCOC(=O)N1C2CCC1[C@@H](N)C2 ; Cl]

To 260 mg (0.413 mmol) of 2-[4-[2-[(4-chlorobenzoyl) amino] ethyl]phenoxy]-2-methylpropanoic acid [[[[(1R,2S,4S)-2-[[(1,1-dimethylethoxy) carbonyl]amino]-7-azabicyclo[2.2.1]heptan-7-yl] carbonyl] oxy] methyl ester, 3 mL of a 4 N hydrochloric acid/dioxane solution was added, and the resulting mixture was allowed to stand for one hour. The solvent was distilled off under reduced pressure, and then hexane was added to the residue. The resulting mixture was stirred for three hours. The precipitated crystals were collected by filtration to obtain 55 mg (24%) of the title compound.

¹H-NMR (DMSO-d₆, δ): 1.39-1.64 (11H, m), 1.88 (1H, dd, J=8, 13 Hz), 2.78 (2H, t, J=Hz), 3.36 (1H, dd, J=3, 8 Hz), 3.41-3.46 (2H, m), 4.26 (2H, br-s), 5.79 (2H, br-s), 6.76 (2H, d, J=9 Hz), 7.13 (2H, d, J=9 Hz), 7.53 (2H, d, J=9 Hz), 7.84 (2H, d, J=9 Hz), 8.09 (3H, br-s), 8.66 (1H, t, J=5 Hz)

### Example 52

### [(1R,2S,4S)-7-[Bezafibrate-methoxy) carbonyl]-7-azabicyclo[2.2.1]heptan-2-yl] amino-chondroitin Sulfate Conjugate

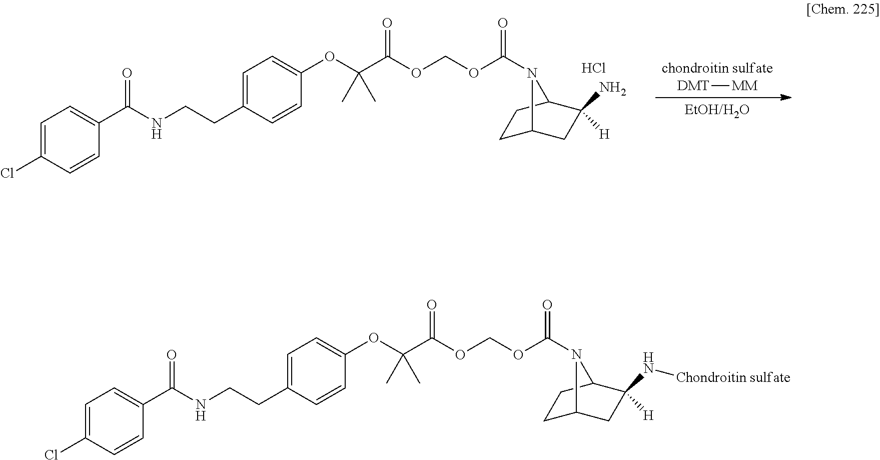

To 2.0 g (0.20 mmol) of a 5% sodium chondroitin sulfate aqueous solution, 1 mL of ethanol was added dropwise under stirring. To the mixed liquid, an ethanol 1.1 mL-water 0.5 mL solution of 23 mg (0.040 mmol) of 2-[4-[2-[(4-chlorobenzoyl) amino]ethyl] phenoxy]-2-methylpropanoic acid [[[(1R,2S,4S)-2-amino-7-azabicyclo[2.2.1]heptan-7-yl] carbonyl] oxy] methyl ester hydrochloride was added. Subsequently, an ethanol 0.4 mL solution of 11 mg (0.040 mmol) of 4-(4,6-dimethoxy-1,3,5-triazin-2-yl)-4-methylmorpholinium chloride (DMT-MM) was immediately added thereto. The resulting mixture was stirred overnight at room temperature. To the reaction solution, 50 μL of a 20% sodium chloride aqueous solution was added, and ethanol was further added dropwise (about 1.5 mL) until immediately before the reaction solution became cloudy. The reaction solution was added dropwise under stirring to 8 mL of 90% ethanol. To the mixed liquid, 4 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 117 mg of the title compound. Based on values of integral in ¹H-NMR, the introduction ratio of bezafibrate per unit of whole disaccharide (glucuronic acid) of chondroitin sulfate was 15%.

### Reference Example 163

### 7-[[(1,1-Dimethylethoxy) carbonyl] amino] heptanoic Acid Chloromethyl Ester

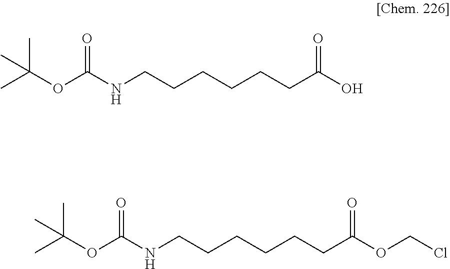 [SMILES: CC(C)(C)OC(=O)NCCCCCCC(=O)O ; CC(C)(C)OC(=O)NCCCCCCC(=O)OCCl]

Under cooling on ice, a methylene chloride solution of 1.21 g (8.15 mol) of chloromethyl chlorosulfonate was added dropwise to a water 15 mL-methylene chloride 15 mL mixed solution of 1.00 g (4.08 mmol) of 7-[[(1,1-dimethylethoxy) carbonyl]amino] heptanoic acid, 139 mg (0.41 mmol) of tetrabutylammonium hydrogen sulfate, and 1.37 g (16.32 mmol) of sodium hydrogen carbonate. The temperature of the reaction solution was returned to room temperature, and the reaction solution was stirred overnight. The methylene chloride layer of the reaction solution was isolated, washed with saturated saline, and then dried with anhydrous sodium sulfate. The solvent was condensed under reduced pressure, and the residue was purified by silica gel column chromatography (ethyl acetate/hexane) to obtain 1.11 g (92%) of the title compound.

¹H-NMR (CDCl₃, δ): 1.32-1.37 (41, m), 1.44-1.51 (11H, m), 1.63-1.67 (2H, m), 2.38 (2H, t, J=7 Hz), 3.10-3.11 (2H, m), 4.50 (1H, br-s), 5.70 (2H, s)

### Reference Example 164

### 2-[4-[2-[(4-Chlorobenzoyl) amino] ethyl] phenoxy]-2-methylpropanoic Acid [7-[[(1,1-dimethylethoxy) carbonyl] amino]-1-oxoheptyloxy] methyl Ester

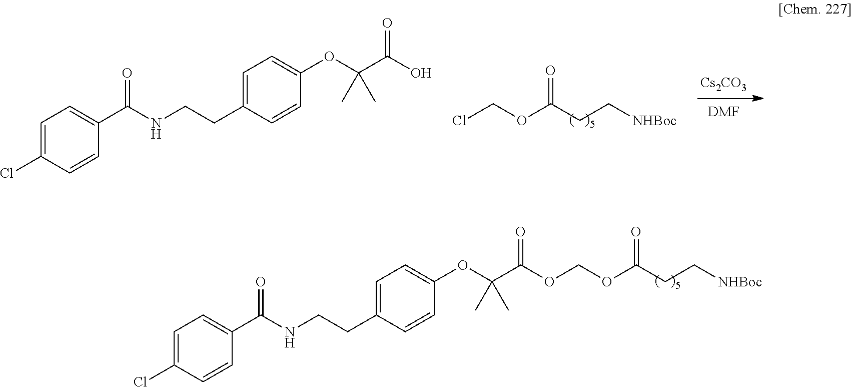

To a methanol suspension of 300 mg (0.829 mmol) of bezafibrate, 135 mg (0.415 mmol) of cesium carbonate was added, and the resulting mixture was stirred at room temperature for 30 minutes. The solvent of the mixed liquid was distilled off under reduced pressure, and the residue was dried using a vacuum pump overnight. The residue was dissolved in N,N-dimethylformamide, and an N,N-dimethylformamide solution of 243 mg (0.829 mmol) of 7-[[(1,1-dimethylethoxy) carbonyl] amino]heptanoic acid chloromethyl ester was added thereto. The resulting mixture was stirred overnight. Saturated sodium bicarbonate water was added to the reaction solution, and the resulting mixture was extracted with ethyl acetate. The obtained organic layer was washed with water and saturated saline, and then dried with anhydrous magnesium sulfate, and the solvent was distilled off under reduced pressure. The residue was purified by silica gel column chromatography to obtain 182 mg (35%) of the title compound.

¹H-NMR (CDCl₃, δ): 1.28-1.31 (4H, m), 1.43-1.46 (11H, m), 1.56-1.59 (8H, m), 2.29 (2H, t, J=7 Hz), 2.86 (2H, t, J=7 Hz), 3.07-3.10 (2H, m), 3.66 (2H, q, J=7 Hz), 4.55 (1H, br-s), 5.82 (2H, s), 6.26 (1H, br-s), 6.82 (2H, d, J=9 Hz), 7.09 (2H, d, J=9 Hz), 7.38 (2H, d, J=9 Hz), 7.64 (2H, d, J=9 Hz)

### Reference Example 165

### 2-[4-[2-[(4-Chlorobenzoyl) amino] ethyl] phenoxy]-2-methylpropanoic Acid (7-amino-1-oxoheptyloxy) methyl Ester Hydrochloride

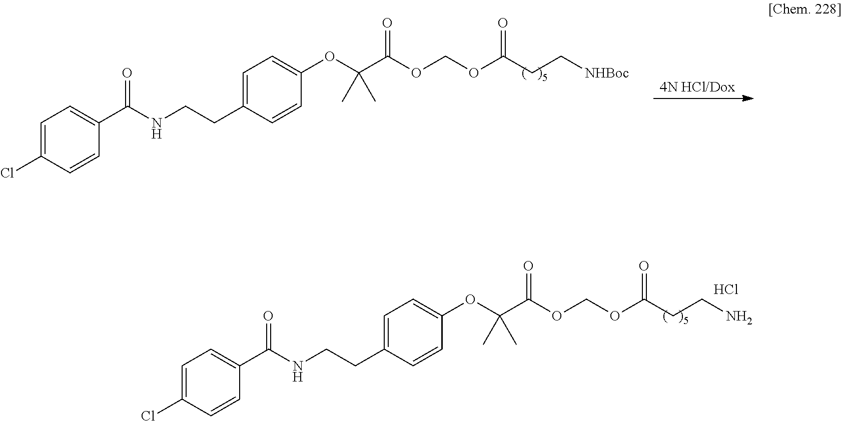

To 165 mg (0.266 mmol) of 2-[4-[2-[(4-chlorobenzoyl) amino] ethyl] phenoxy]-2-methylpropanoic acid [7-[[(1,1-dimethylethoxy) carbonyl] amino]-1-oxoheptyloxy] methyl ester, 3 mL of a 4 N hydrochloric acid/dioxane solution was added, and the resulting mixture was stirred for one hour. The solvent was distilled off under reduced pressure to obtain 132 mg (89%) of the title compound.

¹H-NMR (DMSO-d₆, δ): 1.24-1.31 (4H, m), 1.49-1.55 (10H, m), 2.33 (2H, t, J=8 Hz), 2.72-2.79 (4H, m), 3.42-3.46 (2H, m), 5.79 (2H, s), 6.74 (2H, d, J=9 Hz), 7.13 (2H, d, J=9 Hz), 7.53 (2H, d, J=9 Hz), 7.85 (2H, d, J=9 Hz), 7.88 (3H, br-s), 8.69 (1H, t, J=6 Hz)

### Example 53

### [7-(Bezafibrate-methoxy)-7-oxoheptyl] amino-chondroitin Sulfate Conjugate

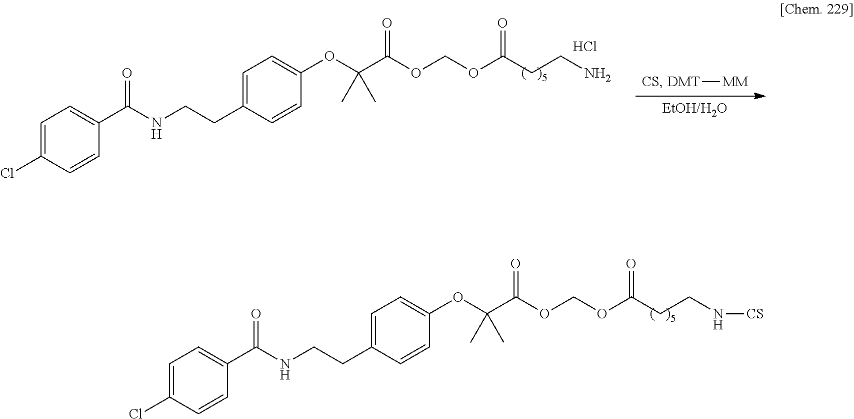

To 4.0 g (0.40 mmol) of a 5% sodium chondroitin sulfate aqueous solution, 2 mL of ethanol was added dropwise under stirring. To the mixed liquid, an ethanol 1 mL-water 1 mL solution of 44 mg (0.080 mmol) of 2-[4-[2-[(4-chlorobenzoyl) amino]ethyl] phenoxy]-2-methylpropanoic acid (7-amino-1-oxoheptyloxy) methyl ester hydrochloride was added. Subsequently, an ethanol 2 mL solution of 22 mg (0.080 mmol) of 4-(4,6-dimethoxy-1,3,5-triazin-2-yl)-4-methylmorpholinium chloride (DMT-MM) was immediately added thereto, and the resulting mixture was stirred overnight at room temperature. To the reaction solution, 100 μL of a 20% sodium chloride aqueous solution was added, and ethanol was further added dropwise (about 2 mL) thereto until immediately before the reaction solution became cloudy. The reaction solution was added dropwise under stirring to 8 mL of 90% ethanol. To the mixed liquid, 9 mL of ethanol was added, and the resulting mixture was stirred for one hour. Precipitates were isolated using a centrifuge, washed with 90% ethanol two times, washed with ethanol two times, and further washed with diethyl ether two times. The obtained precipitates were dried overnight using a vacuum pump to obtain 218 mg of the title compound. Based on values of integral in ¹H-NMR, the introduction ratio of bezafibrate per unit of whole disaccharide (glucuronic acid) of chondroitin sulfate was 8%.

Each of the compounds (conjugates) finally manufactured in all of the above Examples could prepare a 1.5 mg/mL aqueous solution at 25° C.

### Test Example 1 Drug Release Test of Drug-Polymer Conjugate

### [Operation]

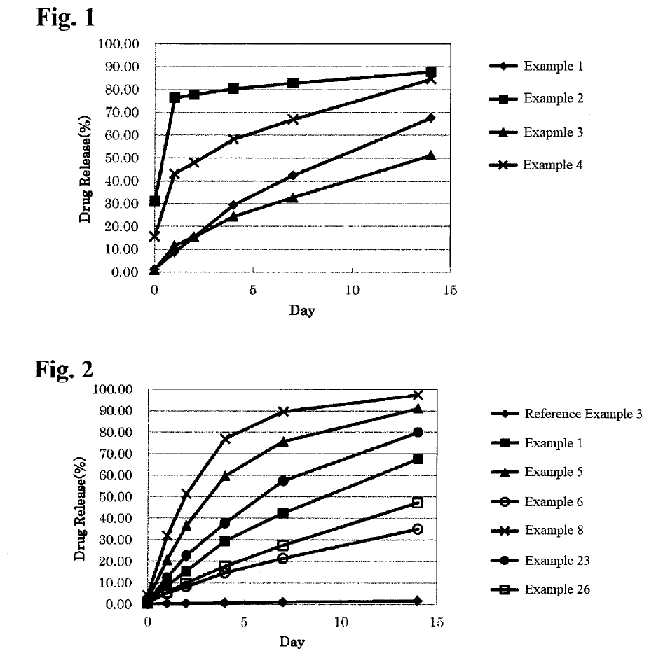

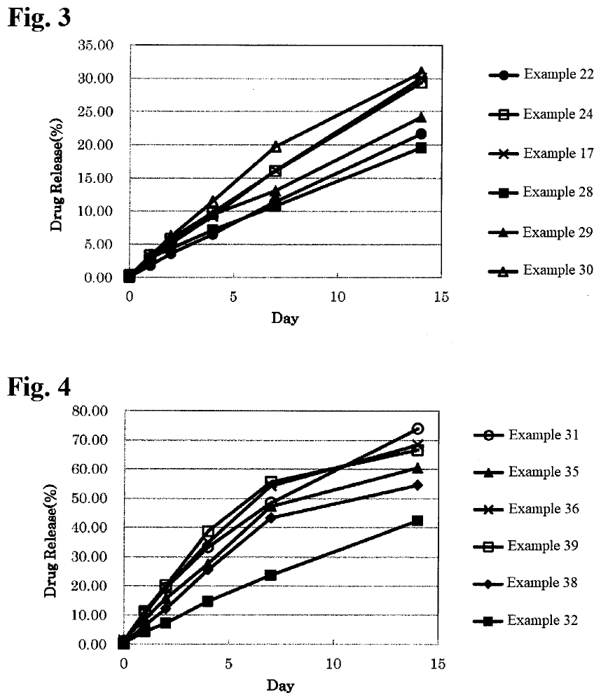

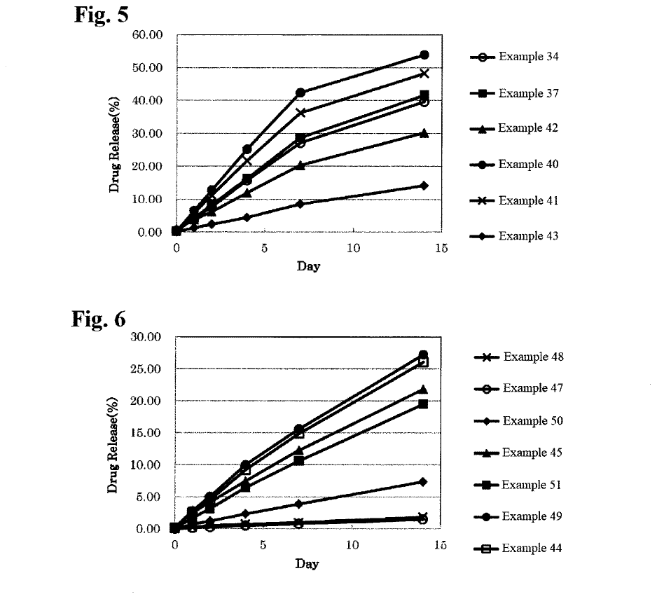

Each of the polymer conjugates to be evaluated in Tables 1 to 6 was dissolved in a sodium phosphate buffer solution of pH 7.0 at a concentration of 1.5 mg/mL at 20° C. to 25° C., and the obtained aqueous solution was dispensed. Immediately after dissolving, the drug-polymer conjugate present in the solution and the release drug amount were analyzed as an initial state (storage 0 day) by SEC-HPLC. Another dispensed liquid was stored under the condition of 36° C. immediately after dissolving, and the drug amount after each time elapsed was analyzed in a similar manner. From a ratio between the release drug amount and the drug-polymer conjugate amount at each time point, obtained in this way, a drug release ratio (%) was calculated. A relationship between time and a drug release ratio is illustrated in FIGS. 1 to 6.

The HPLC conditions are as follows.

Column: TSGgel α-3000 (7.8 mm×300 mm)

Flow rate: 0.5 mL/min

Temperature: 35° C.

Mobile phase: acetonitrile/physiological saline=1/2

### [Result]

As illustrated in FIG. 1, each of the novel conjugate of a carboxylic acid type compound and a polymer having a carboxy group which are the compounds of the present invention increases a drug release amount in a time-dependent manner, and a sustained release property can be imparted thereto regardless of the structure of the bioactive carboxylic acid type compound.

As illustrated in FIG. 2, bezafibrate is a carboxylic acid type compound in which the drug release rate is extremely slow even when an amino alcohol linker exhibiting a high release rate is used in prior art (Reference Example 3). However, in the case of the novel conjugate of a carboxylic acid type compound and a polymer having a carboxy group using the linker of the present invention, the release amount of bezafibrate can be increased largely.

As illustrated in FIGS. 2 to 6, the release rate of the bioactive carboxylic acid type compound can be very finely and widely adjusted by the structure of the linker.

TABLE 1

| Example | | |
|---|---|---|
| NO. | Compound name | Structure |
| Example 1 | [3-(Bezafibrate- methoxy)-3- oxopropyl] amino- chondroitin sulfate conjugate | 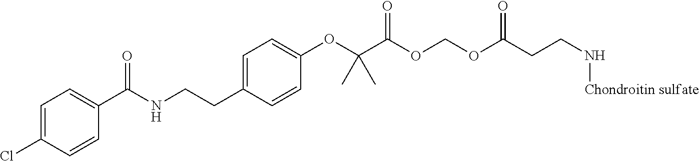 |
| Example 2 | [3-(Ozagrel) methoxy-3- oxopropyl] amino- chondroitin sulfate conjugate | 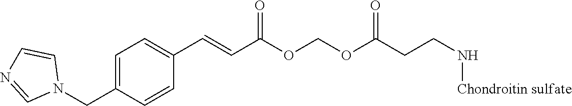 |
| Example 3 | [3-[(Furosemide) methoxy]-3- oxopropyl] amino- chondroitin sulfate conjugate | 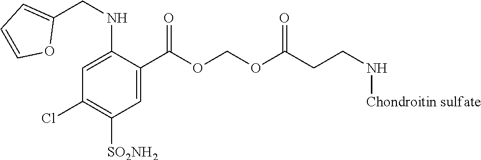 |
| Example 4 | [3-[(Nicotinic acid)-methoxy]-3- oxopropyl] amino- chondroitin sulfate conjugate | 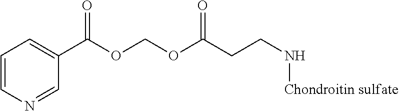 |

TABLE 2

| Example | | |
|---|---|---|
| NO. | Compound name | Structure |
| Reference Example 3 | (2-Bezafibrate- ethyl) amino- chondroitin sulfate conjugate | 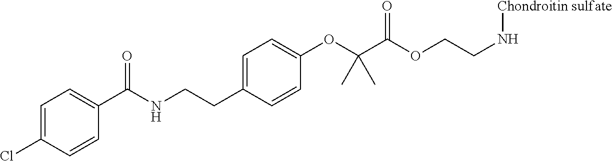 |
| Example 1 | [3-(Bezafibrate- methoxy)-3- oxopropyl] amino- chondroitin sulfate conjugate | 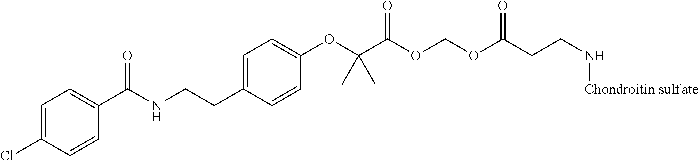 |
| Example 5 | [(S)-[2- (Bezafibrate- methoxy)-1- methyl-2-oxo] ethyl] amino- chondroitin sulfate conjugate | 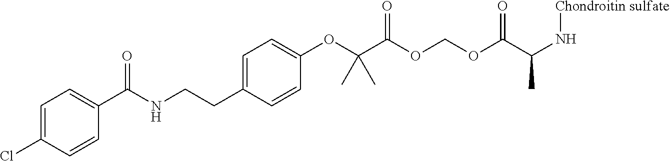 |
| Example 6 | [2-(Bezafibrate- methoxy)-1,1- dimethyl-2- oxoethyl] amino- chondroitin sulfate conjugate | 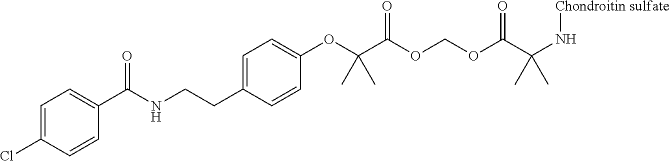 |
| Example 8 | [2-(Bezafibrate- methoxy)-2- oxoethyl] amino- chondroitin sulfate conjugate | 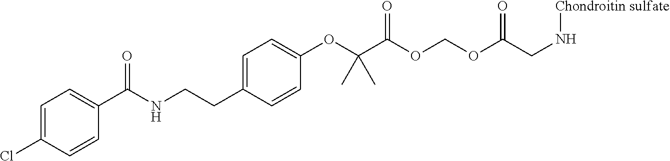 |
| Example 23 | [1-[(Bezafibrate- methoxy) carbonyl] propyl] amino-chondroitin sulfate conjugate | 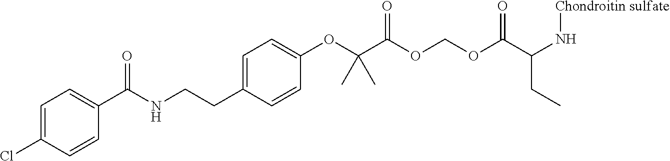 |
| Example 26 | [3-(Bezafibrate- methoxy)-2- methyl-3- oxopropyl] amino- chondroitin sulfate conjugate | 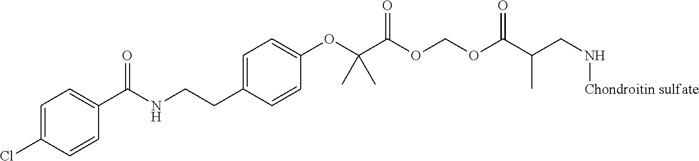 |

TABLE 3

| Example | | |
|---|---|---|
| NO. | Compound name | Structure |
| Example 22 | [2- [(Bezafibrate- methoxy) carbonyl]-2- ethylbutyl] amino- chondroitin sulfate conjugate | 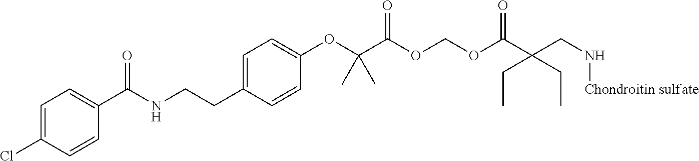 |
| Example 24 | [1- [(Bezafibrate- methoxy) carbonyl] cyclopropyl] methyl] amino- chondroitin sulfate conjugate | 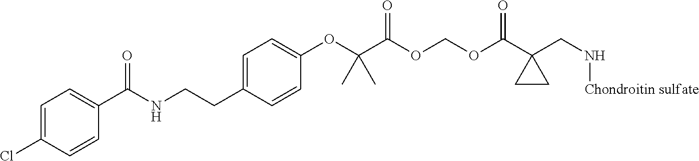 |
| Example 17 | [[1- [(Bezafibrate- methoxy) carbonyl] cyclopentyl] methyl] amino- chondroitin sulfate conjugate | 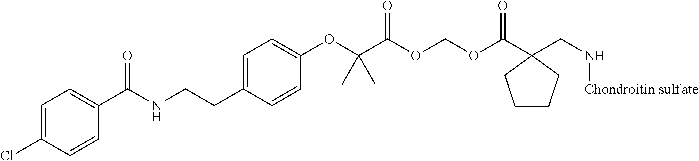 |
| Example 28 | [trans-2- [(Bezafibrate- methoxy) carbonyl] cyclohexyl] amino- chondroitin sulfate conjugate | 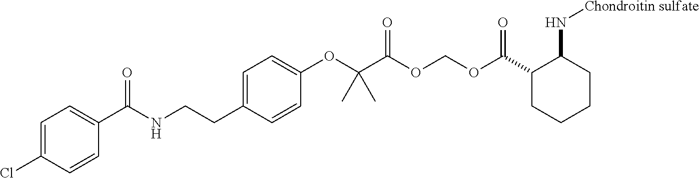 |
| Example 29 | [1- [(Bezafibrate- methoxy) carbonyl]-1- ethylpropyl] amino- chondroitin sulfate conjugate | 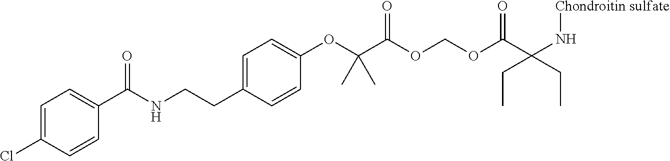 |
| Example 30 | [2- [(Bezafibrate- methoxy) carbonyl] butyl] amino- chondroitin sulfate conjugate | 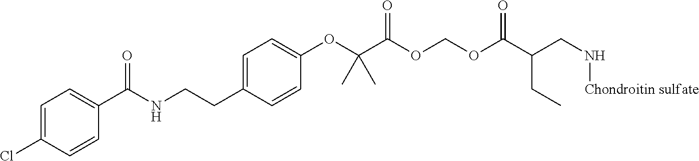 |

TABLE 4

| Example | | |
|---|---|---|
| NO. | Compound name | Structure |
| Example 31 | [2- [[(Bezafibrate- methoxy) carbonyl] oxy] ethyl] amino- chondroitin sulfate conjugate | 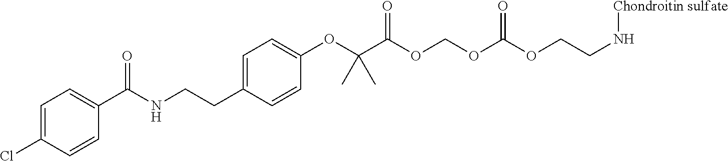 |
| Example 35 | [2-[[(1- Bezafibrate- ethoxy) carbonyl] oxy]- 2-methylpropyl] amino- chondroitin sulfate conjugate | 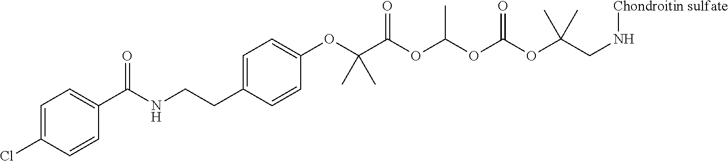 |
| Example 36 | [2-[[(1- Bezafibrate- ethoxy) carbonyl] oxy]- 2-phenylethyl] amino- chondroitin sulfate conjugate | 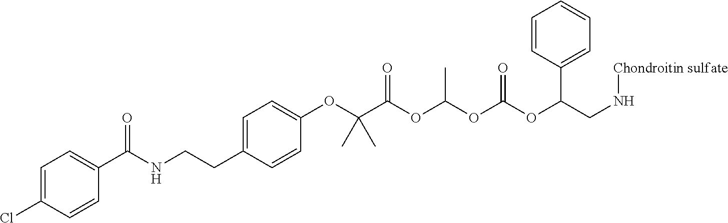 |
| Example 39 | [2-[[(1- Bezafibrate- ethoxy) carbonyl] oxy]- 3,3- dimethylbutyl] amino- chondroitin sulfate conjugate | 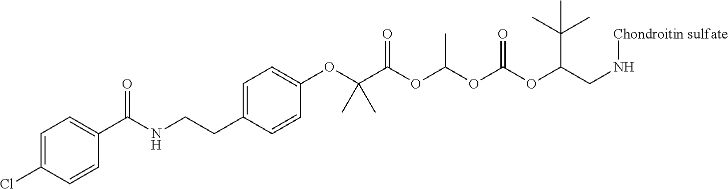 |
| Example 38 | [2-[[(1- Bezafibrate- ethoxy) carbonyl] oxy]- 3-methylbutyl] amino- chondroitin sulfate conjugate | 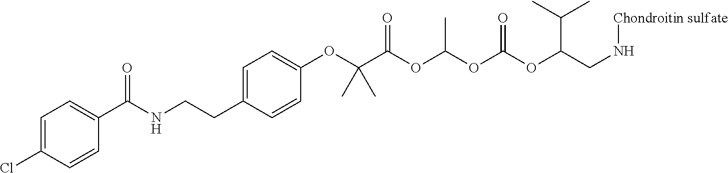 |
| Example 32 | [2-[[(1- Bezafibrate- ethoxy) carbonyl] oxy] ethyl] amino- chondroitin sulfate conjugate | 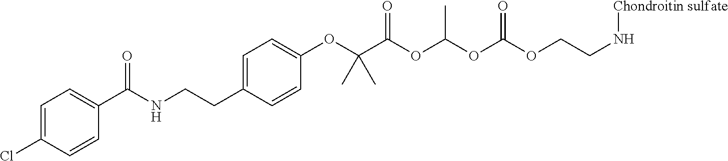 |

TABLE 5

| Example | | |
|---|---|---|
| NO. | Compound name | Structure |
| Example 34 | [2-[[(1- Bezafibrate- ethoxy) carbonyl] oxy] propyl] amino- chondroitin sulfate conjugate | 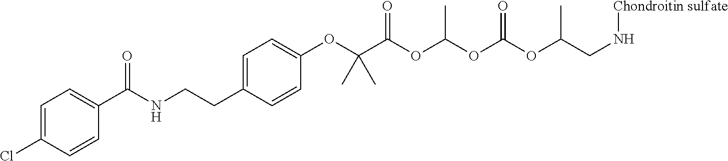 |
| Example 37 | [2-[[(1- Bezafibrate- ethoxy) carbonyl] oxy] butyl] amino- chondroitin sulfate conjugate | 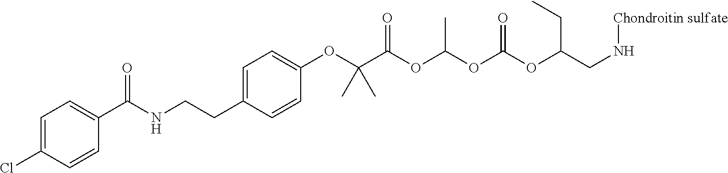 |
| Example 42 | [2-[[(1- Bezafibrate- ethoxy) carbonyl] oxy]- 1-methylethyl] amino- chondroitin sulfate conjugate | 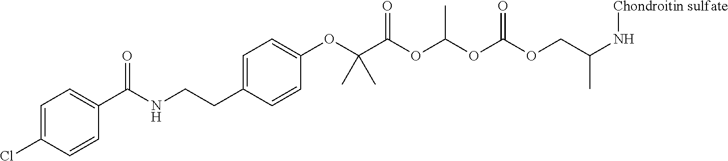 |
| Example 40 | [2-[[(1- Bezafibrate-2- methylpropoxy) carbonyl] oxy]- 3,3- dimethylbutyl] amino- chondroitin sulfate conjugate | 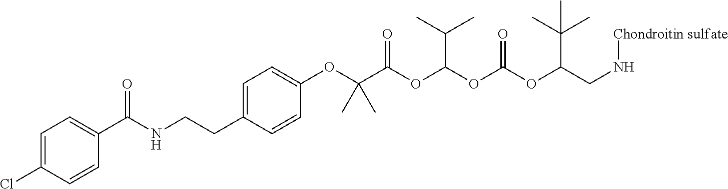 |
| Example 41 | [2-[[(1- Bezafibrate- ethoxy) carbonyl] oxy]- 2- cyclohexylethyl] amino- chondroitin sulfate conjugate | 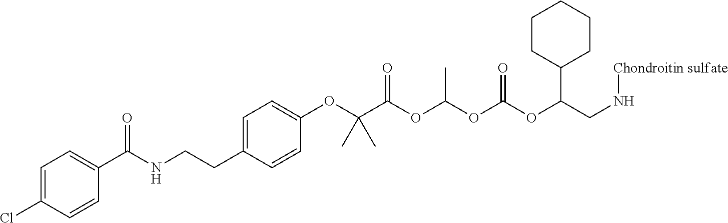 |
| Example 43 | [trans-2-[[(1- Bezafibrate- ethoxy) carbonyl] oxy] cyclohexyl] amino- chondroitin sulfate conjugate | 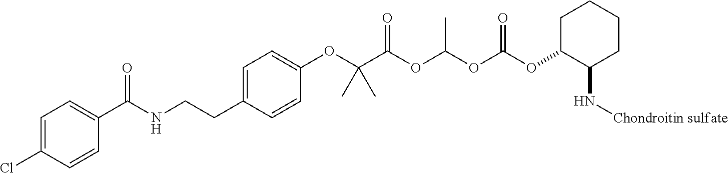 |

TABLE 6

| Example | | |
|---|---|---|
| NO. | Compound name | Structure |
| Example 48 | [2-[[(1- Bezafibrate-2- methylpropoxy) carbonyl] (1- methylethyl) amino] ethyl] amino- chondroitin sulfate conjugate | 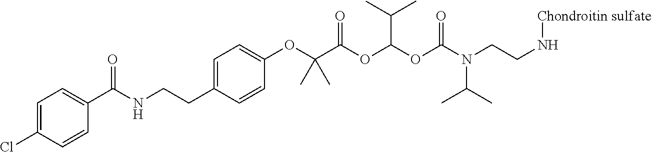 |
| Example 47 | [2-[[(1- Bezafibrate-2- methylpropoxy) carbonyl] methylamino] ethyl] amino- chondroitin sulfate conjugate | 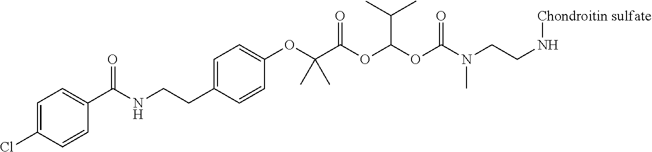 |
| Example 50 | [(3R)-1-[(1- Bezafibrate- ethoxy) carbonyl]-3- pyrrolidinyl] amino- chondroitin sulfate conjugate | 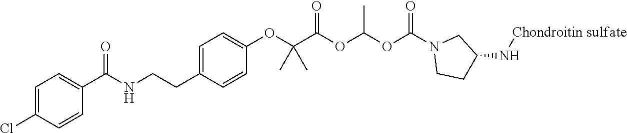 |
| Example 45 | [2- [[(Bezafibrate- methoxy) carbonyl] methylamino] ethyl] amino- chondroitin sulfate conjugate | 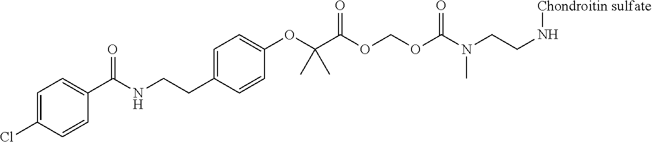 |
| Example 51 | (3R)-[1- [(Bezafibrate- methoxy) carbonyl]-3- piperidinyl] amino- chondroitin sulfate conjugate | 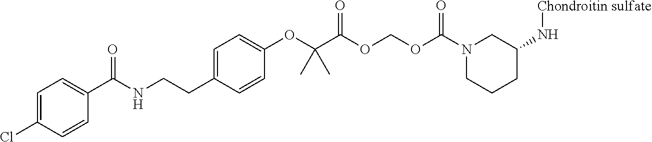 |
| Example 49 | [3- [[(Bezafibrate- methoxy) carbonyl] methylamino] propyl] amino- chondroitin sulfate conjugate | 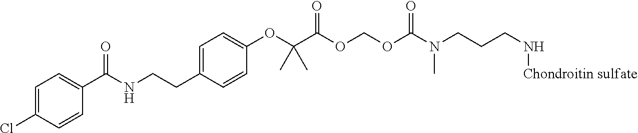 |
| Example 44 | [2- [[(Bezafibrate- methoxy) carbonyl] amino] ethyl] amino- chondroitin sulfate conjugate | 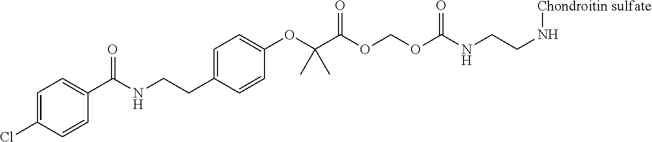 |

1. A compound represented by Formula (I) or a pharmaceutically acceptable salt thereof: 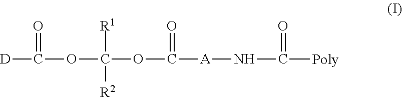
wherein in Formula (I), D is a residue of a carboxylic acid type compound D-CO₂H excluding a carboxy group wherein the carboxylic acid type compound itself or a pharmaceutically acceptable salt thereof is bioactive;
wherein R1 and R2 are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group;
wherein A is a divalent hydrocarbon group optionally having a hetero atom selected from the group consisting of an oxygen atom, a nitrogen atom, and a sulfur atom;
wherein any two or three groups of R1, R2, and A are optionally combined together to form a ring; and
wherein Poly is a residue derived from a polymer having a carboxy group.

2. The compound or a pharmaceutically acceptable salt thereof according to claim 1,
wherein the polymer having a carboxy group and one or more amine forms represented by the following Formula (III) or salts thereof with an inorganic acid or an organic acid form an amide bond: 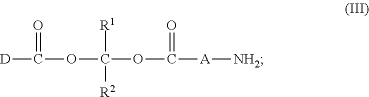
and
wherein in Formula (II), D, R¹, R², and Poly are as defined in claim 1;

3. The compound represented by Formula (I) or a pharmaceutically acceptable salt thereof according to claim 1,
wherein the compound represented by Formula (I) is represented by the follow Formula (II): 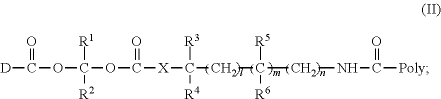
wherein in Formula (II), D, R1, R2, and Poly are as defined in claim 1;
wherein X is a single bond, O, or NR7;
wherein R3, R4, R5, R6, and R7 are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group;
wherein any two or three groups of R1, R2, R3, R4, R5, R6, and R7 are optionally combined together to form a ring; and
wherein l and n are each independently 0, 1, or 2; and
wherein m is 0 or 1.

4. The compound or a pharmaceutically acceptable salt thereof according to claim 3,
wherein the polymer having a carboxy group and one or more amine forms represented by the following Formula (XI) or salts thereof with an inorganic acid or an organic acid form an amide bond, 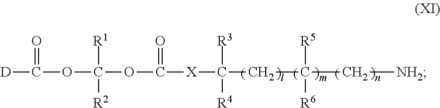
and
wherein in Formula (XI), R¹, R², R³, R⁴, R⁵, R⁶, l, m, n, X, and D are as defined in claim 3.

5. The compound or a pharmaceutically acceptable salt thereof according to claim 3,
wherein in Formula (II), R¹, R², R³, R⁴, R⁵, R⁶, and R⁷ are each independently a hydrogen atom, a substituted or unsubstituted linear or branched alkyl group having carbon number of 1 to 6, a substituted or unsubstituted cycloalkyl group having carbon number of 3 to 8, a substituted or unsubstituted linear or branched alkenyl group having carbon number of 2 to 6, a substituted or unsubstituted cycloalkenyl group having carbon number of 3 to 8, a substituted or unsubstituted linear or branched alkynyl group having carbon number of 2 to 6, a substituted or unsubstituted monocyclic or polycyclic aromatic group having carbon number of 6 to 14, or a substituted or unsubstituted 3- to 8-membered heterocyclic group containing at least one of a nitrogen atom, an oxygen atom, or a sulfur atom as a ring-constituting atom.

6. The compound or a pharmaceutically acceptable salt thereof according to claim 3,
wherein in Formula (II), a substituent of an alkyl, a substituent of a cycloalkyl group, a substituent of an alkenyl group, a substituent of a cycloalkenyl group, a substituent of an alkynyl group, a substituent of an aromatic group, and a substituent of a heterocyclic group in the groups represented by R¹, R², R³, R⁴, R⁵, R⁶, and R⁷ are groups selected from a hydroxy group, an alkyl group, a cycloalkyl group, an alkenyl group, a cycloalkenyl group, an alkynyl group, a halogen atom, an aromatic group, a heterocyclic group, an alkoxy group, a guanidino group, an alkylthio group, an alkoxycarbonyl group, an aryloxy group, an arylthio group, an acyl group, a substituted sulfonyl group, a heterocyclyloxy group, a heterocyclylthio group, an amide group, a ureido group, a carboxy group, a carbamoyl group, an oxo group, a thioxo group, a sulfamoyl group, a sulfo group, a cyano group, a nitro group, an acyloxy group, an azido group, a sulfonamide group, a mercapto group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, an alkoxysulfonyl group, an Rx(Ry)N group, and an Rx(Ry)(Rz)N⁺ group;
wherein Rx, Ry, and Rz are each independently selected from the group consisting of a hydrogen atom, an alkyl group, a cycloalkyl group, an alkenyl group, a cycloalkenyl group, an alkynyl group, an aromatic hydrocarbon group, and a heterocyclic group; and
wherein at least two of Rx, Ry, and Rz are optionally bonded to each other to form a saturated or unsaturated heterocyclic ring, and the heterocyclic ring can also form a condensed ring or a spiro ring with an aliphatic ring or a heterocyclic ring and optionally also form a condensed ring with an aromatic ring.

7. The compound or a pharmaceutically acceptable salt thereof according to claim 1, wherein the polymer having a carboxy group is a water-soluble polymer or a pharmaceutically acceptable salt thereof.

8. The compound or a pharmaceutically acceptable salt thereof according to claim 1, wherein the polymer having a carboxy group is a polysaccharide or a pharmaceutically acceptable salt thereof.

9. The compound or a pharmaceutically acceptable salt thereof according to claim 1, wherein the polymer having a carboxy group is glycosaminoglycan or a pharmaceutically acceptable salt thereof.

10. The compound or a pharmaceutically acceptable salt thereof according to claim 1, wherein the polymer having a carboxy group is chondroitin, chondroitin sulfate, hyaluronic acid, or a pharmaceutically acceptable salt thereof.

11. A method for manufacturing a compound represented by the following Formula (I), the method comprising a step of condensing a compound represented by the following Formula (III) with a polymer having a carboxy group represented by the following Formula (IV): 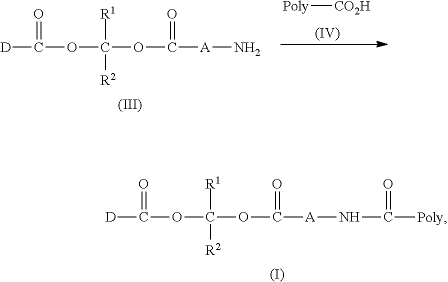
wherein in Formulae (I), (III), and (IV), D, A, R¹, R², and Poly are as defined in claim 1; and
wherein a compound represented by Formula (III) optionally forms a salt with an inorganic acid or an organic acid.

12. (canceled)

13. A method for manufacturing a compound represented by the following Formula (I): 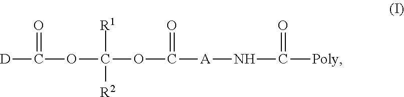
or a pharmaceutically acceptable salt thereof,
wherein in Formula (I), D, A, R¹, R², and Poly are as defined in claim 1;
wherein the method comprising a step of bonding a carboxylic acid type compound in which the carboxylic acid type compound itself or a pharmaceutically acceptable salt thereof is bioactive to a polymer having a carboxy group via a linker represented by the following Formula (V): 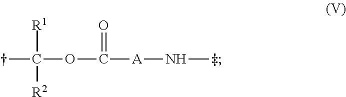
and
wherein the symbol † is a bonding point with a structural moiety of a carboxy group of the carboxylic acid type compound excluding a hydrogen atom, and the symbol ‡ is a bonding point with a structural moiety of a carboxy group of the polymer having a carboxy group excluding a hydroxy group.

14. The compound or a pharmaceutically acceptable salt thereof according to claim 3, wherein the polymer having a carboxy group is a water-soluble polymer or a pharmaceutically acceptable salt thereof.

15. The compound or a pharmaceutically acceptable salt thereof according to claim 3, wherein the polymer having a carboxy group is a polysaccharide or a pharmaceutically acceptable salt thereof.

16. The compound or a pharmaceutically acceptable salt thereof according to claim 3, wherein the polymer having a carboxy group is glycosaminoglycan or a pharmaceutically acceptable salt thereof.

17. The compound or a pharmaceutically acceptable salt thereof according to claim 3, wherein the polymer having a carboxy group is chondroitin, chondroitin sulfate, hyaluronic acid, or a pharmaceutically acceptable salt thereof.
